SEQUENCE LISTING (288 sequences)

SEQ ID NO: 1 - DNA, 348 bp, synthetic construct
  source 1..348: mol_type = other DNA
caggtcaaactgcaggagtctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgctcagggtaactcctggtttccttactggggccaagggaccacggtcaccgtctcctca

SEQ ID NO: 2 - protein, 116 aa, synthetic construct
QVKLQESGAELVKPGASVKLSCKASGYTFTSYWMHWVKQRPGQGLEWIGMIHPNSGSTNYNEKFKSKATLTVDKSSSTAYMQLSSLTSEDSAVYYCAQGNSWFPYWGQGTTVTVSS

SEQ ID NO: 3 - DNA, 21 bp, synthetic construct
  source 1..21: mol_type = other DNA
ggctacactttcaccagctac

SEQ ID NO: 4 - protein, 7 aa, synthetic construct
GYTFTSY

SEQ ID NO: 5 - DNA, 20 bp, synthetic construct
  source 1..20: mol_type = other DNA
catcctaatagtggtagtac

SEQ ID NO: 6 - protein, 6 aa, synthetic construct
HPNSGS

SEQ ID NO: 7 - DNA, 21 bp, synthetic construct
  source 1..21: mol_type = other DNA
ggtaactcctggtttccttac

SEQ ID NO: 8 - protein, 7 aa, synthetic construct
GNSWFPY

SEQ ID NO: 9 - DNA, 318 bp, synthetic construct
  source 1..318: mol_type = other DNA
gacattgtgctgacccagtctccagcaatcatgtctgcatctccaggggagaaggtcaccatgacctgcagtgccagtgcaagtataagttacatgcactggtaccagcagaagtcaggcacctcccccaaaccatggatttatgaaatatccaaactggcttctggagtcccagctcgcttcagtggcagtgggtctgggacctcttactctctcacaatcagcagcatggaggctgaagatgctgccatttattactgtcaacagtggagtagttacccgctcacgttcggtgctgggaccaagctggaaatcaaa

SEQ ID NO: 10 - protein, 106 aa, synthetic construct
DIVLTQSPAIMSASPGEKVTMTCSASASISYMHWYQQKSGTSPKPWIYEISKLASGVPARFSGSGSGTSYSLTISSMEAEDAAIYYCQQWSSYPLTFGAGTKLEIK

SEQ ID NO: 11 - DNA, 30 bp, synthetic construct
  source 1..30: mol_type = other DNA
agtgccagtgcaagtataagttacatgcac

SEQ ID NO: 12 - protein, 10 aa, synthetic construct
SASASISYMH

SEQ ID NO: 13 - DNA, 21 bp, synthetic construct
  source 1..21: mol_type = other DNA
gaaatatccaaactggcttct

SEQ ID NO: 14 - protein, 7 aa, synthetic construct
EISKLAS

SEQ ID NO: 15 - DNA, 27 bp, synthetic construct
  source 1..27: mol_type = other DNA
caacagtggagtagttacccgctcacg

SEQ ID NO: 16 - protein, 9 aa, synthetic construct
QQWSSYPLT

SEQ ID NO: 17 - DNA, 354 bp, synthetic construct
  source 1..354: mol_type = other DNA
caggtcaagctgcagcagtctggagctgagctgatgaagcctggggcctcagtgaagctttcctgcaaggcttctggctacaccttcaccaactactggatgcactgggtgaagcagaggcctggaaaaggccttgagtggattggaatgactcatcctaaaagtagtactactgactacaatgagaagttcgagagcaaggccacactgactgcagacaaatcctccagcacggcctacatgcaactcagcagcctgacttctgaggactctgcggtctattactgtgcaagagggggctacggtagcccctttgactactggggccaagggaccacggtcaccgtctcctca

SEQ ID NO: 18 - protein, 118 aa, synthetic construct
QVKLQQSGAELMKPGASVKLSCKASGYTFTNYWMHWVKQRPGKGLEWIGMTHPKSSTTDYNEKFESKATLTADKSSSTAYMQLSSLTSEDSAVYYCARGGYGSPFDYWGQGTTVTVSS

SEQ ID NO: 19 - DNA, 21 bp, synthetic construct
  source 1..21: mol_type = other DNA
ggctacaccttcaccaactac

SEQ ID NO: 20 - protein, 7 aa, synthetic construct
GYTFTNY

SEQ ID NO: 21 - DNA, 18 bp, synthetic construct
  source 1..18: mol_type = other DNA
catcctaaaagtagtact

SEQ ID NO: 22 - protein, 6 aa, synthetic construct
HPKSST

SEQ ID NO: 23 - DNA, 27 bp, synthetic construct
  source 1..27: mol_type = other DNA
gggggctacggtagcccctttgactac

SEQ ID NO: 24 - protein, 9 aa, synthetic construct
GGYGSPFDY

SEQ ID NO: 25 - DNA, 318 bp, synthetic construct
  source 1..318: mol_type = other DNA
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccacgttcggagggggcaccaagttggaaataaaa

SEQ ID NO: 26 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLEIK

SEQ ID NO: 27 - DNA, 30 bp, synthetic construct
  source 1..30: mol_type = other DNA
agtgccagctcaagtgtaagttacatgcac

SEQ ID NO: 28 - protein, 10 aa, synthetic construct
SASSSVSYMH

SEQ ID NO: 29 - DNA, 21 bp, synthetic construct
  source 1..21: mol_type = other DNA
gacacatccaaactggcttct

SEQ ID NO: 30 - protein, 7 aa, synthetic construct
DTSKLAS

SEQ ID NO: 31 - DNA, 27 bp, synthetic construct
  source 1..27: mol_type = other DNA
cagcagtggagtagtaacccacccacg

SEQ ID NO: 32 - protein, 9 aa, synthetic construct
QQWSSNPPT

SEQ ID NO: 33 - DNA, 357 bp, synthetic construct
  source 1..357: mol_type = other DNA
caggtcaagctgcaggagtcagggggaggcttagtgaagcctggagggtccctgaaactctcctgtgcagcctctggattcactttcagtgactatggaatgcactgggttcgtcaggctccagagaaggggctggagtgggttgcatacattagtagtggcagtagtaccatctactatgcagacacagtgaagggccgattcaccatctccagagacaatgccaagaacaccctgttcctgcaaatgaccagtctgaggtctggggacacggccatgtattactgtgcaaggcacggtagtagctactggtccttcgatgtctggggccaagggaccacggtcaccgtctcctca

SEQ ID NO: 34 - protein, 119 aa, synthetic construct
QVKLQESGGGLVKPGGSLKLSCAASGFTFSDYGMHWVRQAPEKGLEWVAYISSGSSTIYYADTVKGRFTISRDNAKNTLFLQMTSLRSGDTAMYYCARHGSSYWSFDVWGQGTTVTVSS

SEQ ID NO: 35 - DNA, 21 bp, synthetic construct
  source 1..21: mol_type = other DNA
ggattcactttcagtgactat

SEQ ID NO: 36 - protein, 7 aa, synthetic construct
GFTFSDY

SEQ ID NO: 37 - DNA, 18 bp, synthetic construct
  source 1..18: mol_type = other DNA
agtagtggcagtagtacc

SEQ ID NO: 38 - protein, 6 aa, synthetic construct
SSGSST

SEQ ID NO: 39 - DNA, 30 bp, synthetic construct
  source 1..30: mol_type = other DNA
cacggtagtagctactggtccttcgatgtc

SEQ ID NO: 40 - protein, 10 aa, synthetic construct
HGSSYWSFDV

SEQ ID NO: 41 - DNA, 318 bp, synthetic construct
  source 1..318: mol_type = other DNA
gaaaatgtgctcacccagtctccagcaatcatgtctgcatctccaggggagaaggtcaccatatcctgcagtgccagctcaagtgtaaattacatgtactggtacaagcagaagccaggatcctcccccaagccctggatttatcgcacatccaacctggcttctggagtccctgctcgcttcagtggcagtgggtctgggacctcttactctctcacaatcagcagcatggaggccgaagatgctgccacttattactgccagcagtggagtagtaacccgctcacgttcggtgctggcaccaagctggaaatcaaa

SEQ ID NO: 42 - protein, 106 aa, synthetic construct
ENVLTQSPAIMSASPGEKVTISCSASSSVNYMYWYKQKPGSSPKPWIYRTSNLASGVPARFSGSGSGTSYSLTISSMEAEDAATYYCQQWSSNPLTFGAGTKLEIK

SEQ ID NO: 43 - DNA, 30 bp, synthetic construct
  source 1..30: mol_type = other DNA
agtgccagctcaagtgtaaattacatgtac

SEQ ID NO: 44 - protein, 10 aa, synthetic construct
SASSSVNYMY

SEQ ID NO: 45 - DNA, 21 bp, synthetic construct
  source 1..21: mol_type = other DNA
cgcacatccaacctggcttct

SEQ ID NO: 46 - protein, 7 aa, synthetic construct
RTSNLAS

SEQ ID NO: 47 - DNA, 27 bp, synthetic construct
  source 1..27: mol_type = other DNA
cagcagtggagtagtaacccgctcacg

SEQ ID NO: 48 - protein, 9 aa, synthetic construct
QQWSSNPLT

SEQ ID NO: 49 - DNA, 354 bp, synthetic construct
  source 1..354: mol_type = other DNA
caggtcaagctgcaggagtcaggggctgagcttgtgaagcctggggcttcagtgaagctgtcctgcaaggcttctggctacacttttaccagctactggatacactgggtgaagcagaggcctggacaaggccttgagtggattggaatgattcatccaaagagtggaagtattaactacaatgagaagttcaggagcaaggccacactgactgtagacaaatcctccagcacagcctacatgcaactcagcaggctgacatctgaggactctgcggtctatttctgtgcaagcgggggctacggtagtagccttgactactggggccaagggaccacggtcaccgtctcctca

SEQ ID NO: 50 - protein, 118 aa, synthetic construct
QVKLQESGAELVKPGASVKLSCKASGYTFTSYWIHWVKQRPGQGLEWIGMIHPKSGSINYNEKFRSKATLTVDKSSSTAYMQLSRLTSEDSAVYFCASGGYGSSLDYWGQGTTVTVSS

SEQ ID NO: 51 - DNA, 21 bp, synthetic construct
  source 1..21: mol_type = other DNA
ggctacacttttaccagctac

SEQ ID NO: 52 - protein, 7 aa, synthetic construct
GYTFTSY

SEQ ID NO: 53 - DNA, 18 bp, synthetic construct
  source 1..18: mol_type = other DNA
catccaaagagtggaagt

SEQ ID NO: 54 - protein, 6 aa, synthetic construct
HPKSGS

SEQ ID NO: 55 - DNA, 27 bp, synthetic construct
  source 1..27: mol_type = other DNA
gggggctacggtagtagccttgactac

SEQ ID NO: 56 - protein, 9 aa, synthetic construct
GGYGSSLDY

SEQ ID NO: 57 - DNA, 318 bp, synthetic construct
  source 1..318: mol_type = other DNA
caaattgttctcacccagtctccagcaatcatgtccgcatctccaggggagaaggtcaccatgacctgcagtgccagctcaagtgtaagttacatgtactggtaccagcagaagccaagatcctcccccatatcctggatttatctcacatccaacctggcttctggagtccctgctcgcttcagtggcagtgggtctgggacctcttactctctcacaatcagcagcatggaggctgaagatgctgccacttattactgccagcagtggagtagtgacccgctcacgttcggtgctgggaccaagctggagctgaaa

SEQ ID NO: 58 - protein, 106 aa, synthetic construct
QIVLTQSPAIMSASPGEKVTMTCSASSSVSYMYWYQQKPRSSPISWIYLTSNLASGVPARFSGSGSGTSYSLTISSMEAEDAATYYCQQWSSDPLTFGAGTKLELK

SEQ ID NO: 59 - DNA, 30 bp, synthetic construct
  source 1..30: mol_type = other DNA
agtgccagctcaagtgtaagttacatgtac

SEQ ID NO: 60 - protein, 10 aa, synthetic construct
SASSSVSYMY

SEQ ID NO: 61 - DNA, 21 bp, synthetic construct
  source 1..21: mol_type = other DNA
ctcacatccaacctggcttct

SEQ ID NO: 62 - protein, 7 aa, synthetic construct
LTSNLAS

SEQ ID NO: 63 - DNA, 27 bp, synthetic construct
  source 1..27: mol_type = other DNA
cagcagtggagtagtgacccgctcacg

SEQ ID NO: 64 - protein, 9 aa, synthetic construct
QQWSSDPLT

SEQ ID NO: 65 - DNA, 354 bp, synthetic construct
  source 1..354: mol_type = other DNA
caggtgcaactgcagcagtcaggggctgcgctggtaaagcctgggacttcagtgaagttgtcctgcaaggcttctggctacactttcaccagctactggatgcactgggtgaagcagaggcctggacaaggccttgagtggattggaatgattcatcctaaaagtggtagtgttgactacaatgagaaattcacgaacaaggccacactgactggagacaaatcctccggcacagcctacatgcaactcagcagcctgacatctgaagactctgcggtctattactgtgcaagagggggctacggtagcccctttgactactggggccaagggaccacggtcaccgtctcctca

SEQ ID NO: 66 - protein, 118 aa, synthetic construct
QVQLQQSGAALVKPGTSVKLSCKASGYTFTSYWMHWVKQRPGQGLEWIGMIHPKSGSVDYNEKFTNKATLTGDKSSGTAYMQLSSLTSEDSAVYYCARGGYGSPFDYWGQGTTVTVSS

SEQ ID NO: 67 - DNA, 21 bp, synthetic construct
  source 1..21: mol_type = other DNA
ggctacactttcaccagctac

SEQ ID NO: 68 - protein, 7 aa, synthetic construct
GYTFTSY

SEQ ID NO: 69 - DNA, 18 bp, synthetic construct
  source 1..18: mol_type = other DNA
catcctaaaagtggtagt

SEQ ID NO: 70 - protein, 6 aa, synthetic construct
HPKSGS

SEQ ID NO: 71 - DNA, 27 bp, synthetic construct
  source 1..27: mol_type = other DNA
gggggctacggtagcccctttgactac

SEQ ID NO: 72 - protein, 9 aa, synthetic construct
GGYGSPFDY

SEQ ID NO: 73 - DNA, 318 bp, synthetic construct
  source 1..318: mol_type = other DNA
gaaaatgtgctcacccagtctccagcaatcatgtctgcatctccaggggagaaggtcaccatgacctgcagtgccagctcaagtgtaagttacatgtactggtaccagcagaagccaggatcctcccccagactcctgatttatcgcacatccaacctggcttctggagtccctgctcgcttcagtggcagtgggtctgggacctcttactctctcacaatcagcagcatggaggctgaagatgctgccacttattactgccagcagtggagtagtaacccaccgacgttcggtggaggcaccaagttggaaataaaa

SEQ ID NO: 74 - protein, 106 aa, synthetic construct
ENVLTQSPAIMSASPGEKVTMTCSASSSVSYMYWYQQKPGSSPRLLIYRTSNLASGVPARFSGSGSGTSYSLTISSMEAEDAATYYCQQWSSNPPTFGGGTKLEIK

SEQ ID NO: 75 - DNA, 30 bp, synthetic construct; identical to SEQ ID NO: 59
  source 1..30: mol_type = other DNA

SEQ ID NO: 76 - protein, 10 aa, synthetic construct
SASSSVSYMY

SEQ ID NO: 77 - DNA, 21 bp, synthetic construct
  source 1..21: mol_type = other DNA
cgcacatccaacctggcttct

SEQ ID NO: 78 - protein, 7 aa, synthetic construct
RTSNLAS

SEQ ID NO: 79 - DNA, 27 bp, synthetic construct
  source 1..27: mol_type = other DNA
cagcagtggagtagtaacccaccgacg

SEQ ID NO: 80 - protein, 9 aa, synthetic construct
QQWSSNPPT

SEQ ID NO: 81 - DNA, 354 bp, synthetic construct
  source 1..354: mol_type = other DNA
caggtcaaactgcaggagtctggggctgagctggtaaagcctggggcttcagtgaagttgtcctgcaaggcttctggctacacttttaccagctactggatacactgggtgaagcagaggcctggacaaggccttgagtggattggaatgattcatccaaagagtggaagtattaactacaatgagaagttcaggagcaaggccacactgactgtagacaaatcctccagcacagcctacatgcaactcagcaggctgacatctgaggactctgcggtctatttctgtgcaagcgggggctacggtagtagccttgactactggggccaagggaccacggtcaccgtctcctca

SEQ ID NO: 82 - protein, 118 aa, synthetic construct; identical to SEQ ID NO: 50

SEQ ID NO: 83 - DNA, 21 bp, synthetic construct
  source 1..21: mol_type = other DNA
ggctacacttttaccagctac

SEQ ID NO: 84 - protein, 7 aa, synthetic construct
GYTFTSY

SEQ ID NO: 85 - DNA, 18 bp, synthetic construct
  source 1..18: mol_type = other DNA
catccaaagagtggaagt

SEQ ID NO: 86 - protein, 6 aa, synthetic construct
HPKSGS

SEQ ID NO: 87 - DNA, 27 bp, synthetic construct
  source 1..27: mol_type = other DNA
gggggctacggtagtagccttgactac

SEQ ID NO: 88 - protein, 9 aa, synthetic construct
GGYGSSLDY

SEQ ID NO: 89 - DNA, 318 bp, synthetic construct
  source 1..318: mol_type = other DNA
gaaaatgtgctcacccagtctccagcaatcatgtctgcatctccaggggagaaggtcaccatatcctgcagtgccagctcaagtgtaagttacgtgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagctggagctgaaa

SEQ ID NO: 90 - protein, 106 aa, synthetic construct
ENVLTQSPAIMSASPGEKVTISCSASSSVSYVYWYQQKPGSSPKPWIYRTSNLASGVPARFSGSGSGTSYSLTISSMEAEDAATYYCQQWSSNPLTFGAGTKLELK

SEQ ID NO: 91 - DNA, 30 bp, synthetic construct
  source 1..30: mol_type = other DNA
agtgccagctcaagtgtaagttacgtgtac

SEQ ID NO: 92 - protein, 10 aa, synthetic construct
SASSSVSYVY

SEQ ID NO: 93 - DNA, 21 bp, synthetic construct
  source 1..21: mol_type = other DNA
cgcacatccaacctggcttct

SEQ ID NO: 94 - protein, 7 aa, synthetic construct
RTSNLAS

SEQ ID NO: 95 - DNA, 27 bp, synthetic construct
  source 1..27: mol_type = other DNA
cagcagtggagtagtaacccgctcacg

SEQ ID NO: 96 - protein, 9 aa, synthetic construct
QQWSSNPLT

SEQ ID NO: 97 - DNA, 351 bp, synthetic construct
  source 1..351: mol_type = other DNA
caggtgaaactgcaggagtctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagggcaaggccacactgactgcagacaaatcctccagcacagtctacatgcagctcagtagcctgacctctgaggactctgcggtctatttctgtgcaagatcggggggtaattactttgactactggggccaagggaccacggtcaccgtctcctca

SEQ ID NO: 98 - protein, 117 aa, synthetic construct
QVKLQESGAELVKPGASVKLSCKASGYTFTSYWMHWVKQRPGQGLEWIGMIHPNSGSTNYNEKFKGKATLTADKSSSTVYMQLSSLTSEDSAVYFCARSGGNYFDYWGQGTTVTVSS

SEQ ID NO: 99 - DNA, 21 bp, synthetic construct
  source 1..21: mol_type = other DNA
ggctacactttcaccagctac

SEQ ID NO: 100 - protein, 7 aa, synthetic construct
GYTFTSY

SEQ ID NO: 101 - DNA, 18 bp, synthetic construct
  source 1..18: mol_type = other DNA
catcctaatagtggtagt

SEQ ID NO: 102 - protein, 6 aa, synthetic construct
HPNSGS

SEQ ID NO: 103 - DNA, 24 bp, synthetic construct
  source 1..24: mol_type = other DNA
tcggggggtaattactttgactac

SEQ ID NO: 104 - protein, 8 aa, synthetic construct
SGGNYFDY

SEQ ID NO: 105 - DNA, 318 bp, synthetic construct
  source 1..318: mol_type = other DNA
gacattgtgctaacccagtctccagcaatcatgtctgcatctccaggggagaaggtcaccatatcctgcagtgccagctcaagtgtaagttacatgtactggtacaagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtggagtagttacccgctcacgttcggtgctgggaccaagctggaaataaaa

SEQ ID NO: 106 - protein, 106 aa, synthetic construct
DIVLTQSPAIMSASPGEKVTISCSASSSVSYMYWYKQKPGSSPKPWIYRTSNLASGVPARFSGSGSGTSYSLTISSMEAEDAATYYCQQWSSYPLTFGAGTKLEIK

SEQ ID NO: 107 - DNA, 30 bp, synthetic construct; identical to SEQ ID NO: 59
  source 1..30: mol_type = other DNA

SEQ ID NO: 108 - protein, 10 aa, synthetic construct
SASSSVSYMY

SEQ ID NO: 109 - DNA, 21 bp, synthetic construct
  source 1..21: mol_type = other DNA
cgcacatccaacctggcttct

SEQ ID NO: 110 - protein, 7 aa, synthetic construct
RTSNLAS

SEQ ID NO: 111 - DNA, 27 bp, synthetic construct
  source 1..27: mol_type = other DNA
cagcagtggagtagttacccgctcacg

SEQ ID NO: 112 - protein, 9 aa, synthetic construct
QQWSSYPLT

SEQ ID NO: 113 - DNA, 354 bp, synthetic construct
  source 1..354: mol_type = other DNA
caggtcaagctgcaggagtcaggacctgagctggtgaagcctggggcttcagtgaagatatcctgcaaggcttctggctacagcttcacaagctactatatacactgggtgaagcagaggcctggaaaaggccttgagtggattggaatgactcatcctaaaagtagtactactgactacaatgagaagttcgagagcaaggccacactgactgcagacaaatcctccagcacggcctacatgcaactcagcagcctgacttctgaggactctgcggtctattactgtgcaagagggggctacggtagcccctttgactactggggccaagggaccacggtcaccgtctcctca

SEQ ID NO: 114 - protein, 118 aa, synthetic construct
QVKLQESGPELVKPGASVKISCKASGYSFTSYYIHWVKQRPGKGLEWIGMTHPKSSTTDYNEKFESKATLTADKSSSTAYMQLSSLTSEDSAVYYCARGGYGSPFDYWGQGTTVTVSS

SEQ ID NO: 115 - DNA, 21 bp, synthetic construct
  source 1..21: mol_type = other DNA
ggctacagcttcacaagctac

SEQ ID NO: 116 - protein, 7 aa, synthetic construct
GYSFTSY

SEQ ID NO: 117 - DNA, 18 bp, synthetic construct
  source 1..18: mol_type = other DNA
catcctaaaagtagtact

SEQ ID NO: 118 - protein, 6 aa, synthetic construct
HPKSST

SEQ ID NO: 119 - DNA, 27 bp, synthetic construct
  source 1..27: mol_type = other DNA
gggggctacggtagcccctttgactac

SEQ ID NO: 120 - protein, 9 aa, synthetic construct
GGYGSPFDY

SEQ ID NO: 121 - DNA, 318 bp, synthetic construct
  source 1..318: mol_type = other DNA
gaaattgttctcacccagtctccagcaatcatgtctgcatctccaggggagaaggtcaccatgacctgcagtgccacctcaagtgtaagttacatgcactggtaccagcagaagccaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccacccacgttcggctcggggaccaagctggaaataaaa

SEQ ID NO: 122 - protein, 106 aa, synthetic construct
EIVLTQSPAIMSASPGEKVTMTCSATSSVSYMHWYQQKPGTSPKRWIYDTSKLASGVPARFSGSGSGTSYSLTISSMEAEDAATYYCQQWSSNPPTFGSGTKLEIK

SEQ ID NO: 123 - DNA, 30 bp, synthetic construct
  source 1..30: mol_type = other DNA
agtgccacctcaagtgtaagttacatgcac

SEQ ID NO: 124 - protein, 10 aa, synthetic construct
SATSSVSYMH

SEQ ID NO: 125 - DNA, 21 bp, synthetic construct
  source 1..21: mol_type = other DNA
gacacatccaaactggcttct

SEQ ID NO: 126 - protein, 7 aa, synthetic construct
DTSKLAS

SEQ ID NO: 127 - DNA, 27 bp, synthetic construct
  source 1..27: mol_type = other DNA
cagcagtggagtagtaacccacccacg

SEQ ID NO: 128 - protein, 9 aa, synthetic construct
QQWSSNPPT

SEQ ID NO: 129 - DNA, 363 bp, synthetic construct
  source 1..363: mol_type = other DNA
caggtcaaactgcagcagtcagggactgagcttgtgatgcctggggcttcagtgaagctgtcctgcaaggcttccggctacaccttcaccagctactggatgcactgggtaaagcagaggcctggacaaggccttgagtggatcggaatgattgattcttctgatagttatactaactacaatcaaaagttcaagggcaaggccacattgactgtagacacatcctccagcacagcctacatgcagctcagcagcctgacatctgaggactctgcggtctattactgtgcaagaggatactacggtagtagccactatgctatggactactggggccaagggaccacggtcaccgtctcctca

SEQ ID NO: 130 - protein, 121 aa, synthetic construct
QVKLQQSGTELVMPGASVKLSCKASGYTFTSYWMHWVKQRPGQGLEWIGMIDSSDSYTNYNQKFKGKATLTVDTSSSTAYMQLSSLTSEDSAVYYCARGYYGSSHYAMDYWGQGTTVTVSS

SEQ ID NO: 131 - DNA, 21 bp, synthetic construct
  source 1..21: mol_type = other DNA
ggctacaccttcaccagctac

SEQ ID NO: 132 - protein, 7 aa, synthetic construct
GYTFTSY

SEQ ID NO: 133 - DNA, 18 bp, synthetic construct
  source 1..18: mol_type = other DNA
gattcttctgatagttat

SEQ ID NO: 134 - protein, 6 aa, synthetic construct
DSSDSY

SEQ ID NO: 135 - DNA, 36 bp, synthetic construct
  source 1..36: mol_type = other DNA
ggatactacggtagtagccactatgctatggactac

SEQ ID NO: 136 - protein, 12 aa, synthetic construct
GYYGSSHYAMDY

SEQ ID NO: 137 - DNA, 336 bp, synthetic construct
  source 1..336: mol_type = other DNA
gatgttttgatgacccaaactccactctccctgcctgtcagtcttggagatcaagcctccatctcttgcagatctagtcagagccttgtacacagtaatggaaacacctatttagaatggtacctgcagaaaccaggccagtctccaaagctcctaatctacaaagtttccaaccgattttctggggtcccagacaggttcagtggcagtggatcagggacagatttcacactcaagatcagcggagtggaggctgaggatctgggagtttattactgctttcaaggttcacatgttccgtacacgttcggaggggggaccaagctggaaatcaaa

SEQ ID NO: 138 - protein, 112 aa, synthetic construct
DVLMTQTPLSLPVSLGDQASISCRSSQSLVHSNGNTYLEWYLQKPGQSPKLLIYKVSNRFSGVPDRFSGSGSGTDFTLKISGVEAEDLGVYYCFQGSHVPYTFGGGTKLEIK

SEQ ID NO: 139 - DNA, 48 bp, synthetic construct
  source 1..48: mol_type = other DNA
agatctagtcagagccttgtacacagtaatggaaacacctatttagaa

SEQ ID NO: 140 - protein, 16 aa, synthetic construct
RSSQSLVHSNGNTYLE

SEQ ID NO: 141 - DNA, 21 bp, synthetic construct
  source 1..21: mol_type = other DNA
aaagtttccaaccgattttct

SEQ ID NO: 142 - protein, 7 aa, synthetic construct
KVSNRFS

SEQ ID NO: 143 - DNA, 27 bp, synthetic construct
  source 1..27: mol_type = other DNA
tttcaaggttcacatgttccgtacacg

SEQ ID NO: 144 - protein, 9 aa, synthetic construct
FQGSHVPYT

SEQ ID NO: 145 - DNA, 348 bp, synthetic construct
  source 1..348: mol_type = other DNA
caggtgaagctgcagcagtcaggggctgagctggtaaagcctggggcttcagtgaagttgtcctgcaaggctcctggctacacttccaccaactactggatgcactgggtgaagcagaggcctggacaaggccttgagtggattggaatgattcatcccaatagtggtggtaccaactacaatgagaagttcaagagcaaggccacactgactgtagacaaatcctccagcacagcctacatgcaactcagcagcctgacatctgaggactctgcggtctattactgtgcaagcctcggtagtagccttgactattggggccaagggaccacggtcaccgtctcctca

SEQ ID NO: 146 - protein, 116 aa, synthetic construct
QVKLQQSGAELVKPGASVKLSCKAPGYTSTNYWMHWVKQRPGQGLEWIGMIHPNSGGTNYNEKFKSKATLTVDKSSSTAYMQLSSLTSEDSAVYYCASLGSSLDYWGQGTTVTVSS

SEQ ID NO: 147 - DNA, 21 bp, synthetic construct
  source 1..21: mol_type = other DNA
ggctacacttccaccaactac

SEQ ID NO: 148 - protein, 7 aa, synthetic construct
GYTSTNY

SEQ ID NO: 149 - DNA, 18 bp, synthetic construct
  source 1..18: mol_type = other DNA
catcccaatagtggtggt

SEQ ID NO: 150 - protein, 6 aa, synthetic construct
HPNSGG

SEQ ID NO: 151 - DNA, 21 bp, synthetic construct
  source 1..21: mol_type = other DNA
ctcggtagtagccttgactat

SEQ ID NO: 152 - protein, 7 aa, synthetic construct
LGSSLDY

SEQ ID NO: 153 - DNA, 318 bp, synthetic construct
  source 1..318: mol_type = other DNA
gaaaatgtgctcacccagtctccagcaatcatgtctgcatctccaggggagaaggtcaccatgacctgcagtgccagctcaagtataagttacatgcactggtaccagcagaagccaggcacctcccccaaaagatggatttatgacacatccaaactggctcctggagtcccagctcgcttcagtggcagtgggtctgggacctcttactctctcacaatcagcagtgtggaggctgaagatgctgccacttattactgccagcagtacagtggttacccactgacgttcggtggaggcaccaagctggagctgaaa

SEQ ID NO: 154 - protein, 106 aa, synthetic construct
ENVLTQSPAIMSASPGEKVTMTCSASSSISYMHWYQQKPGTSPKRWIYDTSKLAPGVPARFSGSGSGTSYSLTISSVEAEDAATYYCQQYSGYPLTFGGGTKLELK

SEQ ID NO: 155 - DNA, 30 bp, synthetic construct
  source 1..30: mol_type = other DNA
agtgccagctcaagtataagttacatgcac

SEQ ID NO: 156 - protein, 10 aa, synthetic construct
SASSSISYMH

SEQ ID NO: 157 - DNA, 21 bp, synthetic construct
  source 1..21: mol_type = other DNA
gacacatccaaactggctcct

SEQ ID NO: 158 - protein, 7 aa, synthetic construct
DTSKLAP

SEQ ID NO: 159 - DNA, 27 bp, synthetic construct
  source 1..27: mol_type = other DNA
cagcagtacagtggttacccactgacg

SEQ ID NO: 160 - protein, 9 aa, synthetic construct
QQYSGYPLT

SEQ ID NO: 161 - DNA, 354 bp, synthetic construct
  source 1..354: mol_type = other DNA
caggtgcagctgcagcagtcaggggctgagctggtaaagcctggggcttcagtgaagttgtcctgcaaggcttctggctacacttttaccagctactggatacactgggtgaagcagaggcctggacaaggccttgagtggattggaatgattcatccaaagagtggaagtattaactacaatgagaagttcaggagcaaggccacactgactgtagacaaatcctccagcacagcctacatgcaactcagcaggctgacatctgaggactctgcggtctatttctgtgcaagcgggggctacggtagtagccttgactactggggccaagggaccacggtcaccgtctcctca

SEQ ID NO: 162 - protein, 118 aa, synthetic construct
QVQLQQSGAELVKPGASVKLSCKASGYTFTSYWIHWVKQRPGQGLEWIGMIHPKSGSINYNEKFRSKATLTVDKSSSTAYMQLSRLTSEDSAVYFCASGGYGSSLDYWGQGTTVTVSS

SEQ ID NO: 163 - DNA, 21 bp, synthetic construct
  source 1..21: mol_type = other DNA
ggctacacttttaccagctac

SEQ ID NO: 164 - protein, 7 aa, synthetic construct
GYTFTSY

SEQ ID NO: 165 - DNA, 18 bp, synthetic construct
  source 1..18: mol_type = other DNA
catccaaagagtggaagt

SEQ ID NO: 166 - protein, 6 aa, synthetic construct
HPKSGS

SEQ ID NO: 167 - DNA, 27 bp, synthetic construct
  source 1..27: mol_type = other DNA
gggggctacggtagtagccttgactac

SEQ ID NO: 168 - protein, 9 aa, synthetic construct
GGYGSSLDY

SEQ ID NO: 169 - DNA, 318 bp, synthetic construct
  source 1..318: mol_type = other DNA
gaaaatgtgctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgctcgcttcagtggcagtgggtctgggacctcttactctctcacaatcagcagcatggaggctgtagatgctgccacttattactgccagcagtggagtagtgacccgctcacgttcggtgctgggaccaagctggagctgaaa

SEQ ID NO: 170 - protein, 106 aa, synthetic construct
ENVLTQSPAIMSASPGEKVTMTCSASSSVSYMYWYQQKPGSSPRLLIYDTSNLASGVPARFSGSGSGTSYSLTISSMEAVDAATYYCQQWSSDPLTFGAGTKLELK

SEQ ID NO: 171 - DNA, 30 bp, synthetic construct; identical to SEQ ID NO: 59
  source 1..30: mol_type = other DNA

SEQ ID NO: 172 - protein, 10 aa, synthetic construct
SASSSVSYMY

SEQ ID NO: 173 - DNA, 21 bp, synthetic construct
  source 1..21: mol_type = other DNA
gacacatccaacctggcttct

SEQ ID NO: 174 - protein, 7 aa, synthetic construct
DTSNLAS

SEQ ID NO: 175 - DNA, 27 bp, synthetic construct
  source 1..27: mol_type = other DNA
cagcagtggagtagtgacccgctcacg

SEQ ID NO: 176 - protein, 9 aa, synthetic construct
QQWSSDPLT

SEQ ID NO: 177 - DNA, 354 bp, synthetic construct
  source 1..354: mol_type = other DNA
caggtcaagctgcagcagtcaggggctgagctggtaaagcctggggcttcagtgaagttgtcctgcaaggcttctggctacaccttccccaactactggataacctgggtgaagcagaggcctggacaaggccttgagtggattggaatgattcatccaaagagtggaagtattaactacaatgagaagttcaggagcaaggccacactgactgtagacaaatcttccagcacagcctacatgcaactcagcaggctgacatctgaggactctgcggtctattactgtgcaagcgggggctacggtagtagccttgactactggggccaagggaccacggtcaccgtctcctca

SEQ ID NO: 178 - protein, 118 aa, synthetic construct
QVKLQQSGAELVKPGASVKLSCKASGYTFPNYWITWVKQRPGQGLEWIGMIHPKSGSINYNEKFRSKATLTVDKSSSTAYMQLSRLTSEDSAVYYCASGGYGSSLDYWGQGTTVTVSS

SEQ ID NO: 179 - DNA, 21 bp, synthetic construct
  source 1..21: mol_type = other DNA
ggctacaccttccccaactac

SEQ ID NO: 180 - protein, 7 aa, synthetic construct
GYTFPNY

SEQ ID NO: 181 - DNA, 18 bp, synthetic construct
  source 1..18: mol_type = other DNA
catccaaagagtggaagt

SEQ ID NO: 182 - protein, 6 aa, synthetic construct
HPKSGS

SEQ ID NO: 183 - DNA, 27 bp, synthetic construct
  source 1..27: mol_type = other DNA
gggggctacggtagtagccttgactac

SEQ ID NO: 184 - protein, 9 aa, synthetic construct
GGYGSSLDY

SEQ ID NO: 185 - DNA, 318 bp, synthetic construct
  source 1..318: mol_type = other DNA
gaaaatgtgctcacccagtctccagcaatcatgtctgcatctctaggggagaaggtcaccatgagctgcagggccagctcaagtgtaagttacatgtactggtaccagcagaagccaagatcctcccccaaaccctggatttatctcacatccaaactggcttctggagtccctgctcgcttcagtggcagtgggtctgggacctcttactctctcacaatcagcagcatggaggctgaagatgctgccacttattactgccagcagtggagtagtaacccattcacgttcggctcggggaccaagctggaaataaaa

SEQ ID NO: 186 - protein, 106 aa, synthetic construct
ENVLTQSPAIMSASLGEKVTMSCRASSSVSYMYWYQQKPRSSPKPWIYLTSKLASGVPARFSGSGSGTSYSLTISSMEAEDAATYYCQQWSSNPFTFGSGTKLEIK

SEQ ID NO: 187 - DNA, 30 bp, synthetic construct
  source 1..30: mol_type = other DNA
agggccagctcaagtgtaagttacatgtac

SEQ ID NO: 188 - protein, 10 aa, synthetic construct
RASSSVSYMY

SEQ ID NO: 189 - DNA, 21 bp, synthetic construct
  source 1..21: mol_type = other DNA
ctcacatccaaactggcttct

SEQ ID NO: 190 - protein, 7 aa, synthetic construct
LTSKLAS

SEQ ID NO: 191 - DNA, 27 bp, synthetic construct
  source 1..27: mol_type = other DNA
cagcagtggagtagtaacccattcacg

SEQ ID NO: 192 - protein, 9 aa, synthetic construct
QQWSSNPFT

SEQ ID NO: 193 - DNA, 354 bp, synthetic construct
  source 1..354: mol_type = other DNA
caggtgaagctgcagcagtctggggctgagctggtaaagcctggggcttcagtgaagttgtcctgcaaggcttctggctacactttcaccagctactgggtgcactgggtgaagcagaggcctggacaaggccttgagtggattggaatgattcatcctaaaagtggtagtgctgactacaatgagaagttcacgaccaaggccacactgactgcagacaaatcctccagcacagcctacatgcaactcagcagtctgacatctgaggactctgcggtctattactgtgcaagagggggctacggtagcccctttgactactggggccaagggaccacggtcaccgtctcctca

SEQ ID NO: 194 - protein, 118 aa, synthetic construct
QVKLQQSGAELVKPGASVKLSCKASGYTFTSYWVHWVKQRPGQGLEWIGMIHPKSGSADYNEKFTTKATLTADKSSSTAYMQLSSLTSEDSAVYYCARGGYGSPFDYWGQGTTVTVSS

SEQ ID NO: 195 - DNA, 21 bp, synthetic construct
  source 1..21: mol_type = other DNA
ggctacactttcaccagctac

SEQ ID NO: 196 - protein, 7 aa, synthetic construct
GYTFTSY

SEQ ID NO: 197 - DNA, 18 bp, synthetic construct
  source 1..18: mol_type = other DNA
catcctaaaagtggtagt

SEQ ID NO: 198 - protein, 6 aa, synthetic construct
HPKSGS

SEQ ID NO: 199 - DNA, 27 bp, synthetic construct
  source 1..27: mol_type = other DNA
gggggctacggtagcccctttgactac

SEQ ID NO: 200 - protein, 9 aa, synthetic construct
GGYGSPFDY

SEQ ID NO: 201 - DNA, 318 bp, synthetic construct
  source 1..318: mol_type = other DNA
gaaaatgtgctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagcccctgttcgcttcagtggccgtgggtctgggacctcttactctctcacaatcagcagcatggaggctgaagatgctgccacttattactgccagcagtggagtaataaccccctcacgttcggtgctgggaccaagctggagctgaaa

SEQ ID NO: 202 - protein, 106 aa, synthetic construct
ENVLTQSPAIMSASPGEKVTMTCSASSSVSYMYWYQQKPGSSPRLLIYDTSNLASGAPVRFSGRGSGTSYSLTISSMEAEDAATYYCQQWSNNPLTFGAGTKLELK

SEQ ID NO: 203 - DNA, 30 bp, synthetic construct; identical to SEQ ID NO: 59
  source 1..30: mol_type = other DNA

SEQ ID NO: 204 - protein, 10 aa, synthetic construct
SASSSVSYMY

SEQ ID NO: 205 - DNA, 21 bp, synthetic construct
  source 1..21: mol_type = other DNA
gacacatccaacctggcttct

SEQ ID NO: 206 - protein, 7 aa, synthetic construct
DTSNLAS

SEQ ID NO: 207 - DNA, 27 bp, synthetic construct
  source 1..27: mol_type = other DNA
cagcagtggagtaataaccccctcacg

SEQ ID NO: 208 - protein, 9 aa, synthetic construct
QQWSNNPLT

SEQ ID NO: 209 - DNA, 354 bp, synthetic construct
  source 1..354: mol_type = other DNA
caggtgaaactgcagcagtctggggctgagcttgtgatgcctggggcttcagtgacgctgtcctgcaaggcttctggctacaccttcaccagctactggatgcactgggtgaagcagaggcctggaaaaggccttgagtggattggaatgactcatcctaaaagtagtactactgactacaatgagaagttcaagagcaaggccacactgactgcagacaaatcctccagcacggcctacatgcagctcagcagcctgacttctgaggactctgcggtctattactgtgcaagagggggctacggtagcccctttgactactggggccaagggaccacggtcaccgtctcctca

SEQ ID NO: 210 - protein, 118 aa, synthetic construct
QVKLQQSGAELVMPGASVTLSCKASGYTFTSYWMHWVKQRPGKGLEWIGMTHPKSSTTDYNEKFKSKATLTADKSSSTAYMQLSSLTSEDSAVYYCARGGYGSPFDYWGQGTTVTVSS

SEQ ID NO: 211 - DNA, 21 bp, synthetic construct
  source 1..21: mol_type = other DNA
ggctacaccttcaccagctac

SEQ ID NO: 212 - protein, 7 aa, synthetic construct
GYTFTSY

SEQ ID NO: 213 - DNA, 18 bp, synthetic construct
  source 1..18: mol_type = other DNA
catcctaaaagtagtact

SEQ ID NO: 214 - protein, 6 aa, synthetic construct
HPKSST

SEQ ID NO: 215 - DNA, 27 bp, synthetic construct
  source 1..27: mol_type = other DNA
gggggctacggtagcccctttgactac

SEQ ID NO: 216 - protein, 9 aa, synthetic construct
GGYGSPFDY

SEQ ID NO: 217 - DNA, 318 bp, synthetic construct
  source 1..318: mol_type = other DNA
caaattgttctcacccagtctccagcaatcctgtctgcatctccaggggagaaggtcaccatgacctgcagtgccaggtcaagtgtaagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagctggagctgaaa

SEQ ID NO: 218 - protein, 106 aa, synthetic construct
QIVLTQSPAILSASPGEKVTMTCSARSSVSYMYWYQQKPRSSPKPWIYLTSNLASGVPARFSGSGSGTSYSLTISSMEAEDAATYYCQQWSSNPLTFGAGTKLELK

SEQ ID NO: 219 - DNA, 30 bp, synthetic construct
  source 1..30: mol_type = other DNA
agtgccaggtcaagtgtaagttacatgtac

SEQ ID NO: 220 - protein, 10 aa, synthetic construct
SARSSVSYMY

SEQ ID NO: 221 - DNA, 21 bp, synthetic construct
  source 1..21: mol_type = other DNA
ctcacatccaacctggcttct

SEQ ID NO: 222 - protein, 7 aa, synthetic construct
LTSNLAS

SEQ ID NO: 223 - DNA, 26 bp, synthetic construct
  source 1..26: mol_type = other DNA
agcagtggagtagtaacccgctcacg

SEQ ID NO: 224 - protein, 9 aa, synthetic construct
QQWSSNPLT

SEQ ID NO: 225 - DNA, 354 bp, synthetic construct
  source 1..354: mol_type = other DNA
caggtcaaactgcaggagtcaggggctgagcttgtgaagcctggggcttcagtgaagatgtcctgcaaggcttctggctacaccttcaccaactactggattcactgggtgaagcagaggcctggacaaggccttgagtggattggaatgattcatcctaagagtggtagtattacttacaatgagaagttcacgaccaaggccacactgactgtcgacaaatcctccagcacagcctacatgcaactcaacagcctgacatctgaggactctgcggtctattactgtgcaagcgggggctacggtagtagccttgactactggggccaagggaccacggtcaccgtctcctca

SEQ ID NO: 226 - protein, 118 aa, synthetic construct
QVKLQESGAELVKPGASVKMSCKASGYTFTNYWIHWVKQRPGQGLEWIGMIHPKSGSITYNEKFTTKATLTVDKSSSTAYMQLNSLTSEDSAVYYCASGGYGSSLDYWGQGTTVTVSS

SEQ ID NO: 227 - DNA, 21 bp, synthetic construct
  source 1..21: mol_type = other DNA
ggctacaccttcaccaactac

SEQ ID NO: 228 - protein, 7 aa, synthetic construct
GYTFTNY

SEQ ID NO: 229 - DNA, 18 bp, synthetic construct
  source 1..18: mol_type = other DNA
catcctaagagtggtagt

SEQ ID NO: 230 - protein, 6 aa, synthetic construct
HPKSGS

SEQ ID NO: 231 - DNA, 27 bp, synthetic construct
  source 1..27: mol_type = other DNA
gggggctacggtagtagccttgactac

SEQ ID NO: 232 - protein, 9 aa, synthetic construct
GGYGSSLDY

SEQ ID NO: 233 - DNA, 318 bp, synthetic construct
  source 1..318: mol_type = other DNA
gaaaatgtgctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagttggaaataaaa

SEQ ID NO: 234 - protein, 106 aa, synthetic construct
ENVLTQSPAIMSASPGEKVTISCSASSSVSYMYWYQQKPGSSPKPWIYRTSNLASGVPARFSGSGSGTSYSLTISSMEAEDAATYYCQQWSSNPLTFGAGTKLEIK

SEQ ID NO: 235 - DNA, 30 bp, synthetic construct; identical to SEQ ID NO: 59
  source 1..30: mol_type = other DNA

SEQ ID NO: 236 - protein, 10 aa, synthetic construct
SASSSVSYMY

SEQ ID NO: 237 - DNA, 21 bp, synthetic construct
  source 1..21: mol_type = other DNA
cgcacatccaacctggcttct

SEQ ID NO: 238 - protein, 7 aa, synthetic construct
RTSNLAS

SEQ ID NO: 239 - DNA, 27 bp, synthetic construct
  source 1..27: mol_type = other DNA
cagcagtggagtagtaacccgctcacg

SEQ ID NO: 240 - protein, 9 aa, synthetic construct
QQWSSNPLT

SEQ ID NO: 241 - DNA, 354 bp, synthetic construct
  source 1..354: mol_type = other DNA
caggtgaagctgcaggagtctggggctgagcttgtgatgcctggggcttcagtgaagctgtcctgcaaagcttctggctacaccttcaccagctactggatgcactgggtaaagcagaggcctggacaaggccttgagtggatcggaatgattgatccaaagagtggaagtattaactacaatgagaagttcaggagcaaggccacactgactgtagacaagtcctccagcacagcctacatgcaactcagcaggctgacatctgaggactctgcggtctattactgtgcaagcgggggctacggtagtagccttgactactggggccaagggaccacggtcaccgtctcctca

SEQ ID NO: 242 - protein, 118 aa, synthetic construct
QVKLQESGAELVMPGASVKLSCKASGYTFTSYWMHWVKQRPGQGLEWIGMIDPKSGSINYNEKFRSKATLTVDKSSSTAYMQLSRLTSEDSAVYYCASGGYGSSLDYWGQGTTVTVSS

SEQ ID NO: 243 - DNA, 21 bp, synthetic construct
  source 1..21: mol_type = other DNA
ggctacaccttcaccagctac

SEQ ID NO: 244 - protein, 7 aa, synthetic construct
GYTFTSY

SEQ ID NO: 245 - DNA, 18 bp, synthetic construct
  source 1..18: mol_type = other DNA
gatccaaagagtggaagt

SEQ ID NO: 246 - protein, 6 aa, synthetic construct
DPKSGS

SEQ ID NO: 247 - DNA, 27 bp, synthetic construct
  source 1..27: mol_type = other DNA
gggggctacggtagtagccttgactac

SEQ ID NO: 248 - protein, 9 aa, synthetic construct
GGYGSSLDY

SEQ ID NO: 249 - DNA, 318 bp, synthetic construct
  source 1..318: mol_type = other DNA
gaaaatgtgctcacccagtccccagcaatcatgtctgcatctccaggggagaaggtcaccatgacctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtggagtagtgacccgctcacgttcggtgctggcaccaagctggaaatcaaa

SEQ ID NO: 250 - protein, 106 aa, synthetic construct
ENVLTQSPAIMSASPGEKVTMTCSASSSVSYMYWYQQKPGSSPKPWIYRTSNLASGVPARFSGSGSGTSYSLTISSMEAEDAATYYCQQWSSDPLTFGAGTKLEIK

SEQ ID NO: 251 - DNA, 30 bp, synthetic construct; identical to SEQ ID NO: 59
  source 1..30: mol_type = other DNA

SEQ ID NO: 252 - protein, 10 aa, synthetic construct
SASSSVSYMY

SEQ ID NO: 253 - DNA, 21 bp, synthetic construct
  source 1..21: mol_type = other DNA
cgcacatccaacctggcttct

SEQ ID NO: 254 - protein, 7 aa, synthetic construct
RTSNLAS

SEQ ID NO: 255 - DNA, 27 bp, synthetic construct
  source 1..27: mol_type = other DNA
cagcagtggagtagtgacccgctcacg

SEQ ID NO: 256 - protein, 9 aa, synthetic construct
QQWSSDPLT

SEQ ID NO: 257 - DNA, 348 bp, synthetic construct
  source 1..348: mol_type = other DNA
caggtcaaactgcagcagtcaggggctgacctggtaaagcctggggcttcagtgaagttgtcctgcaaggcttctggctacactttcaccagctactggatgcactgggtgaagcagaggcctggacaaggccttgagtggattggaatgattcatcctaatagtgatggtactaactacaatgagaagttcaagaacagggccacactgactgtggacaaatcctccagcacggcctacatgcaactcaacagcctgacatctgaagactctgcggtctattactgtgcaagatggtcctatggtatggactcctggggccaagggaccacggtcaccgtctcctca

SEQ ID NO: 258 - protein, 116 aa, synthetic construct
QVKLQQSGADLVKPGASVKLSCKASGYTFTSYWMHWVKQRPGQGLEWIGMIHPNSDGTNYNEKFKNRATLTVDKSSSTAYMQLNSLTSEDSAVYYCARWSYGMDSWGQGTTVTVSS

SEQ ID NO: 259 - DNA, 21 bp, synthetic construct
  source 1..21: mol_type = other DNA
ggctacactttcaccagctac

SEQ ID NO: 260 - protein, 7 aa, synthetic construct
GYTFTSY

SEQ ID NO: 261 - DNA, 18 bp, synthetic construct
  source 1..18: mol_type = other DNA
catcctaatagtgatggt

SEQ ID NO: 262 - protein, 6 aa, synthetic construct
HPNSDG

SEQ ID NO: 263 - DNA, 21 bp, synthetic construct
  source 1..21: mol_type = other DNA
tggtcctatggtatggactcc

SEQ ID NO: 264 - protein, 7 aa, synthetic construct
WSYGMDS

SEQ ID NO: 265 - DNA, 318 bp, synthetic construct
  source 1..318: mol_type = other DNA
gacattgtgctaacccagtctccagcttctttggctgtgtctctagggcagagggccaccatcacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtcgtaacccactcacgttcggtgctgggaccaagttggaaataaaa

SEQ ID NO: 266 - protein, 106 aa, synthetic construct
DIVLTQSPASLAVSLGQRATITCSASSSVSYMHWYQQKSGTSPKRWIYDTSKLASGVPARFSGSGSGTSYSLTISSMEAEDAATYYCQQWSRNPLTFGAGTKLEIK

SEQ ID NO: 267 - DNA, 30 bp, synthetic construct; identical to SEQ ID NO: 27
  source 1..30: mol_type = other DNA

SEQ ID NO: 268 - protein, 10 aa, synthetic construct
SASSSVSYMH

SEQ ID NO: 269 - DNA, 21 bp, synthetic construct
  source 1..21: mol_type = other DNA
gacacatccaaactggcttct

SEQ ID NO: 270 - protein, 7 aa, synthetic construct
DTSKLAS

SEQ ID NO: 271 - DNA, 27 bp, synthetic construct
  source 1..27: mol_type = other DNA
cagcagtggagtcgtaacccactcacg

SEQ ID NO: 272 - protein, 9 aa, synthetic construct
QQWSRNPLT

SEQ ID NO: 273 - protein, 15 aa, synthetic construct
GGGGSGGGGSGGGGS

SEQ ID NO: 274 - protein, 109 aa, synthetic construct
DIVLTQSPAIMSASPGEKVTISCSASSSVSYMYWYKQKPGSSPKPWIYRTSNLASGVPARFSGSGSGTSYSLTISSMEAEDAATYYCQQWSSYPLTFGAGTKLEIKRAA

SEQ ID NO: 275 - protein, 115 aa, synthetic construct
DVLMTQTPLSLPVSLGDQASISCRSSQSLVHSNGNTYLEWYLQKPGQSPKLLIYKVSNRFSGVPDRFSGSGSGTDFTLKISGVEAEDLGVYYCFQGSHVPYTFGGGTKLEIKRAA

SEQ ID NO: 276 - protein, 109 aa, synthetic construct
ENVLTQSPAIMSASPGEKVTMTCSASSSISYMHWYQQKPGTSPKRWIYDTSKLAPGVPARFSGSGSGTSYSLTISSVEAEDAATYYCQQYSGYPLTFGGGTKLELKRAA

SEQ ID NO: 277 - protein, 109 aa, synthetic construct
ENVLTQSPAIMSASPGEKVTMTCSASSSVSYMYWYQQKPGSSPRLLIYDTSNLASGVPARFSGSGSGTSYSLTISSMEAVDAATYYCQQWSSDPLTFGAGTKLELKRAA

SEQ ID NO: 278 - protein, 109 aa, synthetic construct
QIVLTQSPAILSASPGEKVTMTCSARSSVSYMYWYQQKPRSSPKPWIYLTSNLASGVPARFSGSGSGTSYSLTISSMEAEDAATYYCQQWSSNPLTFGAGTKLELKRAA

SEQ ID NO: 279 - protein, 109 aa, synthetic construct
ENVLTQSPAIMSASPGEKVTMTCSASSSVSYMYWYQQKPGSSPRLLIYDTSNLASGAPVRFSGRGSGTSYSLTISSMEAEDAATYYCQQWSNNPLTFGAGTKLELKRAA

SEQ ID NO: 280 - protein, 109 aa, synthetic construct
ENVLTQSPAIMSASPGEKVTISCSASSSVSYMYWYQQKPGSSPKPWIYRTSNLASGVPARFSGSGSGTSYSLTISSMEAEDAATYYCQQWSSNPLTFGAGTKLEIKRAA

SEQ ID NO: 281 - protein, 109 aa, synthetic construct
ENVLTQSPAIMSASPGEKVTMTCSASSSVSYMYWYQQKPGSSPKPWIYRTSNLASGVPARFSGSGSGTSYSLTISSMEAEDAATYYCQQWSSDPLTFGAGTKLEIKRAA

SEQ ID NO: 282 - protein, 109 aa, synthetic construct
DIVLTQSPASLAVSLGQRATITCSASSSVSYMHWYQQKSGTSPKRWIYDTSKLASGVPARFSGSGSGTSYSLTISSMEAEDAATYYCQQWSRNPLTFGAGTKLEIKRAA

SEQ ID NO: 283 - protein, 109 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKLEIKRAA

SEQ ID NO: 284 - protein, 109 aa, synthetic construct
DIVLTQSPAIMSASPGEKVTMTCSASASISYMHWYQQKSGTSPKPWIYEISKLASGVPARFSGSGSGTSYSLTISSMEAEDAAIYYCQQWSSYPLTFGAGTKLEIKRAA

SEQ ID NO: 285 - protein, 109 aa, synthetic construct
ENVLTQSPAIMSASPGEKVTISCSASSSVNYMYWYKQKPGSSPKPWIYRTSNLASGVPARFSGSGSGTSYSLTISSMEAEDAATYYCQQWSSNPLTFGAGTKLEIKRAA

SEQ ID NO: 286 - protein, 109 aa, synthetic construct
QIVLTQSPAIMSASPGEKVTMTCSASSSVSYMYWYQQKPRSSPISWIYLTSNLASGVPARFSGSGSGTSYSLTISSMEAEDAATYYCQQWSSDPLTFGAGTKLELKRAA

SEQ ID NO: 287 - protein, 109 aa, synthetic construct
ENVLTQSPAIMSASPGEKVTMTCSASSSVSYMYWYQQKPGSSPRLLIYRTSNLASGVPARFSGSGSGTSYSLTISSMEAEDAATYYCQQWSSNPPTFGGGTKLEIKRAA

SEQ ID NO: 288 - protein, 109 aa, synthetic construct
ENVLTQSPAIMSASPGEKVTISCSASSSVSYVYWYQQKPGSSPKPWIYRTSNLASGVPARFSGSGSGTSYSLTISSMEAEDAATYYCQQWSSNPLTFGAGTKLELKRAA

(19) United States
(12) Patent Application Publication
(10) Pub. No.: US 2025/0382369 A1
(43) Pub. Date: Dec. 18, 2025

(54) B7H3 BINDERS

(71) Applicant: UCL BUSINESS LTD, London (GB)

(72) Inventors: Kerry CHESTER, London (GB); John ANDERSON, London (GB); Martin PULE, London (GB); Kathleen BIRLEY, London (GB); Marta BARISA, London (GB)

(21) Appl. No.: 18/881,050

(22) PCT Filed: Jul. 4, 2023
(86) PCT No.: PCT/GB2023/051750

§ 371 Date: Jan. 3, 2025

(30) Foreign Application Priority Data
Jul. 4, 2022 (GB) 2209786.9

(51) Int. Cl. C07K 16/28; G01N 33/574

(52) U.S. Cl. CPC C07K 16/2827; G01N 33/57492; C07K 2317/24; G01N 2333/70532

(57) ABSTRACT

The present invention relates to novel antigen binding molecules which specifically bind to B7H3. The invention also relates to chimeric antigen receptors (CARs) and antibody-drug conjugates (ADCs) comprising the antigen binding molecules. Also provided are uses of the antigen binding molecules, CARs and ADCs, and pharmaceutical compositions comprising the antigen binding molecules, CARs and ADCs.

### FIELD OF THE INVENTION

The present invention relates to novel antigen binding molecules which specifically bind to B7H3. The invention also relates to chimeric antigen receptors (CARs) and antibody-drug conjugates (ADCs) comprising the antigen binding molecules. Also provided are uses of the antigen binding molecules, CARs and ADCs, and pharmaceutical compositions comprising the antigen binding molecules, CARs and ADCs.

### BACKGROUND TO THE INVENTION

Immunotherapy in the form of CAR T cell technology has yielded complete clinical responses and long-term cures in many patients with otherwise refractory B Cell malignancies. Despite this progress, similar successes have not been replicated in solid tumours for several reasons, including relative absence of suitable antigen targets, and challenges of penetrance and persistence in a solid tumour environment. Paediatric solid tumours create additional challenges due to a sparsity of neoantigens and their immunologically “cold”, hostile microenvironments.

B7-H3 (CD276) has emerged as a potential target for cancer immunotherapy in both solid and liquid malignancies arising in adults and children. A member of the immunoglobulin superfamily and the B7 family closely related to PD-L1, B7-H3 is found on most paediatric solid cancers, with a propensity for increased expression on high grade tumours, but B7-H3 is relatively absent from healthy cells.

B7-H3 exists as alternately spliced isoforms. In mice, there is a single isoform comprising two Immunolglobulin domains (2×Ig) C1 and V1. In contrast, human cell can express the 2×Ig C1/V1 but also the larger C1/V1/C2/V2 (4×Ig) which is an almost exact duplication of 2×Ig. 4×Ig is the predominant isoform in human cells including cancer cells.

When originally identified, B7-H3 was thought to be involved in T cell activation, but over time the body of evidence points to its predominant role as an inhibitor of the innate and adaptive immune system. The mechanism through which B7-H3 acts is poorly understood and although some receptors have been implicated, no study has conclusively identified the receptor or receptors through which B7-H3 signals. Additionally, B7-H3 is thought to have non-immunological roles in cancer progression and high expression is associated with increased invasion, metastasis, resistance to chemotherapy and a poorer prognosis.

Two previous anti-B7-H3 CAR T cell products have been reported and translated into clinical trials. Both incorporate a single chain Fv fragment (scFv) adapted from a monoclonal antibody: MGA271 and 376.96 respectively. Preclinical studies of anti B7-H3 CAR-T using these two scFv show their cytotoxic capacity against a range of solid tumours in vitro and in animal models.

There is a need for an improved treatment for cancers such as solid tumours.

### SUMMARY OF THE INVENTION

The present inventors have identified new B7-H3 binders with favourable properties which can be used for cancer treatment, particularly of many solid tumours. They constructed and then screened for binders in an scFv library by ELISA. ScFv libraries were constructed from mice that had been immunised with two domains of the 4×Ig isoform of human B7-H3 (the most proximal fused to the most distal). Some of the scFvs identified in initial screens were cloned into a scFv-Fc protein format and demonstrated specific binding to human 4×Ig B7-H3 expressed on the surface of human cells. In addition, some of these scFvs were produced as chimeric antibodies and showed specific binding to B7-H3 in ELISA and binding to B7-H3 expressed naturally on neuroblastoma cell lines. Selected anti-human B7-H3 scFvs were cloned into e.g. CAR-T and evaluated for anti-tumour reactivity in cytotoxicity, cytokine, and proliferation assays. In addition, further studies have been carried out using antigen binding molecules of the invention (e.g. an scFv) in an antibody-drug conjugate (ADC) or multispecific antigen binding molecule (e.g. bispecific antibodies, bi-specific T-cell engagers (BiTEs) etc.). The antigen binding molecules (e.g. scFvs) of the invention demonstrate superior antigen-specific cytotoxicity and cytokine secretion levels.

Accordingly, in a first aspect, the present invention provides an antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, wherein the antigen binding molecule comprises the complementarity determining region (CDR) sequences of:
(a) the heavy chain variable domain sequence of SEQ ID NO: 2 and the light chain variable domain sequence of SEQ ID NO: 10; or
(b) the heavy chain variable domain sequence of SEQ ID NO: 18 and the light chain variable domain sequence of SEQ ID NO: 26; or
(c) the heavy chain variable domain sequence of SEQ ID NO: 34 and the light chain variable domain sequence of SEQ ID NO: 42; or
(d) the heavy chain variable domain sequence of SEQ ID NO: 50 and the light chain variable domain sequence of SEQ ID NO: 58; or
(e) the heavy chain variable domain sequence of SEQ ID NO: 66 and the light chain variable domain sequence of SEQ ID NO: 74; or
(f) the heavy chain variable domain sequence of SEQ ID NO: 82 and the light chain variable domain sequence of SEQ ID NO: 90; or
(g) the heavy chain variable domain sequence of SEQ ID NO: 98 and the light chain variable domain sequence of SEQ ID NO: 106; or
(h) the heavy chain variable domain sequence of SEQ ID NO: 114 and the light chain variable domain sequence of SEQ ID NO: 122; or
(i) the heavy chain variable domain sequence of SEQ ID NO: 130 and the light chain variable domain sequence of SEQ ID NO: 138; or
(j) the heavy chain variable domain sequence of SEQ ID NO: 146 and the light chain variable domain sequence of SEQ ID NO: 154; or
(k) the heavy chain variable domain sequence of SEQ ID NO: 162 and the light chain variable domain sequence of SEQ ID NO: 170; or
(l) the heavy chain variable domain sequence of SEQ ID NO: 178 and the light chain variable domain sequence of SEQ ID NO: 186; or
(m) the heavy chain variable domain sequence of SEQ ID NO: 194 and the light chain variable domain sequence of SEQ ID NO: 202; or
(n) the heavy chain variable domain sequence of SEQ ID NO: 210 and the light chain variable domain sequence of SEQ ID NO: 218; or
(o) the heavy chain variable domain sequence of SEQ ID NO: 226 and the light chain variable domain sequence of SEQ ID NO: 234; or
(p) the heavy chain variable domain sequence of SEQ ID NO: 242 and the light chain variable domain sequence of SEQ ID NO: 250; or
(q) the heavy chain variable domain sequence of SEQ ID NO: 258 and the light chain variable domain sequence of SEQ ID NO: 266.

The antigen binding molecule may, in one preferred embodiment, comprise CDR sequences of the heavy chain variable domain sequence of SEQ ID NO: 66 and the light chain variable domain sequence of SEQ ID NO: 74.

In a second aspect, the present invention provides an antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, and wherein:
(a) HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; or
(b) HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; or
(c) HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; or
(d) HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; or
(e) HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; or
(f) HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; or
(g) HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; or
(h) HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; or
(i) HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; or
(j) HCDR1 comprises the sequence of SEQ ID NO: 148, HCDR2 comprises the sequence of SEQ ID NO: 150, HCDR3 comprises the sequence of SEQ ID NO: 152,
LCDR1 comprises the sequence of SEQ ID NO: 156, LCDR2 comprises the sequence of SEQ ID NO: 158, and LCDR3 comprises the sequence of SEQ ID NO: 160; or
(k) HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; or
(l) HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; or
(m) HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; or
(n) HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; or
(o) HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; or
(p) HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; or
(q) HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272.

In a third aspect, the present invention provides an antigen binding molecule that comprises a binding domain that specifically binds to B7H3, wherein the binding domain comprises a heavy chain variable domain and/or a light chain variable domain:
(a) wherein the heavy chain variable domain comprises a sequence of SEQ ID NO: 2 or a sequence having at least 90% identity thereto and wherein the light chain variable domain comprises a sequence of SEQ ID NO: 10 or a sequence having at least 90% identity thereto; or
(b) wherein the heavy chain variable domain comprises a sequence of SEQ ID NO: 18 or a sequence having at least 90% identity thereto and wherein the light chain variable domain comprises a sequence of SEQ ID NO: 26 or a sequence having at least 90% identity thereto; or
(c) wherein the heavy chain variable domain comprises a sequence of SEQ ID NO: 34 or a sequence having at least 90% identity thereto and wherein the light chain variable domain comprises a sequence of SEQ ID NO: 42 or a sequence having at least 90% identity thereto; or
(d) wherein the heavy chain variable domain comprises a sequence of SEQ ID NO: 50 or a sequence having at least 90% identity thereto and wherein the light chain variable domain comprises a sequence of SEQ ID NO: 58 or a sequence having at least 90% identity thereto; or
(e) wherein the heavy chain variable domain comprises a sequence of SEQ ID NO: 66 or a sequence having at least 90% identity thereto and wherein the light chain variable domain comprises a sequence of SEQ ID NO: 74 or a sequence having at least 90% identity thereto; or
(f) wherein the heavy chain variable domain comprises a sequence of SEQ ID NO: 82 or a sequence having at least 90% identity thereto and wherein the light chain variable domain comprises a sequence of SEQ ID NO: 90 or a sequence having at least 90% identity thereto; or
(g) wherein the heavy chain variable domain comprises a sequence of SEQ ID NO: 98 or a sequence having at least 90% identity thereto and wherein the light chain variable domain comprises a sequence of SEQ ID NO: 106 or a sequence having at least
(h) wherein the heavy chain variable domain comprises a sequence of SEQ ID NO: 114 or a sequence having at least 90% identity thereto and wherein the light chain variable domain comprises a sequence of SEQ ID NO: 122 or a sequence having at least 90% identity thereto; or
(i) wherein the heavy chain variable domain comprises a sequence of SEQ ID NO: 130 or a sequence having at least 90% identity thereto and wherein the light chain variable domain comprises a sequence of SEQ ID NO: 138 or a sequence having at least 90% identity thereto; or
(j) wherein the heavy chain variable domain comprises a sequence of SEQ ID NO: 146 or a sequence having at least 90% identity thereto and wherein the light chain variable domain comprises a sequence of SEQ ID NO: 154 or a sequence having at least 90% identity thereto; or
(k) wherein the heavy chain variable domain comprises a sequence of SEQ ID NO: 162 or a sequence having at least 90% identity thereto and wherein the light chain variable domain comprises a sequence of SEQ ID NO: 170 or a sequence having at least 90% identity thereto; or
(l) wherein the heavy chain variable domain comprises a sequence of SEQ ID NO: 178 or a sequence having at least 90% identity thereto and wherein the light chain variable domain comprises a sequence of SEQ ID NO: 186 or a sequence having at least 90% identity thereto; or
(m) wherein the heavy chain variable domain comprises a sequence of SEQ ID NO: 194 or a sequence having at least 90% identity thereto and wherein the light chain variable domain comprises a sequence of SEQ ID NO: 202 or a sequence having at least 90% identity thereto; or
(n) wherein the heavy chain variable domain comprises a sequence of SEQ ID NO: 210 or a sequence having at least 90% identity thereto and wherein the light chain variable domain comprises a sequence of SEQ ID NO: 218 or a sequence having at least 90% identity thereto; or
(o) wherein the heavy chain variable domain comprises a sequence of SEQ ID NO: 226 or a sequence having at least 90% identity thereto and wherein the light chain variable domain comprises a sequence of SEQ ID NO: 234 or a sequence having at least (p) wherein the heavy chain variable domain comprises a sequence of SEQ ID NO: 242 or a sequence having at least 90% identity thereto and wherein the light chain variable domain comprises a sequence of SEQ ID NO: 250 or a sequence having at least 90% identity thereto; or
(q) wherein the heavy chain variable domain comprises a sequence of SEQ ID NO: 258 or a sequence having at least 90% identity thereto and wherein the light chain variable domain comprises a sequence of SEQ ID NO: 266 or a sequence having at least 90% identity thereto.

The binding domain may, in one preferred embodiment, comprise a heavy chain variable domain that comprises a sequence of SEQ ID NO: 66 or a sequence having at least 90% identity thereto, and/or a light chain variable domain that comprises a sequence of SEQ ID NO: 74 or a sequence having at least 90% identity thereto.

In a fourth aspect, the present invention provides a chimeric antigen receptor (CAR) or chimeric co-stimulatory receptor (CCR) which comprises an antigen binding molecule as described herein which specifically binds to B7H3.

In a fifth aspect, the present invention provides a cell which comprises the CAR as described herein, wherein the cell is a T cell.

In a sixth aspect, the present invention provides a nucleic acid molecule comprising a nucleotide sequence encoding the antigen binding molecule or CAR as described herein.

In a seventh aspect, the present invention provides a nucleic acid molecule comprising a nucleotide sequence encoding the heavy chain variable domain or the light chain variable domain as described herein.

In an eighth aspect, the present invention provides an expression vector comprising the nucleic acid molecule as described herein.

In a ninth aspect, the present invention provides a host cell comprising the nucleic acid molecule or vector as described herein.

In a tenth aspect, the present invention provides an antibody-drug conjugate (ADC) comprising the antigen binding molecule as described herein linked to a drug.

In a eleventh aspect, the present invention provides a pharmaceutical composition comprising the antigen binding molecule, CAR, cell which comprises the CAR, or the ADC as described herein, and optionally a pharmaceutically acceptable carrier.

In a twelfth aspect, the present invention provides a method of treating cancer, wherein the method comprises administering the antigen binding molecule, CAR, cell which comprises the CAR, ADC, or pharmaceutical composition as described herein, to a subject in need thereof.

In a thirteenth aspect, the present invention provides the antigen binding molecule, the CAR, cell which comprises the CAR, ADC, or pharmaceutical composition as described herein for use in a method of treating cancer.

In a fourteenth aspect, the present invention provides a method of detecting cancer in a subject, comprising: contacting a biological sample from the subject with the antigen binding molecule as described herein and detecting antigen binding molecule bound to the sample, wherein binding of the antigen binding molecule to the sample indicates that the subject has cancer, optionally wherein the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC).

### DESCRIPTION OF THE FIGURES

FIG. 1—Schematic demonstrating strategy for production and panning of the anti-B7-H3 library. A. Production of the phage display library. Mice were immunised with a B7-H3-mouse Fc fusion protein. The spleens were collected from the immunised mice, mRNA extracted reverse transcribed into corresponding VH and VL cDNA. PCR reactions were used to add a linker and myc tag before the scFv-myc was cloned into the pHEN phagemid. Figure produced using Biorender. B. The phage display library was panned against human 4Ig-B7-H3 immobilized on immunotubes or magnetic beads.

FIG. 2—Anti-B7-H3 scFv were identified which showed binding against plate bound and cell bound human 4×Ig B7-H3. A. Bacteria clones demonstrating an anti-B7-H3 response in screening were regrown and retested in triplicate. Following induction of scFv-myc production, bacterial supernatant was tested in an ELISA against recombinant B7-H3 or PBS as a negative control. A commercial anti-B7-H3 monoclonal antibody and the serum from the immunized mouse were used as positive controls and a secondary only (anti-myc) was used as a negative control (Mean and SD, n=3). B. The structures of the scFv-myc fusion used in ELISA and the scFv-Fc fusion proteins used in staining experiments. C. Jurkat cells were transduced with one of three isoforms of B7-H3 shown from left to right: 1) naturally occurring human 4IgB7-H3, 2) construct depicted as T-B7H3 comprising the two membrane proximal domains of 4Ig version, which was used as the immunogen in mouse vaccinations to generate libraries, and 3) naturally occurring 2×Ig domain isoform to raise antibodies. D. The binding of 5 of the ScFv-Fc fusions identified from library against different cell bound isoforms of B7-H3.

FIG. 3—Data on the scFv-Fc fusions TB8, BG4, BD9, BC10, BB5. Using the same approach as in FIG. 2D, the binding of scFv-Fc against different cell bound isoforms of B7-H3 is shown. Representative 1 of 2.

FIG. 4—High level of diversity of the anti-B7-H3 scFv. A. The percentage similarities between 17 different anti-B7-H3 scFv. B. Alignment of the anti-B7-H3 scFv heavy and light chains arranged in alphabetical order as analysed by Abysis (http://www.abysis.org/abysis/). Amino acid differences between binders are indicated. A dot indicates same sequence as the binder listed above. A dashed line indicates absence of an amino acid.

FIG. 5—Specificity of three binders TE9, TC6 and BH6 to distinguish mouse, non-human primate, and human B7H3. A. Staining of mouse 3T3/NA1 fibroblasts with TE9, TC6 and BH6 binders in full antibody format; B. Results of ELISA showing binding of antibodies TE9, TC6 and BH6 against recombinant human B7 family proteins and non-human primate B7-H3; cyno indicates cynomolgus monkey (Macaca fascicularis).

FIG. 6—Chimeric antibodies derived from TE9 and BH6 show specificity for human B7H3 expressed on cell surface. TE9 and BH6 ScFv that had been cloned into full human IgG1 antibodies were used in staining experiments. A. Chimeric human IgG1 full antibodies derived from TE9 and BH6 ScFvs were used to stain 3 neuroblastoma cell lines, LAN-1, Kelly and IMR-32. The top row shows staining with a commercial directly conjugated antibody and the lower row with the chimeric antibodies and an anti-human secondary. B. Chimeric antibodies TE9 and BH6 were used to stain the 3 B7-H3 positive Jurkat cell lines, 4Ig-B7-H3, 2Ig-B7-H3 and T-B7-H3 (see FIG. 2)

FIG. 7—Anti-B7-H3 CAR T cells derived from TE9 and TC6 binders show T cell effector functions similar to anti-GD2 and anti-CD19 CAR T Cells. A. The second-generation CAR design used in this figure incorporating the CD8 hinge and transmembrane (H/Tm) and the CD28-CD3z endodomains. B. 4 hour Cr51 cytotoxicity assay of lead CAR T cells against isogenic B7-H3+/− cell lines and LAN-1 cells. Comparison made with αGD2 CAR T cells against isogenic GD2+/− cell lines and LAN-1. No significant difference in cytotoxicity against LAN-1 between TC6, TE9 and αGD2 CAR T cells (mean and SD n=3). C. Cell lines used as targets with levels of B7-H3 and GD2 expression. Values of antigen density was determined using quantbrite antigen quantification kit (BD Bioscience). D. CAR T cells were cultured with LAN-1, Kelly, or no antigen stimulus. IL-2 and IFN-γ were measured in overnight supernatant by ELISA (mean and SD, n=3-5: brackets indicate statistically significant differences using by 1 way ANOVA with Tukey multiple comparisons correction: **** p<0.00001 *** p<0.0001, ** p<0.001 and *p<0.01). E. Antigen specific cytokine response at the end of the tumour re-challenge assay in an experiment where irradiated target cells are added every 5-7 days for 4 stimulations. Data shown at day 9 (24 hours after second stimulation) and day 26 (24 hours after the fourth stimulation). Mean and range shown. Data is from two donors. Each sample was analysed once in duplicate.

FIGS. 8—TE9-CD28z CAR T cells show superior cytokine production compared with TE9-4-1BBz CAR T cells. A. Diagram of the second-generation CD28 and 4-1BB CAR constructs used in this figure. B. Transduction efficiency of TE9-CD28-CD3ξ (mean and range, n=6) or TE9-4-1BB-CD35 (mean and range, n=3). C. CAR T cells were cultured with cells containing antigen targets (LAN-1 or Kelly) or no antigen targets (Unstimulated) for 18 hours. Cells were stained for CD107a, CD69 and CD25. D. 18-hour culture assay: T Cells were transduced with TE9-285 or TE9-4-1BBξ. CAR-T cells were cultured with LAN-1 or Kelly target cells or no antigen stimulus for 18 hours. Cells were pelleted and supernatant used in ELISA compared with standard values of IL-2 or IFN-γ. (TE9-28ξ, Untransduced, n=6. TE9-BBξ (mean and range, n=3). E. Repeat Stimulation Assay: CARs and UT T-cells were cultured with LAN-1 or Kelly targets, or unstimulated for 7 days then restimulated with fresh antigen targets and incubated for a further 24 hours. IFN-γ and IL-2 production were measured using ELISA (mean and range, n=3). Brackets indicate statistically significant differences using by 1 way ANOVA with Tukey multiple comparisons correction: **** p<0.00001 *** p<0.0001, ** p<0.001 and *p<0.01).

FIG. 9—Second generation CAR T cells with a CD28 H/Tm show higher cytokine production and proliferation compared with CD8 H/Tm in the context of low antigen expression. A. A schematic of the second generation TE9 CAR T cells used in the figure with the CD8 and CD28 hinge and transmembrane (H/Tm). B. Cytokine production by T Cells transduced with second generation CARs containing either a CD8 H/Tm or a CD28 H/Tm or untransduced. T cells were incubated overnight with different concentrations of plate bound B7-H3 and the supernatant analysed for cytokine production (mean with range, n=3): St=stalk and is alternate notation for H/Tm used. C. The transduction efficiency of TE9-CD8 H/Tm and TE9 CD 28 H/Tm (mean with range, n=6). D. TE9 CAR T cells with either a CD28 H/Tm or CD8 H/Tm were incubated with LAN-1, Kelly, K562 or no antigen stimulus for 18 hours. Supernatant was used to quantify cytokine production using ELISA (mean and range, n=6). E. After 7 days of coculture, T cells were restimulated with fresh target cells or no antigen targets. Supernatant from 7-day cocultures was used to quantify cytokine production using ELISA (mean and range, n=4). F. TE9 CAR T cells with either a CD28 H/Tm or CD8 H/Tm were stained with CSFE and incubated with LAN-1, Kelly, K562 or no antigen stimulus for 7 days. Histograms show dilution of CSFE due to proliferation against different targets (representative 1 of 3). The AMFI (Change in Median Fluorescence Intensity) was calculated as the difference between the MFI of the test condition compared with the unstimulated untransduced control (mean and range, n=3). st=stalk ie Hinge/transmembrane: Brackets indicate statistically significant differences using by 1 way ANOVA with Tukey multiple comparisons correction: **** p<0.00001 *** p<0.0001, ** p<0.001 and *p<0.01.

FIGS. 10—TE9-28 & shows superior expansion and cytokine production in long term assays compared with GD2-285. CAR T cells were transduced with either TE9-282 (TE9), TE9-28-ILR22 (TE9-ILR2), or GD2-28 (GD2). A. A schematic of the CAR T cells used in this study. B. CAR T Cells or untransduced cells were cultured with either LAN-1, Kelly, or no antigen stimulus. Each week, cells were given a fresh antigen stimulus, cultured for a further 24 hours then analysed. The production of IFN-γ and IL-2 as determined using ELISA after each antigen stimulus (mean and range, n=4). C. The proliferation as measured by the fold change of CD3+ cells measured using flow cytometry. The significance is shown between cell numbers on day 28 (mean with range, n=4). Brackets indicate statistically significant differences using by 1 way ANOVA with Tukey multiple comparisons correction: **** p<0.00001 *** p<0.0001, ** p<0.001 and *p<0.01.

FIG. 11—In vivo testing of TE9-285, TE9-BBE and αGD2-28 CAR-T constructs. Mice treated with TE9-285 show increased survival and reduced tumour growth compared with other groups A. Experiment plan. B. Survival curve. C. Main graph: Tumour diameter with evaluation of mean tumour size at day 5 (mean, n=6: , ** p<0.001 and *p<0.01. D. Antigen expression after treatment as illustrated by the MFI of fluorophores used to stain antigens in tumour samples (mean and range, mice in untransduced and αGD2-28° C. groups, n=6. Mice in TE9-285 group n=5)

FIGS. 12—TE9-28 CAR T Cells showed greater penetrance and survival within the tumour environment compared with GD2 CAR-T or TE9-BBξ. A. Blood, spleen, and tumor samples were harvested and analyzed for persistence of CD34+ CAR T cells using flow cytometry. Although all mice were analyzed, samples with <100 human CD45 positive cells were excluded from analysis. Live cells were gated into 2 populations: those positive for human CD45 and those positive for mouse CD45. The graphs show the mean and individual values of the percentage of CD45 positive cells positive for human CD45 n=6 for all samples unless marked with * where N=5. B. Cells positive for human CD45 were gated according to CD3 expression. These graphs show the mean and individual values of the percentage of human CD45, human CD3 dual positive cells that were also CD34 positive n=6 for all samples unless marked with * where n=5. C. The number of samples from each CAR with >100 human T cells available for analysis. N=6 for all samples unless marked with * where n=5.

FIGS. 13—TE9-28ξ CAR-T eradicate orthotopic medulloblastoma tumour. Medulloblastoma MED8A cells (B7H3 positive) were injected into hemisphere of NSG mice and 5×10⁶ CAR-T cells or non-transduced control were injected 48 hours later into the lateral ventricles when small tumour established. A. Schematic of experiment. B. Bioluminescence values (flux) of groups of mice. C. Representative bioluminescence images of treated and untreated mice. At the end of follow up no tumours could be identified in the CAR-T treated mice.

FIG. 14—Comparison of TE9 CAR with anti CD33 CAR for reactivity against acute myeloid leukaemia cell lines. Anti CD33 CAR was built based on the ScFv derived from the heavy and light variable domains of gemtuzumab ozogamicin. The TE9 and CD33 CARs were built with CD28 and CD3ξ endodomains and with the hinge/transmembrane being derived from CD8-alpha (denoted CD8), or a CD28 transmembrane fused to the CH2 and CH3 domains of human IgG4 (denoted CH2CH3). MV411, Nomo-1 and THP-1 are human acute myeloid leukaemia cell lines. Jurkat cells and SupT1 human leukaemia cell lines were engineered to express 4Ig human B7H3 or human CD33 respectively. The respective CAR-T cells were labelled with cell trace violet dye and co-cultured at 1:1 ratio with the respective irradiated target cell lines for 7 days. Fold proliferation of the CAR-T cells is shown by the degree of dilution of the cell trace violet determined by flow cytometry after gating on the CAR-T cells.

FIGS. 15—TE9-28ξ CAR-T cells eradicate NOMO-1 acute myeloid leukaemia but do not affect normal haemopoiesis. The top indicates schematic of the experiment. TE9-285 CAR-T cells (CD8 were added at 5:1 effector to target ratio with either NOMO-1 acute myeloid leukaemia cells or with human haemopoietic stem cells in the form of cord blood. Target and effector cells were added to methocult medium that promotes formation of haemopoietic colonies. After 14 days, erythroid and myeloid colonies were counted from cord blood progenitors and leukaemic colonies were counted from the NOMO-1 progenitors. Three independent donors were the source of non-transduced controls and TE9 CAR-T cells respectively.

FIG. 16—TE9 second generation CAR-T comparison with equivalent CAR constructs build from 376.96 and MGA271 antibodies in a repeat stimulation assay. The three ScFv binders were cloned into identical second generation CAR backbones including and CD8-alpha hinge transmembrane and either CD28/CD35 or 41BB/CD35 endodomains. CARs and non-transduced controls were cultured with irradiated B7-H3 positive neuroblastoma targets (LAN-1 or Kelly cell lines) at 1:1 E:T ratio for 7 days then restimulated with fresh antigen targets and incubated for a further 24 hours. IFN-γ and IL-2 production were measured in the 24 hour supernatant following the second stimulation using ELISA. N-3 independent donors; Brackets indicate statistically significant differences using by 1 way ANOVA with Tukey multiple comparisons correction: **** p<0.00001 *** p<0.0001, ** p<0.001 and *p<0.01.

FIGS. 17 and 18—TE9 second generation CAR-T comparison with equivalent CAR constructs build from 376.96 and MGA271 antibodies in a repeat stimulation assay involving 4 target cell rechallenges. The three ScFv binders were cloned into identical second generation CAR backbones including and CD8-alpha hinge transmembrane and either CD28/CD35 or 41BB/CD35 endodomains. CARs and non-transduced controls were cultured at 1:1 ratio with irradiated B7-H3 positive neuroblastoma targets (LAN-1 or Kelly cell lines) for 7 days then restimulated with fresh irradiated antigen targets every 7 days for a total of 4 restimulations. After each restimulation, the supernatant from the co-culture was sampled after 24 hours and evaluated for cytokine secretion (interferon gamma in FIG. 17 and IL-2 in FIG. 18). Means+/−standard deviation from 4 independent donors is shown. Brackets indicate statistically significant differences using by 1 way ANOVA with Tukey multiple comparisons correction: **** p<0.00001 *** p<0.0001, ** p<0.001 and *p<0.01.

FIG. 19—Gamma delta T cells can be transduced with TE9-28ξ and show enhanced effector function in an antigen dependent manner. A. Schematic of the experiment. Human Gamma delta T cells (predominantly V-delta 1 type) were expanded from peripheral blood following depletion of alpha beta T cells and stimulation with anti CD3 OKT3 monoclonal antibody. Expanding cells were transduced with the indicated anti-B7-H3 CAR-T construct in SFG backbone on day 3 following T cell stimulation; Cells were expanded in the presence of IL-15 and functionally evaluated at day 20 of expansion. B. Transduction efficiency of expanded gamma delta T cells was determined by flow cytometry using direct staining of the CAR and the mean transduction efficiency over 9 independent donors is shown. C. Target cells for co-culture assays were evaluated for human B7-H3 expression by flow cytometry. D. Reactivity against the target cells is demonstrated by flow cytometric evaluation of CD107a and intracellular interferon gamma which are shifted to the right following culture. Vδ1 cell accumulation of intracellular IFN-γ and cell surface CD107a was measured after overnight co-culture with targets followed by a 4 h culture in monensin-supplemented media. Marker accumulation was measured in CAR-transduced and non-transduced Vδ1 cells. Shown are representative histograms of marker expression from a representative donor. The red line indicates median fluorescence intensity (MFI) of CAR-negative effectors alone, while the blue line indicates MFI of CAR-positive effectors alone.

FIG. 20—Gamma delta T cells transduced with TE9-28ξ CAR show enhanced cytotoxicity profiles and cytokine production in and antigen dependent manner. Gamma delta T cells were expanded and transduced as per figure (FIG. 19) before evaluation in co-culture experiments. A. IFN-γ and CD107a expression histogram data from 3 separate donors was converted to earth mover's distance (EMD) values, that compared marker expression between CAR-transduced versus non-transduced cells. A score of ‘0.0’ indicates no difference and is indicated by the dotted line (N=3; mean and distribution indicated). B. The same data was analysed using MFI measurements, compared in stratified CAR-positive (CD34+) and non-transduced (CD34−) Vδ1 cells in the same culture (N=3; mean and distribution indicated; statistical significance ascertained using one-way ANOVA).

FIG. 21—Gamma delta T cells transduced with TE9-28ξ CAR show antigen-dependent enhanced proliferation following tumour cell rechallenge. A. Schematic of experimental design: to test expanded CAR-Vδ1 persistence and proliferation, expanded cells were harvested and challenged twice at a 1:1 E:T ratio with irradiated B7H3 antigen-positive and negative Jurkat targets (B). C. CAR-Vδ1 CellTrace Violet dye dilution was measured after 6 days of co-culture. One representative donor matched data is shown. The right hand vertical line indicates the edge of undiluted dye at day 0 of the assay, the middle vertical line indicates dye MFI of CAR-Vδ1 only at day 6 indicative of background proliferation, while the left vertical line indicates dye MFI of CAR-Vδ1 in co-culture with antigen-positive targets. D. To account for ongoing background proliferation, Vδ1 cells were counted pre and post-co-culture using flow cytometric counting beads, and Vδ1 fold-change was normalized to effectors alone (N=3; mean+/−SEM; statistical significance was ascertained using a two-way ANOVA with Sidak's multiple comparison).

FIGS. 22—TE9 binder shows relatively high avidity in CAR-T format. A) Schematic of constructs used in the determination of avidity. All three binders were cloned into the same SFG gamma retroviral backbone and transduced into human primary T cells. B) relative B7H3 expression of the target cell lines used in these avidity experiments as determined by Quantibrite Phycoerythrin Fluorescence quantitation kit (BD Biosciences). C) Representative plots from Lumicks Cell Avidity Analayzer. The target cells are attached to the bottom of microwells and CAR-T cells are added. Increasing sonic forces are applied to dislodge cell. Greater force requirement is indicative of greater avidity. D) Summary of data from CAR-T generated from 4 independent blood donor. The percentage of cells attached at the maximum acoustic force of 1000 pN is shown. Duplicated measurements from the 4 donors are plotted. Significance differences are depicted following analysis by one-way Anova.

FIGS. 23—TE9 anti-B7H3 binder αβ-CAR-T exhibited superior effector functionality to competitor clinical-stage anti-B7H3 binders, MGA.271 and 376.96. All the data shown in this figure is of 3 independent donors, compared statistically with a Two-Way ANOVA. (a) T cells were transduced with a gamma-retroviral construct that encodes a 2nd generation 28 CAR and an RQR8 marker gene, separated by a 2A cleavage sequence. (b) CAR functionality was assessed in a serial re-challenge assay (every 24-48 hours), comparing responses against B7H3-negative SupT1-WT and positive SupT1-B7H3. An unsorted bulk transduced SupT1-B7H3 was used and contained a range of expression from zero to high. (c) A similar level of transduction efficiency >60% was achieved for all constructs tested (data shown for concatenated 3 independent donor transductions). (d) TE9- and 376.96-CAR-T cytotoxicity against antigen-positive targets was superior to that of MGA-CAR-T. (e, f) Following 5 challenges with SupT1-B7H3 targets, tumour B7H3 expression was measured. TE9- and 376.96-CAR-T cells killed all antigen-positive tumour targets, while MGA.271-CAR-T killed only B7H3-medium to bright tumours, enriching for antigen-intermediate tumour targets. (g) TE9- and 376.96-CAR-T cells proliferated substantially more in response to repeat challenge with tumour targets, (h) and produced higher levels of IL-2 and IFN-γ, compared to MGA.271-CAR-T.

FIGS. 24—TE9-based CAR-T show reduced basal activity in the absence of antigenic signal. a) The three anti-B7H3 binders were evaluated in identical second-generation format and expressed from SFG gammaretroviral vector. b) A similar level of transduction efficiency >60% was achieved for all constructs tested (data shown for concatenated 3 independent donor transductions). c) The three respective CAR-T products were manufactured from three independent blood donors following anti-CD3 and anti-CD28 antibody stimulation in the presence of 100 units/ml IL-2. Equivalent starting numbers were plated. 376.96-CAR-T cells proliferated substantially more than TE9-CAR-T during manufacture, consistent with greater autonomous signaling of 376.96-CAR-T. d) following manufacture cells were plated with either with target cells (see previous figure) or control wells to which media-only was added at the indicated time points. In contrast to TE9-, 376.96- and MGA.271-CAR-T proliferated in the absence of target challenge, indicative of potentially autonomous CAR-T signaling. e) spontaneous Cytokine production in the absence of added target cells was measured in the supernatants of cultured the CAR-T products manufactured from the same three donors.

FIG. 25—MART1-TCR-αβ-T cell proliferation, but not cytotoxicity, against peptide-loaded targets was enhanced with an anti-B7H3 CD28-CCR. (a) An illustration of the MART1-TCR, anti-B7H3 TE9 binder CD28-CCR and 2nd generation control TE9-28-ξ CAR constructs in SFG Y-retroviral format. (b) Representative dot plots of transduced T cells from one donor, showing RQR8 CAR/CCR marker gene and MART1-TCR vβ12 chain expression of non-transduced, single transduced or double transduced αβ-T cells, gated on day 7-expanded live singlet CD3+ cells. (c) An illustration of the provision of a MART1 TCR-derived signal 1 and in trans CCR-derived CD28 signal 2 versus the combined in cis signal 1+2 of a conventional 2nd generation TE9-28-& CAR. (d) A representative dot plot of U87 glioblastoma cell line expression of HLA-A2 and B7H3, rendering it sensitive to both targeting by both the MART1-TCR and TE9-targeted chimeric receptors. (e) 51Cr release over 4 h by U87 target cells either pre-pulsed with 5 μg/mL of MART1 peptide or untreated, at a range of E:T ratios (shown are means±SD across T cells sourced from 2 independent donors; two-way ANOVA). (f) Representative histograms show T cell CellTrace Violet proliferation dye dilution over a 7 day co-culture in the presence of 100 IU/mL IL-2, following a single challenge with live U87 target cells. (g) This proliferation data was collated across 4 donor T cells and compared using a proliferation index, which is derived by dividing the histograms into sextiles and recording the percentage of T cells in each sextile, then multiplying that percentage by increasing increments moving leftward to give greater weighting to proliferated T cells. The data was normalized to donor-matched effector-only conditions (N=4; mean±SD; two-way ANOVA). * P<0.05; ** P≤0.01; *** P≤0.001; **** P≤0.0001

FIG. 26—Anti-B7H3 CD28-CCR co-expression with a G115-TCR broadened αβ-T cell cytokine reactivity to targets that were not sufficiently stimulatory with Vγ9Vδ2-TCR alone. (a) An illustration of the provision of a G115 Vγ9Vδ2-TCR-derived signal 1 and in trans TE9 CCR-derived CD28 signal 2. (b) Diagrams showing retroviral constructs encoding either the G115-TCR alone, or the G115-TCR and TE9-CCRs either with a CD28 or 4-1BB signalling endodomain. (c) Representative dot plots of transduced T cells from one donor, showing RQR8 CCR marker gene and G115 Vδ2 chain expression of non-transduced and transduced αβ-T cells, gated on day 7-expanded live singlet CD3+ cells. (d) The G115-TCR conferred αβ-T cells with equivalent cytotoxicity to unmodified, natural γδ-T cells against Vγ9Vδ2-TCR-sensitive target DAUDI cell line, as indicated by DAUDI 51Cr release over 4 h co-culture (shown are means±SD across T cells sourced from 4 independent donors; two-way ANOVA). (e) G115-TCR signalling in transduced αβ-T cells was confirmed using PhosFlow phosphoprotein evaluation in response to stimulating TCR-like signalling with either anti-CD3 mAb (clone OKT-3) or anti-Vδ2 mAb (clone B6) (shown are means±SD across T cells sourced from 3 independent donors; two-way ANOVA). (f) A diagram illustrating the assay setup for differentially transduced T cell cytokine secretion. (g) Target cells were either unmodified or pre-treated with clone 20.1 mAb to convert cell-surface CD2771 from an inactive to an active conformation, boosting the signal that is delivered via the G115 Vγ9Vδ2-TCR. (h) Shown is the key for the different target types for T cell challenge, including highly sensitive DAUDI lymphoma cells, variably sensitive AML target cells MV4-11, NOMO-1 and THP-1, and poorly sensitive Jurkat leukaemia cells, either naturally B7H3-negative wild type (−WT) or transduced to express B7H3 (−B7H3). (i-k) Differentially transduced T cell IL-2 production after overnight culture with DAUDI, AML and Jurkat targets (E:T ratio 1:1; mean±SD across T cells sourced from 3 independent donors; two-way ANOVA). (l-n) Differentially transduced T cell IFN-γ production after overnight culture with DAUDI, AML and Jurkat targets (E:T ratio 1:1; mean±SD across T cells sourced from 3 independent donors; two-way ANOVA). * P<0.05; ** P≤0.01; *** P≤0.001; **** P≤0.0001

FIG. 27—Dependence of G115-αβ-T cell responsiveness on CD28-CCR expression increased upon re-challenge with tumour targets. (a) A diagram illustrating the assay setup for differentially transduced T cell functional testing. All the data in this figure is from the second challenge with tumour targets, and the relevant readouts are indicated. (b) Shown is the key for the different target types for T cell challenge, including highly sensitive DAUDI lymphoma cells, variably sensitive AML target cells MV4-11, NOMO-1 and THP-1, and poorly sensitive Jurkat leukaemia cells, either naturally B7H3-negative wild type (−WT) or transduced to express B7H3 (−B7H3). (c) Unmodified, TCR only and CCR-TCR-modified αβ-T cells were co-cultured with the range of targets, and 51Cr release over 4 h co-culture was measured (shown are means±SD across T cells sourced from 4 independent donors; two-way ANOVA). (d) Differentially transduced T cell IL-2 production after overnight culture with DAUDI, AML and Jurkat targets (E:T ratio 1:1; mean±SD across T cells sourced from 3 independent donors; two-way ANOVA). (e) Differentially transduced T cell IFN-γ production after overnight culture with DAUDI, AML and Jurkat targets (E:T ratio 1:1; mean±SD across T cells sourced from 3 independent donors; two-way ANOVA). (f) T cell proliferation in response to irradiated targets without exogenous IL-2 supplementation was tallied over a period of 7 days. This was done by plating 50,000 T cells and target cells at a 1:1 E:T ratio and then counting the number of T cells at harvest using Precision CountBeads. Resulting T cell numbers are shown (shown are means±SD across T cells sourced from 3 independent donors; two-way ANOVA). * P<0.05; ** P≤0.01; *** P≤0.001; **** P≤0.0001

FIG. 28. Binding specificity assessment of the TE9 and TC6 binders. Binding ELISA with immobilised recombinant B7-H3 isoforms was used to assess binding functionality of the three lead anti-B7-H3 antibodies; (A) Structure of the recombinant B7H3 proteins used to assess binding of the antibodies. (B) Binding properties of TE9 and BH6 antibodies against the respective recombinant human proteins and against mouse and cynomolgus monkey; (C) Binding of TE9 and TC6 antibodies compared against the mouse and cynomolgus monkey proteins.

### DETAILED DESCRIPTION

It is to be understood that different applications of the disclosed products and methods may be tailored to the specific needs in the art. It is also to be understood that the terminology used herein is for the purpose of describing particular embodiments of the invention only, and is not intended to be limiting. All publications, patents and patent applications cited herein, whether supra or infra, are hereby incorporated by reference in their entirety.

As used in this specification and the appended claims, the singular forms “a”, “an”, and “the” include plural referents unless the content clearly dictates otherwise. Thus, for example, reference to “an antigen binding molecule” includes “antigen binding molecules”, and the like.

### B7H3

B7H3 can be used interchangeably with CD276. B7-H3 is a member of the immunoglobulin (Ig) superfamily. The gene is located on chromosome 15 in humans, 9 in mice, and is highly conserved amongst different species. B7H3 most commonly occurs as a transmembrane protein with a residual cytoplasmic domain and no known signalling motifs. Alternative splicing results in the production of a number of isoforms of B7-H3. In humans and most other mammals, isoform 1 (also known as 4Ig-B7-H3) is the most common. This has 4 Ig subunits in its extracellular domain arranged in an Ig-V-1, Ig-C-1, Ig-V-2, Ig-C-2 pattern. Ig-V-1, Ig-C-1 is >96% homologous with Ig-V-2, Ig-C-2 and the repeating pattern is thought to result from exon duplication. The second most common isoform is isoform 2 (also known as 2Ig-B7-H3), a 2Ig membrane bound protein comprised of Ig-V-1-Ig-C-2. A soluble isoform of B7H3 is also present in the tumour microenvironment and serum of patients with cancer. An artificial, truncated isoform, T-B7-H3, also exists and consists of Ig-V-like Type 2 and Ig-C-like Type 2 subunits.

### Antigen Binding Molecule

The term “antigen binding molecule” as referred to herein includes whole antibodies and any antigen binding fragment (i.e., “antigen-binding portion”) or single chains thereof. The antigen binding molecule comprises a binding domain. The binding domain interacts with an antigen. For example, the antigen binding molecule may comprise a binding domain that binds to B7H3.

An antibody refers to a glycoprotein comprising at least two heavy (H) chains and two light (L) chains inter-connected by disulfide bonds. There are two types of light chain, lambda (λ) and kappa (κ). There are five main heavy chain classes (or isotypes) which determine the functional activity of an antibody molecule: IgM, IgD, IgG, IgA and IgE. The disclosed antibodies can be class switched. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR).

The CDRs are primarily responsible for antigen binding. The CDRs of each chain are typically referred to as CDR1, CDR2, and CDR3 (from the N-terminus to C-terminus), and are also typically identified by the chain in which the particular CDR is located. Light chain CDRs can be referred to as LCDR1, LCDR2 and LCDR3. Heavy chain CDRs can be referred to as HCDR1, HCDR2 and HCDR3. The CDR sequences are typically ordered on the light chain variable domain in an N-terminal to C-terminal direction: LCDR1, LCDR2, and LCDR3, and on the heavy chain variable domain in an N-terminal to C-terminal direction: HCDR1, HCDR2, and HCDR3.

The sequences of the framework regions of different light or heavy chains are relatively conserved within a species. The framework region of an antibody, that is the combined framework regions of the constituent light and heavy chains, serves to position and align the CDRs in three-dimensional space. The constant regions of the antibodies may mediate the binding of the immunoglobulin to host tissues or factors, including various cells of the immune system (e.g., effector cells) and the first component (C1q) of the classical complement system.

An antibody may be a “monoclonal antibody”. Monoclonal antibodies are immunoglobulin molecules that are identical to each other and have a single binding specificity and affinity for a particular epitope. They are produced by a single clone of B-lymphocytes or by a cell into which the light and heavy chain genes of a single antibody have been transfected. Monoclonal antibodies (mAbs) can be produced by a variety of techniques, including conventional monoclonal antibody methodology, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982).

An antibody may be a “chimeric” antibody which is an antibody that includes sequences from two different antibodies, which typically are of different species. For example, a chimeric antibody may comprise heavy and light chain variable regions derived from a first species and heavy and light chain constant regions derived from a second species. In other aspects, the variable and constant regions of the light chain may be derived from a first species while the variable region of the heavy chain may be derived from the first species and the constant region of the heavy chain is derived from a second species. In other aspects, the variable and constant regions of the light chain may be derived from a first species while the variable and constant regions of the heavy chain may be derived from a second species.

The term “fragment” of an antibody, typically refers to an “antigen binding fragment” of said antibody, i.e., one or more fragments of an antibody that retain the ability to specifically bind to an antigen. The present antigen binding molecule or antigen binding fragment retains the ability to specifically bind to B7H3, preferably human B7H3. Examples of antigen binding fragments include a Fab, a Fab′, a F(ab)′₂, a Fd, a Fv, a single chain Fab (scFab), a single chain Fv protein (scFv), a tandem scFv protein, a disulfide stabilized Fv protein (dsFv), a scFv-Fc protein, bi, tri or tetra-valent antibody, Bis-scFv, diabody, triabody, tetrabody or epitope-binding fragments of any of the above (see for example Holliger and Hudson, 2005, Nature Biotech. 23 (9): 1126-1136; Adair and Lawson, 2005, Drug Design Reviews-Online 2 (3), 209-217. The antigen binding fragments of the invention include a Fab, a Fab′, a F(ab)′₂, a Fd, a Fv, a single chain Fab (scFab), a single chain Fv protein (scFv), a tandem scFv protein, a disulfide stabilized Fv protein (dsFv), or a scFv-Fc protein that specifically binds human B7H3. These antigen binding fragments may be obtained using conventional techniques known to those of skill in the art. For example, antigen binding fragments can be produced by the modification of whole antibodies or synthesized de novo using recombinant DNA methodologies. In one embodiment, the antigen binding molecule of the invention is preferably an scFv or scFv-Fc protein. A scFv protein is a fusion protein in which a light chain variable region of an immunoglobulin and a heavy chain variable region of an immunoglobulin are bound by a linker. In dsFvs, the chains have been mutated to introduce a disulfide bond to stabilise the association of the chains. The term also includes genetically engineered forms such as chimeric antibodies and heteroconjugate antibodies such as bispecific antibodies. See also, Pierce Catalog and Handbook, 1994-1995 (Pierce Chemical Co., Rockford, IL); Kuby, Immunology, 3^{rd} Ed., W.H. Freeman & Co., New York, 1997.

The term “binding affinity” refers to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule. Typically, the Kd for the antibody with respect to the target will be 2-fold, preferably 5-fold, more preferably 10-fold less than Kd with respect to the other, non-target molecule. More preferably, the Kd will be 50-fold less, even more preferably 100-fold less, and yet more preferably 200-fold less. The value of this dissociation constant can be determined directly by well-known methods, and can be computed even for complex mixtures by methods such as those, for example, set forth in Caceci et al. (Byte 9:340-362, 1984). Methods for the evaluation of binding affinity of the antibodies of the invention for B7H3 preferably include ELISA or Biacore (i.e., surface plasmon resonance).

An antigen binding molecule of the invention binds (e.g., specifically binds) to B7H3 (preferably human B7H3), that is preferably they bind to B7H3 but they do not bind, or bind at a lower affinity, to other molecules. “Specifically binding” means that an antibody binds to B7H3 with greater affinity than to another target. Specific binding can be determined by methods known in the art. An antigen binding molecule of the invention is preferably capable of binding to B7H3 with an affinity that is at least two-fold, 10-fold, 50-fold, 100-fold or greater than its affinity for binding to another non-target molecule. Preferably, an antigen binding molecule of the invention may have a binding affinity (i.e., K_{D}) for B7H3 of 1×10⁻⁹ M or less. In some aspects, the antigen binding molecules specifically bind B7H3 with a K_{D} of about 1×10⁻⁹ M or less, about 1×10⁻¹⁰ M or less, about 1×10⁻¹¹ M or less, or about 1×10⁻¹² M or less. An antigen binding molecule of the invention may have some binding affinity for B7H3 from other mammals, for example primate or murine e.g. mouse or rat B7H3. The binding affinity of the antigen binding molecules of the invention for B7H3 from other species becomes progressively weaker as the binding epitope becomes less conserved with phylogenetic distance. An antigen binding molecule of the invention can bind (e.g. specifically bind) to any isoforms of B7H3. In one embodiment, the antigen molecule specifically binds to human B7H3. In one embodiment, the antigen binding molecule specifically binds to the human B7H3 isoform 4IgB7-H3. In one embodiment, the antigen binding molecule specifically binds to the human B7H3 isoform 2IgB7-H3. In one embodiment, the antigen binding molecule has specificity for both human B7H3 isoform 4IgB7-H3 and human B7H3 isoform 2IgB7-H3. In one embodiment, the antigen binding molecule specifically binds to isoform T-B7-H3. In one embodiment, the antigen binding molecule has specificity for T-B7-H3, human 4IgB7-H3 and human 2IgB7-H3. In one embodiment, the antigen binding molecule specifically binds to the human B7H3 isoform 4IgB7-H3 and does not bind or has a lower binding affinity for other targets. In one embodiment, the antigen binding molecule specifically binds to the human B7H3 isoform 2IgB7-H3 and does not bind or has a lower binding affinity for other targets. In one embodiment, the antigen binding molecule has specificity for both human B7H3 isoform 4IgB7-H3 and human B7H3 isoform 2IgB7-H3, and does not bind or has a lower binding affinity for other targets. In one embodiment, the antigen binding molecule specifically binds to isoform T-B7-H3, and does not bind or has a lower binding affinity for other targets. In one embodiment, the antigen binding molecule has specificity for T-B7-H3, human 4IgB7-H3 and human 2IgB7-H3, and does not bind or has a lower binding affinity for other targets.

An antigen binding molecule of the invention typically binds to the same epitope as the antigen binding molecule having heavy and light chain variable region sequences of (i) SEQ ID NOs: 2 and 10, respectively, (ii) SEQ ID NOs: 18 and 26, respectively; (iii) SEQ ID NOs: 34 and 42, respectively, (iv) SEQ ID NOs: 34 and 42, respectively, (v) SEQ ID NOs: 66 and 74, respectively, (vi) SEQ ID NOs: 82 and 90, respectively, (vii) SEQ ID NOs: 98 and 106, respectively, (viii) SEQ ID NOs: 114 and 122, respectively, (ix) SEQ ID NOs: 130 and 138, respectively, (×) SEQ ID NOs: 146 and 154, respectively, (xi) SEQ ID NOs: 162 and 170, respectively, (xii) SEQ ID NOs: 178 and 186, respectively, (xiii) SEQ ID NOs: 194 and 202, respectively, (xiv) SEQ ID NOs: 210 and 218, respectively, (xv) SEQ ID NOs: 226 and 234, respectively, (xvi) SEQ ID NOs: 242 and 250, respectively, or (xvii) SEQ ID NOs: 258 and 266, respectively. For instance, an antigen binding molecule of the invention may bind to the same epitope as the antigen binding molecule having heavy and light chain variable region sequences of SEQ ID NOs: 66 and 74, respectively. As used herein, the term “epitope” generally refers to the site on a target antigen which is recognised by an antibody. The location of an epitope may be identified by routine methods. For example, the general location of an epitope may be determined by assessing the ability of an antibody to bind to different fragments or variant B7H3 polypeptides, and for example by measuring binding following mutagenesis of particular residues in B7H3. Additionally, the antibody and target molecule may be combined and the antibody/target complex may be crystallised. The crystal structure of the complex may be determined and used to identify specific sites of interaction between the antibody and its target. An antigen binding molecule of the invention may cross-compete for binding to human B7H3 with another antigen binding molecule of the invention, preferably an antigen binding molecule having heavy and light chain variable region sequences of (i) SEQ ID NOs: 2 and 10, respectively, (ii) SEQ ID NOs: 18 and 26, respectively; (iii) SEQ ID NOs: 34 and 42, respectively, (iv) SEQ ID NOs: 34 and 42, respectively, (v) SEQ ID NOs: 66 and 74, respectively, (vi) SEQ ID NOs: 82 and 90, respectively, (vii) SEQ ID NOs: 98 and 106, respectively, (viii) SEQ ID NOs: 114 and 122, respectively, (ix) SEQ ID NOs: 130 and 138, respectively, (x) SEQ ID NOs: 146 and 154, respectively, (xi) SEQ ID NOs: 162 and 170, respectively, (xii) SEQ ID NOs: 178 and 186, respectively, (xiii) SEQ ID NOs: 194 and 202, respectively, (xiv) SEQ ID NOs: 210 and 218, respectively, (xv) SEQ ID NOs: 226 and 234, respectively, (xvi) SEQ ID NOs: 242 and 250, respectively, or (xvii) SEQ ID NOs: 258 and 266, respectively. For instance, an antigen binding molecule of the invention may cross-compete for binding to human B7H3 with another antigen binding molecule of the invention, preferably an antigen binding molecule having heavy and light chain variable region sequences of (i) SEQ ID NOs: 2 and 10, respectively, (ii) SEQ ID NOs: 18 and 26, respectively; (iii) SEQ ID NOs: 34 and 42, respectively, (iv) SEQ ID NOs: 34 and 42, respectively, (v) SEQ ID NOs: 66 and 74, respectively. Such cross-competing antigen binding molecules can be identified based on their ability to cross-compete with a known antigen binding molecule of the invention in standard binding assays, such as Biacore analysis, ELISA assays and flow cytometry.

The CDRs of SEQ ID NOs: 2, 18, 34, 50, 66, 82, 98, 114, 130, 146, 162, 178, 194, 210, 226, 242, and 258 and SEQ ID NOs: 10, 26, 42, 58, 74, 90, 106, 122, 138, 154, 170, 186, 202, 218, 234, 250, and 266 (i.e., the CDR sequences found within the respective heavy chain variable domain sequences and light chain variable domain sequences of said SEQ ID NOs) may be identified by any suitable method known in the art, for example using any suitable antibody numbering scheme. In some aspects the CDRs are identified using any of the Kabat numbering scheme (Kabat et al., U.S. Department of Health and Human Services, 1991), the Chothia numbering scheme (Chothia C, Lesk A M. J Mol Biol. (1987) 196:901-17), or the IMGT numbering scheme (Giudicelli V, et al. Nucleic Acids Res. (1997) 25:206-11; Lefranc M P. Immunol Today (1997) 18:509). The skilled person will appreciate that these different CDR labelling systems can give slightly different results, but in each case the CDRs can be easily identified by the skilled person. The CDR sequences set out in SEQ ID NOs: 4, 6, and 8, and 12, 14, and 16 are the HCDR1-3 and LCDR1-3 sequences respectively of SEQ ID NOs: 2 and 10 as defined using the Kabat numbering scheme. The CDR sequences set out in SEQ ID NOs: 20, 22, and 24, and 28, 30, and 32 are the HCDR1-3 and LCDR1-3 sequences respectively of SEQ ID NOs: 18 and 26 as defined using the Kabat numbering scheme. The CDR sequences set out in SEQ ID NOs: 36, 38, and 40, and 44, 46, and 48 are the HCDR1-3 and LCDR1-3 sequences respectively of SEQ ID NOs: 34 and 42 as defined using the Kabat numbering scheme. The CDR sequences set out in SEQ ID NOs: 52, 54, and 56, and 60, 62, and 64 are the HCDR1-3 and LCDR1-3 sequences respectively of SEQ ID NOs: 50 and 58 as defined using the Kabat numbering scheme. The CDR sequences set out in SEQ ID NOs: 68, 70, and 72, and 76, 78, and 80 are the HCDR1-3 and LCDR1-3 sequences respectively of SEQ ID NOs: 66 and 74 as defined using the Kabat numbering scheme. The CDR sequences set out in SEQ ID NOs: 84, 86, and 88, and 92, 94, and 96 are the HCDR1-3 and LCDR1-3 sequences respectively of SEQ ID NOs: 82 and 90 as defined using the Kabat numbering scheme. The CDR sequences set out in SEQ ID NOs: 100, 102, and 104, and 108, 110, and 112 are the HCDR1-3 and LCDR1-3 sequences respectively of SEQ ID NOs: 98 and 106 as defined using the Kabat numbering scheme. The CDR sequences set out in SEQ ID NOs: 116, 118, and 120, and 124, 126, and 128 are the HCDR1-3 and LCDR1-3 sequences respectively of SEQ ID NOs: 114 and 122 as defined using the Kabat numbering scheme. The CDR sequences set out in SEQ ID NOs: 132, 134, and 136, and 140, 142, and 144 are the HCDR1-3 and LCDR1-3 sequences respectively of SEQ ID NOs: 130 and 138 as defined using the Kabat numbering scheme. The CDR sequences set out in SEQ ID NOs: 148, 150, and 152, and 156, 158, and 160 are the HCDR1-3 and LCDR1-3 sequences respectively of SEQ ID NOs: 146 and 154 as defined using the Kabat numbering scheme. The CDR sequences set out in SEQ ID NOs: 164, 166, and 168, and 172, 174, and 176 are the HCDR1-3 and LCDR1-3 sequences respectively of SEQ ID NOs: 162 and 170 as defined using the Kabat numbering scheme. The CDR sequences set out in SEQ ID NOs: 180, 182, and 184, and 188, 190, and 192 are the HCDR1-3 and LCDR1-3 sequences respectively of SEQ ID NOs: 178 and 186 as defined using the Kabat numbering scheme. The CDR sequences set out in SEQ ID NOs: 196, 198, and 200, and 204, 206, and 208 are the HCDR1-3 and LCDR1-3 sequences respectively of SEQ ID NOs: 194 and 202 as defined using the Kabat numbering scheme. The CDR sequences set out in SEQ ID NOs: 212, 214 and 216, and 220, 222, and 224 are the HCDR1-3 and LCDR1-3 sequences respectively of SEQ ID NOs: 210 and 218 as defined using the Kabat numbering scheme. The CDR sequences set out in SEQ ID NOs: 228, 230, and 232, and 236, 238, and 240 are the HCDR1-3 and LCDR1-3 sequences respectively of SEQ ID NOs: 226 and 234 as defined using the Kabat numbering scheme. The CDR sequences set out in SEQ ID NOs: 244, 246, and 248, and 252, 254, and 256 are the HCDR1-3 and LCDR1-3 sequences respectively of SEQ ID NOs: 242 and 250 as defined using the Kabat numbering scheme. The CDR sequences set out in SEQ ID NOs: 260, 262, and 264, and 268, 270, and 272 are the HCDR1-3 and LCDR1-3 sequences respectively of SEQ ID NOs: 258 and 266 as defined using the Kabat numbering scheme.

The present invention relates to antigen binding molecules that comprise a binding domain that specifically binds to B7H3 (e.g. human B7H3). In some aspects, the binding domain comprises a heavy chain variable domain and/or a light chain variable domain. In some aspects, the binding domain comprises a heavy chain variable domain. In some aspects, the binding domain comprises a light chain variable domain. In some aspects, the binding domain comprises a heavy chain variable domain and a light chain variable domain. In some aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 2 and the light chain variable domain sequence of SEQ ID NO: 10. Typically, the antigen binding molecule comprises all six CDR sequences of SEQ ID NO: 2 and SEQ ID NO: 10, preferably the CDR sequences will be arranged as in SEQ ID NOs: 2 and 10 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 4, HCDR2 consists of the sequence of SEQ ID NO: 6, HCDR3 consists of the sequence of SEQ ID NO: 8, LCDR1 consists of the sequence of SEQ ID NO: 12, LCDR2 consists of the sequence of SEQ ID NO: 14, and LCDR3 consists of the sequence of SEQ ID NO:161.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 18 and the light chain variable domain sequence of SEQ ID NO: 26. Typically, the antigen binding molecule comprises all six CDR sequences of SEQ ID NO: 18 and SEQ ID NO: 26, preferably the CDR sequences will be arranged as in SEQ ID NOs: 18 and 26 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 20, HCDR2 consists of the sequence of SEQ ID NO: 22, HCDR3 consists of the sequence of SEQ ID NO: 24, LCDR1 consists of the sequence of SEQ ID NO: 28, LCDR2 consists of the sequence of SEQ ID NO: 30, and LCDR3 consists of the sequence of SEQ ID NO: 32.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 34 and the light chain variable domain sequence of SEQ ID NO: 42. Typically, the antigen binding molecule comprises all six CDR sequences of SEQ ID NO: 34 and SEQ ID NO: 42, preferably the CDR sequences will be arranged as in SEQ ID NOs: 34 and 42 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 36, HCDR2 consists of the sequence of SEQ ID NO: 38, HCDR3 consists of the sequence of SEQ ID NO: 40, LCDR1 consists of the sequence of SEQ ID NO: 44, LCDR2 consists of the sequence of SEQ ID NO: 46, and LCDR3 consists of the sequence of SEQ ID NO: 48.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 50 and the light chain variable domain sequence of SEQ ID NO: 58. Typically, the antigen binding molecule comprises all six CDR sequences of SEQ ID NO: 50 and SEQ ID NO: 58, preferably the CDR sequences will be arranged as in SEQ ID NOs: 50 and 58 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 52, HCDR2 consists of the sequence of SEQ ID NO: 54, HCDR3 consists of the sequence of SEQ ID NO: 56, LCDR1 consists of the sequence of SEQ ID NO: 60, LCDR2 consists of the sequence of SEQ ID NO: 62, and LCDR3 consists of the sequence of SEQ ID NO: 64.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 66 and the light chain variable domain sequence of SEQ ID NO: 74. Typically, the antigen binding molecule comprises all six CDR sequences of SEQ ID NO: 66 and SEQ ID NO: 74, preferably the CDR sequences will be arranged as in SEQ ID NOs: 66 and 74 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 68, HCDR2 consists of the sequence of SEQ ID NO: 70, HCDR3 consists of the sequence of SEQ ID NO: 72, LCDR1 consists of the sequence of SEQ ID NO: 76, LCDR2 consists of the sequence of SEQ ID NO: 78, and LCDR3 consists of the sequence of SEQ ID NO: 80.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 82 and the light chain variable domain sequence of SEQ ID NO: 90. Typically, the antigen binding molecule comprises all six CDR sequences of SEQ ID NO: 82 and SEQ ID NO: 90, preferably the CDR sequences will be arranged as in SEQ ID NOs: 82 and 90 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 84, HCDR2 consists of the sequence of SEQ ID NO: 86, HCDR3 consists of the sequence of SEQ ID NO: 88, LCDR1 consists of the sequence of SEQ ID NO: 92, LCDR2 consists of the sequence of SEQ ID NO: 94, and LCDR3 consists of the sequence of SEQ ID NO: 96.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 98 and the light chain variable domain sequence of SEQ ID NO: 106. Typically, the antigen binding molecule comprises all six CDR sequences of SEQ ID NO: 98 and SEQ ID NO: 106, preferably the CDR sequences will be arranged as in SEQ ID NOs: 98 and 106 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 100, HCDR2 consists of the sequence of SEQ ID NO: 102, HCDR3 consists of the sequence of SEQ ID NO: 104, LCDR1 consists of the sequence of SEQ ID NO: 108, LCDR2 consists of the sequence of SEQ ID NO: 110, and LCDR3 consists of the sequence of SEQ ID NO: 112.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 114 and the light chain variable domain sequence of SEQ ID NO: 122. Typically, the antigen binding molecule comprises all six CDR sequences of SEQ ID NO: 114 and SEQ ID NO: 122, preferably the CDR sequences will be arranged as in SEQ ID NOs: 114 and 122 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 116, HCDR2 consists of the sequence of SEQ ID NO: 118, HCDR3 consists of the sequence of SEQ ID NO: 120, LCDR1 consists of the sequence of SEQ ID NO: 124, LCDR2 consists of the sequence of SEQ ID NO: 126, and LCDR3 consists of the sequence of SEQ ID NO: 128.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 130 and the light chain variable domain sequence of SEQ ID NO: 138. Typically, the antigen binding molecule comprises all six CDR sequences of SEQ ID NO: 130 and SEQ ID NO: 138, preferably the CDR sequences will be arranged as in SEQ ID NOs: 130 and 138 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 132, HCDR2 consists of the sequence of SEQ ID NO: 134, HCDR3 consists of the sequence of SEQ ID NO: 136, LCDR1 consists of the sequence of SEQ ID NO: 140, LCDR2 consists of the sequence of SEQ ID NO: 142, and LCDR3 consists of the sequence of SEQ ID NO: 144.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 146 and the light chain variable domain sequence of SEQ ID NO: 154. Typically, the antigen binding molecule comprises all six CDR sequences of SEQ ID NO: 146 and SEQ ID NO: 154, preferably the CDR sequences will be arranged as in SEQ ID NOs: 146 and 154 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 148, HCDR2 consists of the sequence of SEQ ID NO: 150, HCDR3 consists of the sequence of SEQ ID NO: 152, LCDR1 consists of the sequence of SEQ ID NO: 156, LCDR2 consists of the sequence of SEQ ID NO: 158, and LCDR3 consists of the sequence of SEQ ID NO: 160.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 162 and the light chain variable domain sequence of SEQ ID NO: 170. Typically, the antigen binding molecule comprises all six CDR sequences of SEQ ID NO: 162 and SEQ ID NO: 170, preferably the CDR sequences will be arranged as in SEQ ID NOs: 162 and 170 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 164, HCDR2 consists of the sequence of SEQ ID NO: 166, HCDR3 consists of the sequence of SEQ ID NO: 168, LCDR1 consists of the sequence of SEQ ID NO: 172, LCDR2 consists of the sequence of SEQ ID NO: 174, and LCDR3 consists of the sequence of SEQ ID NO: 176.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 178 and the light chain variable domain sequence of SEQ ID NO: 186. Typically, the antigen binding molecule comprises all six CDR sequences of SEQ ID NO: 178 and SEQ ID NO: 186, preferably the CDR sequences will be arranged as in SEQ ID NOs: 178 and 186 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 180, HCDR2 consists of the sequence of SEQ ID NO: 182, HCDR3 consists of the sequence of SEQ ID NO: 184, LCDR1 consists of the sequence of SEQ ID NO: 188, LCDR2 consists of the sequence of SEQ ID NO: 190, and LCDR3 consists of the sequence of SEQ ID NO: 192.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 194 and the light chain variable domain sequence of SEQ ID NO: 202. Typically, the antigen binding molecule comprises all six CDR sequences of SEQ ID NO: 194 and SEQ ID NO: 202, preferably the CDR sequences will be arranged as in SEQ ID NOs: 194 and 202 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 196, HCDR2 consists of the sequence of SEQ ID NO: 198, HCDR3 consists of the sequence of SEQ ID NO: 200, LCDR1 consists of the sequence of SEQ ID NO: 204, LCDR2 consists of the sequence of SEQ ID NO: 206, and LCDR3 consists of the sequence of SEQ ID NO: 208.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 210 and the light chain variable domain sequence of SEQ ID NO: 218. Typically, the antigen binding molecule comprises all six CDR sequences of SEQ ID NO: 210 and SEQ ID NO: 218, preferably the CDR sequences will be arranged as in SEQ ID NOs: 210 and 218 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO:224. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 212, HCDR2 consists of the sequence of SEQ ID NO: 214, HCDR3 consists of the sequence of SEQ ID NO: 216, LCDR1 consists of the sequence of SEQ ID NO: 220, LCDR2 consists of the sequence of SEQ ID NO: 222, and LCDR3 consists of the sequence of SEQ ID NO: 224.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 226 and the light chain variable domain sequence of SEQ ID NO: 234. Typically, the antigen binding molecule comprises all six CDR sequences of SEQ ID NO: 226 and SEQ ID NO: 234, preferably the CDR sequences will be arranged as in SEQ ID NOs: 226 and 234 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 228, HCDR2 consists of the sequence of SEQ ID NO: 230, HCDR3 consists of the sequence of SEQ ID NO: 232, LCDR 1 consists of the sequence of SEQ ID NO: 236, LCDR2 consists of the sequence of SEQ ID NO: 238, and LCDR3 consists of the sequence of SEQ ID NO: 240.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 242 and the light chain variable domain sequence of SEQ ID NO: 250. Typically, the antigen binding molecule comprises all six CDR sequences of SEQ ID NO: 242 and SEQ ID NO: 250, preferably the CDR sequences will be arranged as in SEQ ID NOs: 242 and 250 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 244, HCDR2 consists of the sequence of SEQ ID NO: 246, HCDR3 consists of the sequence of SEQ ID NO: 248, LCDR1 consists of the sequence of SEQ ID NO: 252, LCDR2 consists of the sequence of SEQ ID NO: 254, and LCDR3 consists of the sequence of SEQ ID NO: 256.

In some other aspects, the heavy chain variable domain comprises a HCDR1, a HCDR2, and a HCDR3, and the light chain variable domain comprises a LCDR1, a LCDR2, and a LCDR3, and wherein the antigen binding molecule comprises the CDR sequences of the heavy chain variable domain sequence of SEQ ID NO: 258 and the light chain variable domain sequence of SEQ ID NO: 266. Typically, the antigen binding molecule comprises all six CDR sequences of SEQ ID NO: 258 and SEQ ID NO: 266, preferably the CDR sequences will be arranged as in SEQ ID NOs: 258 and 266 on the heavy and light chains of the antigen binding molecule and in the same order from N- to C-termini. These CDR sequences are defined using the Kabat numbering scheme. As will be appreciated by the skilled person, the exact CDR sequences may differ depending on the numbering scheme used (e.g., Kabat, Chothia or IMGT). In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 260, HCDR2 consists of the sequence of SEQ ID NO: 262, HCDR3 consists of the sequence of SEQ ID NO: 264, LCDR1 consists of the sequence of SEQ ID NO: 268, LCDR2 consists of the sequence of SEQ ID NO: 270, and LCDR3 consists of the sequence of SEQ ID NO: 272.

The present invention relates to antigen binding molecules that comprise a binding domain that specifically binds to B7H3 (e.g. human B7H3). In some aspects, the binding domain comprises a heavy chain variable domain and/or a light chain variable domain. In some aspects, the binding domain comprises a heavy chain variable domain. In some aspects, the binding domain comprises a light chain variable domain. In some aspects, the binding domain comprises a heavy chain variable domain and a light chain variable domain. In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 4, HCDR2 consists of the sequence of SEQ ID NO: 6, HCDR3 consists of the sequence of SEQ ID NO: 8, LCDR1 consists of the sequence of SEQ ID NO: 12, LCDR2 consists of the sequence of SEQ ID NO: 14, and LCDR3 consists of the sequence of SEQ ID NO: 16.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 20, HCDR2 consists of the sequence of SEQ ID NO: 22, HCDR3 consists of the sequence of SEQ ID NO: 24, LCDR1 consists of the sequence of SEQ ID NO: 28, LCDR2 consists of the sequence of SEQ ID NO: 30, and LCDR3 consists of the sequence of SEQ ID NO: 32.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 36, HCDR2 consists of the sequence of SEQ ID NO: 38, HCDR3 consists of the sequence of SEQ ID NO: 40, LCDR1 consists of the sequence of SEQ ID NO: 44, LCDR2 consists of the sequence of SEQ ID NO: 46, and LCDR3 consists of the sequence of SEQ ID NO: 48.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 52, HCDR2 consists of the sequence of SEQ ID NO: 54, HCDR3 consists of the sequence of SEQ ID NO: 56, LCDR1 consists of the sequence of SEQ ID NO: 60, LCDR2 consists of the sequence of SEQ ID NO: 62, and LCDR3 consists of the sequence of SEQ ID NO: 64.

In some preferred aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80. In some preferred aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 68, HCDR2 consists of the sequence of SEQ ID NO: 70, HCDR3 consists of the sequence of SEQ ID NO:72, LCDR1 consists of the sequence of SEQ ID NO: 76, LCDR2 consists of the sequence of SEQ ID NO: 78 and LCDR3 consists of the sequence of SEQ ID NO: 80.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 84, HCDR2 consists of the sequence of SEQ ID NO: 86, HCDR3 consists of the sequence of SEQ ID NO: 88, LCDR1 consists of the sequence of SEQ ID NO: 92, LCDR2 consists of the sequence of SEQ ID NO: 94, and LCDR3 consists of the sequence of SEQ ID NO: 96.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 100, HCDR2 consists of the sequence of SEQ ID NO: 102, HCDR3 consists of the sequence of SEQ ID NO: 104, LCDR1 consists of the sequence of SEQ ID NO: 108, LCDR2 consists of the sequence of SEQ ID NO: 110, and LCDR3 consists of the sequence of SEQ ID NO: 112.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 116, HCDR2 comprises the sequence of SEQ ID NO: 118, HCDR3 comprises the sequence of SEQ ID NO: 120, LCDR 1 comprises the sequence of SEQ ID NO: 124, LCDR2 comprises the sequence of SEQ ID NO: 126, and LCDR3 comprises the sequence of SEQ ID NO: 128. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 116, HCDR2 consists of the sequence of SEQ ID NO: 118, HCDR3 consists of the sequence of SEQ ID NO: 120, LCDR1 consists of the sequence of SEQ ID NO: 124, LCDR2 consists of the sequence of SEQ ID NO: 126, and LCDR3 consists of the sequence of SEQ ID NO: 128.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 132, HCDR2 consists of the sequence of SEQ ID NO: 134, HCDR3 consists of the sequence of SEQ ID NO: 136, LCDR1 consists of the sequence of SEQ ID NO: 140, LCDR2 consists of the sequence of SEQ ID NO: 142, and LCDR3 consists of the sequence of SEQ ID NO: 144.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 148, HCDR2 consists of the sequence of SEQ ID NO: 150, HCDR3 consists of the sequence of SEQ ID NO: 152, LCDR1 consists of the sequence of SEQ ID NO: 156, LCDR2 consists of the sequence of SEQ ID NO: 158, and LCDR3 consists of the sequence of SEQ ID NO: 160.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 164, HCDR2 consists of the sequence of SEQ ID NO: 166, HCDR3 consists of the sequence of SEQ ID NO: 168, LCDR1 consists of the sequence of SEQ ID NO: 172, LCDR2 consists of the sequence of SEQ ID NO: 174, and LCDR3 consists of the sequence of SEQ ID NO: 176.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 180, HCDR2 consists of the sequence of SEQ ID NO: 182, HCDR3 consists of the sequence of SEQ ID NO: 184, LCDR1 consists of the sequence of SEQ ID NO: 188, LCDR2 consists of the sequence of SEQ ID NO: 190, and LCDR3 consists of the sequence of SEQ ID NO: 192.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 196, HCDR2 consists of the sequence of SEQ ID NO: 198, HCDR3 consists of the sequence of SEQ ID NO: 200, LCDR1 consists of the sequence of SEQ ID NO: 204, LCDR2 consists of the sequence of SEQ ID NO: 206, and LCDR3 consists of the sequence of SEQ ID NO: 208.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 212, HCDR2 consists of the sequence of SEQ ID NO: 214, HCDR3 consists of the sequence of SEQ ID NO: 216, LCDR1 consists of the sequence of SEQ ID NO: 220, LCDR2 consists of the sequence of SEQ ID NO: 222, and LCDR3 consists of the sequence of SEQ ID NO: 224.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 228, HCDR2 consists of the sequence of SEQ ID NO: 230, HCDR3 consists of the sequence of SEQ ID NO: 232, LCDR1 consists of the sequence of SEQ ID NO: 236, LCDR2 consists of the sequence of SEQ ID NO: 238, and LCDR3 consists of the sequence of SEQ ID NO: 240.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 244, HCDR2 consists of the sequence of SEQ ID NO: 246, HCDR3 consists of the sequence of SEQ ID NO: 248, LCDR1 consists of the sequence of SEQ ID NO: 252, LCDR2 consists of the sequence of SEQ ID NO: 254, and LCDR3 consists of the sequence of SEQ ID NO: 256.

In some aspects, the heavy chain variable domain comprises a HCDR1, a HCDR2, and a HCDR3, and the light chain variable domain comprises a LCDR1, a LCDR2, and a LCDR3, and, wherein HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272. In some aspects, the binding domain comprises a heavy chain variable domain comprising a HCDR1, a HCDR2 and a HCDR3 and a light chain variable domain comprising a LCDR1, a LCDR2 and a LCDR3, and, wherein HCDR1 consists of the sequence of SEQ ID NO: 260, HCDR2 consists of the sequence of SEQ ID NO: 262, HCDR3 consists of the sequence of SEQ ID NO: 264, LCDR1 consists of the sequence of SEQ ID NO: 268, LCDR2 consists of the sequence of SEQ ID NO: 270, and LCDR3 consists of the sequence of SEQ ID NO: 272.

The present invention relates to antigen binding molecules that comprise a binding domain that specifically binds to B7H3 (e.g. human B7H3). In some aspects, the binding domain comprises a heavy chain variable domain and/or a light chain variable domain. In some aspects, the binding domain comprises a heavy chain variable domain. In some aspects, the binding domain comprises a light chain variable domain. In some aspects, the binding domain comprises a heavy chain variable domain and a light chain variable domain. In some aspects, the heavy chain variable domain comprises a sequence having at least 80%, at least 85%, at least 90%, at least 95%, at least 98%, at least 99%, or at least 99.5% identity to the sequence of SEQ ID NO: 2 and the light chain variable domain comprises a sequence having at least 80%, at least 85%, at least 90%, at least 95%, at least 98%, at least 99%, or at least 99.5% identity to the sequence of SEQ ID NO: 10. In some aspects, the heavy chain variable domain comprises a sequence having at least 90% identity to the sequence of SEQ ID NO: 2 and the light chain variable domain comprises a sequence having at least 90% to the sequence of SEQ ID NO: 10. In some aspects, the heavy chain variable domain comprises a sequence having at least 95% identity to the sequence of SEQ ID NO: 2 and the light chain variable domain comprises a sequence having at least 95% to the sequence of SEQ ID NO: 10. In some aspects, the heavy chain variable domain comprises a sequence of SEQ ID NO: 2 and the light chain variable domain comprises a sequence of SEQ ID NO: 10. In some aspects, the heavy chain variable domain consists of a sequence of SEQ ID NO: 2 and the light chain variable domain consists of a sequence of SEQ ID NO: 10. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 2. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 10. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 2; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 10. The heavy chain variable domain may comprise, or consist of, a sequence of SEQ ID NO: 2. The light chain variable domain may comprise, or consist of, a sequence of SEQ ID NO: 10. In some aspects, the heavy chain variable domain comprises a sequence of SEQ ID NO: 2 and the light chain variable domain comprises a sequence of SEQ ID NO: 10. In some aspects, the heavy chain variable domain consists of a sequence of SEQ ID NO: 2, and the light chain variable domain consists of a sequence of SEQ ID NO: 10.

In some aspects, the heavy chain variable domain comprises a sequence having at least 80%, at least 85%, at least 90%, at least 95%, at least 98%, at least 99%, or at least 99.5% identity to the sequence of SEQ ID NO: 18 and the light chain variable domain comprises a sequence having at least 80%, at least 85%, at least 90%, at least 95%, at least 98%, at least 99%, or at least 99.5% identity to the sequence of SEQ ID NO: 26. In some aspects, the heavy chain variable domain comprises a sequence having at least 90% identity to the sequence of SEQ ID NO: 18 and the light chain variable domain comprises a sequence having at least 90% to the sequence of SEQ ID NO: 26. In some aspects, the heavy chain variable domain comprises a sequence having at least 95% identity to the sequence of SEQ ID NO: 18 and the light chain variable domain comprises a sequence having at least 95% to the sequence of SEQ ID NO: 26. In some aspects, the heavy chain variable domain comprises a sequence of SEQ ID NO: 18 and the light chain variable domain comprises a sequence of SEQ ID NO: 26. In some aspects, the heavy chain variable domain consists of a sequence of SEQ ID NO: 18 and the light chain variable domain consists of a sequence of SEQ ID NO: 26. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 18. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 26. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 18; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 26. The heavy chain variable domain may comprise, or consist of, a sequence of SEQ ID NO: 18. The light chain variable domain may comprise, or consist of, a sequence of SEQ ID NO: 26. In some aspects, the heavy chain variable domain comprises a sequence of SEQ ID NO: 18 and the light chain variable domain comprises a sequence of SEQ ID NO: 26. In some aspects, the heavy chain variable domain consists of a sequence of SEQ ID NO: 18, and the light chain variable domain consists of a sequence of SEQ ID NO: 26.

In some aspects, the heavy chain variable domain comprises a sequence having at least 80%, at least 85%, at least 90%, at least 95%, at least 98%, at least 99%, or at least 99.5% identity to the sequence of SEQ ID NO: 34 and the light chain variable domain comprises a sequence having at least 80%, at least 85%, at least 90%, at least 95%, at least 98%, at least 99%, or at least 99.5% identity to the sequence of SEQ ID NO: 42. In some aspects, the heavy chain variable domain comprises a sequence having at least 90% identity to the sequence of SEQ ID NO: 34 and the light chain variable domain comprises a sequence having at least 90% to the sequence of SEQ ID NO: 42. In some aspects, the heavy chain variable domain comprises a sequence having at least 95% identity to the sequence of SEQ ID NO: 34 and the light chain variable domain comprises a sequence having at least 95% to the sequence of SEQ ID NO: 42. In some aspects, the heavy chain variable domain comprises a sequence of SEQ ID NO: 34 and the light chain variable domain comprises a sequence of SEQ ID NO: 42. In some aspects, the heavy chain variable domain consists of a sequence of SEQ ID NO: 34 and the light chain variable domain consists of a sequence of SEQ ID NO: 42. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 34. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 42. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 34; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 42. The heavy chain variable domain may comprise, or consist of, a sequence of SEQ ID NO: 34. The light chain variable domain may comprise, or consist of, a sequence of SEQ ID NO: 42. In some aspects, the heavy chain variable domain comprises a sequence of SEQ ID NO: 34 and the light chain variable domain comprises a sequence of SEQ ID NO: 42. In some aspects, the heavy chain variable domain consists of a sequence of SEQ ID NO: 34, and the light chain variable domain consists of a sequence of SEQ ID NO: 42.

In some aspects, the heavy chain variable domain comprises a sequence having at least 80%, at least 85%, at least 90%, at least 95%, at least 98%, at least 99%, or at least 99.5% identity to the sequence of SEQ ID NO: 50 and the light chain variable domain comprises a sequence having at least 80%, at least 85%, at least 90%, at least 95%, at least 98%, at least 99%, or at least 99.5% identity to the sequence of SEQ ID NO: 58. In some aspects, the heavy chain variable domain comprises a sequence having at least 90% identity to the sequence of SEQ ID NO: 50 and the light chain variable domain comprises a sequence having at least 90% to the sequence of SEQ ID NO: 58. In some aspects, the heavy chain variable domain comprises a sequence having at least 95% identity to the sequence of SEQ ID NO: 50 and the light chain variable domain comprises a sequence having at least 95% to the sequence of SEQ ID NO: 58. In some aspects, the heavy chain variable domain comprises a sequence of SEQ ID NO: 50 and the light chain variable domain comprises a sequence of SEQ ID NO: 58. In some aspects, the heavy chain variable domain consists of a sequence of SEQ ID NO: 50 and the light chain variable domain consists of a sequence of SEQ ID NO: 58. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 50. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 58. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 50; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 58. The heavy chain variable domain may comprise, or consist of, a sequence of SEQ ID NO: 50. The light chain variable domain may comprise, or consist of, a sequence of SEQ ID NO: 58. In some aspects, the heavy chain variable domain comprises a sequence of SEQ ID NO: 50 and the light chain variable domain comprises a sequence of SEQ ID NO: 58. In some aspects, the heavy chain variable domain consists of a sequence of SEQ ID NO: 50, and the light chain variable domain consists of a sequence of SEQ ID NO: 58.

In some preferred aspects, the heavy chain variable domain comprises a sequence having at least 80%, at least 85%, at least 90%, at least 95%, at least 98%, at least 99%, or at least 99.5% identity to the sequence of SEQ ID NO: 66 and the light chain variable domain comprises a sequence having at least 80%, at least 85%, at least 90%, at least 95%, at least 98%, at least 99%, or at least 99.5% identity to the sequence of SEQ ID NO: 74. In some preferred aspects, the heavy chain variable domain comprises a sequence having at least 90% identity to the sequence of SEQ ID NO: 66 and the light chain variable domain comprises a sequence having at least 90% to the sequence of SEQ ID NO: 74. In some preferred aspects, the heavy chain variable domain comprises a sequence having at least 95% identity to the sequence of SEQ ID NO: 66 and the light chain variable domain comprises a sequence having at least 95% to the sequence of SEQ ID NO: 74. In some preferred aspects, the heavy chain variable domain comprises a sequence of SEQ ID NO: 66 and the light chain variable domain comprises a sequence of SEQ ID NO: 74. In some preferred aspects, the heavy chain variable domain consists of a sequence of SEQ ID NO: 66 and the light chain variable domain consists of a sequence of SEQ ID NO: 74. In some preferred aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 66. In some preferred aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 74. In some preferred instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 66; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 74. The heavy chain variable domain may comprise, or consist of, a sequence of SEQ ID NO: 66. The light chain variable domain may comprise, or consist of, a sequence of SEQ ID NO: 74. In some preferred aspects, the heavy chain variable domain comprises a sequence of SEQ ID NO: 66 and the light chain variable domain comprises a sequence of SEQ ID NO: 74. In some preferred aspects, the heavy chain variable domain consists of a sequence of SEQ ID NO: 66, and the light chain variable domain consists of a sequence of SEQ ID NO: 74.

In some aspects, the heavy chain variable domain comprises a sequence having at least 80%, at least 85%, at least 90%, at least 95%, at least 98%, at least 99%, or at least 99.5% identity to the sequence of SEQ ID NO: 82 and the light chain variable domain comprises a sequence having at least 80%, at least 85%, at least 90%, at least 95%, at least 98%, at least 99%, or at least 99.5% identity to the sequence of SEQ ID NO: 90. In some aspects, the heavy chain variable domain comprises a sequence having at least 90% identity to the sequence of SEQ ID NO: 82 and the light chain variable domain comprises a sequence having at least 90% to the sequence of SEQ ID NO: 90. In some aspects, the heavy chain variable domain comprises a sequence having at least 95% identity to the sequence of SEQ ID NO: 82 and the light chain variable domain comprises a sequence having at least 95% to the sequence of SEQ ID NO: 90. In some aspects, the heavy chain variable domain comprises a sequence of SEQ ID NO: 82 and the light chain variable domain comprises a sequence of SEQ ID NO: 90. In some aspects, the heavy chain variable domain consists of a sequence of SEQ ID NO: 82 and the light chain variable domain consists of a sequence of SEQ ID NO: 90. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 82. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 90. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 82; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 90. The heavy chain variable domain may comprise, or consist of, a sequence of SEQ ID NO: 82. The light chain variable domain may comprise, or consist of, a sequence of SEQ ID NO: 90. In some aspects, the heavy chain variable domain comprises a sequence of SEQ ID NO: 82 and the light chain variable domain comprises a sequence of SEQ ID NO: 90. In some aspects, the heavy chain variable domain consists of a sequence of SEQ ID NO: 82, and the light chain variable domain consists of a sequence of SEQ ID NO: 90.

In some aspects, the heavy chain variable domain comprises a sequence having at least 80%, at least 85%, at least 90%, at least 95%, at least 98%, at least 99%, or at least 99.5% identity to the sequence of SEQ ID NO: 98 and the light chain variable domain comprises a sequence having at least 80%, at least 85%, at least 90%, at least 95%, at least 98%, at least 99%, or at least 99.5% identity to the sequence of SEQ ID NO: 106. In some aspects, the heavy chain variable domain comprises a sequence having at least 90% identity to the sequence of SEQ ID NO: 98 and the light chain variable domain comprises a sequence having at least 90% to the sequence of SEQ ID NO: 106. In some aspects, the heavy chain variable domain comprises a sequence having at least 95% identity to the sequence of SEQ ID NO: 98 and the light chain variable domain comprises a sequence having at least 95% to the sequence of SEQ ID NO: 106. In some aspects, the heavy chain variable domain comprises a sequence of SEQ ID NO: 98 and the light chain variable domain comprises a sequence of SEQ ID NO: 106. In some aspects, the heavy chain variable domain consists of a sequence of SEQ ID NO: 98 and the light chain variable domain consists of a sequence of SEQ ID NO: 106. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 98. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 106. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 98; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 106. The heavy chain variable domain may comprise, or consist of, a sequence of SEQ ID NO: 98. The light chain variable domain may comprise, or consist of, a sequence of SEQ ID NO: 106. In some aspects, the heavy chain variable domain comprises a sequence of SEQ ID NO: 98 and the light chain variable domain comprises a sequence of SEQ ID NO: 106. In some aspects, the heavy chain variable domain consists of a sequence of SEQ ID NO: 98, and the light chain variable domain consists of a sequence of SEQ ID NO: 106.

In some aspects, the heavy chain variable domain comprises a sequence having at least 80%, at least 85%, at least 90%, at least 95%, at least 98%, at least 99%, or at least 99.5% identity to the sequence of SEQ ID NO: 114 and the light chain variable domain comprises a sequence having at least 80%, at least 85%, at least 90%, at least 95%, at least 98%, at least 99%, or at least 99.5% identity to the sequence of SEQ ID NO: 122. In some aspects, the heavy chain variable domain comprises a sequence having at least 90% identity to the sequence of SEQ ID NO: 114 and the light chain variable domain comprises a sequence having at least 90% to the sequence of SEQ ID NO: 122. In some aspects, the heavy chain variable domain comprises a sequence having at least 95% identity to the sequence of SEQ ID NO: 114 and the light chain variable domain comprises a sequence having at least 95% to the sequence of SEQ ID NO: 122. In some aspects, the heavy chain variable domain comprises a sequence of SEQ ID NO: 114 and the light chain variable domain comprises a sequence of SEQ ID NO: 122. In some aspects, the heavy chain variable domain consists of a sequence of SEQ ID NO: 114 and the light chain variable domain consists of a sequence of SEQ ID NO: 122. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 114. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 122. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 114; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 122. The heavy chain variable domain may comprise, or consist of, a sequence of SEQ ID NO: 114. The light chain variable domain may comprise, or consist of, a sequence of SEQ ID NO: 122. In some aspects, the heavy chain variable domain comprises a sequence of SEQ ID NO: 114 and the light chain variable domain comprises a sequence of SEQ ID NO: 122. In some aspects, the heavy chain variable domain consists of a sequence of SEQ ID NO: 114, and the light chain variable domain consists of a sequence of SEQ ID NO: 122.

In some aspects, the heavy chain variable domain comprises a sequence having at least 80%, at least 85%, at least 90%, at least 95%, at least 98%, at least 99%, or at least 99.5% identity to the sequence of SEQ ID NO: 130 and the light chain variable domain comprises a sequence having at least 80%, at least 85%, at least 90%, at least 95%, at least 98%, at least 99%, or at least 99.5% identity to the sequence of SEQ ID NO: 138. In some aspects, the heavy chain variable domain comprises a sequence having at least 90% identity to the sequence of SEQ ID NO: 130 and the light chain variable domain comprises a sequence having at least 90% to the sequence of SEQ ID NO: 138. In some aspects, the heavy chain variable domain comprises a sequence having at least 95% identity to the sequence of SEQ ID NO: 130 and the light chain variable domain comprises a sequence having at least 95% to the sequence of SEQ ID NO: 138. In some aspects, the heavy chain variable domain comprises a sequence of SEQ ID NO: 130 and the light chain variable domain comprises a sequence of SEQ ID NO: 138. In some aspects, the heavy chain variable domain consists of a sequence of SEQ ID NO: 130 and the light chain variable domain consists of a sequence of SEQ ID NO: 138. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 130. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 138. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 130; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 138. The heavy chain variable domain may comprise, or consist of, a sequence of SEQ ID NO: 130. The light chain variable domain may comprise, or consist of, a sequence of SEQ ID NO: 138. In some aspects, the heavy chain variable domain comprises a sequence of SEQ ID NO: 130 and the light chain variable domain comprises a sequence of SEQ ID NO: 138. In some aspects, the heavy chain variable domain consists of a sequence of SEQ ID NO: 130, and the light chain variable domain consists of a sequence of SEQ ID NO: 138.

In some aspects, the heavy chain variable domain comprises a sequence having at least 80%, at least 85%, at least 90%, at least 95%, at least 98%, at least 99%, or at least 99.5% identity to the sequence of SEQ ID NO: 146 and the light chain variable domain comprises a sequence having at least 80%, at least 85%, at least 90%, at least 95%, at least 98%, at least 99%, or at least 99.5% identity to the sequence of SEQ ID NO: 154. In some aspects, the heavy chain variable domain comprises a sequence having at least 90% identity to the sequence of SEQ ID NO: 146 and the light chain variable domain comprises a sequence having at least 90% to the sequence of SEQ ID NO: 154. In some aspects, the heavy chain variable domain comprises a sequence having at least 95% identity to the sequence of SEQ ID NO: 146 and the light chain variable domain comprises a sequence having at least 95% to the sequence of SEQ ID NO: 154. In some aspects, the heavy chain variable domain comprises a sequence of SEQ ID NO: 146 and the light chain variable domain comprises a sequence of SEQ ID NO: 154. In some aspects, the heavy chain variable domain consists of a sequence of SEQ ID NO: 146 and the light chain variable domain consists of a sequence of SEQ ID NO: 154. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 146. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 154. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 146; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 154. The heavy chain variable domain may comprise, or consist of, a sequence of SEQ ID NO: 146. The light chain variable domain may comprise, or consist of, a sequence of SEQ ID NO: 154. In some aspects, the heavy chain variable domain comprises a sequence of SEQ ID NO: 146 and the light chain variable domain comprises a sequence of SEQ ID NO: 154. In some aspects, the heavy chain variable domain consists of a sequence of SEQ ID NO: 146, and the light chain variable domain consists of a sequence of SEQ ID NO: 154.

In some aspects, the heavy chain variable domain comprises a sequence having at least 80%, at least 85%, at least 90%, at least 95%, at least 98%, at least 99%, or at least 99.5% identity to the sequence of SEQ ID NO: 162 and the light chain variable domain comprises a sequence having at least 80%, at least 85%, at least 90%, at least 95%, at least 98%, at least 99%, or at least 99.5% identity to the sequence of SEQ ID NO: 170. In some aspects, the heavy chain variable domain comprises a sequence having at least 90% identity to the sequence of SEQ ID NO: 162 and the light chain variable domain comprises a sequence having at least 90% to the sequence of SEQ ID NO: 170. In some aspects, the heavy chain variable domain comprises a sequence having at least 95% identity to the sequence of SEQ ID NO: 162 and the light chain variable domain comprises a sequence having at least 95% to the sequence of SEQ ID NO: 170. In some aspects, the heavy chain variable domain comprises a sequence of SEQ ID NO: 162 and the light chain variable domain comprises a sequence of SEQ ID NO: 170. In some aspects, the heavy chain variable domain consists of a sequence of SEQ ID NO: 162 and the light chain variable domain consists of a sequence of SEQ ID NO: 170. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 162. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 170. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 162; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 170. The heavy chain variable domain may comprise, or consist of, a sequence of SEQ ID NO: 162. The light chain variable domain may comprise, or consist of, a sequence of SEQ ID NO: 170. In some aspects, the heavy chain variable domain comprises a sequence of SEQ ID NO: 162 and the light chain variable domain comprises a sequence of SEQ ID NO: 170. In some aspects, the heavy chain variable domain consists of a sequence of SEQ ID NO: 162, and the light chain variable domain consists of a sequence of SEQ ID NO: 170.

In some aspects, the heavy chain variable domain comprises a sequence having at least 80%, at least 85%, at least 90%, at least 95%, at least 98%, at least 99%, or at least 99.5% identity to the sequence of SEQ ID NO: 178 and the light chain variable domain comprises a sequence having at least 80%, at least 85%, at least 90%, at least 95%, at least 98%, at least 99%, or at least 99.5% identity to the sequence of SEQ ID NO: 186. In some aspects, the heavy chain variable domain comprises a sequence having at least 90% identity to the sequence of SEQ ID NO: 178 and the light chain variable domain comprises a sequence having at least 90% to the sequence of SEQ ID NO: 186. In some aspects, the heavy chain variable domain comprises a sequence having at least 95% identity to the sequence of SEQ ID NO: 178 and the light chain variable domain comprises a sequence having at least 95% to the sequence of SEQ ID NO: 186. In some aspects, the heavy chain variable domain comprises a sequence of SEQ ID NO: 178 and the light chain variable domain comprises a sequence of SEQ ID NO: 186. In some aspects, the heavy chain variable domain consists of a sequence of SEQ ID NO: 178 and the light chain variable domain consists of a sequence of SEQ ID NO: 186. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 178. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 186. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 178; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 186. The heavy chain variable domain may comprise, or consist of, a sequence of SEQ ID NO: 178. The light chain variable domain may comprise, or consist of, a sequence of SEQ ID NO: 186. In some aspects, the heavy chain variable domain comprises a sequence of SEQ ID NO: 178 and the light chain variable domain comprises a sequence of SEQ ID NO: 186. In some aspects, the heavy chain variable domain consists of a sequence of SEQ ID NO: 178, and the light chain variable domain consists of a sequence of SEQ ID NO: 186.

In some aspects, the heavy chain variable domain comprises a sequence having at least 80%, at least 85%, at least 90%, at least 95%, at least 98%, at least 99%, or at least 99.5% identity to the sequence of SEQ ID NO: 194 and the light chain variable domain comprises a sequence having at least 80%, at least 85%, at least 90%, at least 95%, at least 98%, at least 99%, or at least 99.5% identity to the sequence of SEQ ID NO: 202. In some aspects, the heavy chain variable domain comprises a sequence having at least 90% identity to the sequence of SEQ ID NO: 194 and the light chain variable domain comprises a sequence having at least 90% to the sequence of SEQ ID NO: 202. In some aspects, the heavy chain variable domain comprises a sequence having at least 95% identity to the sequence of SEQ ID NO: 194 and the light chain variable domain comprises a sequence having at least 95% to the sequence of SEQ ID NO: 202. In some aspects, the heavy chain variable domain comprises a sequence of SEQ ID NO: 194 and the light chain variable domain comprises a sequence of SEQ ID NO: 202. In some aspects, the heavy chain variable domain consists of a sequence of SEQ ID NO: 194 and the light chain variable domain consists of a sequence of SEQ ID NO: 202. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 194. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 202. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 194; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 202. The heavy chain variable domain may comprise, or consist of, a sequence of SEQ ID NO: 194. The light chain variable domain may comprise, or consist of, a sequence of SEQ ID NO: 202. In some aspects, the heavy chain variable domain comprises a sequence of SEQ ID NO: 194 and the light chain variable domain comprises a sequence of SEQ ID NO: 202. In some aspects, the heavy chain variable domain consists of a sequence of SEQ ID NO: 194, and the light chain variable domain consists of a sequence of SEQ ID NO: 202.

In some aspects, the heavy chain variable domain comprises a sequence having at least 80%, at least 85%, at least 90%, at least 95%, at least 98%, at least 99%, or at least 99.5% identity to the sequence of SEQ ID NO: 210 and the light chain variable domain comprises a sequence having at least 80%, at least 85%, at least 90%, at least 95%, at least 98%, at least 99%, or at least 99.5% identity to the sequence of SEQ ID NO: 218. In some aspects, the heavy chain variable domain comprises a sequence having at least 90% identity to the sequence of SEQ ID NO: 210 and the light chain variable domain comprises a sequence having at least 90% to the sequence of SEQ ID NO: 218. In some aspects, the heavy chain variable domain comprises a sequence having at least 95% identity to the sequence of SEQ ID NO: 210 and the light chain variable domain comprises a sequence having at least 95% to the sequence of SEQ ID NO: 218. In some aspects, the heavy chain variable domain comprises a sequence of SEQ ID NO: 210 and the light chain variable domain comprises a sequence of SEQ ID NO: 218. In some aspects, the heavy chain variable domain consists of a sequence of SEQ ID NO: 210 and the light chain variable domain consists of a sequence of SEQ ID NO: 218. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 210. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 218. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 210; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 218. The heavy chain variable domain may comprise, or consist of, a sequence of SEQ ID NO: 210. The light chain variable domain may comprise, or consist of, a sequence of SEQ ID NO: 218. In some aspects, the heavy chain variable domain comprises a sequence of SEQ ID NO: 210 and the light chain variable domain comprises a sequence of SEQ ID NO: 218. In some aspects, the heavy chain variable domain consists of a sequence of SEQ ID NO: 210, and the light chain variable domain consists of a sequence of SEQ ID NO: 218.

In some aspects, the heavy chain variable domain comprises a sequence having at least 80%, at least 85%, at least 90%, at least 95%, at least 98%, at least 99%, or at least 99.5% identity to the sequence of SEQ ID NO: 226 and the light chain variable domain comprises a sequence having at least 80%, at least 85%, at least 90%, at least 95%, at least 98%, at least 99%, or at least 99.5% identity to the sequence of SEQ ID NO: 234. In some aspects, the heavy chain variable domain comprises a sequence having at least 90% identity to the sequence of SEQ ID NO: 226 and the light chain variable domain comprises a sequence having at least 90% to the sequence of SEQ ID NO: 234. In some aspects, the heavy chain variable domain comprises a sequence having at least 95% identity to the sequence of SEQ ID NO: 226 and the light chain variable domain comprises a sequence having at least 95% to the sequence of SEQ ID NO: 234. In some aspects, the heavy chain variable domain comprises a sequence of SEQ ID NO: 226 and the light chain variable domain comprises a sequence of SEQ ID NO: 234. In some aspects, the heavy chain variable domain consists of a sequence of SEQ ID NO: 226 and the light chain variable domain consists of a sequence of SEQ ID NO: 234. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 226. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 234. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 226; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 234. The heavy chain variable domain may comprise, or consist of, a sequence of SEQ ID NO: 226. The light chain variable domain may comprise, or consist of, a sequence of SEQ ID NO: 234. In some aspects, the heavy chain variable domain comprises a sequence of SEQ ID NO: 226 and the light chain variable domain comprises a sequence of SEQ ID NO: 234. In some aspects, the heavy chain variable domain consists of a sequence of SEQ ID NO: 226, and the light chain variable domain consists of a sequence of SEQ ID NO: 234.

In some aspects, the heavy chain variable domain comprises a sequence having at least 80%, at least 85%, at least 90%, at least 95%, at least 98%, at least 99%, or at least 99.5% identity to the sequence of SEQ ID NO: 242 and the light chain variable domain comprises a sequence having at least 80%, at least 85%, at least 90%, at least 95%, at least 98%, at least 99%, or at least 99.5% identity to the sequence of SEQ ID NO: 250. In some aspects, the heavy chain variable domain comprises a sequence having at least 90% identity to the sequence of SEQ ID NO: 242 and the light chain variable domain comprises a sequence having at least 90% to the sequence of SEQ ID NO: 250. In some aspects, the heavy chain variable domain comprises a sequence having at least 95% identity to the sequence of SEQ ID NO: 242 and the light chain variable domain comprises a sequence having at least 95% to the sequence of SEQ ID NO: 250. In some aspects, the heavy chain variable domain comprises a sequence of SEQ ID NO: 242 and the light chain variable domain comprises a sequence of SEQ ID NO: 250. In some aspects, the heavy chain variable domain consists of a sequence of SEQ ID NO: 242 and the light chain variable domain consists of a sequence of SEQ ID NO: 250. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 242. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 250. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 242; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 250. The heavy chain variable domain may comprise, or consist of, a sequence of SEQ ID NO: 242. The light chain variable domain may comprise, or consist of, a sequence of SEQ ID NO: 250. In some aspects, the heavy chain variable domain comprises a sequence of SEQ ID NO: 242 and the light chain variable domain comprises a sequence of SEQ ID NO: 250. In some aspects, the heavy chain variable domain consists of a sequence of SEQ ID NO: 242, and the light chain variable domain consists of a sequence of SEQ ID NO: 250.

In some aspects, the heavy chain variable domain comprises a sequence having at least 80%, at least 85%, at least 90%, at least 95%, at least 98%, at least 99%, or at least 99.5% identity to the sequence of SEQ ID NO: 258 and the light chain variable domain comprises a sequence having at least 80%, at least 85%, at least 90%, at least 95%, at least 98%, at least 99%, or at least 99.5% identity to the sequence of SEQ ID NO: 266. In some aspects, the heavy chain variable domain comprises a sequence having at least 90% identity to the sequence of SEQ ID NO: 258 and the light chain variable domain comprises a sequence having at least 90% to the sequence of SEQ ID NO: 266. In some aspects, the heavy chain variable domain comprises a sequence having at least 95% identity to the sequence of SEQ ID NO: 258 and the light chain variable domain comprises a sequence having at least 95% to the sequence of SEQ ID NO: 266. In some aspects, the heavy chain variable domain comprises a sequence of SEQ ID NO: 258 and the light chain variable domain comprises a sequence of SEQ ID NO: 266. In some aspects, the heavy chain variable domain consists of a sequence of SEQ ID NO: 258 and the light chain variable domain consists of a sequence of SEQ ID NO: 266. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272; and wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 258. In some aspects, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 266. In some instances, the heavy chain variable domain comprises a HCDR1, a HCDR2, and a HCDR3, and the light chain variable domain comprises a LCDR1, a LCDR2, and a LCDR3, wherein HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272; wherein the heavy chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 258; and wherein the light chain variable domain comprises, or consists of, a sequence having at least 80%, at least 85%, at least 90%, at least 95%, at least 98%, at least 99%, at least 95%, at least 98%, at least 99%, at least 99.5% sequence identity to the sequence of SEQ ID NO: 266. The heavy chain variable domain may comprise, or consist of, a sequence of SEQ ID NO: 258. The light chain variable domain may comprise, or consist of, a sequence of SEQ ID NO: 266. In some aspects, the heavy chain variable domain comprises a sequence of SEQ ID NO: 258 and the light chain variable domain comprises a sequence of SEQ ID NO: 266. In some aspects, the heavy chain variable domain consists of a sequence of SEQ ID NO: 258, and the light chain variable domain consists of a sequence of SEQ ID NO: 266.

Sequence identity, including determination of sequence complementarity for nucleic acid or polynucleotide sequences, may be determined by sequence comparison and alignment algorithms known in the field. To determine the percent identity of two nucleic acid sequences (or polynucleotide sequences), the sequences are aligned for optimal comparison purposes (e.g., gaps can be introduced in the first sequence or second sequence for optimal alignment). The nucleotides at corresponding nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions*100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilised for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimised by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25 (17): 3389-3402. In another embodiment, the alignment is optimised by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including glycine and both the D or L optical isomers, and amino acid analogs and peptidomimetics.

An antigen binding molecule of the invention may alternatively comprise a variant of one or more of the specified sequences. A ‘variant’ may be a substitution, deletion or addition variant of any of the above amino acid sequences. A variant may comprise 1, 2, 3, 4, 5, up to 10, up to 20, up to 30 or more amino acid substitutions and/or deletions from the specific sequences and fragments discussed above, whilst maintaining the activity of the antigen binding molecules described herein. “Deletion” variants may comprise the deletion of, for example, 1, 2, 3, 4 or 5 individual amino acids. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, neutral |
| Asp | polar, hydrophilic, | Pro | hydrophobic, neutral |
| | charged (−) | | |
| Glu | polar, hydrophilic, | Gln | polar, hydrophilic, neutral |
| | charged (−) | | |
| Phe | aromatic, hydrophobic, | Arg | polar, hydrophilic, |
| | neutral | | charged (+) |
| Gly | aliphatic, neutral | Ser | polar, hydrophilic, neutral |
| His | aromatic, polar, | Thr | polar, hydrophilic, neutral |
| | hydrophilic, charged (+) | | |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

The substituents may also be selected from the amino acids selenocysteine and pyrrolysine. Preferred “derivatives” or “variants” include those in which instead of the naturally occurring amino acid the amino acid which appears in the sequence is a structural analog thereof. Amino acids used in the sequences may also be derivatized or modified, e.g. labelled, providing the function of the antigen binding molecule is not significantly adversely affected. Derivatives and variants as described above may be prepared during synthesis of the antigen binding molecule or by post-production modification, or when the antigen binding molecule is in recombinant form using the known techniques of site-directed mutagenesis, random mutagenesis, or enzymatic cleavage and/or ligation of nucleic acids. For example, an antigen binding molecule can be labelled with a radiolabeled amino acid. Examples of radiolabels include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I. The radiolabel may be used for both diagnostic and therapeutic purposes.

The antigen binding molecules described herein can be derivatized or linked to another molecule (such as another peptide or protein). In general, the antigen binding molecule is derivatized such that the binding to B7H3 is not affected adversely by the derivatization or labelling. For example, the antigen binding molecule can be functionally linked, for example, by chemical coupling, genetic fusion, noncovalent association or otherwise to one or more other molecular entities, such as another antibody (for example, a bispecific antibody or a diabody), a detection agent, a pharmaceutical agent, and/or a protein or peptide that can mediate associate of the antibody or antibody portion with another molecule (such as a streptavidin core region or a polyhistidine tag). An antigen binding domain that specifically binds to B7H3 can be labelled with a detectable moiety or marker as described herein.

Means of detecting labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the coloured label.

An antigen binding molecule can also be derivatized with a chemical group such as polyethylene glycol (PEG), a methyl or ethyl group, or a carbohydrate group. These groups may be useful to improve the biological characteristics of the antigen binding domain, such as to increase serum half-life or to increase tissue binding.

### Other Features of the Antigen Binding Molecules of the Invention

The antigen binding molecule may be human or humanised. A “humanised” antigen binding molecule includes a human framework region and one or more CDRs from a non-human antigen binding molecule such as an antibody (e.g., a monkey, mouse, rat, or synthetic antibody). The non-human antigen binding molecule providing the CDRs is the “donor”, and the human antigen binding molecule providing the framework is the “acceptor”. Preferably, all six CDR sequences in the humanised antigen binding molecule are from the antigen binding molecule. The humanised antigen binding molecule may not comprise a constant region. If constant regions are present in the humanised antigen binding molecule they are typically substantially identical to human antigen binding molecule constant regions, such as having at least 85%, at least 90%, at least 95%, at least 98%, or about 100% sequence identity with a human constant region; preferably having at least 90%, or most preferably having at least 95%, sequence identity with a human constant region. Thus, in preferred aspects all parts of a humanised antigen binding molecule, except the CDRs, are substantially identical to (i.e., have at least 90% and preferably at least 95% sequence identity with) corresponding parts of natural human antigen binding molecule sequences. A “humanised antigen binding molecule” can include a humanised light chain and a humanised heavy chain. A humanised antigen binding molecule binds to the same antigen as the donor antigen binding molecule that provides the CDRs. The acceptor framework of a humanised antigen binding molecule may have a limited number of substitutions (typically between about 1-50, 1-40, 1-30, 1-20, 1-10 or 1-5 substitutions, preferably 1-20 and most preferably 1-10 substitutions) with amino acids taken from the donor framework. Humanised antigen binding molecules or other monoclonal antibodies can have additional conservative amino acid substitutions which have substantially no effect on antigen binding or other immunoglobulin functions. Humanised immunoglobulins can be constructed by means of genetic engineering (for example, see U.S. Pat. No. 5,585,089). In some aspects, the binding domain of the antigen binding molecule of the invention may be human or humanised. In some aspects, the heavy chain variable domain and/or light chain variable domain may be human or humanised. In some aspects, the antigen binding domain is a humanised antigen binding domain and comprises one or more human framework regions.

In some aspects the antigen binding molecule may be an antibody fragment or a single chain antibody, optionally wherein, the fragment is a Fab fragment, a Fab′ fragment, a F(ab)′2 fragment, a single chain Fab (scFab) fragment, a single chain Fv protein (scFv), a tandem scFv protein, or a disulfide stabilized Fv protein (dsFv), a scFv-Fc protein, bi, tri or tetra-valent antibody, Bis-scFv, diabody, triabody, tetrabody or epitope-binding fragments of any of the above (see for example Holliger and Hudson, 2005, Nature Biotech. 23 (9): 1126-1136; Adair and Lawson, 2005, Drug Design Reviews-Online 2 (3), 209-217). The methods for creating and manufacturing antibody fragments are well known in the art (see for example Verma et al., 1998, Journal of Immunological Methods, 216, 165-181).

In some preferred aspects, the antigen binding molecule is an scFv or a scFv-Fc. A scFv protein is a fusion protein in which a light chain variable region (LCVR) of an immunoglobulin and a heavy chain variable region (HCVR) of an immunoglobulin are bound by a linker. In some aspects the scFv has a linker sequence of GGGGSGGGGSGGGGS (SEQ ID NO: 273). In some aspects, the scFv comprises the linker of SEQ ID NO: 273 and the heavy chain variable domain is connected to the light chain variable domain via the linker. In some aspects, the components of the scFv are arranged in the order of 5′-HCVR-Linker-LCVR-3′. In some aspects, the scFv has a heavy chain variable domain comprising a sequence of SEQ ID NO: 2 that is connected to a light chain variable domain comprising a sequence of SEQ ID NO: 10 via a linker having a sequence of SEQ ID NO: 273. In some aspects, the scFv has a heavy chain variable domain comprising a sequence of SEQ ID NO: 18 that is connected to a light chain variable domain comprising a sequence of SEQ ID NO: 26 via a linker having a sequence of SEQ ID NO: 273. In some aspects, the scFv has a heavy chain variable domain comprising a sequence of SEQ ID NO: 34 that is connected to a light chain variable domain comprising a sequence of SEQ ID NO: 42 via a linker having a sequence of SEQ ID NO: 273. In some aspects, the scFv has a heavy chain variable domain comprising a sequence of SEQ ID NO: 50 that is connected to a light chain variable domain comprising a sequence of SEQ ID NO: 58 via a linker having a sequence of SEQ ID NO: 273. In some preferred aspects, the scFv has a heavy chain variable domain comprising a sequence of SEQ ID NO: 66 that is connected to a light chain variable domain comprising a sequence of SEQ ID NO: 74 via a linker having a sequence of SEQ ID NO: 273. In some aspects, the scFv has a heavy chain variable domain comprising a sequence of SEQ ID NO: 82 that is connected to a light chain variable domain comprising a sequence of SEQ ID NO: 90 via a linker having a sequence of SEQ ID NO: 273. In some aspects, the scFv has a heavy chain variable domain comprising a sequence of SEQ ID NO: 98 that is connected to a light chain variable domain comprising a sequence of SEQ ID NO: 106 via a linker having a sequence of SEQ ID NO: 273. In some aspects, the scFv has a heavy chain variable domain comprising a sequence of SEQ ID NO: 114 that is connected to a light chain variable domain comprising a sequence of SEQ ID NO: 122 via a linker having a sequence of SEQ ID NO: 273. In some aspects, the scFv has a heavy chain variable domain comprising a sequence of SEQ ID NO: 130 that is connected to a light chain variable domain comprising a sequence of SEQ ID NO: 138 via a linker having a sequence of SEQ ID NO: 273. In some aspects, the scFv has a heavy chain variable domain comprising a sequence of SEQ ID NO: 146 that is connected to a light chain variable domain comprising a sequence of SEQ ID NO: 154 via a linker having a sequence of SEQ ID NO: 273. In some aspects, the scFv has a heavy chain variable domain comprising a sequence of SEQ ID NO: 162 that is connected to a light chain variable domain comprising a sequence of SEQ ID NO: 170 via a linker having a sequence of SEQ ID NO: 273. In some aspects, the scFv has a heavy chain variable domain comprising a sequence of SEQ ID NO: 178 that is connected to a light chain variable domain comprising a sequence of SEQ ID NO: 186 via a linker having a sequence of SEQ ID NO: 273. In some aspects, the scFv has a heavy chain variable domain comprising a sequence of SEQ ID NO: 194 that is connected to a light chain variable domain comprising a sequence of SEQ ID NO: 202 via a linker having a sequence of SEQ ID NO: 273. In some aspects, the scFv has a heavy chain variable domain comprising a sequence of SEQ ID NO: 210 that is connected to a light chain variable domain comprising a sequence of SEQ ID NO: 218 via a linker having a sequence of SEQ ID NO: 273. In some aspects, the scFv has a heavy chain variable domain comprising a sequence of SEQ ID NO: 226 that is connected to a light chain variable domain comprising a sequence of SEQ ID NO: 234 via a linker having a sequence of SEQ ID NO: 273. In some aspects, the scFv has a heavy chain variable domain comprising a sequence of SEQ ID NO: 242 that is connected to a light chain variable domain comprising a sequence of SEQ ID NO: 250 via a linker having a sequence of SEQ ID NO: 273. In some aspects, the scFv has a heavy chain variable domain comprising a sequence of SEQ ID NO: 258 that is connected to a light chain variable domain comprising a sequence of SEQ ID NO: 266 via a linker having a sequence of SEQ ID NO: 273. In a preferred aspect, the scFv has a heavy chain variable domain comprising a sequence of SEQ ID NO: 50 that is connected to a light chain variable domain comprising a sequence of SEQ ID NO: 58 via a linker having a sequence of SEQ ID NO: 273. In a further preferred aspect, the scFv has a heavy chain variable domain comprising a sequence of SEQ ID NO: 258 that is connected to a light chain variable domain comprising a sequence of SEQ ID NO: 266 via a linker having a sequence of SEQ ID NO: 273. In a more preferred aspect, the scFv has a heavy chain variable domain comprising a sequence of SEQ ID NO: 66 that is connected to a light chain variable domain comprising a sequence of SEQ ID NO: 74 via a linker having a sequence of SEQ ID NO: 273. Optionally, the components of the scFv may be arranged in the order of 5′-LCVR-Linker-HCVR-3′.

In some aspects, the antigen binding molecule is a multispecific molecule. In some aspects, the antigen binding molecule is a bispecific molecule, for example comprising a first and a second antigen binding domain, wherein the first antigen binding domain corresponds to an antigen binding molecule of the invention and specifically binds B7H3, and wherein the second antigen binding domain specifically binds a different target antigen. In some aspects, the antigen binding molecule may be a biparatopic molecule, for example comprising a first antigen binding domain and a second antigen binding domain, wherein the first antigen binding domain corresponds to an antigen binding molecule of the invention, and wherein the second antigen binding domain is from a second, different antigen binding molecule of the invention, wherein the first and second antigen binding domains specifically bind B7H3 and recognise distinct, non-overlapping epitopes. In some aspects, the antigen binding molecule is a trispecific molecule, for example comprising a first, a second and a third antigen binding domain, wherein the first antigen binding domain corresponds to an antigen binding molecule of the invention and specifically binds B7H3, and wherein the second (and third) antigen binding domain specifically binds a different target antigen.

In some aspects, the antigen binding molecule is a bi-specific T-cell engager (BiTE). In some aspects, the BiTE comprises a first and a second binding domain, wherein the first antigen binding domain corresponds to an antigen binding molecule of the invention and specifically binds B7H3, and wherein the second antigen binding domain specifically binds to CD3 on the surface of a T cell. For instance, the second antigen binding domain specifically binds to the CD3 subunit of the T-Cell Receptor (TCR). Antigen binding molecules comprising such binding domains are well known to those skilled in the art. For example, anti-CD3 monoclonal antibodies contain such a binding domain and are readily obtainable. In some aspects, the second antigen binding domain specifically binds to other T cell-specific surface molecules.

In one aspect, the antigen binding molecule of the invention is a bispecific or BiTE molecule comprising a first antigen binding domain and a second antigen binding domain, wherein the first antigen binding domain specifically binds B7H3, and wherein the first antigen binding domain comprises a heavy chain variable domain and a light chain variable domain, wherein the heavy chain variable domain comprises a HCDR1 comprising the sequence of SEQ ID NO: 68, a HCDR2 comprising the sequence of SEQ ID NO: 70, and a HCDR3 comprising the sequence of SEQ ID NO: 72, and wherein the light chain variable domain comprises a LCDR1 comprising the sequence of SEQ ID NO: 76, a LCDR2 comprising the sequence of SEQ ID NO: 78, and a LCDR3 comprising the sequence of SEQ ID NO: 80; preferably wherein the heavy chain variable domain comprises a sequence of SEQ ID NO: 66 and the light chain variable domain comprises a sequence of SEQ ID NO: 74.

A bispecific, trispecific or multispecific molecule can be produced by cross-linking two or more antigen binding domains. Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). In some aspects, the bispecific (e.g. BiTE) or biparatopic molecule described herein comprises two scFvs. For example, for a BiTE molecule, the first antigen binding domain may be an scFv of the invention that specifically binds B7H3 and the second CD3 antigen binding domain may also be an scFv. The two scFv antibodies may for example be covalently linked using a short peptide linker of between 5 and 20 amino acids.

In some aspects, the antigen binding molecule may be linked to an effector molecule; for example, the antigen binding molecule that specifically binds B7H3 may be covalently linked to an effector molecule or to a toxin. The linkage can be by chemical or recombinant means (e.g., a peptide linker). Where the linkage is chemical, a reaction may have occurred to produce a covalent bond linking the antibody or fragment thereof with the effector molecule. The linkage may comprise a peptide linker comprising, for example, 1-50, 1-40, 1-30, 1-20, or preferably 1-10 amino acids. The antigen binding molecule, optionally linked to an effector molecule, may be further linked to a lipid, protein, polypeptide or carbohydrate that increases or preferably decreases its half-life in the body. The effector molecule may be selected from the group consisting of: an anti-cancer agent, a cytotoxic agent, a cytostatic agent, a drug, a radioisotope, a detectable label, an enzyme, a fluorophore, a fluorescent protein, a chemiluminescent agent, a radioactive label, a heavy metal, a tracer molecule, or any other detectable compound known to the skilled person. In preferred aspects, the antibody may be conjugated to an anti-cancer agent, a cytotoxic agent, or a cytostatic agent. In some aspects, the antigen binding molecule may be conjugated to pyrrolobenzodiazepine (PBD) or monomethyl auristatin E (MMAE). MMAE is a synthetic antineoplastic agent that has the following structure:

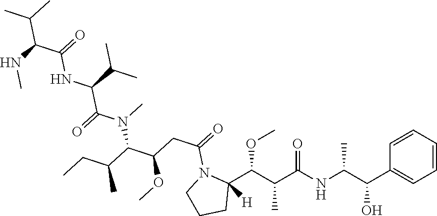 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

PBDs are of the general structure:

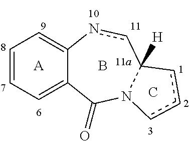

PBDs may differ in the number, type and position of substituents, in the aromatic A ring and the pyrrolo C ring, and in the degree of saturation of the C ring. In the B-ring there is either an imine (N═C), a carbinolamine (NH—CH(OH)), or a carbinolamine methyl ether (NH—CH(OMe)) at the N10-C11 position. PBDs typically have an(S)-configuration at the chiral C11a position, which provides a right-handed twist when viewed from the C ring towards the A ring. A number of naturally occurring PBDs have been identified, and over 10 synthetic routes have been developed to a variety of analogues (see, e.g., Thurston, et al., Chem. Rev. 1994, 433-465 (1994); Antonow, D. and Thurston, D. E., Chem. Rev. 2011 111 (4), 2815-2864). The term “PBD” may be understood to include PBD dimers.

Chimeric Antigen Receptors (CARs), Chimeric Co-Stimulatory Receptors (CCRs) and Cells Comprising them

The present invention provides a CAR which comprises an antigen binding molecule described herein which specifically binds to B7H3. CARs are engineered receptors, which are able to graft an arbitrary specificity onto an immune effector cell. In a classical CAR, the specificity of an antibody is grafted onto a T cell. CAR-encoding nucleic acids may be transferred to T cells using, for example, retroviral vectors. In this way, a large number of cancer-specific T cells can be generated for adoptive cell transfer.

CARs are modular and typically comprise an extracellular target antigen binding domain, a hinge region (or spacer), a transmembrane domain, and one or more intracellular signalling domains (or intracellular domains, or endodomains). The antigen-binding capability of the CAR is defined by the extracellular target antigen binding domain. Typically the extracellular target antigen binding domain is an scFv. The transmembrane domain anchors the CAR to the cell membrane, and the intracellular signalling domains transmit activation signals. The intracellular signalling domain may comprise one or more costimulatory domains. In some aspects, the CAR further comprises a hinge region, a transmembrane domain, and an intracellular signalling domain.

The extracellular target antigen binding domain of a CAR is commonly fused via a spacer (or hinge) and transmembrane domain to an intracellular signalling domain (or endodomain), which comprises or associates with an intracellular T cell signalling domain. When the CAR binds the target antigen, this results in the transmission of an activating signal to e.g. the T cell on which it is expressed. The hinge provides flexibility to the CAR (e.g. scFv) to access the targeted antigen. Longer hinges provide extra flexibility and allow for better access to membrane-proximal epitopes, whereas short hinges allow more effective binding of membrane-distal epitopes. A skilled person will appreciate that any suitable hinge or spacer sequence can be used. The hinge may be an IgG-based hinge derived from IgG1, IgG2, or IgG4. The hinge may be derived from native CD28 or CD8. The hinge or spacer sequence may, for example, comprise a short flexible linker, an IgG1 Fc region, an IgG1 hinge or a CD8 stalk, or a combination thereof. The linker may alternatively comprise an alternative linker sequence which has similar length and/or domain spacing properties as an IgG1 Fc region, an IgG1 hinge or a CD8 stalk. In some aspects, the hinge region of the CAR of the invention is derived from CD8.

The CAR may also comprise a transmembrane domain which spans the membrane. It may comprise a hydrophobic alpha helix. The transmembrane domain may be derived from CD4, CD8α (sometimes denoted as CD8 herein), or CD28, for example. The transmembrane domain may be an ICOS transmembrane. The transmembrane domain from the most membrane-proximal component of the intracellular signalling domain is often used, but different transmembrane domains can be used. In some aspects, the transmembrane domain of the CAR of the invention is derived from CD8 (sometimes denoted as CD8a herein) or CD28.

The CAR may comprise an intracellular signalling domain (or an endodomain) which is the portion of the CAR involved in signal transmission. The intracellular signalling domain either comprises or associates with an intracellular T cell signalling domain. After antigen recognition, receptors cluster and an activation signal is transmitted to the cell. The most commonly used T cell signalling component is that of CD3-zeta (or CD3ξ) which contains 3 ITAMs (Immunoreceptor tyrosine-based activation motifs). This transmits an activation signal to the T cell after antigen is bound. In some aspects, the intracellular signalling domain comprises a CD3-zeta. CD3-zeta alone may not provide a fully competent activation signal and additional co-stimulatory signalling may be needed. One or more co-stimulatory molecules may be employed. The co-stimulatory molecules may be from the CD28 family (including CD28 and ICOS) or the tumour necrosis factor receptor family (including 4-1BB, OX40, or CD27). A skilled person will appreciate that any suitable co-stimulatory domains can be used. Depending on the number of costimulatory domains, CARs can be classified into first (CD3ξ only), second (one costimulatory domain+CD3ξ), or third generation CARs (more than one costimulatory domain+CD3ξ). In some aspects, the intracellular signalling domain comprises a co-stimulatory domain. In some aspects, the intracellular signalling domain comprises a co-stimulatory domain derived from CD28 or 4-1BB.

The present invention further provides a chimeric costimulatory receptor (CCR) which comprises an antigen binding molecule described herein which specifically binds to B7H3. CCRs are similar to CARs, and typically comprise an extracellular target antigen binding domain, a hinge region (or spacer), a transmembrane domain, and one or more intracellular signalling domains (or intracellular domains, or endodomains). The antigen-binding capability of the CCR is defined by the extracellular target antigen binding domain. Typically the extracellular target antigen binding domain is an scFv. The transmembrane domain anchors the CCR to the cell membrane, and the intracellular signalling domains transmit activation signals. However, in contrast to a CAR, the intracellular signalling domain of a CCR is incapable of T cell signalling. That is, the intracellular signalling domain of a CCR does not confer signal 1 (the signal generated after interaction of an endogenous TCR with its ligand, i.e. a T-cell activating signal). Rather, the intracellular signalling domain of a CCR confers signal 2 (the signal generated by interaction between co-stimulatory molecules on an antigen presenting cell and cognate receptors on a T cell). In thus, a CCR provides signal 2 but not signal 1. A CCR provides co-stimulation but not TCR signalling. Accordingly, the intracellular signalling domain of a CCR lacks an intracellular T cell signalling domain, and does not associate with an intracellular T cell signalling domain. For instance, the intracellular signalling domain of a CCR may lack CD3-zeta, and may not associate with CD3-zeta. The intracellular signalling domain of a CCR may comprise or consist of one or more co-stimulatory domains. The one or more co-stimulatory domain may be any co-stimulatory domain known in the art. For instance, the intracellular signalling domain of a CCR may comprise or consist of (i) a co-stimulatory domain derived from CD28 and/or (ii) a co-stimulatory domain derived from 4-1BB. That is, the intracellular signalling domain of a CCR may comprise or consist of a CD28 co-stimulatory domain derived from and/or a 4-1BB co-stimulatory domain.

The extracellular target antigen binding domain of a CCR is commonly fused via a spacer (or hinge) and transmembrane domain to the intracellular signalling domain (or endodomain). When the CCR binds the target antigen, this results in the transmission of co-stimulatory signal to e.g. the T cell on which it is expressed. The hinge provides flexibility to the CCR (e.g. scFv) to access the targeted antigen. Longer hinges provide extra flexibility and allow for better access to membrane-proximal epitopes, whereas short hinges allow more effective binding of membrane-distal epitopes. A skilled person will appreciate that any suitable hinge or spacer sequence can be used. The hinge may be an IgG-based hinge derived from IgG1, IgG2, or IgG4. The hinge may be derived from native CD28 or CD8. The hinge or spacer sequence may, for example, comprise a short flexible linker, an IgG1 Fc region, an IgG1 hinge or a CD8 stalk, or a combination thereof. The linker may alternatively comprise an alternative linker sequence which has similar length and/or domain spacing properties as an IgG1 Fc region, an IgG1 hinge or a CD8 stalk. In some aspects, the hinge region of the CAR of the invention is derived from CD8.

The CCR may also comprise a transmembrane domain which spans the membrane. It may comprise a hydrophobic alpha helix. The transmembrane domain may be derived from CD4, CD8a (sometimes denoted as CD8 herein), or CD28, for example. The transmembrane domain may be an ICOS transmembrane. The transmembrane domain from the most membrane-proximal component of the intracellular signalling domain is often used, but different transmembrane domains can be used. In some aspects, the transmembrane domain of the CCR of the invention is derived from CD8 (sometimes denoted as CD8a herein) or CD28.

A CAR or CCR of the invention described herein may comprise an antigen binding molecule described herein which specifically binds to B7H3. In some aspects, the CAR or CCR of the invention comprises an extracellular target antigen binding domain which is an antigen binding molecule as described herein. All of the description of the antigen binding molecule of the invention may be applied directly to the CARs and CCRs described herein. Any of the features of the antigen binding molecule of the invention described herein applies to the CARs and CCRs described herein. Specifically, any of the antigen binding molecules described herein may be incorporated into the CARs or CCR described herein. The CAR or CCR comprises an extracellular target antigen binding domain that is the antigen binding molecule of the invention.

A CAR or CCR of the invention described herein may comprise a signal peptide such that when the CAR or CCR is expressed inside a cell, such as a T cell, the nascent protein is directed to the endoplasmic reticulum and subsequently to the cell surface, where it is expressed.

For example, the CAR or CCR of the invention may preferably comprise an antigen binding molecule of the invention that comprises a binding domain that specifically binds B7H3, and wherein the binding domain comprises a heavy chain variable domain and a light chain variable domain, wherein the heavy chain variable domain comprises a HCDR1 comprising the sequence of SEQ ID NO: 68, a HCDR2 comprising the sequence of SEQ ID NO: 70, and a HCDR3 comprising the sequence of SEQ ID NO: 72, and wherein the light chain variable domain comprises a LCDR1 comprising the sequence of SEQ ID NO: 76, a LCDR2 comprising the sequence of SEQ ID NO: 78, and a LCDR3 comprising the sequence of SEQ ID NO: 80; more preferably wherein the heavy chain variable domain comprises a sequence of SEQ ID NO: 66 and the light chain variable domain comprises a sequence of SEQ ID NO: 74. Preferably, the CAR of the invention may comprise the scFv described herein that has a heavy chain variable domain comprising a sequence of SEQ ID NO: 66 that is connected to a light chain variable domain comprising a sequence of SEQ ID NO: 74 via a linker having a sequence of SEQ ID NO: 273. Preferably, the CAR of the invention may comprise a hinge and transmembrane domain derived from CD8, an intracellular signalling domain comprising CD3-zeta, and also a co-stimulatory domain derived from CD28.

The present invention also provides a cell, preferably a T cell, which comprises the CAR described herein (i.e. a CAR-T cell). In some aspects, the T cell is an alpha-beta T cell. In some aspects, the T cell is a gamma-delta T cell. In some aspects, the cell can be a NK cell or an iPS cell. The present invention further provides a cell, preferably a T cell, which comprises the CCR described herein. In some aspects, the T cell is an alpha-beta T cell. In some aspects, the T cell is a gamma-delta T cell. In some aspects, the cell can be a NK cell or an iPS cell.

The T cell may be a T cell or T lymphocyte which are a type of lymphocyte that play a central role in cell-mediated immunity. They can be distinguished from other lymphocytes, such as B cells and natural killer cells (NK cells), by the presence of a T cell receptor (TCR) on the cell surface. There are various types of T cell, such as helper T cells, cytolytic T cells, memory T cells, and regulatory T cells. Any type of T cell may be used to produce a CAR-T cell or a T cell expressing a CCR.

T Helper T cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumour cells, and are also implicated in transplant rejection. CTLs express CD8 at their surface. These cells recognise their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described: naturally occurring Treg cells and adaptive Treg cells. Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX. Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner. NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation. NK cells may use the CAR constructs designed for CAR-T cells, and form CAR-NK cells. NK cells may use the CCR constructs designed for T cells, and form NK cells expressing the CCR.

The T cell (or NK cell) comprising a CAR or CCR of the invention may be made by:
(i) isolation of a T cell (or NK cell)-containing sample from a subject or from the other sources listed below; and
(ii) transduction or transfection of the T cell (or NK cell) with a nucleic acid sequence(s) encoding a CAR or CCR of the invention.

T cells (or NK cells) comprising or expressing a CAR or CCR according to the invention may be created ex vivo either from a patient's own peripheral blood (1st party, autologous treatment), or in the setting of a haematopoietic stem cell transplant from donor peripheral blood (2nd party), or from peripheral blood from an unconnected donor (3rd party, allogeneic treatment). T cells (or NK cells) expressing a CAR or CCR according to invention may also be derived from ex vivo differentiation of inducible progenitor cells or embryonic progenitor cells to T cells (or NK cells). An immortalised T-cell line which retains its lytic function and could act as a therapeutic may be used.

Leukocytes in the blood sample may be isolated using e.g. a blood cell separator (leukocyte apheresis). Peripheral blood mononuclear cells (PMBCs) may be separated and collected from the sample. The T cells (or NK cells) in the PMBCs may be activated and/or expanded prior to being transduced or transfected with a nucleic acid encoding a CAR or CCR according to the invention, for example by treatment with an anti-CD3 monoclonal antibody or IL-2.

The cell which comprises a CAR or CCR of the invention may be generated by introducing DNA or RNA coding for the CAR or CCR by one of many means including transduction with a viral vector or transfection with DNA or RNA. For example, the expanded T cells are purified and then transduced or transfected with a nucleic acid sequence encoding the CAR or CCR of the invention via e.g. a retroviral vector (e.g. an integrating gammaretrovirus (RV) or a lentiviral (LV) vector), or through use of the CRISPR/Cas9 system.

The T cells (or NK cells) may then by purified, for example, selected on the basis of expression of the CAR or CCR.

### Nucleic Acid Molecules, Vectors and Host Cells

A nucleic acid molecule comprising a nucleotide sequence encoding the antigen binding molecule or CAR according to invention is provided. A nucleic acid molecule comprising a nucleotide sequence encoding a heavy chain variable domain or a light chain variable domain described herein is also provided.

A nucleic acid molecule (or polynucleotide) may encode all or part of an antigen binding molecule of the invention. Thus, a nucleic acid molecule of the invention may encode “all”, i.e. the full length of, any antigen binding molecule, variant or fragment as described herein. “Part” of an antigen binding molecule typically means a heavy chain or a light chain, or a region of either thereof, such as the variable region. For example, “part” of an antigen binding molecule may mean a heavy chain variable domain or a light chain variable domain. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A nucleic acid molecule of the invention may be provided in isolated or purified form.

A polynucleotide sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such polynucleotide sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

In one aspect, a nucleic acid molecule of the invention comprises a nucleotide sequence which encodes a heavy chain variable domain or light chain variable domain sequence as described herein. Such a nucleic acid molecule may consist of or comprise a nucleotide sequence of SEQ ID NOs: 1, 17, 33, 49, 65, 81, 97, 113, 129, 145, 161, 177, 193, 209, 225, 241, or 257, or SEQ ID NOs: 9, 25, 41, 57, 73, 89, 105, 121, 137, 153, 169, 185, 201, 217, 233, 249 or 265, corresponding to the VHs and VLs respectively. A nucleic acid molecule of the invention may comprise or consist of both the nucleotide sequences of SEQ ID NOs: 1 and 9. A nucleic acid molecule of the invention may comprise or consist of both the nucleotide sequences of SEQ ID NOs: 17 and 25. A nucleic acid molecule of the invention may comprise or consist of both the nucleotide sequences of SEQ ID NOs: 33 and 41. A nucleic acid molecule of the invention may comprise or consist of both the nucleotide sequences of SEQ ID NOs: 49 and 57. A nucleic acid molecule of the invention may comprise or consist of both the nucleotide sequences of SEQ ID NOs: 65 and 73. A nucleic acid molecule of the invention may comprise or consist of both the nucleotide sequences of SEQ ID NOs: 81 and 89. A nucleic acid molecule of the invention may comprise or consist of both the nucleotide sequences of SEQ ID NOs: 97 and 105. A nucleic acid molecule of the invention may comprise or consist of both the nucleotide sequences of SEQ ID NOs: 113 and 121. A nucleic acid molecule of the invention may comprise or consist of both the nucleotide sequences of SEQ ID NOs: 129 and 137. A nucleic acid molecule of the invention may comprise or consist of both the nucleotide sequences of SEQ ID NOs: 145 and 153. A nucleic acid molecule of the invention may comprise or consist of both the nucleotide sequences of SEQ ID NOs: 161 and 169. A nucleic acid molecule of the invention may comprise or consist of both the nucleotide sequences of SEQ ID NOs: 177 and 185. A nucleic acid molecule of the invention may comprise or consist of both the nucleotide sequences of SEQ ID NOs: 193 and 201. A nucleic acid molecule of the invention may comprise or consist of both the nucleotide sequences of SEQ ID NOs: 209 and 217. A nucleic acid molecule of the invention may comprise or consist of both the nucleotide sequences of SEQ ID NOs: 225 and 233. A nucleic acid molecule of the invention may comprise or consist of both the nucleotide sequences of SEQ ID NOs: 241 and 249. A nucleic acid molecule of the invention may comprise or consist of both the nucleotide sequences of SEQ ID NOs: 257 and 265. A nucleic acid molecule of the invention may preferably encode an antigen binding molecule comprising the heavy chain variable domain amino acid sequence of SEQ ID NO: 66 and a light chain variable domain amino acid sequence of SEQ ID NO: 74, or a variant or fragment thereof as described above, for example. A nucleic acid molecule of the invention may comprise a nucleotide sequence encoding the CAR according to invention.

To create a scFv, the heavy chain variable domain and light chain variable domain-encoding DNA fragments may be operatively linked to another fragment encoding a flexible linker, e.g., encoding the amino acid sequence (Gly4-Ser)₃, such that the heavy chain variable domain and light chain variable domain sequences can be expressed as a contiguous single-chain protein, with the heavy chain variable domain and light chain variable domain joined by the flexible linker. Optionally, a cleavage site can be included in a linker, such as a furin cleavage site. The nucleic acid encoding the VH and/or the VL optionally encodes an Fc domain (immunoadhesin). The Fc domain can be an IgA, IgM or IgG Fc domain. The Fc domain can be an optimised Fc domain. In one example, the immunoadhesin is an IgG1 Fc.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 60, up to 70, up to 80, up to 90, or up to 100 or more nucleic acid substitutions and/or deletions from the sequences given in the sequence listing.

Suitable variants may be at least 70% homologous to a polynucleotide of any one of the nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Calculation of homology is also described in the preceding sections.

A variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

Polynucleotide “fragments” according to the invention may be made by truncation, e.g. by removal of one or more nucleotides from one or both ends of a polynucleotide. Up to 10, up to 20, up to 30, up to 40, up to 50, up to 60, up to 70, up to 80, up to 90, or up to 100 or more amino acids may be removed from the 3′ and/or 5′ end of the polynucleotide in this way. Fragments may also be generated by one or more internal deletions. Such fragments may be derived from a sequence as described herein or may be derived from a variant polynucleotide as described herein. Preferably such fragments are between 90 and 1000 residues in length, e.g. 90 to 300, 90 to 500, 100 to 800, 200 to 900 or 300 to 100 residues. Alternatively, fragments of the invention may be longer sequences, for example comprising at least 50%, at least 60%, at least 70%, at least 80% or at least 90% of a full length polynucleotide of the invention.

An antigen binding molecule of the invention may thus be produced from or delivered in the form of a polynucleotide which encodes, and is capable of expressing, it. Where the antibody comprises two or more chains, a polynucleotide of the invention may encode one or more antibody chains. For example, a polynucleotide of the invention may encode an antibody light chain variable domain, an antibody heavy chain variable domain, or both. Two polynucleotides may be provided, one of which encodes an antibody light chain variable domain and the other of which encodes the corresponding antibody heavy chain variable domain. Such a polynucleotide or pair of polynucleotides may be expressed together such that an antigen binding molecule of the invention is generated.

Polynucleotides of the invention can be synthesised according to methods well known in the art, as described by way of example in Sambrook et al (1989, Molecular Cloning—a laboratory manual; Cold Spring Harbor Press).

The nucleic acid molecules of the present invention may be provided in the form of an expression cassette which includes control sequences operably linked to the inserted sequence, thus allowing for expression of the antigen binding molecule of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus also provides an expression vector comprising the nucleic acid molecule described herein. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a polypeptide of the invention. Other suitable vectors would be apparent to persons skilled in the art. By way of further example in this regard we refer to Sambrook et al.

The present invention also provides host cells comprising the nucleic acid molecule or expression vector described herein. The cells are modified to express an antigen binding molecule of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for an antigen binding molecule of the invention include mammalian HEK293T, CHO, HeLa, NS0 and COS cells. Cell lines available as hosts for expression of antigen binding molecules are well known in the art. Preferably the cell line selected will be one which is not only stable, but also allows for mature glycosylation. Such cell lines of the invention may be cultured using routine methods to produce an antigen binding molecule of the invention.

### Antibody-Drug Conjugates (ADCs)

The present invention provides an antibody-drug conjugate (ADC) comprising the antigen binding molecule described herein linked to a drug. In some aspects, the drug is an anti-cancer agent, a cytotoxic agent or a cytostatic agent. In some aspects, the drug is selected from a pyrrolobenzodiazepine (PBD) and monomethyl auristatin E (MMAE). In some aspects, the drug is a fluorophore or tracer molecule.

An antibody-drug conjugate of formula (I) is also described herein:

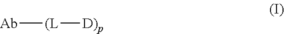

wherein Ab is an antigen binding molecule of the invention as described herein;
wherein L is a linker connecting Ab to D;
wherein D is an anti-cancer agent, a cytotoxic agent or a cytostatic agent; and
wherein p is preferably from 1 to 8.

In some aspects, L may be absent, or simply a covalent bond between the antibody (Ab) and the drug (D).

Antibody-drug conjugates (i.e., immunoconjugates) allow for targeted delivery of cytotoxic or cytostatic agents (i.e., drugs that kill or inhibit growth and division of cells, such as drugs useful in the treatment of cancer) to cells, such as cancer cells. The antigen binding molecule portion allows selective binding to the target tumour. The ADCs may then be internalised by the target cells, typically cancer cells, resulting in intracellular accumulation of the drug. ADCs may comprise a linker that connects the antibody and drug payload. Once the ADC is internalised by the target cell, the linker may then be cleaved, thus releasing the payload into the cytoplasm. Systemic administration of the unconjugated drugs typically results in unacceptable levels of toxicity to normal or non-target cells. Thus, the present invention provides antibody-drug conjugates that target delivery of drugs to B7H3-positive cells. The B7H3-positive cells may be cancer cells. The present invention therefore provides antibody-drug conjugates comprising an antigen binding molecule of the invention that specifically binds B7H3, as defined above, and a drug, wherein the drug is an anti-cancer agent, a cytotoxic agent or a cytostatic agent.

The antibody-drug conjugates described herein may have several advantageous features. For example, following administration to a subject in need thereof, the antibody-drug conjugates may be rapidly cleared from the subject's system, in order to minimise the residual toxicity. The Ab may be otherwise selected, modified, or engineered to have a short half-life in the subject of interest. The antibody-drug conjugates described herein are highly specific, targeted and potent agents.

### Antibodies (Ab)

In the antibody-drug conjugates described herein, Ab may be any antigen binding molecule of the invention, as described herein. All of the description of the antigen binding molecule of the invention may be applied directly to the Ab within the antibody-drug conjugates described herein. Any of the features of the antigen binding molecule of the invention described herein applies to the Ab of the antibody-drug conjugates described herein. Specifically any of the antigen binding molecule described herein may be incorporated into the antibody-drug conjugates described herein. The Ab of the antibody-drug conjugate of the invention is the antigen binding molecule of the invention.

For example, the Ab may be an antigen binding molecule of the invention that comprises a binding domain that specifically binds B7H3, and wherein the binding domain comprises a heavy chain variable domain and a light chain variable domain, wherein the heavy chain variable domain comprises a HCDR1 comprising the sequence of SEQ ID NO: 68, a HCDR2 comprising the sequence of SEQ ID NO: 70, and a HCDR3 comprising the sequence of SEQ ID NO: 72, and wherein the light chain variable domain comprises a LCDR1 comprising the sequence of SEQ ID NO: 76, a LCDR2 comprising the sequence of SEQ ID NO: 78, and a LCDR3 comprising the sequence of SEQ ID NO: 80; preferably wherein the heavy chain variable domain comprises a sequence of SEQ ID NO: 66 and the light chain variable domain comprises a sequence of SEQ ID NO: 74.

### Drug Units

The antibody-drug conjugates may comprise any antibody described herein (Ab) conjugated to (i.e., linked to or fused to) an anti-cancer agent, a cytotoxic agent or a cytostatic agent (D). Thus, D is an anti-cancer agent, a cytotoxic agent or a cytostatic agent. An anticancer agent (also called an antineoplastic agent) is any agent, small molecule or biologic, that is effective in the treatment of cancer. A cytotoxic agent is any agent that results in cell killing, preferably cancer cell killing, and tumour shrinkage. A cytostatic agent is any agent that inhibits, reduces or prevents cell growth or division, preferably of cancer cells, and inhibits tumour growth. D may be a known anti-cancer therapeutic with proven anti-cancer, cytotoxic or cytostatic properties.

The drug loading is the average number of drug units (D) per antibody (Ab), and is represented by p. The average number of drugs per antibody in preparations of antibody drug conjugates from conjugation reactions may be characterized by conventional means such as UV, reverse phase HPLC, HIC, mass spectroscopy, ELISA assay, and electrophoresis. In some instances, separation, purification, and characterization of homogeneous antibody-drug conjugates where p is a certain value from antibody-drug conjugates with other drug loadings may be achieved by means such as reverse phase HPLC or electrophoresis. Drug loading (p) is typically limited by the number of attachment sites for the drugs and linkers on the antibody. An attachment site may be understood to mean the site on the antibody at which a drug unit is attached usually via a linker. For example, the antibody may have 1, 2, 3, 4, 5, 6, 7 or 8 attachment sites to which the drug linker may be attached. In some aspects, the antibody has 1-8, 1-6, 1-4 or 1-2 such attachment sites, preferably 1-8 and most preferably 1-4 such attachment sites. Typically, fewer than the theoretical maximum of drug moieties are conjugated to an antibody during a conjugation reaction. The loading (drug/antibody ratio) of an ADC may be controlled in several different manners, including: (i) limiting the molar excess of drug-linker intermediate (D-L) or linker reagent relative to antibody, and (ii) limiting the conjugation reaction time or temperature. Antibody-drug conjugate compositions are described herein which include mixtures of antibody-drug conjugates where the antibody has one or more drug units attached and where the drug units may be attached to the antibody at various different sites, such as at different amino acid residues. The drug unit is typically attached to the antibody through a linker. Suitable linkers are described further herein. Suitable means for attaching or conjugating the antibody to the linker are also described further herein.

In some aspects, the average number of drug units (D) per antibody (Ab) in the antibody drug conjugates of the invention is in the range of 1 to 8. In some aspects, the range is selected from 1 to 4, 2 to 4, 1 to 3, 2 to 3, or 1 to 2, preferably 1 to 4. In some aspects, there are one or two drug units (D) per antibody (Ab) in the antibody drug conjugates of the invention. In some aspects p is 1 to 8, preferably 1 to 4. In some aspects p is about 2.

In some aspects, each D is independently selected from the group consisting of: an anti-cancer agent, a cytotoxic agent, a cytostatic agent, a drug, a radioisotope, a detectable label, an enzyme, a fluorophore, a fluorescent protein, a chemiluminescent agent, a radioactive label, a heavy metal or any other detectable compound known to the skilled person. In preferred aspects, D is an anti-cancer agent, a cytotoxic agent, or a cytostatic agent. In further preferred aspects, D is pyrrolobenzodiazepine (PBD) or monomethyl auristatin E (MMAE).

### Linkers

L is a linker connecting the antibody Ab to the drug D. L may be any linker suitable for connecting, covalently linking or conjugating the antibody Ab to the drug D. The linker L may be cleavable or non-cleavable. The linker L is preferably stable extracellularly. Thus, before transport or delivery into a cell, the antibody-drug conjugate of the invention is preferably stable and remains intact, i.e. the antibody Ab remains linked to the drug D. In some aspects, the linker L is stable outside the target cell (i.e., in an extracellular environment), but is cleaved inside the cell (i.e., in an intracellular environment), to release the drug D from the antibody Ab. Thus, the antibody Ab targets the anticancer, cytotoxic and/or cytostatic drug D to the target cells expressing B7H3. Typically the cleavage of the linker occurs at a fast enough rate to allow the drug to have an anti-cancer, cytotoxic or cytostatic effect on the target cell. The linker may be cleaved at any point following internalisation of the antibody drug conjugate by the target cell. In some aspects the linker may be cleaved preferentially in a particular intracellular compartment within the target cell. For example, the linker L may be preferentially cleaved within the lysozyme. An effective linker will: (i) maintain the specific binding properties of the antibody; (ii) allow intracellular delivery of the conjugate and/or drug; (iii) remain stable and intact, i.e. not cleaved, until the conjugate and/or drug has been delivered or transported to its target site; and (iv) maintain a cytotoxic, anti-cancer, cell-killing and/or cytostatic effect of the drug D. Stability of the antibody-drug conjugate may be measured by standard analytical techniques such as mass spectroscopy, HPLC, and the separation/analysis technique LC/MS.

The linker may be a non-cleavable linker, i.e., a linker that is not readily cleavable for example by enzyme activity, such as protease activity, or under specific conditions, such as acidic conditions. In some preferred aspects, L is a cleavable linker, i.e., a linker that is susceptible to cleavage when in the presence of a suitable cleavage moiety or under particular conditions. L may be selected from the group consisting of an acid-cleavable linker, a protease-cleavable linker, a disulfide linker, an enzyme cleavable linker, a pH-sensitive linker, a thiol-sensitive linker, or a reactive oxygen species sensitive linker. L may be any suitable linker that allows for targeted delivery of the drug unit to a B7H3-positive cell. Suitable linkers are described for example in Yang et al. Med Res Rev. 2020; 1-32, and the skilled person would be capable of selecting a suitable linker.

The conjugates of antibodies and cytotoxic agents may be prepared using any suitable methods as disclosed in the art, e.g., in “Bioconjugate Techniques”, G. T. Hermanson, 3rd Ed., Elsevier Inc., 2013. The linker may be conjugated to the antibody (Ab) using for example cleavable disulphide or non-cleavable thioether linker chemistry. The linker may be attached to a lysine residue in the antibody, the lysine may be native or engineered. The linker may be attached to a cysteine residue in the antibody. The cysteine may be native, for example a cysteine of one of the interchain disulphide bridges in the antibody, or the cysteine may be engineered, i.e., site-specifically inserted into the antibody sequence at the desired conjugation site. The linker may be attached to an unnatural amino acid (such as acetyl-phenylalanine, p-acetyl-L-phenylalanine (pAcF), selenocysteine, or para-azidomethyl-1-phenylalanine) in the antibody, for example by an oxime linkage. The antibody may be engineered to include an unnatural amino acid at the desired conjugation site. Chemoenzymatic site direct conjugation may also be used. For example, an azide group may be formed, for example at an asparagine residue in the antibody constant region, and linked with a drug unit using for example a copper-mediated click reaction. An azide group may be formed in a selective hydrolysis reaction mediated by an Endo-beta-N-acetylglucosaminidase (EndoS) chemoenzyme. Other strategies for site-specific conjugation of linkers and their attached drug units are known in the art and are extensively covered in G. T. Hermanson “Bioconjugate Techniques”, 2013, Elsevier Inc.

### Pharmaceutical Compositions, Methods and Uses

The present invention also provides pharmaceutical compositions comprising the antigen binding molecule, the CAR, the cell (e.g. T cell), or the ADC as described herein. Preferably, the pharmaceutical compositions further comprise a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Remington's Pharmaceutical Sciences, by E. W. Martin, Mack Publishing Co., Easton, PA, 19th Edition, 1995, describes compositions and formulations suitable for pharmaceutical delivery of the antigen binding molecule, the CAR, the cell comprising the CAR, or the ADC as described herein.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include aqueous dextrose, glycerol, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration. Sterile injectable solutions can be prepared by incorporating the active agent (e.g. antibody) in the required amount in an appropriate solvent with one or a combination of ingredients enumerated above, as required, followed by sterilization microfiltration. Pharmaceutical compositions of the invention may comprise additional active ingredients as well as an antigen binding molecule, CAR, cell comprising the CAR, or ADC of the invention. For example, the pharmaceutical compositions may further comprise additional therapeutic or prophylactic agents.

The present invention further provides a method of treating cancer, wherein the method comprises administering the antigen binding molecule, CAR, cell comprising the CAR, ADC, or the pharmaceutical composition described herein, to a subject in need thereof. An effective amount of the antigen binding molecule, CAR, cell comprising the CAR, ADC, or the pharmaceutical composition described herein may be administered. An “effective amount” refers to an amount effective, at dosages and for periods of time necessary, to achieve the desired therapeutic result. The present invention also provides the antigen binding molecule, CAR, cell comprising the CAR, ADC, or the pharmaceutical composition described herein for use in a method of treating cancer.

In some instances, the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC).

When a disease or disorder (e.g. cancer such as a solid tumour) is “treated” as discussed herein (for example in the methods or uses of the invention), this means that one or more symptoms of the disease or disorder (e.g. cancer such as a solid tumour) are ameliorated. It does not mean that the symptoms of the disease or disorder (e.g. cancer such as a solid tumour) are completely remedied so that they are no longer present in the patient, although in some methods, this may be the case. Thus, in all instances the term “treatment” or “treating” can be replaced with the term “amelioration” or “ameliorating”, respectively.

The methods or uses of the invention (such as the methods of treatment or treating) may result in one or more of the symptoms of the disease or disorder (e.g. cancer such as a solid tumour) being less severe than before treatment.

An antigen binding molecule, CAR, cell comprising the CAR, or ADC of the present invention, or a pharmaceutical composition comprising the antigen binding molecule, CAR, cell comprising the CAR, or ADC of the present invention, may be administered via one or more routes of administration using one or more of a variety of methods known in the art. As will be appreciated by the skilled artisan, the route and/or mode of administration will vary depending upon the desired results. Preferably, the antigen binding molecule, CAR, cell comprising the CAR, or ADC, or pharmaceutical compositions of the invention may be administered by parenteral administration. The phrase “parenteral administration” as used herein means modes of administration other than enteral and topical administration, usually by injection. Preferred routes of administration for an antigen binding molecule, CAR, cell comprising the CAR, ADC, or compositions of the invention include intravenous, intramuscular, intradermal, intraperitoneal, subcutaneous, spinal or other parenteral routes of administration, for example by injection or infusion. Alternatively, an antigen binding molecule, CAR, cell comprising the CAR, ADC, or pharmaceutical composition of the invention can be administered via a non-parenteral route, such as a topical, epidermal or mucosal route of administration. Local administration is also possible, including peritumoral, juxtatumoral, intratumoral, intralesional, perilesional, intra cavity infusion, intravesicle administration, and inhalation.

A suitable dosage of an antigen binding molecule, CAR, cell comprising the CAR, or ADC of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody employed, the route of administration, the time of administration, the rate of excretion of the antibody, the duration of the treatment, other drugs, compounds and/or materials used in combination with the particular compositions employed, the age, sex, weight, condition, general health and prior medical history of the patient being treated, and like factors well known in the medical arts.

A suitable dose of an antigen binding molecule, CAR, cell comprising the CAR, or ADC of the invention may be, for example, in the range of from about 100 ng/kg to about 25 mg/kg body weight of the patient to be treated per day. For example, a suitable dosage may be from about 1 μg/kg to about 10 mg/kg body weight per week, from about 100 μg/kg to about 10 mg/kg body weight per week or from about 10 μg/kg to about 5 mg/kg body weight per week. A suitable dosage may be from about 1 μg/kg to about 10 mg/kg body weight per day, from about 100 μg/kg to about 10 mg/kg body weight per day or from about 10 μg/kg to about 5 mg/kg body weight per day. In some aspects, 1×10⁶ or 5×10⁶ CAR T cells per kg may be administered.

Dosage regimens may be adjusted to provide the optimum desired response (e.g., a therapeutic response). For example, a single dose may be administered, several divided doses may be administered over time or the dose may be proportionally reduced or increased as indicated by the requirements of the therapeutic situation. It is especially advantageous to formulate compositions for parenteral administration in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic or conditioning effect in association with the required pharmaceutical carrier.

The antigen binding molecule, CAR, cell comprising the CAR, or ADC described herein may be administered in a single dose or in multiple doses. The multiple doses may be administered via the same or different routes and to the same or different locations. Alternatively, the antigen binding molecules can be administered as a sustained release formulation, in which case less frequent administration is required. Dosage and frequency may vary depending on the half-life of the antigen binding molecule in the patient and the duration of treatment that is desired.

The pharmaceutical composition may comprise any antigen binding molecule, CAR, cell comprising the CAR, or ADC of the invention as described herein. In some aspects, the pharmaceutical composition may comprise a single antigen binding molecule, CAR, cell comprising the CAR, or ADC of the invention. In some aspects, the pharmaceutical composition may comprise two or more different species of the antigen binding molecule, CAR, cell comprising the CAR, or ADC of the invention within the same composition. The present invention also provides for the concurrent administration of two different pharmaceutical compositions each comprising a single but different species of antigen binding molecules, CAR, cell comprising the CAR, or ADC of the invention.

### Methods of Detecting Cancer

The present invention also provides a method of detecting cancer in a subject, comprising: contacting a biological sample from the subject with the antigen binding molecule described herein and detecting antigen binding molecule bound to the sample, wherein binding of the antigen binding molecule to the sample indicates that the subject has cancer. In some instances, the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC). In some instances, the antigen binding molecule specifically binds to human B7H3, and wherein the binding of the antigen binding molecule indicates that the subject has cancer

The invention is illustrated by the following Examples:

### EXAMPLES

### Example 1—Materials and Methods

### Cells and Culture Conditions

The following cell lines were used in this study: Jurkat, 293T, CHO, MEXi 293E, 293F, LAN-1, Kelly, SupT1, K562. CHO were cultured in CHO culture medium (Gibco)+8 mM GlutaMAX (Gibco)+0.4 mM Hypoxnthine+0.32 mM Thymidine (Gibco). MEXi 293E Cells were cultured in MEXi Cultivation Medium (IBA)+50 mg/l geneticin and 8 mM GlutaMAX. 293F cells were cultured in Freestyle 293 Expression medium (Thermofisher). All three of these cells were cultured at 37° C. in 5% CO₂ in an orbital shaker. A temperature of 32° C. was used for protein production. 293T cells were grown in IMDM (Sigma) with 10% FCS (Gibco) and 100 U Penicillin/0.1 mg streptomycin/1. They were grown at 37° C. in 5% CO₂. The remainder of the cells were grown in RPMI (Sigma)+10% FCS 100 U Penicillin/0.1 mg streptomycin/l at 37° C. in 5% CO₂.

### γ-Retroviral Transduction

293T cells were plated at 1.5×10⁶, 24 hours prior to transfection. Cells were transduced using GeneJuice (Merck) with GOI (gene of interest) expression cassette and helper plasmids env (RD114), gagpol (PegPam-env). Supernatant containing retrovirus was harvested at 48 and 72 hours. For stable transduction, target cells were plated on 24 well plates coated in retronectin (Takara) and incubated with retroviral supernatant for 72 hours.

### Production of the B7-H3 Phage Display Library

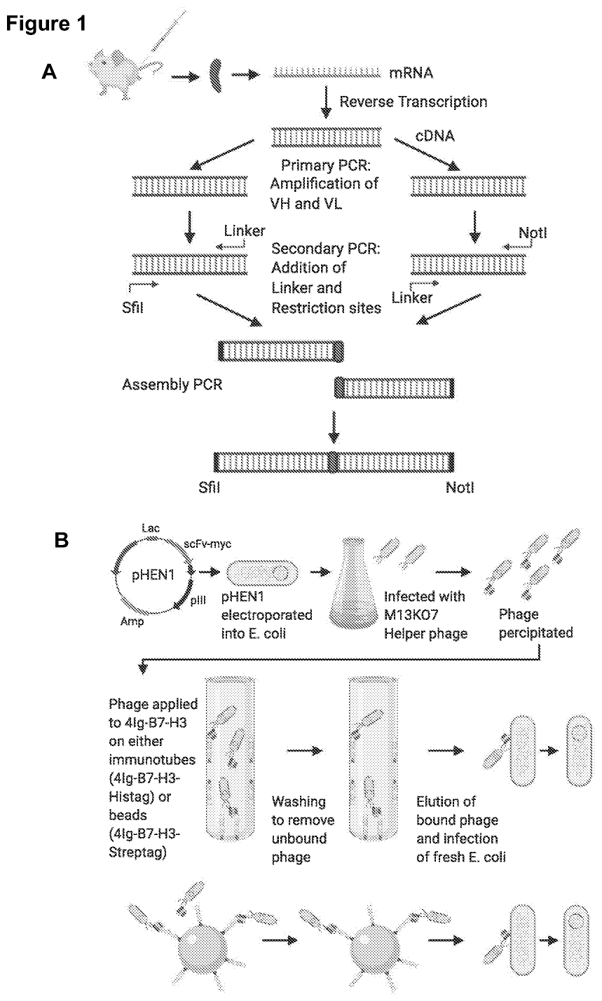

Jurkat cells were stably transduced using γ-retroviral transduction to produce recombinant B7-H3-mouseFc fusion protein. Protein was produced in a bioreactor and purified on protein A columns. 3 BALB/cJ mice were injected with recombinant protein (FIG. 1A). Serial serum extractions confirmed seroconversion by flow cytometry. Splenic mRNA was extracted using RNeasy Mini Kit (QIAGEN). mRNA was reverse transcribed (Superscript III Reverse Transcriptase, Invitrogen) then amplified via PCR (Amplitaq Polymerase, Applied Biosystems). Further PCR reactions were used to connect heavy and light chain DNA with a serine glycine linker. Amplified DNA was first cloned into an intermediary pSP73 vector before being cloned into the pHEN vector. E. coli were transduced using electroporation (FIG. 1).

### Panning of the B7-H3 Library

4Ig-B7-H3 cDNA was purchased (Sinobiological) and cloned into 2 vectors to produce B7-H3-Histag and B7-H3-Streptag. CHO cells and MEXi293E cells respectively were transiently transduced with these constructs. Cells were cultured until there was a drop in their viability and protein filtered from cell supernatant using HiTrap MabSelect Protein-A Columns (Cytiva) or Strep-Tactin XT: Twin Strep-tag purification columns (IBA).

2TY media was inoculated with E. coli. Bacteria were cultured until the OD was 0.5. Bacteria were infected with the M13KO7 Helper Phage (New England Biolabs) and incubated overnight. Bacteria were removed by centrifugation and phage particles in the supernatant precipitated with PEG 600/2.5M NaCl. After washing, the phage were re-suspended in sterile water.

Immunotubes were incubated with B7-H3-Histag and MagStrep “type 3” XT beads (IBA) were incubated with B7-H3-Streptag overnight at 4° C. to coat tubes/beads. Tubes/beads were washed ×3 in PBS and blocked by incubating with Marvel Milk for 2 hours at room temperature. Precipitated phage was blocked with Marvel Milk separately. Tubes/beads were washed ×3 with PBS. Blocked phage was applied to coated and blocked tubes and incubated for 2 hours at room temperature. Tubes/beads were washed, and bound phage was eluted with 1 ml 100 μM Triethylamine. Eluted phage was incubated with TG1 E. coli with an O.D. 0.5 for 40 minutes. Bacteria were pelleted and plated on agar plates (FIG. 1B)

Selection of Anti-B7-H3 scFv

Selected colonies from panned libraries were grown in 96 well plates. Positive binders were identified using ELISA against immobilized recombinant B7-H3. Bound scFv-myc was detected with anti-myc (Sigma) followed by an anti-Rabbit HRP (Sigma). Selected binders were cloned into scFv-Fc format in a pcDNA3.1 expression vector. 293F Cells were transiently transfected with PEI (Sigma). Cells were cultured until viability dropped and supernatant harvested. Protein was purified on HiTrap MabSelect Protein-A Columns (Cytiva). Purified, diluted protein was used to stain cell bound B7-H3 on Jurkats and the binding analyzed using flow cytometry.

### Production of B7-H3 Positive Jurkat Cells

A truncated B7-H3 (T-B7-H3) in an SFG γ-retroviral expression cassette was used. A 4Ig-B7-H3 isoform of B7-H3 was purchased (Sinobiological) and cloned into a γ-retroviral expression cassette. 4Ig-B7-H3 was digested to produce 2Ig-B7-H3. Retroviral transduction was used to stably transduce Jurkat cells with each of these three isoforms of B7-H3.

### PBMC and T Cell Isolation

Leukapheresis cones were acquired from NHS blood and transplant. PBMCs were separated through ficoll centrifugation using Lymphoprep (Stemcell Technologies). PBMCs were washed and residual red cells lysed with ACK Lysis buffer (Thermofisher). NK Cells were depleted using magnetic CD56 depletion beads (Miltenyi Biotec) and LD depletion columns (Miltenyi Biotec).

### Generation of CAR-T Constructs

Geneblocks for each of the anti-B7-H3 CAR T cells were designed and cloned into a previously described expression vector scFv-CH2-CH3-CD28-CD3z γ-retroviral CAR expression cassette with an RQR8 marker gene for selection/elimination (Philip et al., 2014), using restriction sites at the 3′prime and 5′prime ends of the CAR (Thermofisher). These geneblocks included TE9-CD8H/Tm-CD28-CD3z, TC6-CD8H/Tm-CD28-CD3z, TF9-CD8H/Tm-CD28-CD3z, BF9-CD8H/Tm-CD28-CD3z, BH6-CD8H/Tm-CD28-CD3z, TE9-CD8H/Tm-4-1BB-CD3z, TE9-CD28H/Tm-CD28-CD3z, TE9-CD8H/Tm-CD28-ILR2-CD3z.

### CAR T Cell Transduction

PBMCs were suspended in RPMI containing FCS and L-Glutamine at a concentration of 1×10⁶ cells/ml. They were activated with 0.5 μg/ml of anti-CD3 (Miltenyi Biotec) and anti-CD28 antibodies (Miltenyi Biotec). 48 hrs before transduction and on the day of transduction, 100 IU/ml recombinant human IL-2 (Proleukin, Novartis) was added. T cells were transduced using γ-retroviral transduction.

### T Cell Functional Assays

In co-culture assays with CAR-T cells, targets were LAN-1, Kelly, K562 or AML target cells (MV411, NOMO1, THP1) or no antigen stimulus in 48 well plates at an effector:target ratio of 2:1. In most experiments control target cells were Jurkat or SupT1 cells transduced stably to express target antigen 4×Ig human B7H3. For the 18-hour co-culture assay, CAR T cells were co-cultured with targets. After 18 hours, supernatant was removed for ELISA and cells incubated with monensin (BioLegend); activation markers CD69 and CD25, and the degranulation marker CD107a, were detected by flow cytometry. For the 7-day co-culture, CAR T cells were labelled with CSFE or cell trace violet co-cultured with LAN-1, Kelly, K562 or AML target cells (MV411, NOMO1, THP1) or no antigen target in a 24 well plate for 6 days at an effector:target ratio of 2:1. On the 6th day, plates were centrifuged to pellet cells, 1 ml media was removed and 1 ml added containing fresh target cells. After a further 24 hours, the supernatant was removed for ELISA and the cells pelleted and the levels of exhaustion markers Tim3, Lag-3 and PD-1 and proliferation as measured by CSFE dilution were examined using flow cytometry. To evaluate the proliferative capacity of the IL-2RB-modified CAR construct, CAR T cells were labelled with CellTrace Violet (ThermoFisher) and co-cultured with wild-type Jurkats, B7-H3-expressing Jurkats, or no target cells for 6 days at an effector:target ratio of 1:1 in 48-well plates. Cells were plated with either no cytokine, 70 ng/mL IL-15 (PeproTech), or 100 IU/mL IL-2 (Proleukin, Novartis), respectively, and were fed with fresh target cells on days 2 and 4 of co-culture. Cell proliferation and fold expansion was evaluated on the 6th day by flow cytometric analysis using Precision Count Beads (BioLegend). For the 28-day co-culture assay, CAR T cells were co-cultured with irradiated LAN-1, Kelly or no target cells in 24 well plates at an effector:target ratio of 2:1. Cell media was replenished every 2-3 days. CAR T cells were challenged with irradiated target cells every 6 days, cultured for a further 24 h and analyzed. Cells were pelleted and supernatant was removed every week for ELISA. CAR-T cell proliferation was measured weekly by flow cytometry using Precision Count Beads (BioLegend). The levels of cytokines IL-2 and IFN-γ were quantified using ELISA MAX Deluxe Set Human IL-2 and ELISA MAX Deluxe Set Human IFN-γ (BioLegend). Cytotoxicity was tested using a Cr⁵¹ release cytotoxicity assay. Target cells were incubated with Cr⁵¹ for 1 hour then washed and plated in 96 well plates.

CAR T cells or untransduced cells were plated at effector:target ratios of 10:1, 5:1, 2.5:1, and 1.25:1. The plates were incubated for 4 hours at 37° C. then the supernatant removed and incubated with scintillation fluid (Perkin Elmer) overnight at room temperature. Cr⁵¹ released into the supernatant was measured using a 1450 MicroBeta TriLux (Perkin Elmer). Activity of CAR T cells against decreasing concentrations of B7-H3 protein was measured using a plate-based assay. ELISA plates were coated in decreasing concentrations of recombinant B7-H3 and incubated overnight at 4° C. Plates were washed and CAR T cells or untransduced cells added. Plates were incubated overnight at 37° C., cells were pelleted, and the supernatant removed for use in ELISA.

### Haemopoietic Colony Assays

Clonogenic assay [also known as colony forming cell (CFC) assay, colony forming unit (CFU) assay and methylcellulose assay] is an in vitro assay used in the study of hematopoietic stem cells. The assay is based on the ability of individual hematopoietic progenitor cells called colony-forming units (CFU) to proliferate and differentiate into colonies in a semi-solid media in response to cytokine stimulation. The colonies formed can be enumerated and characterised according to their unique morphology. The assay was used to investigate colony formation from cord blood (CB) and NOMO-1 leukemic cells after they were treated with the TE9-CD8-28ξ CAR T cells. Untransduced T cells were used as control.

H4434 Classic Methocult media (STEMCELL Technologies) was used in this assay as semi-solid matrix: it contains rh SCF (stem cell factor), rh GM-CSF (Granulocyte macrophage colony-stimulating factor), rh IL-3, rh EPO and allows the growth of CFU-E (Erythroid progenitor cells), BFU-E (burst-forming unit-erythroid), CFU-GM (Granulocyte and/or macrophage progenitor cells) and CFU-GEMM (multi-potential progenitor cells) in CB as well as leukemic colonies.

Prior to the experiment, Methocult media was aliquoted in Sterilin™ 7 ml Bijou (Thermo Scientific) with 2 ml/aliquot and stored at −20° C. Briefly, effector cells (untransduced T cells and TE9-CD8-282 CAR T) and target cells (CB and NOMO-1) were co-cultured in a tissue culture treated 48 well plate at 5:1 E:T ratio for 18 hours at 37° C.

Post incubation, the cells in each co-culture condition was collected separately and washed in Iscove's MDM with 2% FBS (STEMCELL Technologies) which is the recommended media for preparing and washing samples for CFU assay. Cells were then resuspended again in Iscove's media at the desired concentration calculated estimating to have 20,000 cells of CB in 40 μl of cell suspension and 2,000 cells of NOMO-1 cells in 40 μl of the cell suspension, if any lysis did not occur.

40 μl of cell suspension was then transferred to one 2 ml Methocult aliquot and vigorously shaken in order to distribute evenly the cells in the Methocult. 1 ml of Methocult containing the desired number of cells (10,000 for CB and 1,000 for NOMO-1) were then plated in a well of a tissue culture treated 6-well plate using a 16 Gauge Blunt-End Needle (STEMCELL Technologies).

These seeding densities were chosen in order for NOMO-1 and CB conditions to be as comparable as possible, with the approximation that only 1% of cells from the CB are hemopoietic progenitors colony forming units. Other densities were tested but they either were crowding the well too much or did not produce enough colonies to be quantified properly (data not shown).

The plates were surrounded by PBS to maintain high humidity levels and prevent drying up of the Methocult and they were incubated at 37° C. for 14 days. Numbers and morphology of the colonies were assessed by microscopy on day 14 and then, in order for the colonies to show up on a photograph, the colonies were stained dark purple with p-iodonitrotetrazolium violet (Sigma).

### Antibodies and Flow Cytometry Analysis

The following antibodies were used in this study: anti-B7-H3 (FM276, Miltenyi Biotech), anti-GD2 (14.G2a, BD Biosciences), Human Ig (Polyclonal, Thermofisher), anti-mouse IgG (polyclonal, R&D), anti-CD3 (UCHT1, BioLegend), anti-HisTag (J095G45, BioLegend), anti-CD34 (QBEnd10, R&D), anti-αβ-TCR (IP26, BioLegend), anti-CD107a (H4A3, BioLegend), anti-cD25 (BC96, BioLegend), anti-CD69 (FN50, BioLegend), anti-Tim3 (F38-2E2, BioLegend), anti-Lag3 (11C3C65, BioLegend), anti-PD-1 (EH12.1, BD Biosciences), anti-Mouse CD45 (30-F11, BioLegend), anti-Human CD45 (HI30, BioLegend), Ghost Red™ 780 (Tonbo Biosciences), Zombie Yellow Viability Dye (BioLegend), Propidium Iodide (Gibco), Cell Trace Violet (ThermoFisher), Precision Count Beads (BioLegend).

### Cross Reactivity of TC6, TE9 and BH6 Whole Antibodies

TC6, TF9 and BH6 were produced as chimeric antibodies with a human IgG1 Fc domain. Antibodies were purified on protein A columns (Cytiva) and were tested in ELISA against plate bound antigen, with detection using goat anti-human IgG (H+L) (SeraCare). Cross reactivity against mouse B7-H3 was tested using flow cytometry against the mouse cell line 3T3/NA1.

### In Vivo LAN-1 Neuroblastoma and Med8A Medulloblastoma Models

Animal protocols were approved by local institutional research committees and in accordance with UK Home Office guidelines. Male NSG mice aged between 6 and 8 weeks were supplied by UCL. All experiments were carried out under UK home office licenses project license number 15981/01, personal license number 12972. For the Neuroblastoma LAN-1 experiments, NSG mice were injected with 1×10⁶ LAN-1-BFP/Luc in Geltrex™ (Thermofisher) subcutaneously into the flank. 1×10⁶ CAR T cells were injected intravenously into the tail vain at day 10. Tumour size was monitored twice a week with digital calipers. Mice were given 200 μl luciferin into the scruff and imaged using a PhotonIMAGER™ optical imaging system (Biospace Lab) weekly. When tumours reached threshold size, mice were sacrificed and blood, spleen and tumour samples taken. Cells were disaggregated using a cell strainer and residual red blood cells removed using ACK Lysis buffer (ThermoFisher). Cells were stained and markers analysed using flow cytometry. For the Med8A medulloblastoma experiments, 1×10⁶ med8A medulloblastoma cells, stably transduced with luciferase were stereotactically implanted in 3-5 microlitre volume into the hemisphere. 48 hours later 5×10⁶ CAR-T cells or non-transduced control were injected into the lateral ventricle. Tumour growth was evaluated by bioluminescence imaging.

### Gamma Delta T Cell Expansion

PBMC were isolated from purchased whole blood leucocyte cones via density gradient centrifugation using Lymphoprep (Stemcell) according to manufacturer's instruction. PBMC were either cryopreserved in 90% FBS 10% DMSO or re-suspended in complete T cell culture media for further processing. Complete T cell culture media consisted of xeno- and serum-free CTS-OpTmizer (Thermo Fisher) with 10% synthetic serum replacement (Thermo Fisher) and GlutaMAX (Thermo Fisher), all of which are available to research as well as GMP-grade from Thermo Fisher with the following product catalogue numbers: research-grade CTS-OpTmizer (A1048501) and GMP-compatible alternative GMP-grade OpTmizer-CTS (A3705003), synthetic immune cell serum replacement that is compatible with both manufacturing standards (A2596101) and GlutaMAX also compatible with both standards (35050061). If starting with cryopreserved material, PBMC were thawed and rested at 10×10⁶ cells/mL in complete pre-warmed media overnight before further processing to avoid over-stressing the lymphocytes and to enhance depletion quality. PBMC at 2-4×10⁶ cells/mL density were then either stimulated in standard cell culture plates right away or first depleted of αβ T cells using the TCRα/β Product Line (Miltenyi Biotec) according to manufacturer's instructions concurrently with depletion of CD56-positive cells using CD56 MicroBeads (Miltenyi Biotec) according to manufacturer's instructions. Briefly, cells were first labelled with anti-TCRα/β-biotin, then a mix of anti-biotin microbeads and anti-CD56 beads, and then depleted using MACS Cell Separation LD Columns (Miltenyi Biotec). If cultured in G-Rex vessels (Wilson Wolf), depleted PBMC were initiated at 2-4×10⁶ cells/cm². Thus-prepared PBMC were stimulated with either 1 μg/mL OKT-3 (Miltenyi Biotec Cat #130-093-387, RRID: AB_1036144) or 1 μg/mL PHA (Merck) and various cytokine combinations: (i) 100 IU/mL IL-2 aldesleukin (Proleukin; Novartis), (ii) 70 ng/ml IL-15 (Peprotech), (iii) 20 ng/mL rhIL-7 (Peprotech), or the (iv) ‘DOT protocol’ cytokine cocktail, which consisted of a first culture in 100 ng/mL rIL-4, 70 ng/ml rIFN-γ, 7 ng/ml rIL-21 and 15 ng/ml rIL-1ß followed by a second culture in 70 ng/mL rIL-15 and 30 ng/mL IFN-γ (all from Peprotech). When comparing the full ‘DOT protocol’ to test expansion protocols, the methodology described by Almeida et al. (2016) was used, albeit with the omission of a positive selection step using OKT-3 following the alpha beta TCR depletion. Briefly, depleted PBMC were stimulated for a first cytokine culture with 70 ng/mL OKT-3, and then a second cytokine culture with 1 μg/mL OKT-3. Live cells before and during expansion were counted using Trypan Blue exclusion, an automatic cell counter (Invitrogen) and flow cytometry-based Precision Count Beads (Biolegend).

Vδ2 γδT cells were depleted from PBMC at one of three stages of expansion: pre-initiation, at midway split or at harvest. All depletions were done using anti-TCR/Vδ2 mAb clone B6 (BioLegend Cat #331404, RRID: AB_1089228) at a concentration of 0.5 μg/10⁶ PBMC. When depleting at initiation Vδ2 cell initiation was incorporated into the αβ TCR/CD56 depletion process. This was done as follows: PBMC were co-incubated with αβ TCR-biotin mAb and Vδ2 (clone: B6)-biotin mAb, washed, and then co-incubated with anti-biotin and anti-CD56 microbeads according to manufacturer's protocol, then washed and depleted using Miltenyi LD magnetic column separation, as above and according to manufacturer's protocol. If depleting at midway split or final harvest, expanding cells were harvested, washed and labelled with 0.5 μg clone B6/10⁶ PBMC, incubated for 20 min, washed and incubated and depleted using Miltenyi anti-biotin microbeads and LD columns.

Gamma Delta T Cell Viral Transduction with CAR-T

293T cells (ATCC Cat #CRL-3216, RRID: CVCL_0063) were plated at 1.5×10⁶ cells per 10 cm² plate (Corning) in 10 mL 10% foetal bovine serum (FBS)-supplemented Gibco IMDM (Thermo Fisher). At 70% confluence, 293T cells were transfected using GeneJuice (Merck) according to manufacturer's protocol. Triple plasmid transient transfection was carried out using SFG-gammaretroviral vectors (RRID: Addgene_22493), including the B7H3-CAR, gag+pol (RRID: Addgene_8449) and RD114 envelope (RRID: Addgene_17576) plasmids at equimolar ratio. Retroviral supernatant was harvested at 48 and 72 hours following transfection and used immediately for T cell transduction. Briefly, non-tissue culture treated 24 well plates (Costar) were coated with RetroNectin (Takara) in PBS (final concentration of 1 mg/mL) and incubated at 4° C. for 24 hours. The retronectin was removed and 1.5 mL of retroviral supernatant was added to each retronectin coated well. Following this, 3×10⁵ stimulated T cells in 500 μL was added and plates were centrifuged at 1000×g for 40 minutes, at room temperature before incubation in complete T cell culture media at 37° C., supplemented with IL-15 to a final concentration of 70 ng/ml (˜140 IU/mL). Transduced T cells were harvested after three days, washed and re-suspended for expansion in specified cytokine-supplemented complete T cell culture medium. Transduction efficiency was assessed by flow cytometric detection of the CD34 marker gene.

### Statistical Analysis

All statistical analyses were performed in GraphPad Prism v8. Unless otherwise stated, data is expressed as mean±range. Statistical analyses of in vitro assays were undertaken by 1 way ANOVA with Tukey multiple comparisons, except for the Cr⁵¹ cytotoxicity assay where a 2-way ANOVA was used. For the in vivo analysis, tumour size and ROI were compared using the Kruskal-Wallis test and survival analysed by the log-rank (Mantle-Cox) test. *** p<0.0001, ** p<0.001 and *p<0.01.

### Example 2—Development of Novel Anti-B7-H3 Antibodies in Single Chain Format

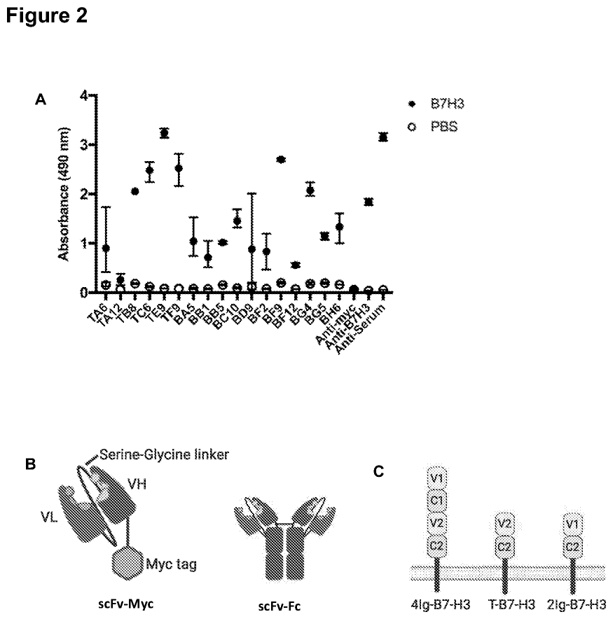

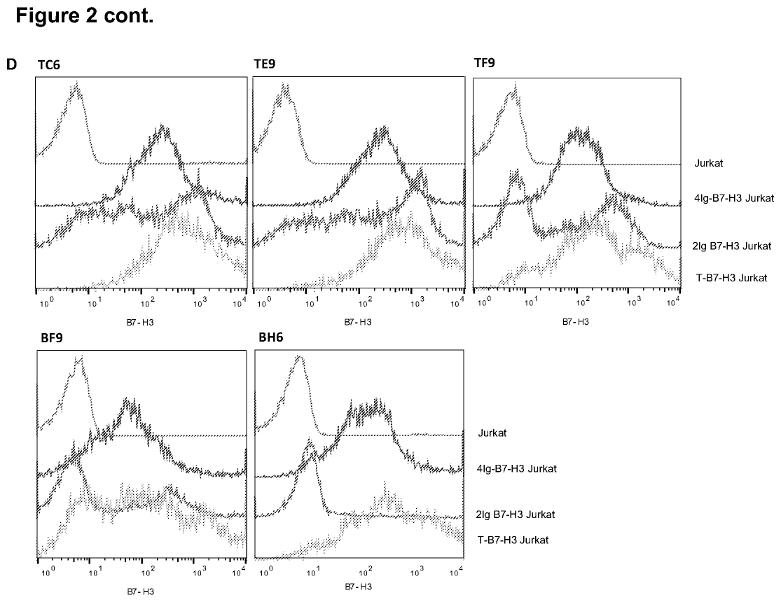

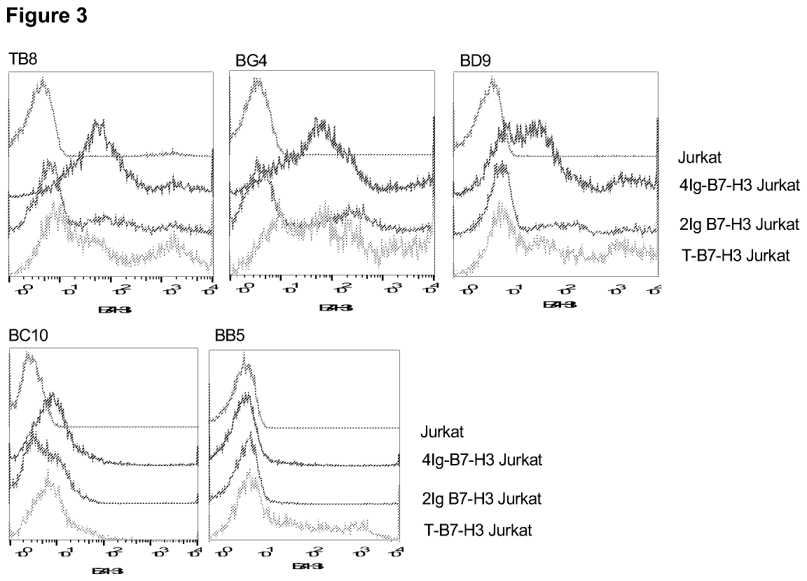

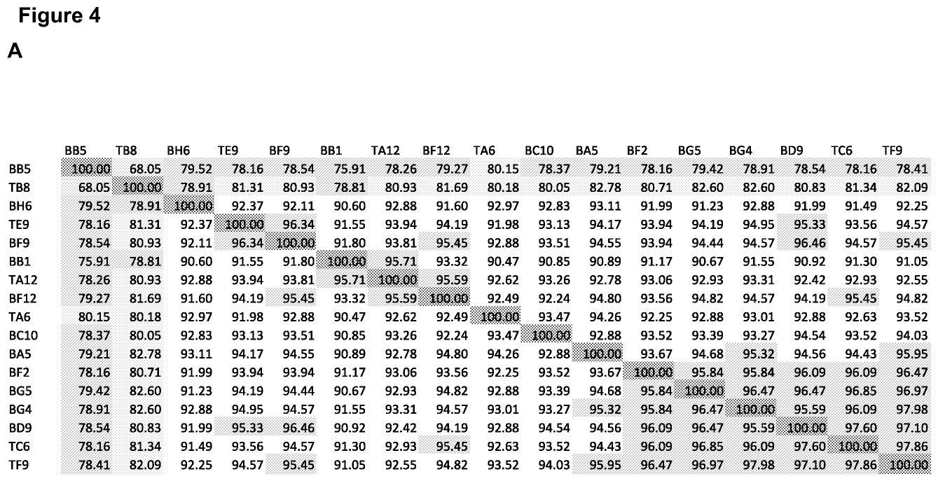

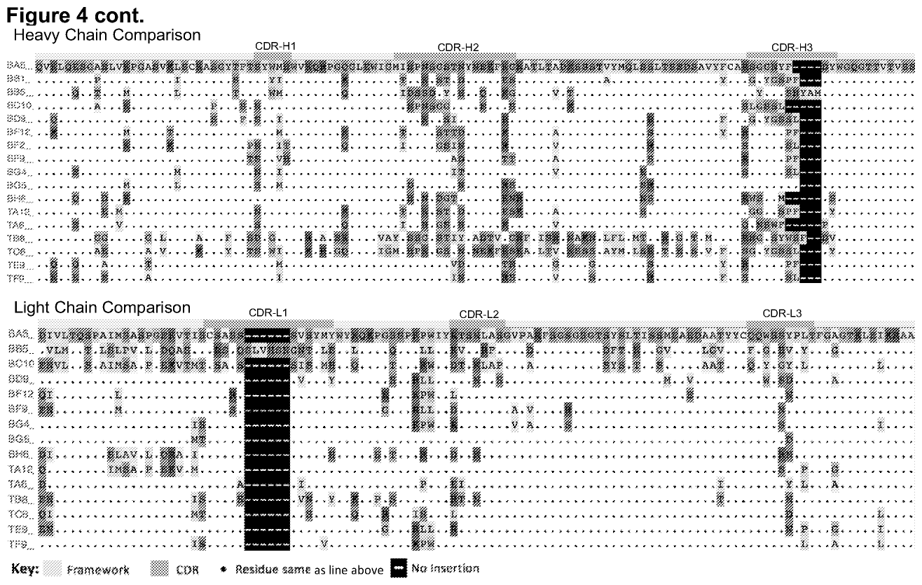

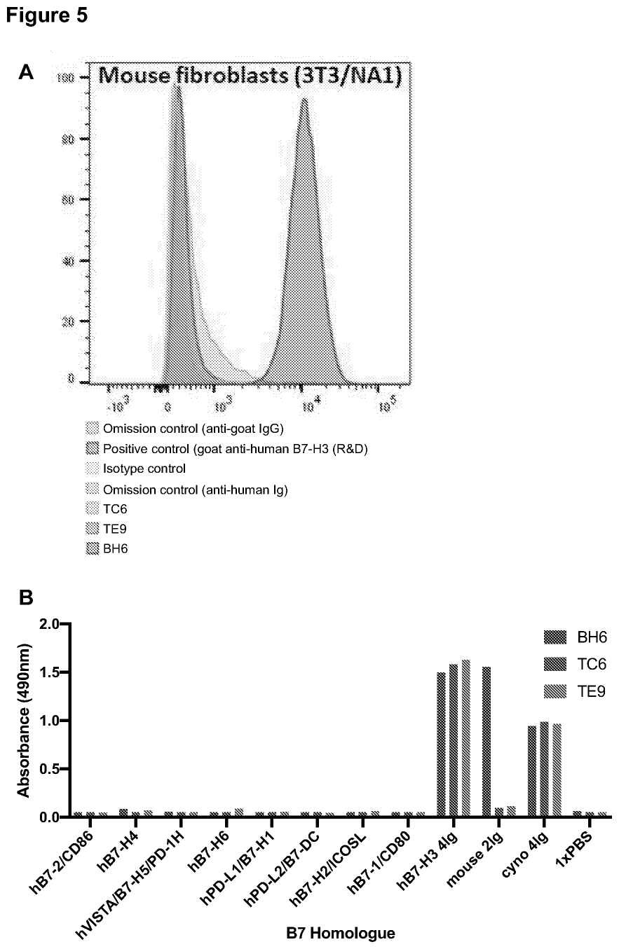

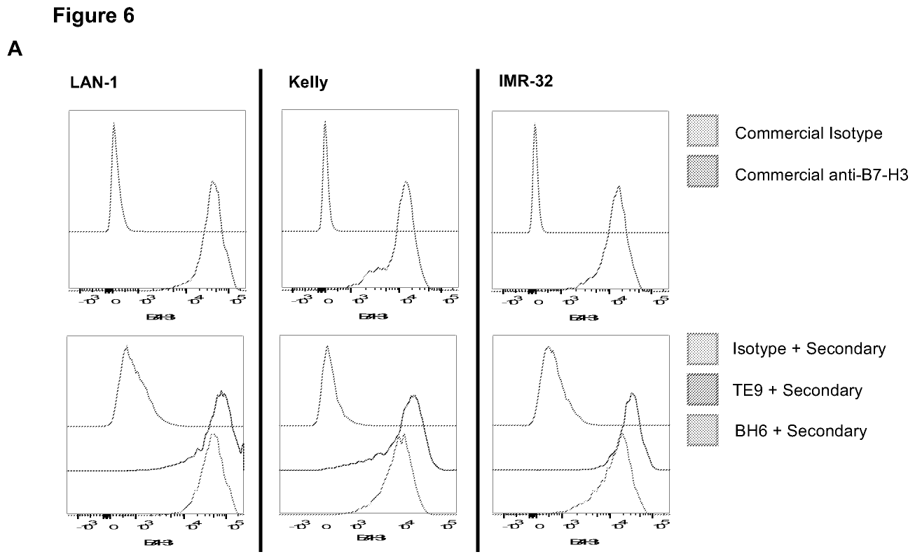

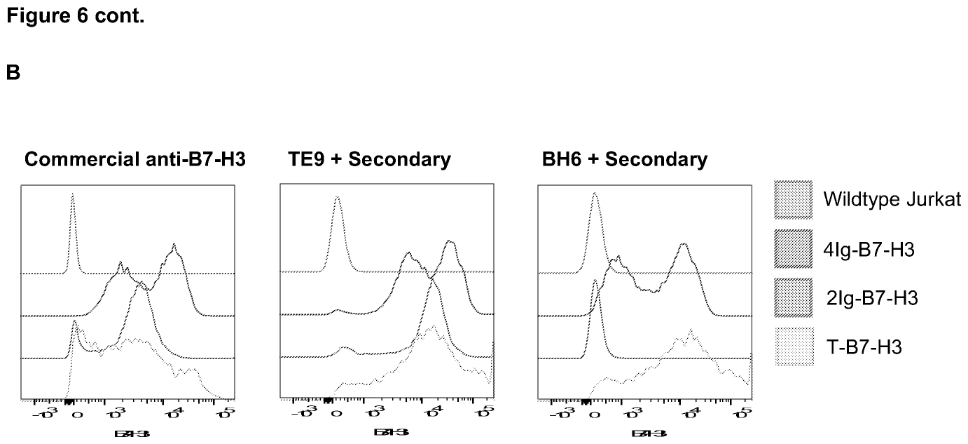

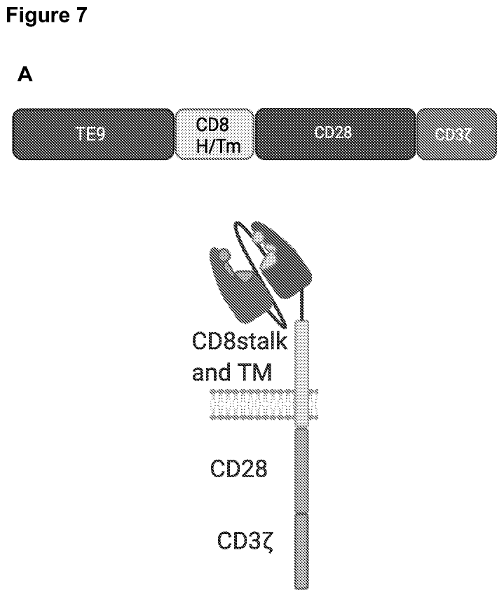

The targeting of B7-H3 cancer antigen by T cells engineered to express CARs has shown great promise in preclinical models and is being translated into clinical studies. Thus far, most studies have used repurposed antibodies in which scFv have been derived from existing monoclonal antibodies. To generate novel B7-H3 binders potentially more finely tuned for CAR-T applications, mice were immunised with recombinant B7H3-Fc fusion protein and spleen RNA from immunised mice served as substrates for generation of an ScFv library in bacteriophage (FIG. 1A). Individual scFvs were derived from phage library by panning with human B7-H3, and were screened as CAR-T binding elements by cloning directly into CAR-T format for empirical comparison of CAR-T effector function (FIG. 1B). 17 binders were identified by ELISA screen (FIG. 2). Ten scFv were selected for production in scFv-Fc format based on the ELISA and genetic heterogeneity of the clones. Of 10 scFv-Fc fusion proteins, five (TE9, TC6, BH6, TF9 and BF9) were selected for further evaluation in CAR-T format based on strength and specificity of binding to B7-H3 isoforms (isoform 1, isoform 2 or the artificially truncated isoform T-B7-H3 that had been used as the immunogen) by flow cytometry (FIG. 2). Four of the anti-B7-H3 binders (TE9, TC6, TF9 and BF9) bound to both human isoforms of B7-H3 but BH6 showed specificity for 4Ig-B7-H3 (FIG. 2D). Five other binders (TB8, BG4, BD9, BC10, BB5) showed weaker overall binding and lack of reactivity against physiological target isoforms 4Ig B7H3 or 2Ig B7H3 (FIG. 3). All binders from the library selected on the original ELISA were sequenced and evaluated for degree of sequence similarity between each other, disclosing a high degree of variance between the selected binders (FIG. 4). The binders, TE9, TC6 and BH6 were produced in whole antibody format. These antibodies showed specific binding against B7-H3 but not against other members of the human B7 family in ELISA assays. BH6 bound both human and mouse B7-H3 but TE9 and TC6 were specific for human and cynomolgus monkey (FIG. 5) and showed similar antigenic specificities to neuroblastoma and synthetic cell lines as commercial anti-B7-H3 monoclonal antibody (FIG. 6).

### Example 3-B7—H3 Binders in CAR-T Format Display a Range of Antigen-Specific Effector Function

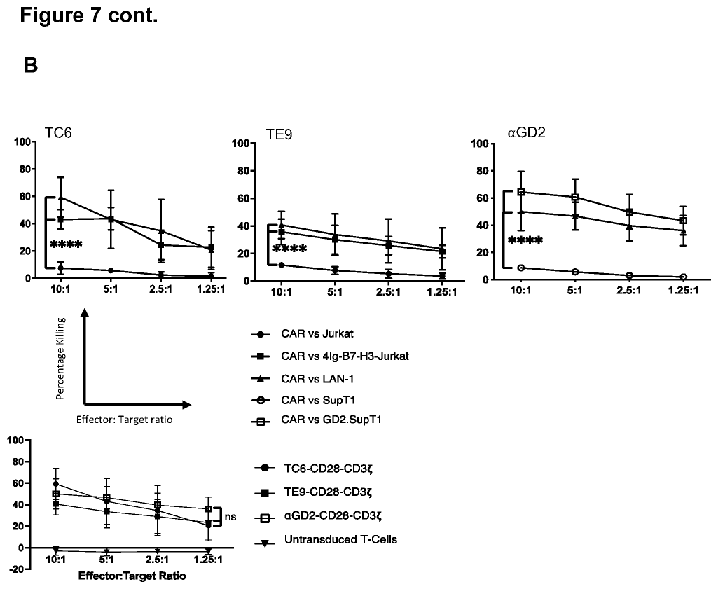

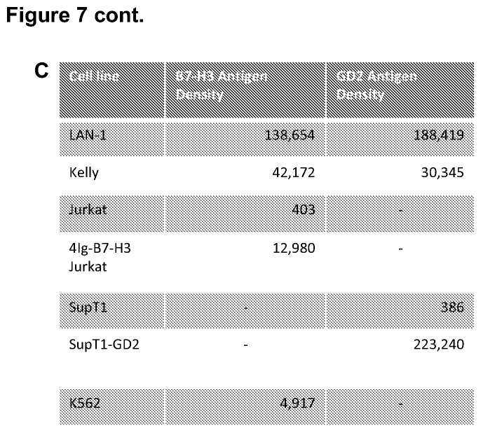

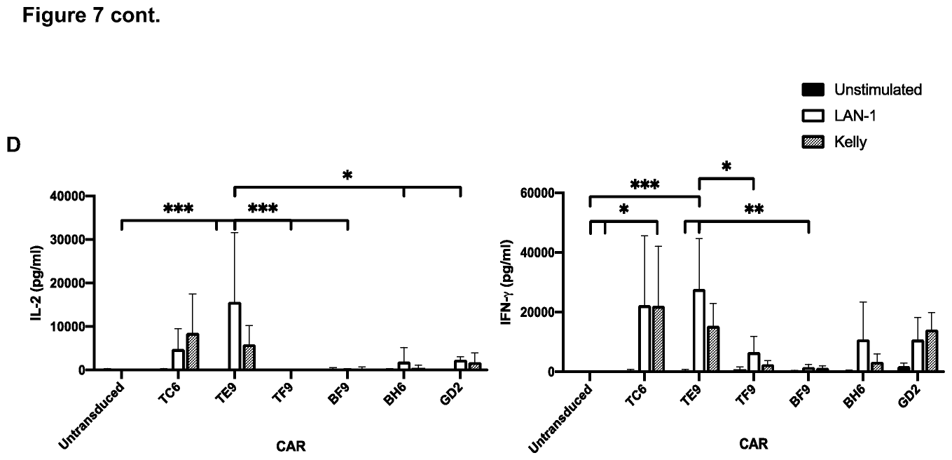

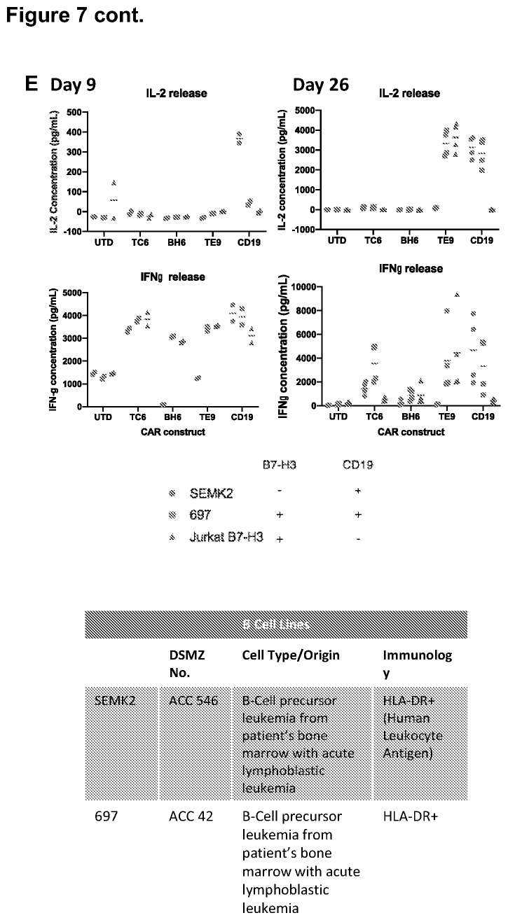

The 5 candidate scFv sequences were evaluated for ability to confer antigen-specific T cell function in second generation CD8H/Tm-CD28-CD35 (285) CAR format comprising the CD8-alpha hinge and transmembrane (H/Tm) sequences with CD28 and CD35 signaling domains, by assessing cytotoxicity and cytokine secretion (FIG. 7). CARs were evaluated for effector function by culturing with neuroblastoma cells naturally expressing B7-H3 (FIG. 7C). All five CAR-T constructs showed similar transduction efficiencies in human T cells (data not shown). Two binders (TE9, TC6) showed significant cytotoxicity specific for B7-H3-expressing target cells in four hour killing assays (FIG. 7B) and they also showed the greatest degree of cytokine response to neuroblastoma targets. However, binder BH6 showed less B7-H3-specific cytokine production in 24-hour assays (FIG. 7D).

To determine how CARs behaved during longer term co-cultures, they were evaluated as CAR-T in a repeat antigenic challenge assay during which CAR T cells received 4 stimulations of irradiated tumour cells over 4 weeks. Three of the anti-B7-H3 binders were compared with the FMC63 anti-CD19 CAR-T constructs. These experiments showed that TE9-28ξ and CD19-28ξ CAR-T cells had sustained capacity to produce IL-2 in response to a 4^{th} rechallenge with B7-H3 positive leukaemia cells (FIG. 7E). The TE9 binder was therefore selected for further optimisation of CAR-T function.

### Example 4—CD28 Costimulation and CD8 Hinge/Transmembrane Provide Optimal Long-Term Persistence to TE9 CAR-T Cells

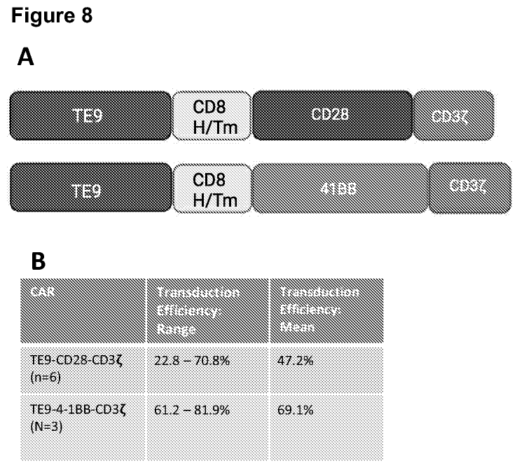

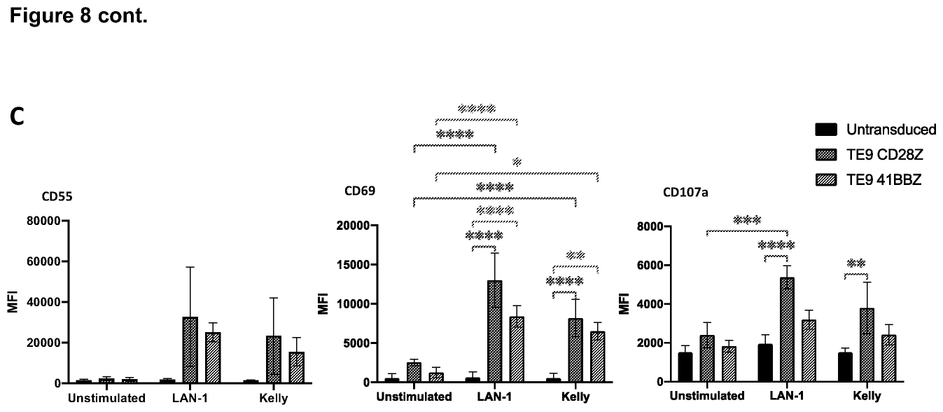

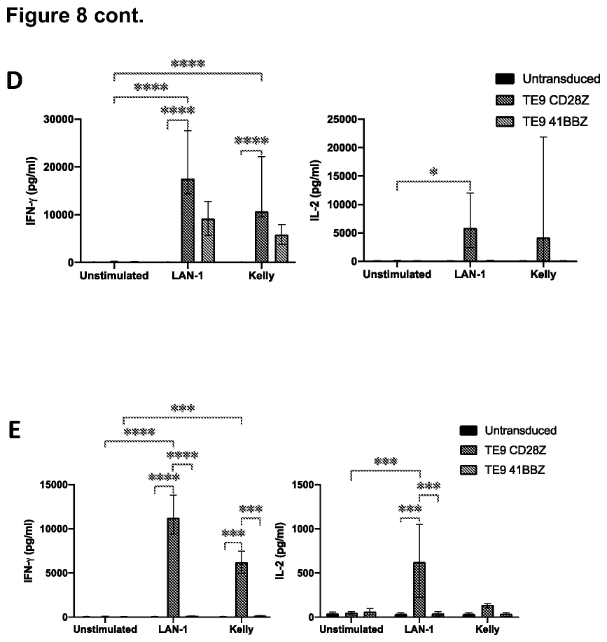

We next compared CD28 and 4-1BB endodomains combined with a CD8 hinge and transmembrane (H/Tm: FIG. 8A) by assaying effector function against B7-H3-expressing neuroblastoma cells. Similar levels of transduction efficiency were observed with the two constructs (FIG. 8B). Cytotoxic degranulation as determined by CD107a as well as upregulation of CD25 and CD69 activation markers following addition of targets, were non-significantly higher in CD28ξ than 4-1BBξ constructs (FIG. 8C). In short term co-cultures, TE9-28ξ generated more interferon-γ (IFN-γ) and significantly greater IL-2 than its 4-1BB counterpart (FIG. 8D). 4-1BB endodomains in CAR-T cells have been well described to confer ability for longer term effector function on antigen rechallenge. Thus, cytokine production following rechallenge with neuroblastoma cells 7 days after initial antigenic challenge was assessed. Here the TE9-4-1BBξ CARs induced significantly lower levels of both IL-2 and IFN-γ following neuroblastoma repeat challenge (FIG. 8E). The greater activation with the CD28 endodomain was reflected in significantly higher upregulation of activation/exhaustion markers (data not shown).

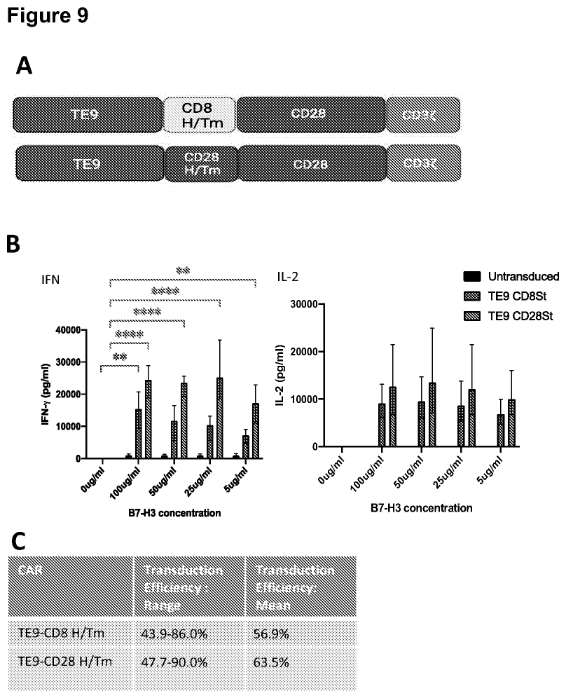

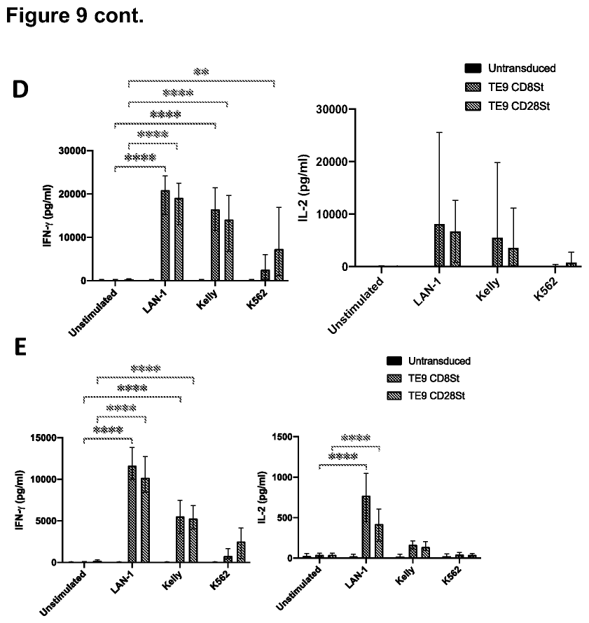

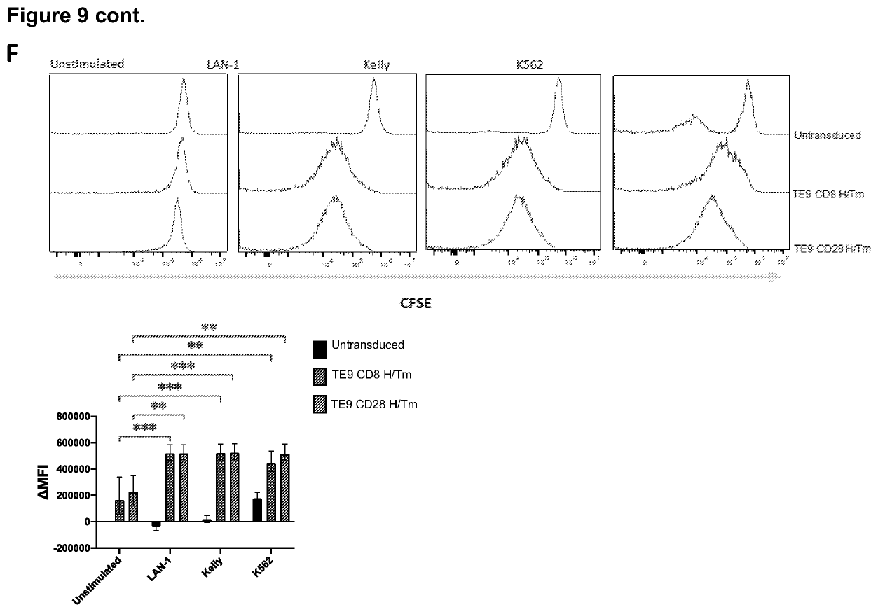

Previous studies have indicated that a CD28 H/Tm confers significantly greater sensitivity to target antigens than CD8 H/Tm (Majzner et al., 2020 & Muller et al., 2021). Thus, TE9-28z was compared using the two different H/Tm arrangements, both of which were expressed on T cells at similar levels (FIG. 9A, 9C). The respective CAR-T constructs were stimulated with decreasing concentrations of recombinant B7-H3 and demonstrated only a marginal enhanced IFN-γ and IL-2 response of the CD28 H/Tm construct, which was most marked for IFN-γ and at lowest antigen concentrations (FIG. 9B). Cytokine production was evaluated after 18 hours co-culture with neuroblastoma cells LAN-1 and Kelly which have different levels of B7-H3 expression, and K562 cells which have a low expression of B7-H3. Cytokine production and proliferation after 7 days incubation followed by an antigen rechallenge was evaluated, with cytokine analysis 24 hours later. No statistically significant difference was noted between the CARs, both of which demonstrated antigen-specific effector function although the CD28 H/Tm led to a higher mean IFN-γ response in the presence of the B7-H3-low K562 cells following 24 hours (FIG. 9D) and 7 days (FIG. 9E) of co-culture with targets. Hence in contrast to previous studies with other binders, it was presently observed that the CD28 H/Tm only demonstrated marginal increased sensitivity to a low antigen target.

### Example 5—TE9-28ξ with CD8 Hinge and Transmembrane Shows Enhanced Long-Term Proliferation and In Vivo Effector Function in Neuroblastoma Models

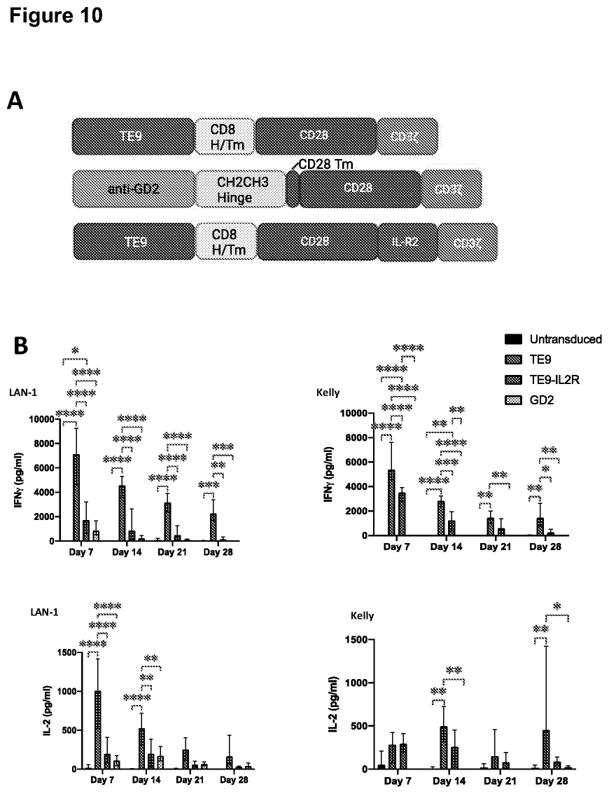

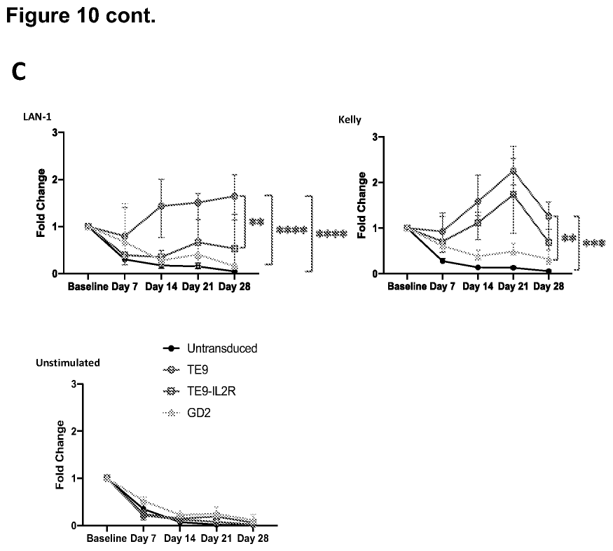

It was confirmed that TE9 with CD8 H/Tm was effective in long-term stimulation in stress conditions by challenging at weekly intervals with irradiated neuroblastoma cells and comparing with CAR-T cell survival of an anti-GD2 second generation CAR that has been shown to have clinical function but limited in vivo persistence (Straathof et al., 2020). Previous work has demonstrated that incorporation of IL-2 receptor beta chain into second generation anti-CD19 CAR endodomains combined with mutation of CD3ξ residues to enhance STAT3 phosphorylation, leads to longer term persistence (Kagoya et al., 2018). It was evaluated whether these modifications could enhance persistence to the TE9 anti-B7-H3 CAR-T cells in response to neuroblastoma challenge (FIG. 10A). Cytokine responses to both Kelly and LAN-1 neuroblastoma targets which express both GD2 and B7-H3 were assessed. Whilst the anti-GD2 CAR-T cells failed to expand or to produce cytokines after the second stimulation, the TE9-28ξ CAR-T persisted for 4 weeks and continued to generate both IL-2 and IFN-γ at levels significantly above background (FIGS. 10B & 10C). It was observed that with the addition of the cytokine signaling domains, the results were consistently inferior to conventional second generation TE9-28 cells for both persistence (and cytokine production (FIGS. 10B & 10C).

### Example 6—TE9-28ξ with CD8 Hinge and Transmembrane Shows Enhanced Long-Term Proliferation and In Vivo Effector Function in Neuroblastoma and Medulloblastoma Models

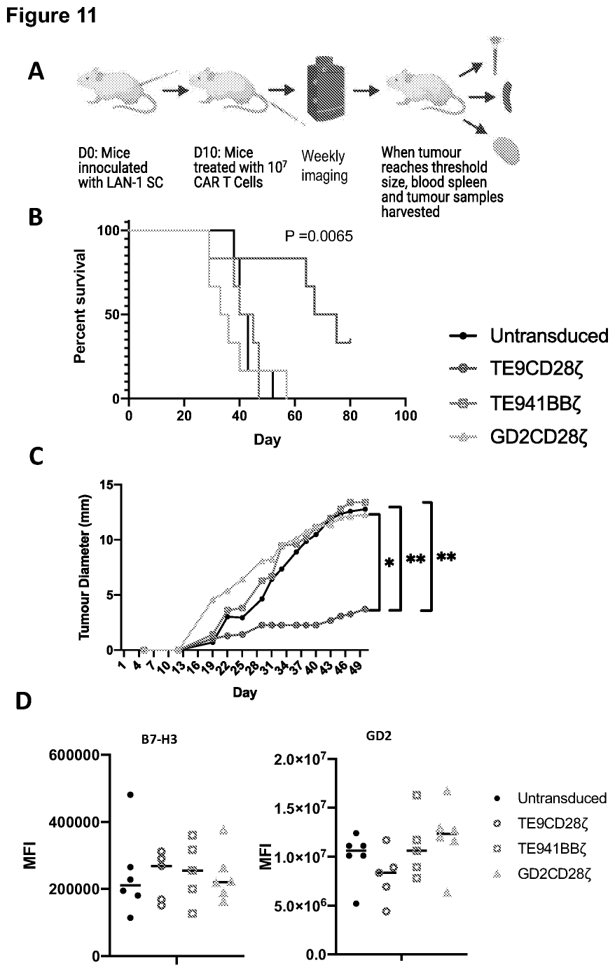

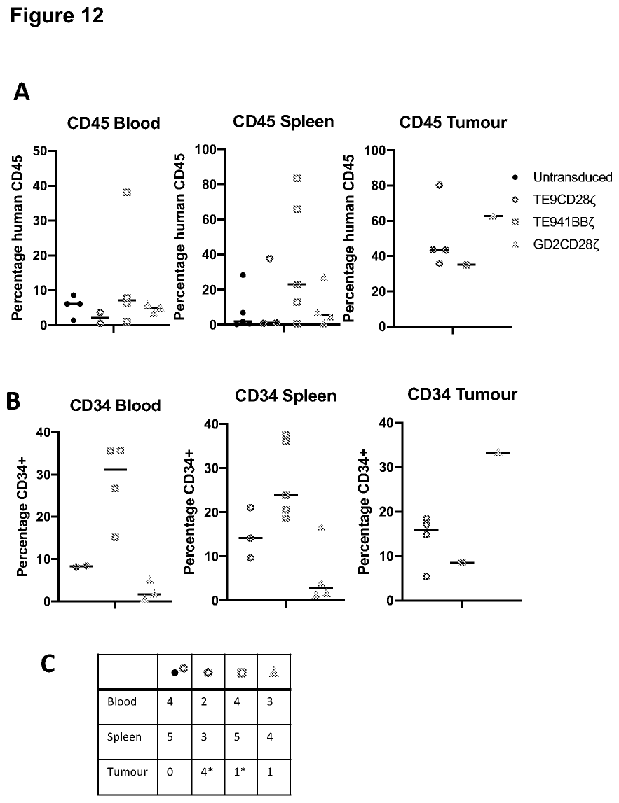

To determine if the long-term persistence of TE9-28ξ during repeat stimulation in stress conditions translated into effective in vivo function the LAN-1 subcutaneous neuroblastoma model was used, which was demonstrated previously to have been resistant to growth inhibition by GD2-28ξ CAR-T cells (data not shown). Mice treated with TE9-28ξ CAR T cells showed significantly enhanced survival and shrinkage of established tumours compared to TE9-BBξ and anti-GD2 CAR-T treated mice (FIG. 11). Tumours growing out from GD2-28ξ CAR-T treated mice showed no evidence of B7H3 antigen loss as evaluated by flow cytometry (FIG. 11C). In one tumour that eventually grew out of TE9-28ξ treated mice, populations of surviving CAR-T cells were observed which were not reproducibly seen in the TE9-BBξ and GD2-28ξ treated tumours (FIG. 12).

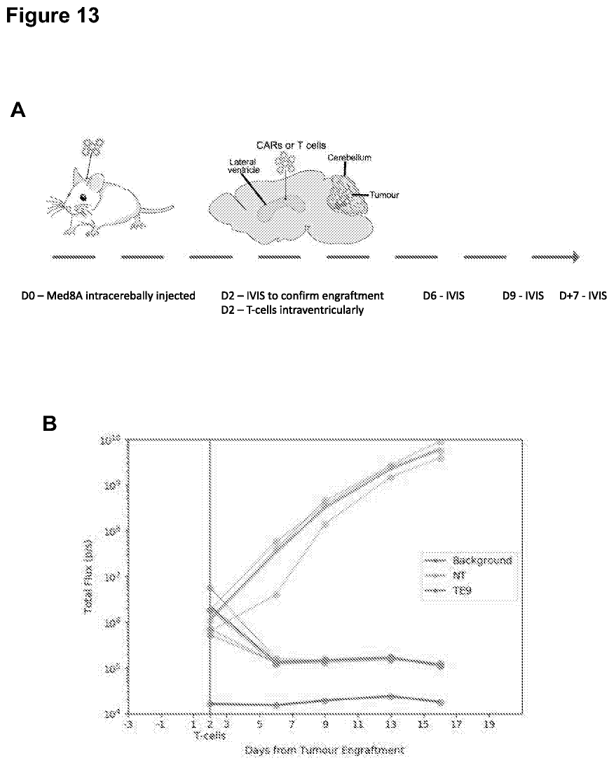

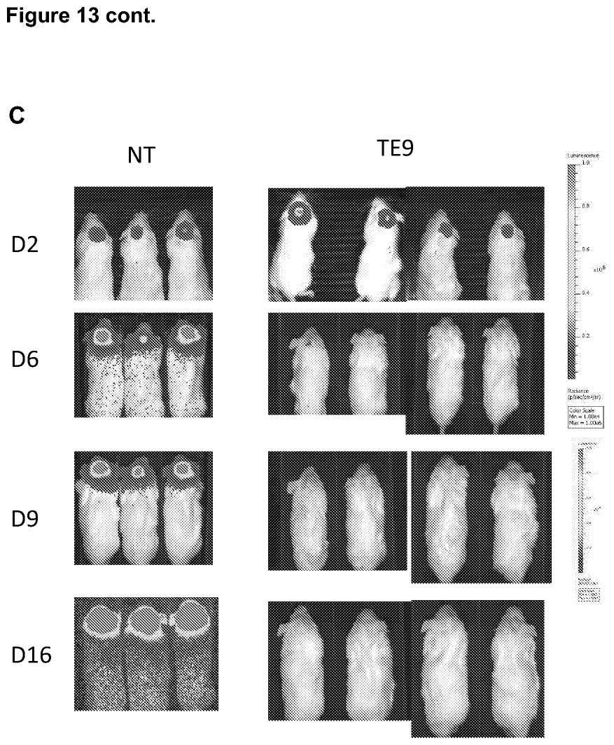

Capacity of TE9-28ξ CAR-T cells to control tumour growth was also evaluated in an orthotopic model of medulloblastoma in which immunodeficient mice were implanted with B7H3 positive medulloblastoma cells Med8A. Tumour bearing mice were treated by intraventricular administration of TE9-28ξ CAR-T cells which led to complete eradication of tumours (FIG. 13).

Taken together the data indicate the ability of TE9-28ξ CAR-T cells to effect tumour shrinkage of an established and treatment refractory neuroblastoma model without evidence of the emergence of antigen loss variants.

### Example 7—TE9-28ξ with CD8 Hinge and Transmembrane Shows Capacity for Selective Killing of Acute Myeloid Leukaemias Cells

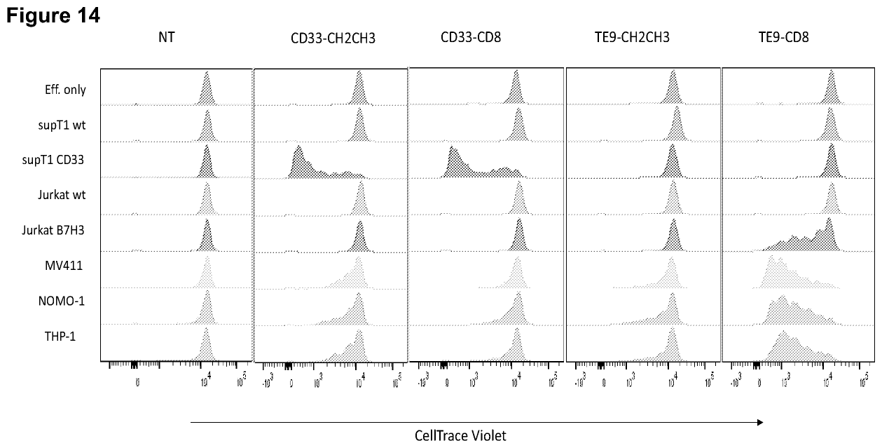

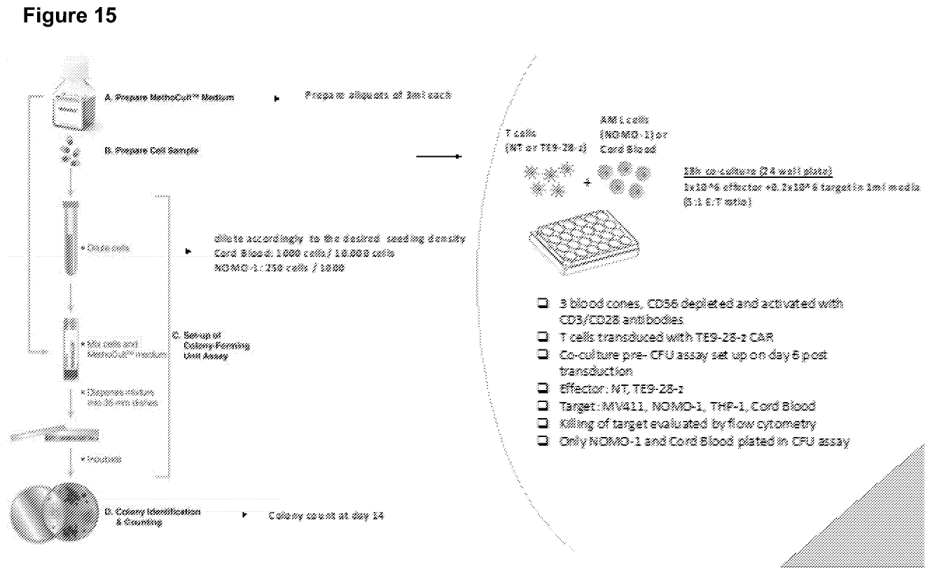

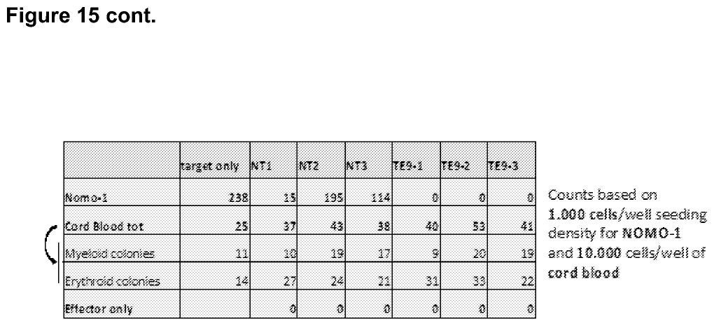

B7-H3 has been identified as a putative therapeutic target for acute myeloid leukaemia by virtue of its strong expression in this malignancy and lack of expression in healthy myeloid cells. In co-culture experiments with a range of target cells lines expressing the antigens CD33 and B7H3, proliferation of CAR-T cells was used as a read out. The TE9 binder was compared with an anti CD33 binder derived from the monoclonal antibody gemtuzumab ozogamicin; and both binders were compared in CAR-T format with either CD8 H/TM or a CH2/CH3 Fc domain used as spacer. The TE9-28ξ CAR-T cells with CD8 H/TM, in contrast to other constructs, showed consistent antigen dependent reactivity against three AML cell lines MV411, NOMO-1, and THP-1 (FIG. 14). To evaluate relative cytototoxicity against AML NOMO1 cells and healthy haemopoietic cells, a cord blood stem cell cytotoxicity assay was performed with haemopoietic or AML colony number as read out. The TE9-28ξ CAR-T eradicated leukaemic colonies whilst having no effect on normal haemopoiesis (FIG. 15).

### Example 8—TE9 Based CAR-T Cells Show Favourable Reactivity Against Neuroblastoma Target Cells Compared with Alternative Anti-B7H3 Binders in Repeat Stimulation Assays

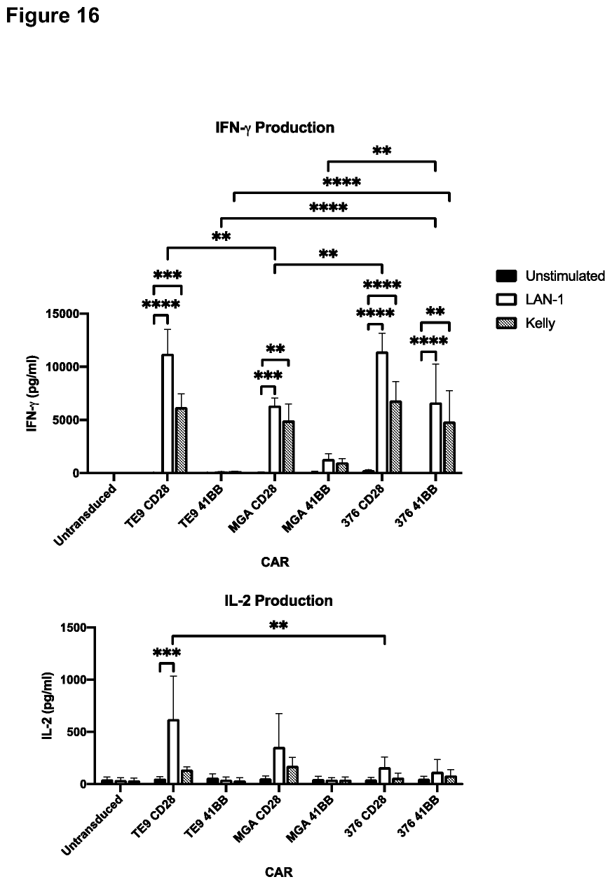

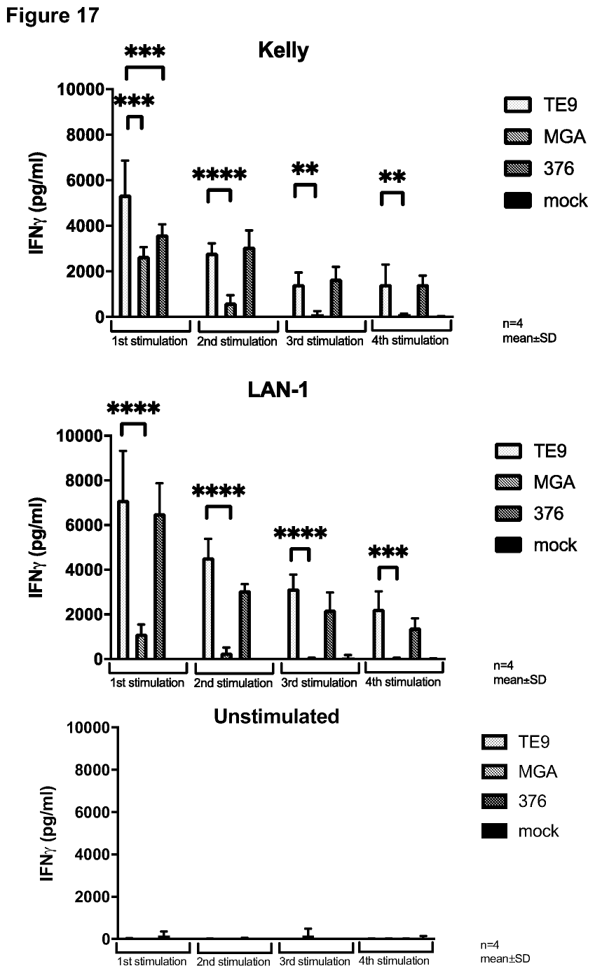

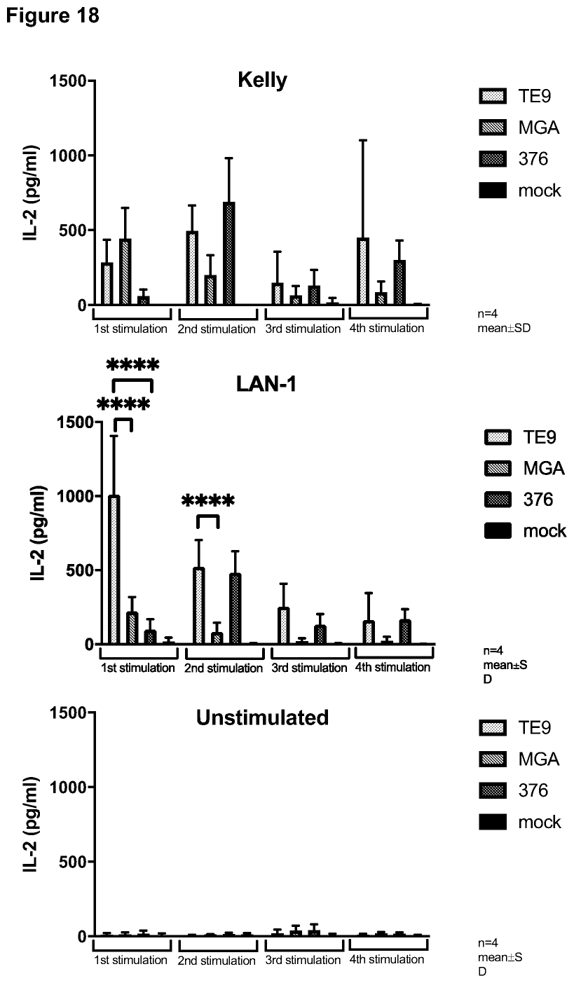

To benchmark the capacity of the TE9 binder to promote sustained anti-tumour reactivity, it was compared with two other binders MGA271 and 376-96, both of which have been described previously in CAR-T format. All three binders were cloned into the same gammaretroviral expression construct with CD8 H/TM domains. The neuroblastoma target cells Kelly and LAN-1 were selected based on their B7H3 expression patterns (FIG. 7). In both short term stimulation (FIG. 16) and repeat stimulation (FIGS. 17 and 18), the TE9 based construct showed highest tumour reactivity in terms of inflammatory cytokine production consistent with ongoing capacity for antigen-specific response.

### Example 9—Studies Using Gamma-Delta T Cells

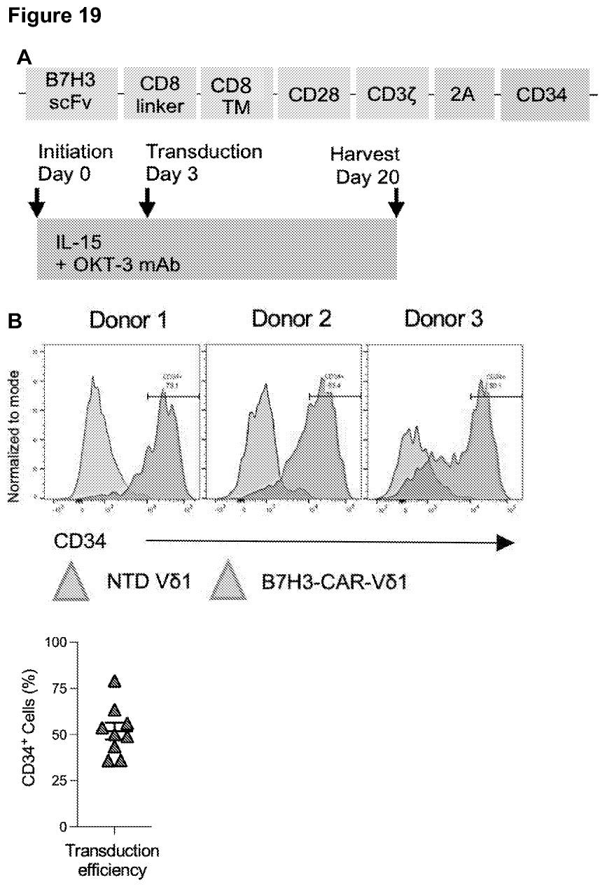

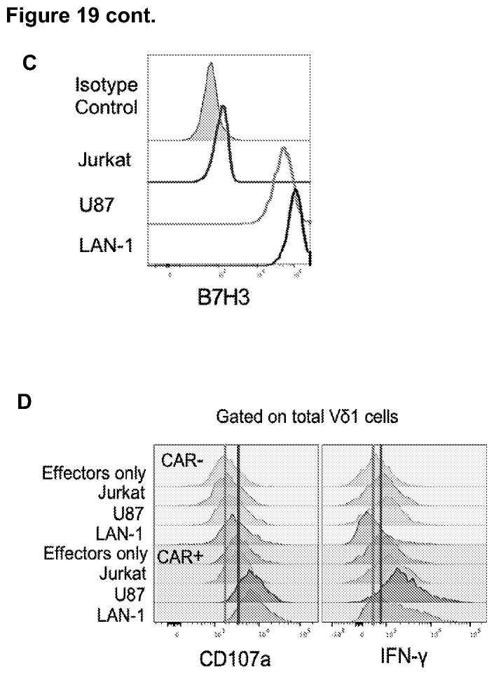

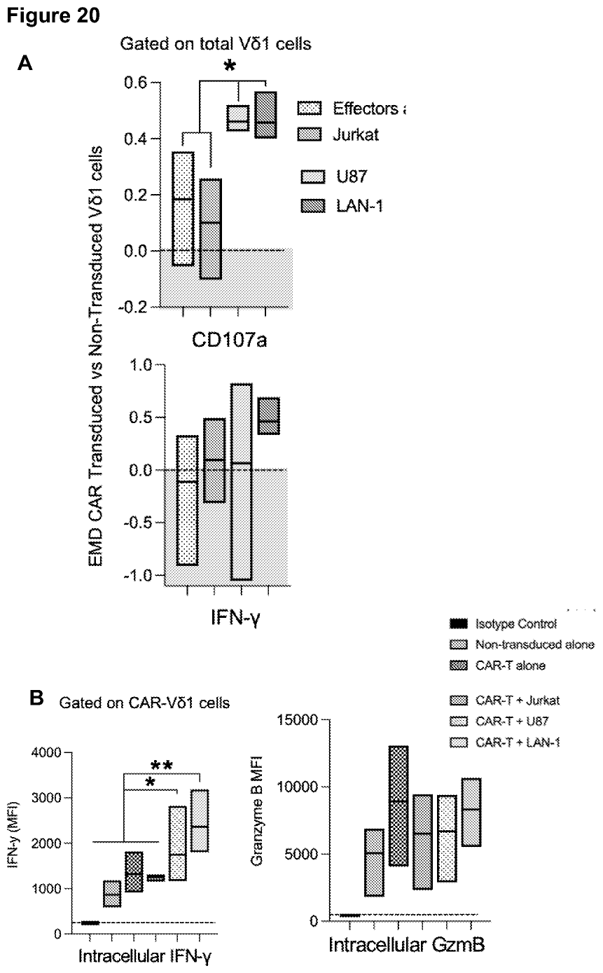

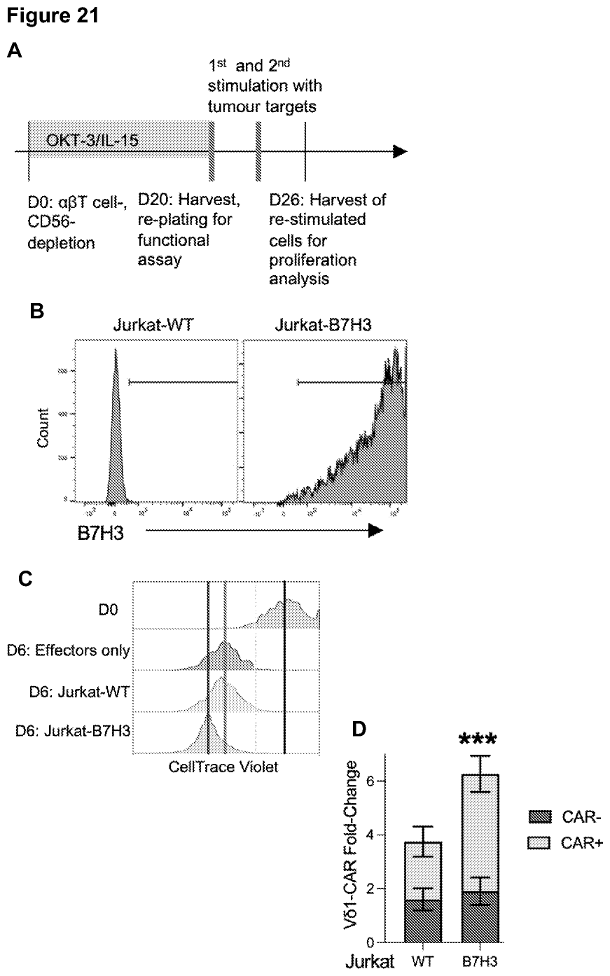

The TE9-28ξ CAR-T was evaluated for antigen dependent reactivity in the context of gamma delta T lymphocytes. In co-cultures with Jurkat (leukaemia), U87 (Glioma) and LAN-1 (neuroblastoma) targets, there was enhanced CD107a and interferon gamma response in the CAR transduced gamma delta T cells (FIGS. 19 and 20). Moreover, there was also enhanced proliferation (FIG. 21)

### Example 10—Respective Avidities of the B7H3 Binders Affects Performance of CAR-T Against Low Antigen Density Targets

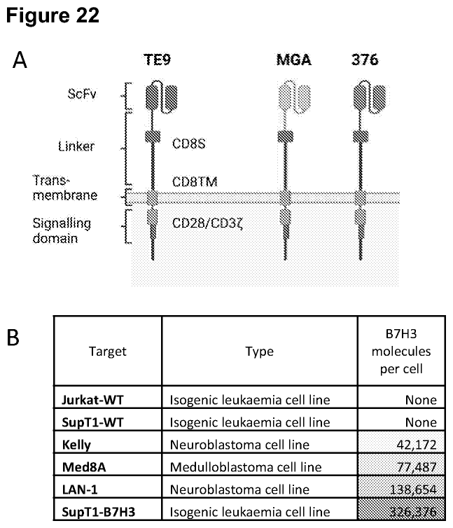

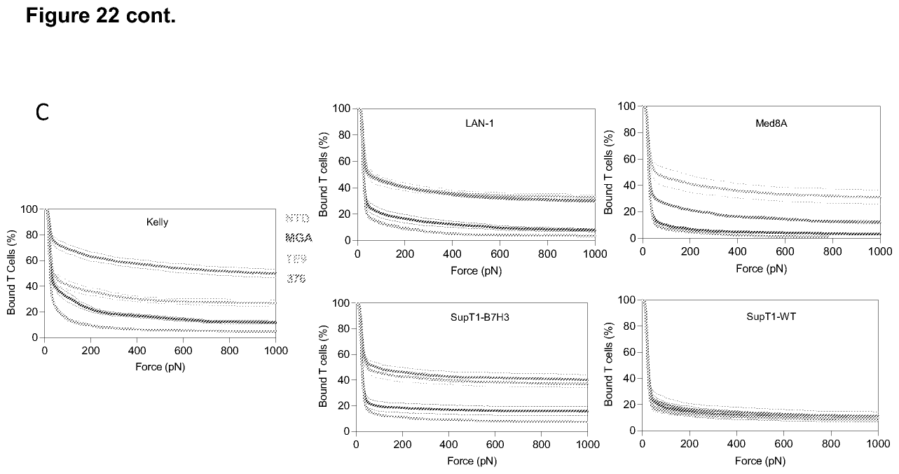

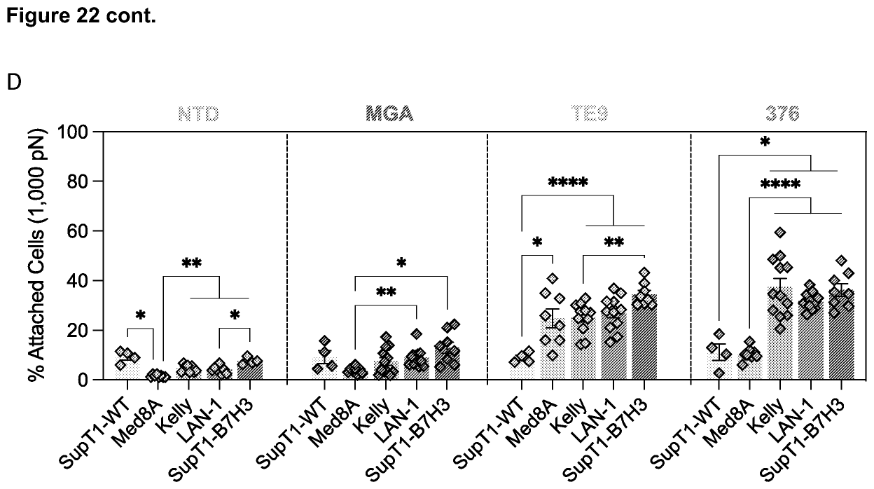

To compare functional avidity across CAR constructs, the novel ScFv binder TE9 as well as comparative anti-B7H3 binders MGA271 and 376.96 were cloned into the same SFG gammaretroviral CAR backbone incorporating second generation CD28 and CD3-zeta signalling domains (FIG. 22a). Avidity was evaluated by measuring force required (Lumicks technology) to dislodge CAR-T cells from a range of target cells expressing a range of B7H3 target densities (FIG. 22b). The binders TE9 and 376.96 in CAR-T format had significantly higher avidity than MGA271 in all the antigen-positive cells (FIG. 22c-d).

### Example 11—Avidity Differences of CAR-T Binders are Associated with Differential Function Against Target Cells of Different Antigen Densities

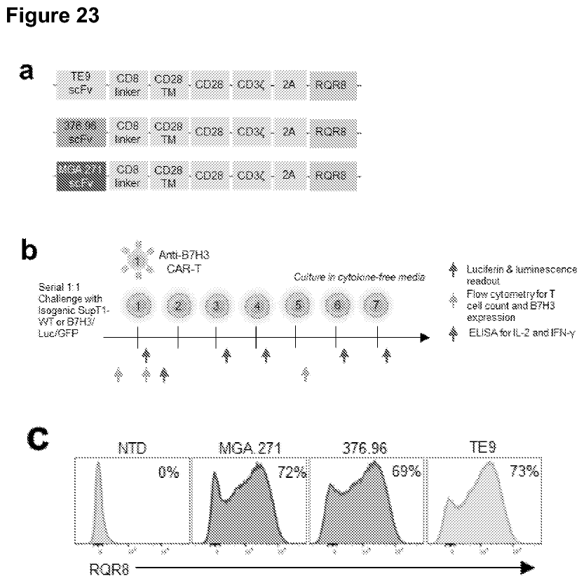

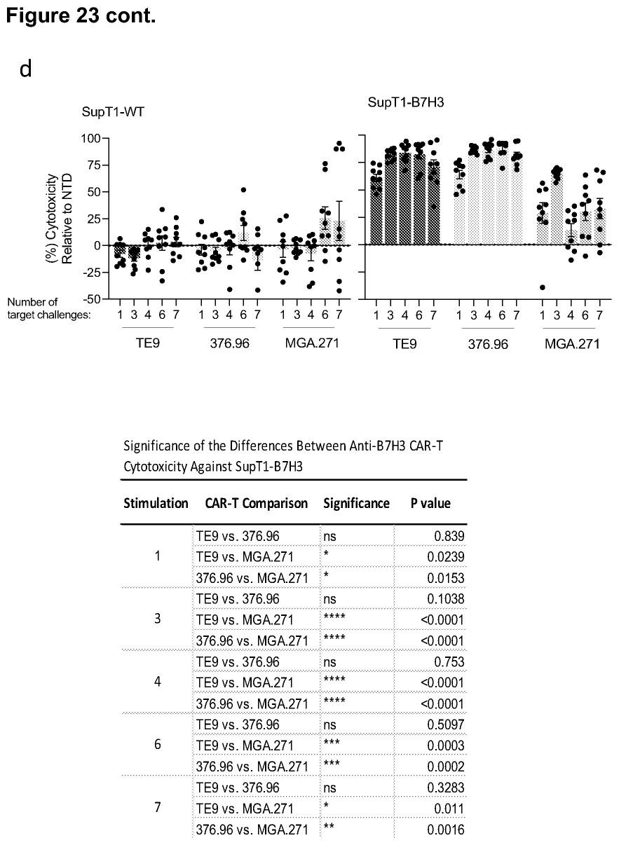

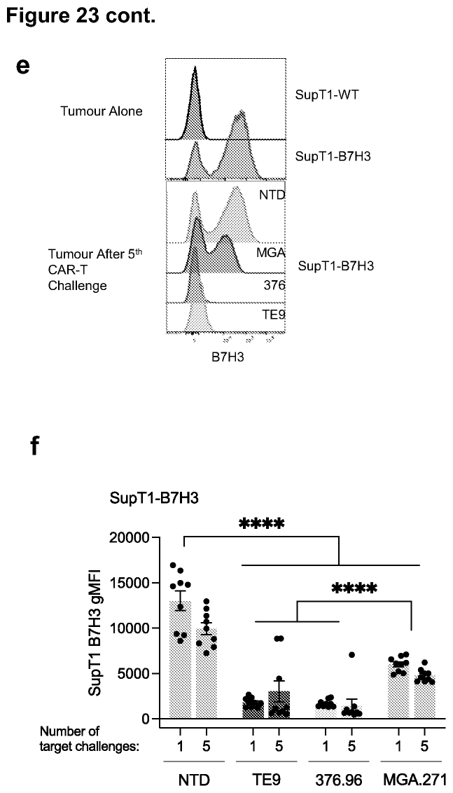

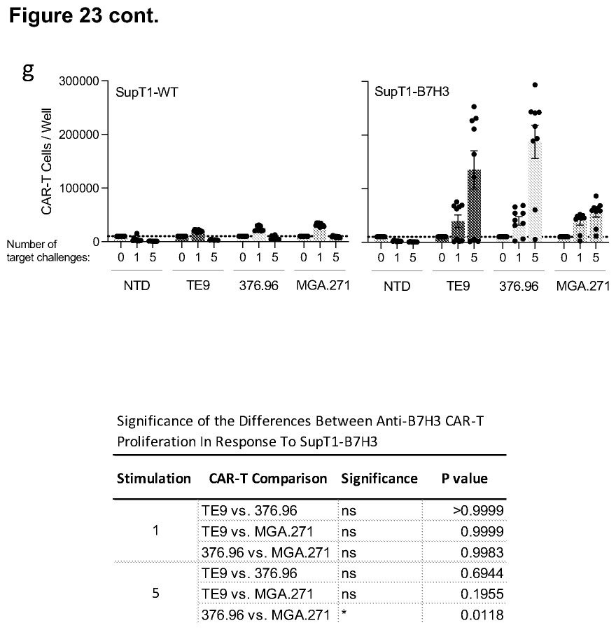

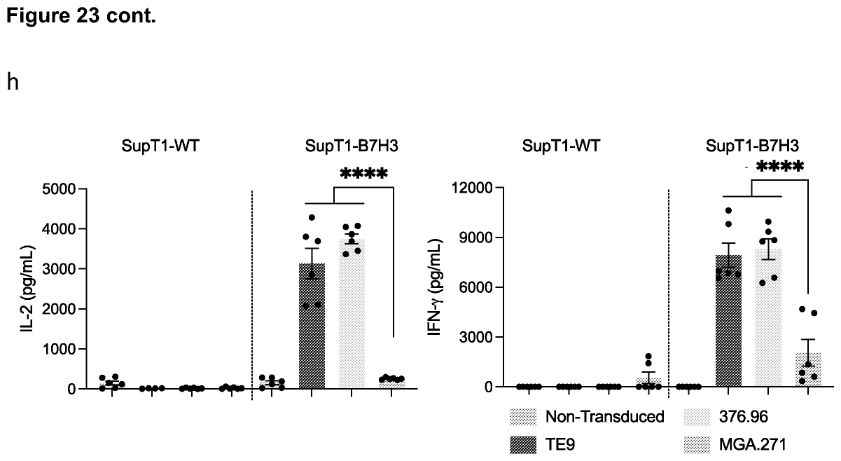

To test the hypothesis that higher avidity of CAR-T will translate into better control of antigen-dim targets, an artificial cell line was established by transducing B7H3 negative SupT1 cells with a range of multiplicities of infection of gamma retrovirus expressing human B7H3. A mixed population of SupT1 cells expressing a broad range of B7H3 was obtained (FIG. 23e untreated control). The experimental set-up to test the avidity hypothesis was a repeat stimulation assay in which the mixed population of B7H3-positive SupT1 cells was added to the respective CAR-T populations every 1-2 days (FIG. 23a-c). After repeat antigen challenges, the MGA271 but not the TE9 or 376.96 CAR-T cells demonstrated failure to control tumour growth (FIG. 23d). The treatment failure of the MGA271 cells was associated with outgrowth of an antigen-dim population, whilst the TE9 and 376.96 binders were effective to eliminate both the bright and dim SupT1 populations (FIG. 23e-f). Consistent with the enhanced killing by the TE9 and 376.96-based CARs, they expanded significantly more than the MGA271-CAR (FIG. 23g) as well as producing more inflammatory cytokines (FIG. 23h).

### Example 12-TE9—Based CAR-T Show Reduced Basal Activity in the Absence of Antigenic Signal

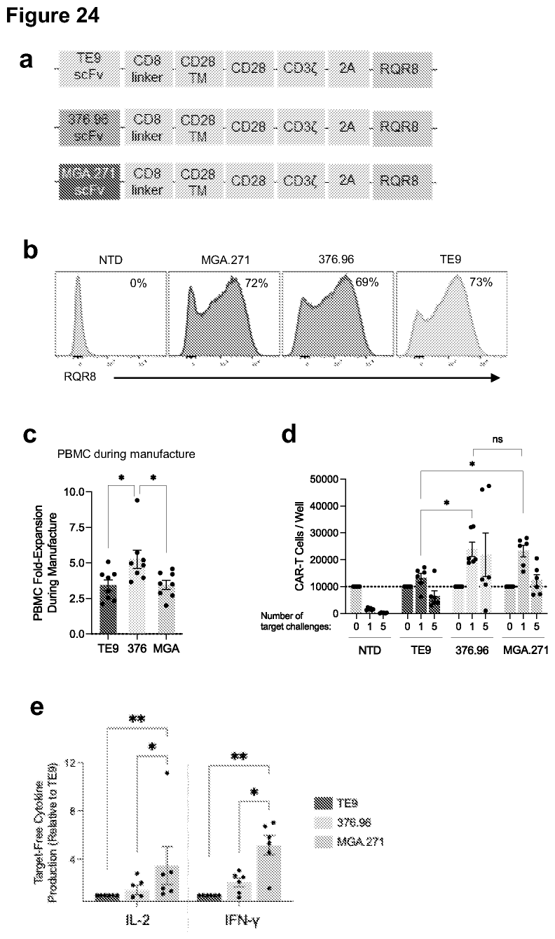

A recognised limitation of CAR-T cells is exhaustion, induced by tonic signalling (signalling downstream from the CAR in the absence of target antigen). Factors that can influence the extent of tonic signalling include the signalling domains and transmembrane as well as the ectodomain. Increased tonic signalling from CAR could in theory lead to greater expansion during manufacture but paradoxically could lead to decreased subsequent CAR-T efficacy. To specifically evaluate the contribution of the TE9 binder it was benchmarked against the other anti-B7H3 antibodies with identical transmembrane and endodomains (FIG. 24a) and with equivalent transduction efficiency (FIG. 24b). The expansion of CAR-T cells was evaluated during manufacture in the absence of antigen, and the 376.96-containing CAR was found to expand significantly greater than TE9 and MGA271, the difference between which was non-significant. Following manufacture, the respective CAR-T cells were cryopreserved at the same time and were next evaluated for reactivity in the absence of target antigen. Over one week of culture conditions in the absence of antigen, the TE9 based CAR showed no significant proliferation whilst there was a detectable increase in numbers of 376.96 and MGA271 cells compared with TE9-based CAR-T cells evaluated at the same timepoint (FIG. 24d). Further evidence for absence of tonic signalling with the TE9-based binder was obtained through evaluation of inflammatory cytokine IL-2 and interferon gamma released into culture supernatant in the absence of target antigen. Both MGA271 and 376.96 CARs produced more cytokine than TE9 CAR and this reached statistical significance comparing TE9 with MGA271.

### Example 13—A TE9 Chimeric Costimulatory Receptor Providing CD28 Signalling Confers Additional T Cell Effector Function to T Cells Receiving Signal 1 Though a T Cell Receptor

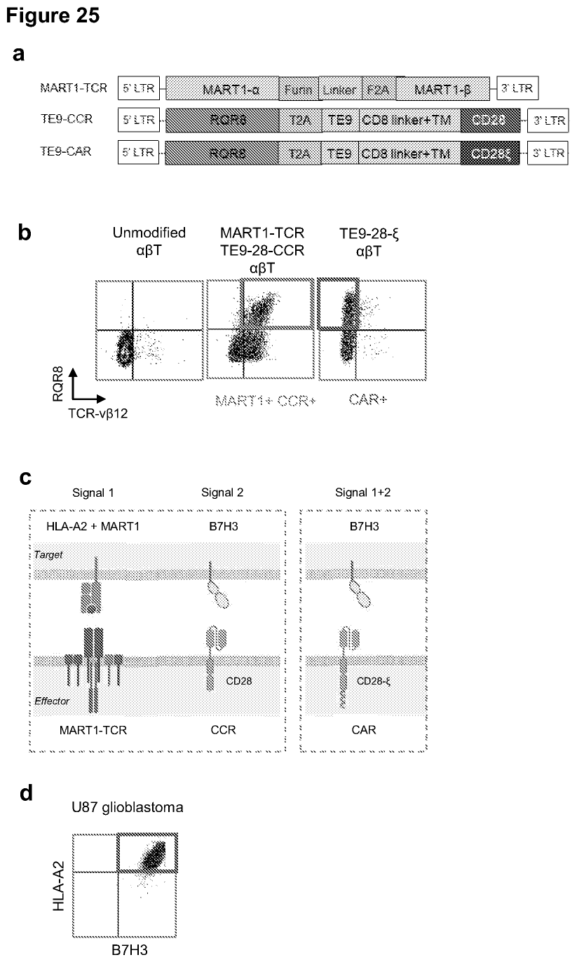

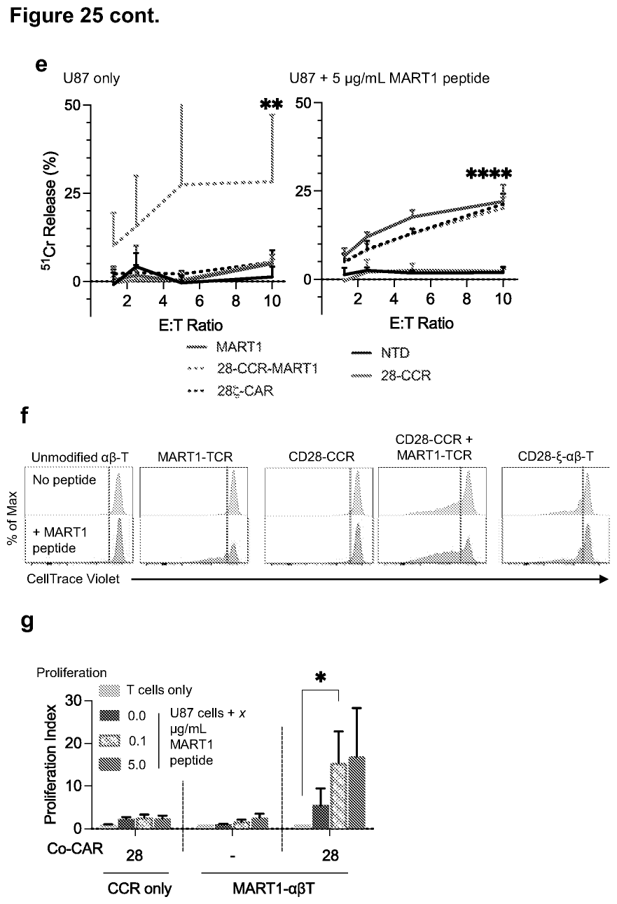

The concept of a chimeric costimulatory receptor (CCR) is a conventional CAR that lacks the signalling endodomains conferring signal 1 (i.e. CD3zeta) but does have domains providing co-stimulation (e.g. CD28 and 41-BB) (FIG. 25c). A TE9 CCR with CD28 costimulation was generated to provide signal 2 whilst a construct encoding a conventional alpha beta TCR recognising a peptide antigen from MART-1 was generated in the same viral vector backbone (FIG. 25a). Through co-transduction of human primary T cells with the two vectors it was possible obtain a population of cells expressing both receptors, whilst conventional TE9-28Z CAR was expressed as a control (FIG. 25b). The MART1 peptide which the TRC reacts with is restricted to presentation of HLA-A201. Hence, a target line U87 glioblastoma which expresses B7H3 and is HA-A201 positive is a suitable target for evaluation of the construct although U87 cells are also MART1 negative and so will present to the MART1 TCR only if pulsed with the TCR-specific peptide (FIG. 25d). In short-term 4 hour chromium release assay, as expected, expression of the TE9 CCR did not confer additional cytotoxic activity upon the TCR engaged T cells (FIG. 25e). In contrast, the presence and engagement of the TE9 CCR led to significantly enhanced proliferation response which was also dependent on engagement of the TCR (FIG. 25f-g). Taken together the data confirm proof of concept that a CD28 conferring TE9 chimeric costimulatory receptor gives essential proliferative signal in trans when combined with a TCR signal. This data identifies a potential therapeutic application of TE9 antibody binders to provide AND gate signals to TCR.

### Example 14—TE9 Based Anti-B7H3 CD28-Chimeric Costimulatory Receptor (CCR) Co-Expression with a G115-TCR Broadened αβ-T Cell Cytokine Reactivity to Targets that were not Sufficiently Stimulatory with Vγ9Vδ2-TCR Alone

One example of a TCR signal that could be costimulated by the TE9 CCR is the gamma delta TCR derived from Vγ9Vδ2 T cells since its binding properties are specific for malignantly transformed or infected cells. Moreover, recognition by the Vγ9Vδ2 TCR is independent of MHC and so broadly applicable to human cancer patients. One previously characterised Vγ9Vδ2 TCR is called G115 and is known to be of intermediate affinity and to provide an intermediate signal strength on engagement. This G115 TCR was therefore chosen to evaluate the TE9 CCR costimulation concept.

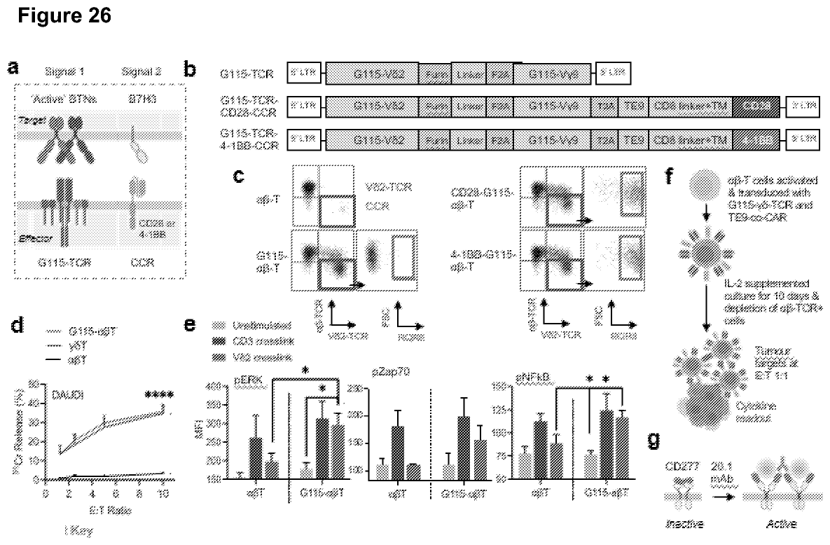

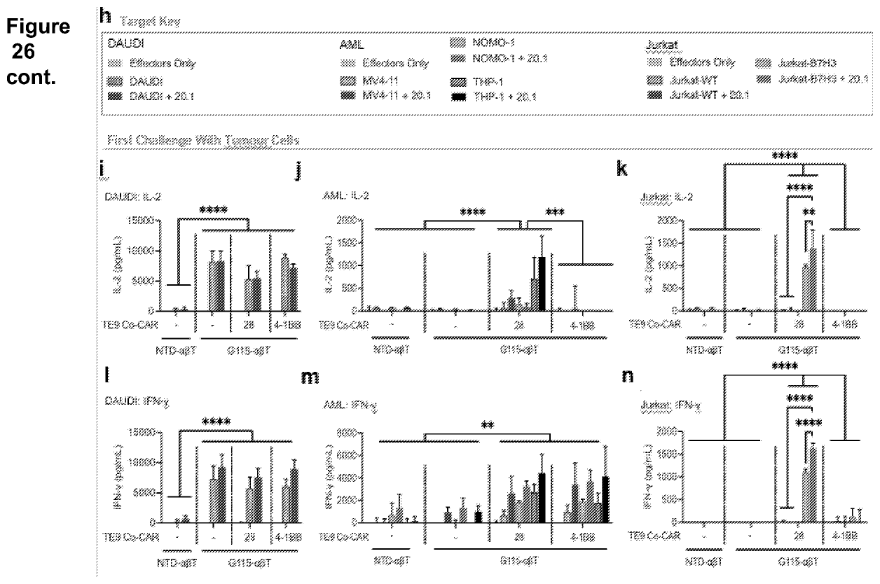

The mechanism of engagement of the G115 TCR is through binding of butyrophilin molecules (most likely BTN2A1 and BTN3A1 dimers) in an active conformational configuration (FIG. 26a). To test TE9 induced costimulation in combination with this TCR, gammaretroviral constructs were generated in which G115 TCR could be co-expressed with the TE9 CCR (either CD28 or 41-BB versions of CCR-FIG. 26b). Transduction of peripheral blood mononuclear cells derived from healthy donors led to transduced cells that expressed both the G115 TCR and the CCR as determined by flow cytometry (FIG. 26c). In a 4 cytotoxicity assay evaluating killing of B7H3 positive Daudi cells, as had been observed with the MART TCR, engagement of the CCR does not alter the short-term killing induced by the transgenic TCR (FIG. 26d). To provide confirmatory evidence that the G115 TCR was signalling in response to engagement of the receptor, cross-linking with either anti CD3 or anti V-delta2 specific antibody was performed and phosphorylation of downstream signalling molecules was documented by phospho-flow cytometry (FIG. 26e). To test whether the TE9-CCRs provide functional activity to transduced alpha beta T cells, an experimental set up was devised in which transduced cells were co-cultured with tumour targets treated or not with monoclonal antibody 20.1 which is thought to act by converting BTN3A1 (CD277) to an active conformation (FIG. 26f-g).

G115-αβ-T cell cytokine production in response to DAUDI lymphoma stimulation could not be further enhanced with either addition of CCR or 20.1 mAb (FIG. 26I,L). This suggests maximal baseline DAUDI G115-TCR sensitivity, possibly due to saturating levels of conformationally active CD277 on the target cell surface and/or to expression of co-stimulatory ligands for the CD28 or 41BB costimulatory receptors. In contrast, all three AML cell lines tested failed to induce substantial G115-αβ-T cytokine responses, with IFN-γ levels substantially enhanced only upon addition of CCR (FIG. 26J,M). The CD28-CCR was particularly effective to enhance IL-2 response. 20.1 mAb provided a modest boost to G115-αβ-T responsiveness.

Dual TE9CCR-G115-transduced αβ-T cells substantially outperformed single-transduced G115-αβ-T-cells, even when targets were pre-treated with TCR-sensitising 20.1 mAb, suggesting that appropriate provision of a co-stimulatory signal may reduce the threshold for target TCR sensitisation. The result was even more striking with Jurkat T leukaemia targets, whereupon no cytokine was produced in any of the tested conditions, with the exception of the CD28-CCR, identifying differences between the 4-1BB- and CD28-CCRs (FIG. 26k,n). Consistent with this being an antigen-specific CCR-mediated effect, such responses were seen only in response to challenge with Jurkat-B7H3 but not Jurkat-WT cells. The addition of 20.1 mAb had a significantly enhancing effect on CD28-CCR-mediated IFN-γ and IL-2 release.

These data suggest a model whereby strong TCR engagement by BTN-rich, conformationally-active targets that may also express additional co-stimulatory molecules, such as DAUDI cells, obviates the requirement for a CCR. In other types of targets, such as AML or Jurkat T leukaemia, further activation of G115-αβ-T cells can be achieved through use of a TE9-CD28-CCR and addition of CD277-potentiating combination treatments, such as 20.1 mAb, whereby provision of co-stimulation may reduce the requirement for target TCR sensitisation.

### Example 15—Dependence of G115-αβ-T Cell Responsiveness on TE9-CCR Expression Increased Upon Re-Challenge with Tumour Targets

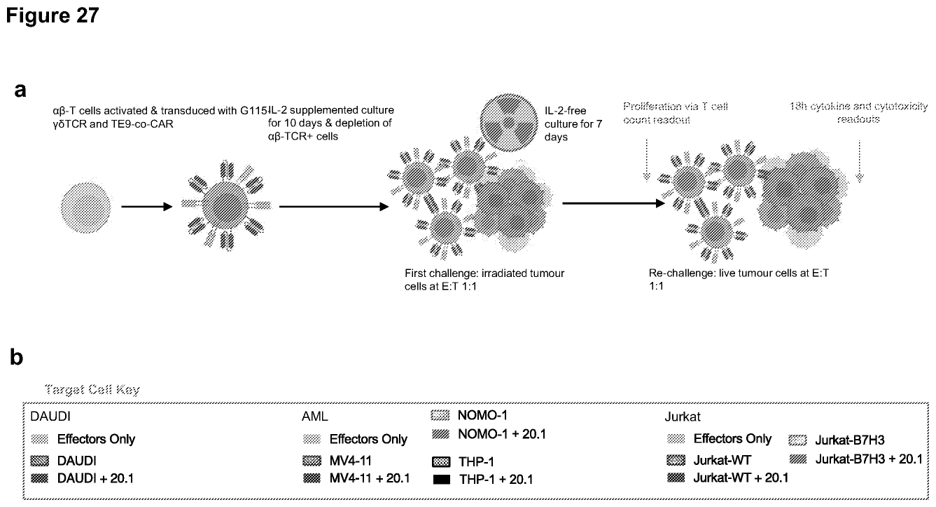

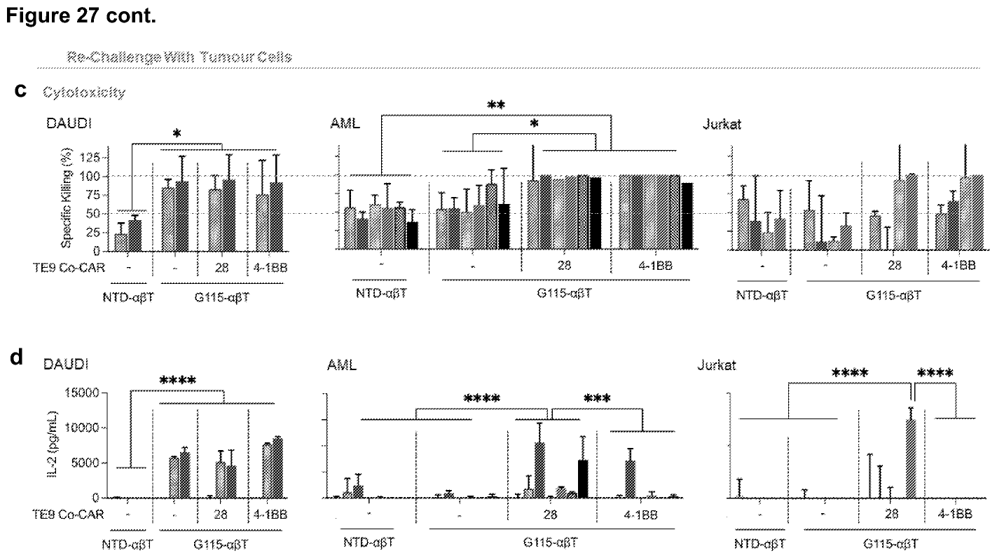

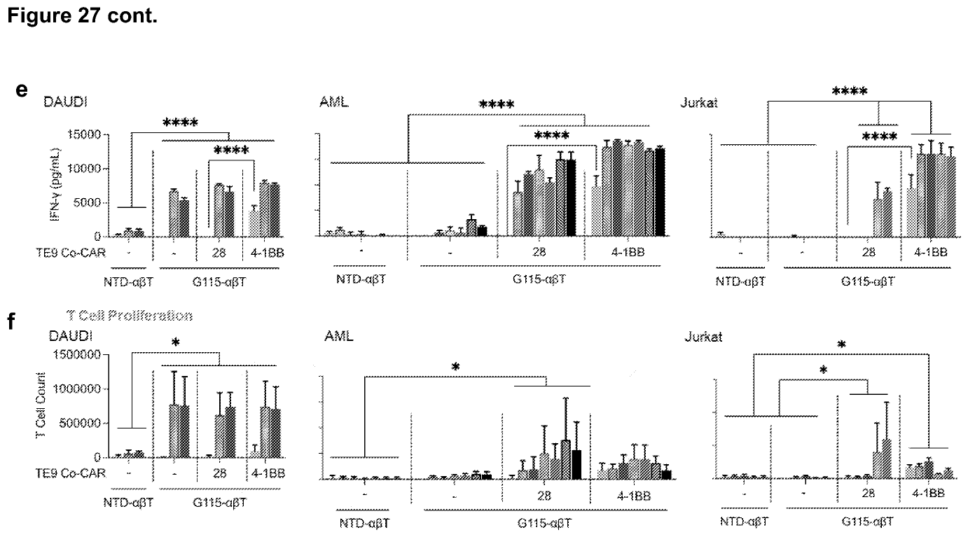

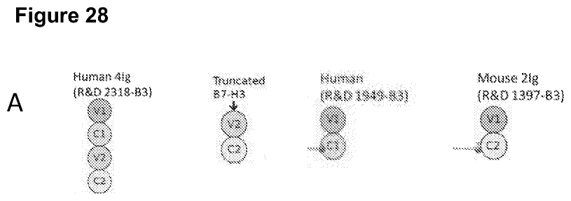

To evaluate the effect of antigen re-challenge on CTE9-CR-G115-αβ T cells—as a read-out of serial killing ability, the T cells were stimulated with irradiated tumour cells to provide an initial proliferative stimulus, seven days following which T cells were re-challenged with live tumour targets to enable evaluation of cytotoxicity as well as cytokine production (FIG. 27A,B). Data from the rechallenge experiments is displayed in FIG. 27c-f. The addition of either TE9-CCR led to increased cytotoxicity upon re-challenge with AML and Jurkat-B7H3 targets (FIG. 27C). Following T cell re-challenge with targets, DAUDI cells again induced potent G115-TCR-dependent cytokine release that neither co-stimulation nor 20.1 antibody could enhance (FIG. 27-D,E). 4-1BB—but not CD28 version of the TE9-CCR induced significant levels of target-independent IFN-γ that was not seen at first challenge with target cells. This suggests TE9-28 is the more favourable configuration of the TE9-CCR to evaluate in combination with a transgenic TCR. As with the primary challenge, Jurkat cells presented the greatest dependency on co-stimulation, and showed a similar pattern upon tumour rechallenge where IL-2 production was only observed when both CD28 co-stimulation and 20.1 antibody stimulation were deployed. This was in contrast to the initial challenge with Jurkat-B7H3 cells, where IL-2 production could be boosted with 20.1 mAb treatment but was not entirely dependent on it. DAUDI cells induced strong G115-TCR-dependent responses such that co-stimulation did not boost proliferation, while AML and Jurkat targets were dependent on CD28 co-simulation as well as B7H3 Ag expression for maximal proliferation (FIG. 27F). Interestingly, significant boosting was not seen upon addition of the 20.1 mAb, suggesting that additional TCR signalling could not further boost proliferation, consistent with the requirement for the provision of signal 2 for optimal ongoing responses. Indicative of tonic signalling of the 4-1BB-CCR, a level of target-independent proliferation was seen in 4-1BB-CCR but not CD28-CCR G115-αβ-T cells.

Taken together, these data indicate that the TE9 CD28-CCR enhances G115-TCR-T immunotherapeutic persistence and reactivity. The combination of these generalizable targeting moieties may offer a route forward for developing broadly-reactive and MHC-unrestricted TCR-T therapy.

### Example 16. TE9 Antibody Displays Binding Against Both Membrane Proximal and Membrane Distal Portions of the Human B7H3 Protein

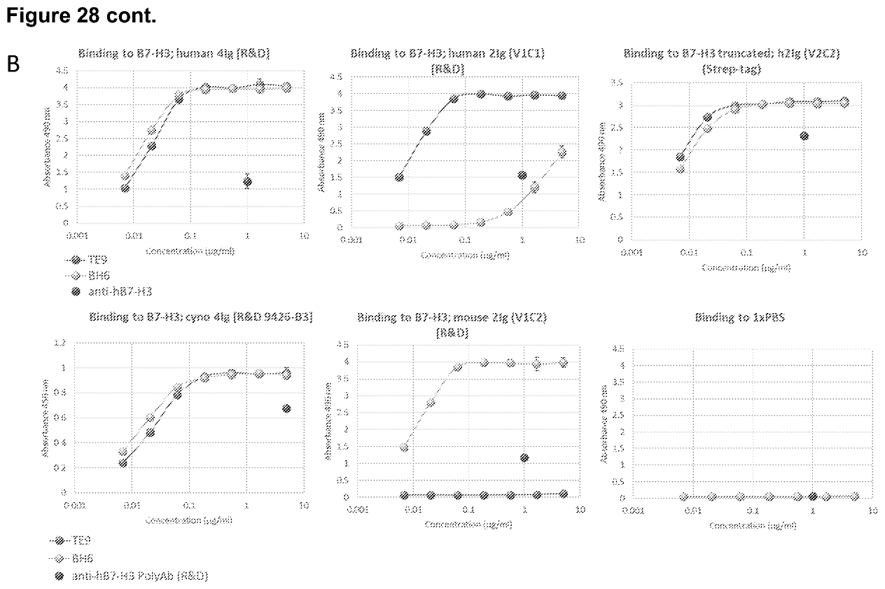

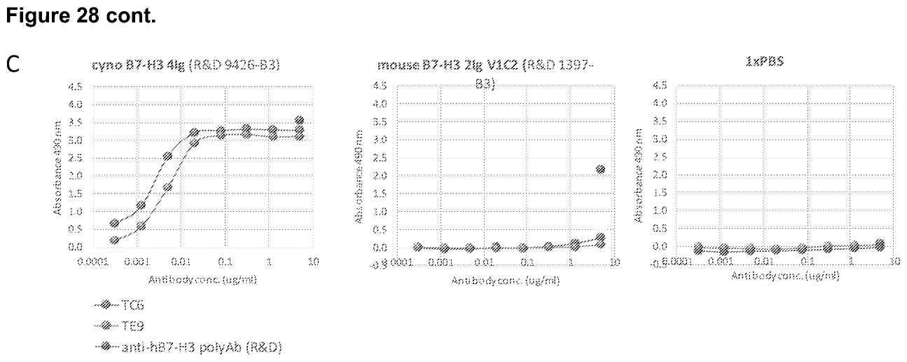

The TE9 antibody was derived from an ScFv library originally raised against the two most membrane proximal immunoglobulin domains of 4×Ig human B7H3 (V2C2-FIG. 28A). Binding studies however indicate that TE9 binds equivalently to the membrane distal V1C1 domains and to the full length Human 4Ig (FIG. 28B) and also cross reacts with cynomolgus monkey B7H3 but not with mouse B7H3 (FIG. 28C).

### REFERENCES

Kagoya, Y., et al., A novel chimeric antigen receptor containing a JAK-STAT signaling domain mediates superior antitumor effects. Nat Med, 2018. 24 (3): p. 352-359.
Majzner, R. G., et al., Tuning the Antigen Density Requirement for CAR T-cell Activity. Cancer Discov, 2020. 10 (5): p. 702-723.
Muller, Y. D., et al., The CD28-Transmembrane Domain Mediates Chimeric Antigen Receptor Heterodimerization With CD28. Front Immunol, 2021. 12: p. 639818.
Philip, B., et al., A highly compact epitope-based marker/suicide gene for easier and safer T-cell therapy. Blood, 2014. 124 (8): p. 1277-87.
Straathof, K., et al., Antitumor activity without on-target off-tumor toxicity of GD2-chimeric antigen receptor T cells in patients with neuroblastoma. Science Translational Medicine, 2020. 12 (571): p. eabd6169.
Almeida, A. R., et al., Delta One T Cells for Immunotherapy of Chronic Lymphocytic Leukemia: Clinical-Grade Expansion/Differentiation and Preclinical Proof of Concept. Am Assoc Cancer Res, 2016, 22, p. 5795 5804.

Sequences
- HCVR nucleic acid sequence of TA6
SEQ ID NO: 1
CAGGTCAAACTGCAGGAGTCTGGGGCTGAGCTGGTAAAGCCTGGGGCTTCAGTGAAGTTGTCCTGC
AAGGCTTCTGGCTACACTTTCACCAGCTACTGGATGCACTGGGTGAAGCAGAGGCCTGGACAAGGC
CTTGAGTGGATTGGAATGATTCATCCTAATAGTGGTAGTACTAACTACAATGAGAAGTTCAAGAGC
AAGGCCACACTGACTGTAGACAAATCCTCCAGCACAGCCTACATGCAACTCAGCAGCCTGACATCT
GAGGACTCTGCGGTCTATTACTGTGCTCAGGGTAACTCCTGGTTTCCTTACTGGGGCCAAGGGACC
ACGGTCACCGTCTCCTCA
- HCVR amino acid sequence of TA6 (CDRs 1-3 based on
the Kabat system underlined)
SEQ ID NO: 2
QVKLQESGAELVKPGASVKLSCKASGYTFTSYWMHWVKQRPGQGLEWIGMIHPNSGSTNYNEKFKS
KATLTVDKSSSTAYMQLSSLTSEDSAVYYCAQGNSWFPYWGQGTTVTVSS
- HCDR1 nucleic acid sequence of TA6
SEQ ID NO: 3
GGCTACACTTTCACCAGCTAC
- HCDR1 amino acid sequence of TA6 (as defined by the
Kabat system)
SEQ ID NO: 4
GYTFTSY
- HCDR2 nucleic acid sequence of TA6
SEQ ID NO: 5
CATCCTAATAGTGGTAGTAC
- HCDR2 amino acid sequence of TA6 (as defined by the
Kabat system)
SEQ ID NO: 6
HPNSGS
- HCDR3 nucleic acid sequence of TA6
SEQ ID NO: 7
GGTAACTCCTGGTTTCCTTAC
- HCDR3 amino acid sequence of TA6 (as defined by the
Kabat system)
SEQ ID NO: 8
GNSWFPY
- LCVR nucleic acid sequence of TA6
SEQ ID NO: 9
GACATTGTGCTGACCCAGTCTCCAGCAATCATGTCTGCATCTCCAGGGGAGAAGGTCACCATGACC
TGCAGTGCCAGTGCAAGTATAAGTTACATGCACTGGTACCAGCAGAAGTCAGGCACCTCCCCCAAA
CCATGGATTTATGAAATATCCAAACTGGCTTCTGGAGTCCCAGCTCGCTTCAGTGGCAGTGGGTCT
GGGACCTCTTACTCTCTCACAATCAGCAGCATGGAGGCTGAAGATGCTGCCATTTATTACTGTCAA
CAGTGGAGTAGTTACCCGCTCACGTTCGGTGCTGGGACCAAGCTGGAAATCAAA
- LCVR amino acid sequence of TA6 (CDRs 1-3 based on
Kabat system underlined)
SEQ ID NO: 10
DIVLTQSPAIMSASPGEKVTMTCSASASISYMHWYQQKSGTSPKPWIYEISKLASGVPARFSGSGS
GTSYSLTISSMEAEDAAIYYCQQWSSYPLTFGAGTKLEIK
- LCDR1 nucleic acid sequence of TA6
SEQ ID NO: 11
AGTGCCAGTGCAAGTATAAGTTACATGCAC
- LCDR1 amino acid sequence of TA6 (as defined by
the Kabat system)
SEQ ID NO: 12
SASASISYMH
- LCDR2 nucleic acid sequence of TA6
SEQ ID NO: 13
GAAATATCCAAACTGGCTTCT
- LCDR2 amino acid sequence of TA6 (as defined by
the Kabat system)
SEQ ID NO: 14
EISKLAS
- LCDR3 nucleic acid sequence of TA6
SEQ ID NO: 15
CAACAGTGGAGTAGTTACCCGCTCACG
- LCDR3 amino acid sequence of TA6 (as defined by
the Kabat system)
SEQ ID NO: 16
QQWSSYPLT
- HCVR nucleic acid sequence of TA12
SEQ ID NO: 17
CAGGTCAAGCTGCAGCAGTCTGGAGCTGAGCTGATGAAGCCTGGGGCCTCAGTGAAGCTTTCCTGC
AAGGCTTCTGGCTACACCTTCACCAACTACTGGATGCACTGGGTGAAGCAGAGGCCTGGAAAAGGC
CTTGAGTGGATTGGAATGACTCATCCTAAAAGTAGTACTACTGACTACAATGAGAAGTTCGAGAGC
AAGGCCACACTGACTGCAGACAAATCCTCCAGCACGGCCTACATGCAACTCAGCAGCCTGACTTCT
GAGGACTCTGCGGTCTATTACTGTGCAAGAGGGGGCTACGGTAGCCCCTTTGACTACTGGGGCCAA
GGGACCACGGTCACCGTCTCCTCA
- HCVR amino acid sequence of TA12 (CDRs 1-3 based
on the Kabat system underlined)
SEQ ID NO: 18
QVKLQQSGAELMKPGASVKLSCKASGYTFTNYWMHWVKQRPGKGLEWIGMTHPKSSTTDYNEKFES
KATLTADKSSSTAYMQLSSLTSEDSAVYYCARGGYGSPFDYWGQGTTVTVSS
- HCDR1 nucleic acid sequence of TA12
SEQ ID NO: 19
GGCTACACCTTCACCAACTAC
- HCDR1 amino acid sequence of TA12 (as defined by
the Kabat system)
SEQ ID NO: 20
GYTFTNY
- HCDR2 nucleic acid sequence of TA12
SEQ ID NO: 21
CATCCTAAAAGTAGTACT
- HCDR2 amino acid sequence of TA12 (as defined by
the Kabat system)
SEQ ID NO: 22
HPKSST
- HCDR3 nucleic acid sequence of TA12
SEQ ID NO: 23
GGGGGCTACGGTAGCCCCTTTGACTAC
- HCDR3 amino acid sequence of TA12 (as defined by
the Kabat system)
SEQ ID NO: 24
GGYGSPFDY
- LCVR nucleic acid sequence of TA12
SEQ ID NO: 25
CAAATTGTTCTCACCCAGTCTCCAGCAATCATGTCTGCATCTCCAGGGGAGAAGGTCACCATGACC
TGCAGTGCCAGCTCAAGTGTAAGTTACATGCACTGGTACCAGCAGAAGTCAGGCACCTCCCCCAAA
AGATGGATTTATGACACATCCAAACTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCACCCACGTTCGGAGGGGGCACCAAGTTGGAAATAAAA
- LCVR amino acid sequence of TA12 (CDRs 1-3 based
on Kabat system underlined)
SEQ ID NO: 26
QIVLTQSPAIMSASPGEKVTMTCSASSSVSYMHWYQQKSGTSPKRWIYDTSKLASGVPARFSGSGS
GTSYSLTISSMEAEDAATYYCQQWSSNPPTFGGGTKLEIK
- LCDR1 nucleic acid sequence of TA12
SEQ ID NO: 27
AGTGCCAGCTCAAGTGTAAGTTACATGCAC
- LCDR1 amino acid sequence of TA12 (as defined by
the Kabat system)
SEQ ID NO: 28
SASSSVSYMH
- LCDR2 nucleic acid sequence of TA12
SEQ ID NO: 29
GACACATCCAAACTGGCTTCT
- LCDR2 amino acid sequence of TA12 (as defined by
the Kabat system)
SEQ ID NO: 30
DTSKLAS
- LCDR3 nucleic acid sequence of TA12
SEQ ID NO: 31
CAGCAGTGGAGTAGTAACCCACCCACG
- LCDR3 amino acid sequence of TA12 (as defined by
the Kabat system)
SEQ ID NO: 32
QQWSSNPPT
- HCVR nucleic acid sequence of TB8
SEQ ID NO: 33
CAGGTCAAGCTGCAGGAGTCAGGGGGAGGCTTAGTGAAGCCTGGAGGGTCCCTGAAACTCTCCTGT
GCAGCCTCTGGATTCACTTTCAGTGACTATGGAATGCACTGGGTTCGTCAGGCTCCAGAGAAGGGG
CTGGAGTGGGTTGCATACATTAGTAGTGGCAGTAGTACCATCTACTATGCAGACACAGTGAAGGGC
CGATTCACCATCTCCAGAGACAATGCCAAGAACACCCTGTTCCTGCAAATGACCAGTCTGAGGTCT
GGGGACACGGCCATGTATTACTGTGCAAGGCACGGTAGTAGCTACTGGTCCTTCGATGTCTGGGGC
CAAGGGACCACGGTCACCGTCTCCTCA
- HCVR amino acid sequence of TB8 (CDRs 1-3 based on
the Kabat system underlined)
SEQ ID NO: 34
QVKLQESGGGLVKPGGSLKLSCAASGFTFSDYGMHWVRQAPEKGLEWVAYISSGSSTIYYADTVKG
RFTISRDNAKNTLFLQMTSLRSGDTAMYYCARHGSSYWSFDVWGQGTTVTVSS
- HCDR1 nucleic acid sequence of TB8
SEQ ID NO: 35
GGATTCACTTTCAGTGACTAT
- HCDR1 amino acid sequence of TB8 (as defined by
the Kabat system)
SEQ ID NO: 36
GFTFSDY
- HCDR2 nucleic acid sequence of TB8
SEQ ID NO: 37
AGTAGTGGCAGTAGTACC
- HCDR2 amino acid sequence of TB8 (as defined by
the Kabat system)
SEQ ID NO: 38
SSGSST
- HCDR3 nucleic acid sequence of TB8
SEQ ID NO: 39
CACGGTAGTAGCTACTGGTCCTTCGATGTC
- HCDR3 amino acid sequence of TB8 (as defined by
the Kabat system)
SEQ ID NO: 40
HGSSYWSFDV
- LCVR nucleic acid sequence of TB8
SEQ ID NO: 41
GAAAATGTGCTCACCCAGTCTCCAGCAATCATGTCTGCATCTCCAGGGGAGAAGGTCACCATATCC
TGCAGTGCCAGCTCAAGTGTAAATTACATGTACTGGTACAAGCAGAAGCCAGGATCCTCCCCCAAG
CCCTGGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCCGAAGATGCTGCCACTTATTACTGCCAG
CAGTGGAGTAGTAACCCGCTCACGTTCGGTGCTGGCACCAAGCTGGAAATCAAA
- LCVR amino acid sequence of TB8 (CDRs 1-3 based on
Kabat system underlined)
SEQ ID NO: 42
ENVLTQSPAIMSASPGEKVTISCSASSSVNYMYWYKQKPGSSPKPWIYRTSNLASGVPARFSGSGS
GTSYSLTISSMEAEDAATYYCQQWSSNPLTFGAGTKLEIK
- LCDR1 nucleic acid sequence of TB8
SEQ ID NO: 43
AGTGCCAGCTCAAGTGTAAATTACATGTAC
- LCDR1 amino acid sequence of TB8 (as defined by
the Kabat system)
SEQ ID NO: 44
SASSSVNYMY
- LCDR2 nucleic acid sequence of TB8
SEQ ID NO: 45
CGCACATCCAACCTGGCTTCT
- LCDR2 amino acid sequence of TB8 (as defined by
the Kabat system)
SEQ ID NO: 46
RTSNLAS
- LCDR3 nucleic acid sequence of TB8
SEQ ID NO: 47
CAGCAGTGGAGTAGTAACCCGCTCACG
- LCDR3 amino acid sequence of TB8 (as defined by
the Kabat system)
SEQ ID NO: 48
QQWSSNPLT
- HCVR nucleic acid sequence of TC6
SEQ ID NO: 49
CAGGTCAAGCTGCAGGAGTCAGGGGCTGAGCTTGTGAAGCCTGGGGCTTCAGTGAAGCTGTCCTGC
AAGGCTTCTGGCTACACTTTTACCAGCTACTGGATACACTGGGTGAAGCAGAGGCCTGGACAAGGC
CTTGAGTGGATTGGAATGATTCATCCAAAGAGTGGAAGTATTAACTACAATGAGAAGTTCAGGAGC
AAGGCCACACTGACTGTAGACAAATCCTCCAGCACAGCCTACATGCAACTCAGCAGGCTGACATCT
GAGGACTCTGCGGTCTATTTCTGTGCAAGCGGGGGCTACGGTAGTAGCCTTGACTACTGGGGCCAA
GGGACCACGGTCACCGTCTCCTCA
- HCVR amino acid sequence of TC6 (CDRs 1-3 based on
the Kabat system underlined)
SEQ ID NO: 50
QVKLQESGAELVKPGASVKLSCKASGYTFTSYWIHWVKQRPGQGLEWIGMIHPKSGSINYNEKFRS
KATLTVDKSSSTAYMQLSRLTSEDSAVYFCASGGYGSSLDYWGQGTTVTVSS
- HCDR1 nucleic acid sequence of TC6
SEQ ID NO: 51
GGCTACACTTTTACCAGCTAC
- HCDR1 amino acid sequence of TC6 (as defined by
the Kabat system)
SEQ ID NO: 52
GYTFTSY
- HCDR2 nucleic acid sequence of TC6
SEQ ID NO: 53
CATCCAAAGAGTGGAAGT
- HCDR2 amino acid sequence of TC6 (as defined by
the Kabat system)
SEQ ID NO: 54
HPKSGS
- HCDR3 nucleic acid sequence of TC6
SEQ ID NO: 55
GGGGGCTACGGTAGTAGCCTTGACTAC
- HCDR3 amino acid sequence of TC6 (as defined by
the Kabat system)
SEQ ID NO: 56
GGYGSSLDY
- LCVR nucleic acid sequence of TC6
SEQ ID NO: 57
CAAATTGTTCTCACCCAGTCTCCAGCAATCATGTCCGCATCTCCAGGGGAGAAGGTCACCATGACC
TGCAGTGCCAGCTCAAGTGTAAGTTACATGTACTGGTACCAGCAGAAGCCAAGATCCTCCCCCATA
TCCTGGATTTATCTCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTGACCCGCTCACGTTCGGTGCTGGGACCAAGCTGGAGCTGAAA
- LCVR amino acid sequence of TC6 (CDRs 1-3 based on
Kabat system underlined)
SEQ ID NO: 58
QIVLTQSPAIMSASPGEKVTMTCSASSSVSYMYWYQQKPRSSPISWIYLTSNLASGVPARFSGSGS
GTSYSLTISSMEAEDAATYYCQQWSSDPLTFGAGTKLELK
- LCDR1 nucleic acid sequence of TC6
SEQ ID NO: 59
AGTGCCAGCTCAAGTGTAAGTTACATGTAC
- LCDR1 amino acid sequence of TC6 (as defined by
the Kabat system)
SEQ ID NO: 60
SASSSVSYMY
- LCDR2 nucleic acid sequence of TC6
SEQ ID NO: 61
CTCACATCCAACCTGGCTTCT
- LCDR2 amino acid sequence of TC6 (as defined by
the Kabat system)
SEQ ID NO: 62
LTSNLAS
- LCDR3 nucleic acid sequence of TC6
SEQ ID NO: 63
CAGCAGTGGAGTAGTGACCCGCTCACG
- LCDR3 amino acid sequence of TC6 (as defined by
the Kabat system)
SEQ ID NO: 64
QQWSSDPLT
- HCVR nucleic acid sequence of TE9
SEQ ID NO: 65
CAGGTGCAACTGCAGCAGTCAGGGGCTGCGCTGGTAAAGCCTGGGACTTCAGTGAAGTTGTCCTGC
AAGGCTTCTGGCTACACTTTCACCAGCTACTGGATGCACTGGGTGAAGCAGAGGCCTGGACAAGGC
CTTGAGTGGATTGGAATGATTCATCCTAAAAGTGGTAGTGTTGACTACAATGAGAAATTCACGAAC
AAGGCCACACTGACTGGAGACAAATCCTCCGGCACAGCCTACATGCAACTCAGCAGCCTGACATCT
GAAGACTCTGCGGTCTATTACTGTGCAAGAGGGGGCTACGGTAGCCCCTTTGACTACTGGGGCCAA
GGGACCACGGTCACCGTCTCCTCA
- HCVR amino acid sequence of TE9 (CDRs 1-3 based on
the Kabat system underlined)
SEQ ID NO: 66
QVQLQQSGAALVKPGTSVKLSCKASGYTFTSYWMHWVKQRPGQGLEWIGMIHPKSGSVDYNEKFTN
KATLTGDKSSGTAYMQLSSLTSEDSAVYYCARGGYGSPFDYWGQGTTVTVSS
- HCDR1 nucleic acid sequence of TE9
SEQ ID NO: 67
GGCTACACTTTCACCAGCTAC
- HCDR1 amino acid sequence of TE9 (as defined by
the Kabat system)
SEQ ID NO: 68
GYTFTSY
- HCDR2 nucleic acid sequence of TE9
SEQ ID NO: 69
CATCCTAAAAGTGGTAGT
- HCDR2 amino acid sequence of TE9 (as defined by
the Kabat system)
SEQ ID NO: 70
HPKSGS
- HCDR3 nucleic acid sequence of TE9
SEQ ID NO: 71
GGGGGCTACGGTAGCCCCTTTGACTAC
- HCDR3 amino acid sequence of TE9 (as defined by
the Kabat system)
SEQ ID NO: 72
GGYGSPFDY
- LCVR nucleic acid sequence of TE9
SEQ ID NO: 73
GAAAATGTGCTCACCCAGTCTCCAGCAATCATGTCTGCATCTCCAGGGGAGAAGGTCACCATGACC
TGCAGTGCCAGCTCAAGTGTAAGTTACATGTACTGGTACCAGCAGAAGCCAGGATCCTCCCCCAGA
CTCCTGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCACCGACGTTCGGTGGAGGCACCAAGTTGGAAATAAAA
- LCVR amino acid sequence of TE9 (CDRs 1-3 based on
Kabat system underlined)
SEQ ID NO: 74
ENVLTQSPAIMSASPGEKVTMTCSASSSVSYMYWYQQKPGSSPRLLIYRTSNLASGVPARFSGSGS
GTSYSLTISSMEAEDAATYYCQQWSSNPPTFGGGTKLEIK
- LCDR1 nucleic acid sequence of TE9
SEQ ID NO: 75
AGTGCCAGCTCAAGTGTAAGTTACATGTAC
- LCDR1 amino acid sequence of TE9 (as defined by
the Kabat system)
SEQ ID NO: 76
SASSSVSYMY
- LCDR2 nucleic acid sequence of TE9
SEQ ID NO: 77
CGCACATCCAACCTGGCTTCT
- LCDR2 amino acid sequence of TE9 (as defined by
the Kabat system)
SEQ ID NO: 78
RTSNLAS
- LCDR3 nucleic acid sequence of TE9
SEQ ID NO: 79
CAGCAGTGGAGTAGTAACCCACCGACG
- LCDR3 amino acid sequence of TE9 (as defined by
the Kabat system)
SEQ ID NO: 80
QQWSSNPPT
- HCVR nucleic acid sequence of TF9
SEQ ID NO: 81
CAGGTCAAACTGCAGGAGTCTGGGGCTGAGCTGGTAAAGCCTGGGGCTTCAGTGAAGTTGTCCTGC
AAGGCTTCTGGCTACACTTTTACCAGCTACTGGATACACTGGGTGAAGCAGAGGCCTGGACAAGGC
CTTGAGTGGATTGGAATGATTCATCCAAAGAGTGGAAGTATTAACTACAATGAGAAGTTCAGGAGC
AAGGCCACACTGACTGTAGACAAATCCTCCAGCACAGCCTACATGCAACTCAGCAGGCTGACATCT
GAGGACTCTGCGGTCTATTTCTGTGCAAGCGGGGGCTACGGTAGTAGCCTTGACTACTGGGGCCAA
GGGACCACGGTCACCGTCTCCTCA
- HCVR amino acid sequence of TF9 (CDRs 1-3 based on
the Kabat system underlined)
SEQ ID NO: 82
QVKLQESGAELVKPGASVKLSCKASGYTFTSYWIHWVKQRPGQGLEWIGMIHPKSGSINYNEKFRS
KATLTVDKSSSTAYMQLSRLTSEDSAVYFCASGGYGSSLDYWGQGTTVTVSS
- HCDR1 nucleic acid sequence of TF9
SEQ ID NO: 83
GGCTACACTTTTACCAGCTAC
- HCDR1 amino acid sequence of TF9 (as defined by
the Kabat system)
SEQ ID NO: 84
GYTFTSY
- HCDR2 nucleic acid sequence of TF9
SEQ ID NO: 85
CATCCAAAGAGTGGAAGT
- HCDR2 amino acid sequence of TF9 (as defined by
the Kabat system)
SEQ ID NO: 86
HPKSGS
- HCDR3 nucleic acid sequence of TF9
SEQ ID NO: 87
GGGGGCTACGGTAGTAGCCTTGACTAC
- HCDR3 amino acid sequence of TF9 (as defined by
the Kabat system)
SEQ ID NO: 88
GGYGSSLDY
- LCVR nucleic acid sequence of TF9
SEQ ID NO: 89
GAAAATGTGCTCACCCAGTCTCCAGCAATCATGTCTGCATCTCCAGGGGAGAAGGTCACCATATCC
TGCAGTGCCAGCTCAAGTGTAAGTTACGTGTACTGGTACCAGCAGAAGCCAGGATCCTCCCCCAAA
CCCTGGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCGCTCACGTTCGGTGCTGGGACCAAGCTGGAGCTGAAA
- LCVR amino acid sequence of TF9 (CDRs 1-3 based on
Kabat system underlined)
SEQ ID NO: 90
ENVLTQSPAIMSASPGEKVTISCSASSSVSYVYWYQQKPGSSPKPWIYRTSNLASGVPARFSGSGS
GTSYSLTISSMEAEDAATYYCQQWSSNPLTFGAGTKLELK
- LCDR1 nucleic acid sequence of TF9
SEQ ID NO: 91
AGTGCCAGCTCAAGTGTAAGTTACGTGTAC
- LCDR1 amino acid sequence of TF9 (as defined by
the Kabat system)
SEQ ID NO: 92
SASSSVSYVY
- LCDR2 nucleic acid sequence of TF9
SEQ ID NO: 93
CGCACATCCAACCTGGCTTCT
- LCDR2 amino acid sequence of TF9 (as defined by
the Kabat system)
SEQ ID NO: 94
RTSNLAS
- LCDR3 nucleic acid sequence of TF9
SEQ ID NO: 95
CAGCAGTGGAGTAGTAACCCGCTCACG
- LCDR3 amino acid sequence of TF9 (as defined by
the Kabat system)
SEQ ID NO: 96
QQWSSNPLT
- HCVR nucleic acid sequence of BA5
SEQ ID NO: 97
CAGGTGAAACTGCAGGAGTCTGGGGCTGAGCTGGTAAAGCCTGGGGCTTCAGTGAAGTTGTCCTGC
AAGGCTTCTGGCTACACTTTCACCAGCTACTGGATGCACTGGGTGAAGCAGAGGCCTGGACAAGGC
CTTGAGTGGATTGGAATGATTCATCCTAATAGTGGTAGTACTAACTACAATGAGAAGTTCAAGGGC
AAGGCCACACTGACTGCAGACAAATCCTCCAGCACAGTCTACATGCAGCTCAGTAGCCTGACCTCT
GAGGACTCTGCGGTCTATTTCTGTGCAAGATCGGGGGGTAATTACTTTGACTACTGGGGCCAAGGG
ACCACGGTCACCGTCTCCTCA
- HCVR amino acid sequence of BA5 (CDRs 1-3 based on
the Kabat system underlined)
SEQ ID NO: 98
QVKLQESGAELVKPGASVKLSCKASGYTFTSYWMHWVKQRPGQGLEWIGMIHPNSGSTNYNEKFKG
KATLTADKSSSTVYMQLSSLTSEDSAVYFCARSGGNYFDYWGQGTTVTVSS
- HCDR1 nucleic acid sequence of BA5
SEQ ID NO: 99
GGCTACACTTTCACCAGCTAC
- HCDR1 amino acid sequence of BA5 (as defined by
the Kabat system)
SEQ ID NO: 100
GYTFTSY
- HCDR2 nucleic acid sequence of BA5
SEQ ID NO: 101
CATCCTAATAGTGGTAGT
- HCDR2 amino acid sequence of BA5 (as defined by
the Kabat system)
SEQ ID NO: 102
HPNSGS
- HCDR3 nucleic acid sequence of BA5
SEQ ID NO: 103
TCGGGGGGTAATTACTTTGACTAC
- HCDR3 amino acid sequence of BA5 (as defined by
the Kabat system)
SEQ ID NO: 104
SGGNYFDY
- LCVR nucleic acid sequence of BA5
SEQ ID NO: 105
GACATTGTGCTAACCCAGTCTCCAGCAATCATGTCTGCATCTCCAGGGGAGAAGGTCACCATATCC
TGCAGTGCCAGCTCAAGTGTAAGTTACATGTACTGGTACAAGCAGAAGCCAGGATCCTCCCCCAAA
CCCTGGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTTACCCGCTCACGTTCGGTGCTGGGACCAAGCTGGAAATAAAA
- LCVR amino acid sequence of BA5 (CDRs 1-3 based
on Kabat system underlined)
SEQ ID NO: 106
DIVLTQSPAIMSASPGEKVTISCSASSSVSYMYWYKQKPGSSPKPWIYRTSNLASGVPARFSGSGS
GTSYSLTISSMEAEDAATYYCQQWSSYPLTFGAGTKLEIK
- LCDR1 nucleic acid sequence of BA5
SEQ ID NO: 107
AGTGCCAGCTCAAGTGTAAGTTACATGTAC
- LCDR1 amino acid sequence of BA5 (as defined by
the Kabat system)
SEQ ID NO: 108
SASSSVSYMY
- LCDR2 nucleic acid sequence of BA5
SEQ ID NO: 109
CGCACATCCAACCTGGCTTCT
- LCDR2 amino acid sequence of BA5 (as defined by
the Kabat system)
SEQ ID NO: 110
RTSNLAS
- LCDR3 nucleic acid sequence of BA5
SEQ ID NO: 111
CAGCAGTGGAGTAGTTACCCGCTCACG
- LCDR3 amino acid sequence of BA5 (as defined by
the Kabat system)
SEQ ID NO: 112
QQWSSYPLT
- HCVR nucleic acid sequence of BB1
SEQ ID NO: 113
CAGGTCAAGCTGCAGGAGTCAGGACCTGAGCTGGTGAAGCCTGGGGCTTCAGTGAAGATATCCTGC
AAGGCTTCTGGCTACAGCTTCACAAGCTACTATATACACTGGGTGAAGCAGAGGCCTGGAAAAGGC
CTTGAGTGGATTGGAATGACTCATCCTAAAAGTAGTACTACTGACTACAATGAGAAGTTCGAGAGC
AAGGCCACACTGACTGCAGACAAATCCTCCAGCACGGCCTACATGCAACTCAGCAGCCTGACTTCT
GAGGACTCTGCGGTCTATTACTGTGCAAGAGGGGGCTACGGTAGCCCCTTTGACTACTGGGGCCAA
GGGACCACGGTCACCGTCTCCTCA
- HCVR amino acid sequence of BB1 (CDRs 1-3 based
on the Kabat system underlined)
SEQ ID NO: 114
QVKLQESGPELVKPGASVKISCKASGYSFTSYYIHWVKQRPGKGLEWIGMTHPKSSTTDYNEKFES
KATLTADKSSSTAYMQLSSLTSEDSAVYYCARGGYGSPFDYWGQGTTVTVSS
- HCDR1 nucleic acid sequence of BB1
SEQ ID NO: 115
GGCTACAGCTTCACAAGCTAC
- HCDR1 amino acid sequence of BB1 (as defined by
the Kabat system)
SEQ ID NO: 116
GYSFTSY
- HCDR2 nucleic acid sequence of BB1
SEQ ID NO: 117
CATCCTAAAAGTAGTACT
- HCDR2 amino acid sequence of BB1 (as defined by
the Kabat system)
SEQ ID NO: 118
HPKSST
- HCDR3 nucleic acid sequence of BB1
SEQ ID NO: 119
GGGGGCTACGGTAGCCCCTTTGACTAC
- HCDR3 amino acid sequence of BB1 (as defined by
the Kabat system)
SEQ ID NO: 120
GGYGSPFDY
- LCVR nucleic acid sequence of BB1
SEQ ID NO: 121
GAAATTGTTCTCACCCAGTCTCCAGCAATCATGTCTGCATCTCCAGGGGAGAAGGTCACCATGACC
TGCAGTGCCACCTCAAGTGTAAGTTACATGCACTGGTACCAGCAGAAGCCAGGCACCTCCCCCAAA
AGATGGATTTATGACACATCCAAACTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCACCCACGTTCGGCTCGGGGACCAAGCTGGAAATAAAA
- LCVR amino acid sequence of BB1 (CDRs 1-3 based
on Kabat system underlined)
SEQ ID NO: 122
EIVLTQSPAIMSASPGEKVTMTCSATSSVSYMHWYQQKPGTSPKRWIYDTSKLASGVPARFSGSGS
GTSYSLTISSMEAEDAATYYCQQWSSNPPTFGSGTKLEIK
- LCDR1 nucleic acid sequence of BB1
SEQ ID NO: 123
AGTGCCACCTCAAGTGTAAGTTACATGCAC
- LCDR1 amino acid sequence of BB1 (as defined by
the Kabat system)
SEQ ID NO: 124
SATSSVSYMH
- LCDR2 nucleic acid sequence of BB1
SEQ ID NO: 125
GACACATCCAAACTGGCTTCT
- LCDR2 amino acid sequence of BB1 (as defined by
the Kabat system)
SEQ ID NO: 126
DTSKLAS
- LCDR3 nucleic acid sequence of BB1
SEQ ID NO: 127
CAGCAGTGGAGTAGTAACCCACCCACG
- LCDR3 amino acid sequence of BB1 (as defined by
the Kabat system)
SEQ ID NO: 128
QQWSSNPPT
- HCVR nucleic acid sequence of BB5
SEQ ID NO: 129
CAGGTCAAACTGCAGCAGTCAGGGACTGAGCTTGTGATGCCTGGGGCTTCAGTGAAGCTGTCCTGC
AAGGCTTCCGGCTACACCTTCACCAGCTACTGGATGCACTGGGTAAAGCAGAGGCCTGGACAAGGC
CTTGAGTGGATCGGAATGATTGATTCTTCTGATAGTTATACTAACTACAATCAAAAGTTCAAGGGC
AAGGCCACATTGACTGTAGACACATCCTCCAGCACAGCCTACATGCAGCTCAGCAGCCTGACATCT
GAGGACTCTGCGGTCTATTACTGTGCAAGAGGATACTACGGTAGTAGCCACTATGCTATGGACTAC
TGGGGCCAAGGGACCACGGTCACCGTCTCCTCA
- HCVR amino acid sequence of BB5 (CDRs 1-3 based
on the Kabat system underlined)
SEQ ID NO: 130
QVKLQQSGTELVMPGASVKLSCKASGYTFTSYWMHWVKQRPGQGLEWIGMIDSSDSYTNYNQKFKG
KATLTVDTSSSTAYMQLSSLTSEDSAVYYCARGYYGSSHYAMDYWGQGTTVTVSS
- HCDR1 nucleic acid sequence of BB5
SEQ ID NO: 131
GGCTACACCTTCACCAGCTAC
- HCDR1 amino acid sequence of BB5 (as defined by
the Kabat system)
SEQ ID NO: 132
GYTFTSY
- HCDR2 nucleic acid sequence of BB5
SEQ ID NO: 133
GATTCTTCTGATAGTTAT
- HCDR2 amino acid sequence of BB5 (as defined by
the Kabat system)
SEQ ID NO: 134
DSSDSY
- HCDR3 nucleic acid sequence of BB5
SEQ ID NO: 135
GGATACTACGGTAGTAGCCACTATGCTATGGACTAC
- HCDR3 amino acid sequence of BB5 (as defined by
the Kabat system)
SEQ ID NO: 136
GYYGSSHYAMDY
- LCVR nucleic acid sequence of BB5
SEQ ID NO: 137
GATGTTTTGATGACCCAAACTCCACTCTCCCTGCCTGTCAGTCTTGGAGATCAAGCCTCCATCTCT
TGCAGATCTAGTCAGAGCCTTGTACACAGTAATGGAAACACCTATTTAGAATGGTACCTGCAGAAA
CCAGGCCAGTCTCCAAAGCTCCTAATCTACAAAGTTTCCAACCGATTTTCTGGGGTCCCAGACAGG
TTCAGTGGCAGTGGATCAGGGACAGATTTCACACTCAAGATCAGCGGAGTGGAGGCTGAGGATCTG
GGAGTTTATTACTGCTTTCAAGGTTCACATGTTCCGTACACGTTCGGAGGGGGGACCAAGCTGGAA
ATCAAA
- LCVR amino acid sequence of BB5 (CDRs 1-3 based
on Kabat system underlined)
SEQ ID NO: 138
DVLMTQTPLSLPVSLGDQASISCRSSQSLVHSNGNTYLEWYLQKPGQSPKLLIYKVSNRFSGVPDR
FSGSGSGTDFTLKISGVEAEDLGVYYCFQGSHVPYTFGGGTKLEIK
- LCDR1 nucleic acid sequence of BB5
SEQ ID NO: 139
AGATCTAGTCAGAGCCTTGTACACAGTAATGGAAACACCTATTTAGAA
- LCDR1 amino acid sequence of BB5 (as defined by
the Kabat system)
SEQ ID NO: 140
RSSQSLVHSNGNTYLE
- LCDR2 nucleic acid sequence of BB5
SEQ ID NO: 141
AAAGTTTCCAACCGATTTTCT
- LCDR2 amino acid sequence of BB5 (as defined by
the Kabat system)
SEQ ID NO: 142
KVSNRFS
- LCDR3 nucleic acid sequence of BB5
SEQ ID NO: 143
TTTCAAGGTTCACATGTTCCGTACACG
- LCDR3 amino acid sequence of BB5 (as defined by
the Kabat system)
SEQ ID NO: 144
FQGSHVPYT
- HCVR nucleic acid sequence of BC10
SEQ ID NO: 145
CAGGTGAAGCTGCAGCAGTCAGGGGCTGAGCTGGTAAAGCCTGGGGCTTCAGTGAAGTTGTCCTGC
AAGGCTCCTGGCTACACTTCCACCAACTACTGGATGCACTGGGTGAAGCAGAGGCCTGGACAAGGC
CTTGAGTGGATTGGAATGATTCATCCCAATAGTGGTGGTACCAACTACAATGAGAAGTTCAAGAGC
AAGGCCACACTGACTGTAGACAAATCCTCCAGCACAGCCTACATGCAACTCAGCAGCCTGACATCT
GAGGACTCTGCGGTCTATTACTGTGCAAGCCTCGGTAGTAGCCTTGACTATTGGGGCCAAGGGACC
ACGGTCACCGTCTCCTCA
- HCVR amino acid sequence of BC10 (CDRs 1-3 based
on the Kabat system underlined)
SEQ ID NO: 146
QVKLQQSGAELVKPGASVKLSCKAPGYTSTNYWMHWVKQRPGQGLEWIGMIHPNSGGTNYNEKFKS
KATLTVDKSSSTAYMQLSSLTSEDSAVYYCASLGSSLDYWGQGTTVTVSS
- HCDR1 nucleic acid sequence of BC10
SEQ ID NO: 147
GGCTACACTTCCACCAACTAC
- HCDR1 amino acid sequence of BC10 (as defined by
the Kabat system)
SEQ ID NO: 148
GYTSTNY
- HCDR2 nucleic acid sequence of BC10
SEQ ID NO: 149
CATCCCAATAGTGGTGGT
- HCDR2 amino acid sequence of BC10 (as defined by
the Kabat system)
SEQ ID NO: 150
HPNSGG
- HCDR3 nucleic acid sequence of BC10
SEQ ID NO: 151
CTCGGTAGTAGCCTTGACTAT
- HCDR3 amino acid sequence of BC10 (as defined by
the Kabat system)
SEQ ID NO: 152
LGSSLDY
- LCVR nucleic acid sequence of BC10
SEQ ID NO: 153
GAAAATGTGCTCACCCAGTCTCCAGCAATCATGTCTGCATCTCCAGGGGAGAAGGTCACCATGACC
TGCAGTGCCAGCTCAAGTATAAGTTACATGCACTGGTACCAGCAGAAGCCAGGCACCTCCCCCAAA
AGATGGATTTATGACACATCCAAACTGGCTCCTGGAGTCCCAGCTCGCTTCAGTGGCAGTGGGTCT
GGGACCTCTTACTCTCTCACAATCAGCAGTGTGGAGGCTGAAGATGCTGCCACTTATTACTGCCAG
CAGTACAGTGGTTACCCACTGACGTTCGGTGGAGGCACCAAGCTGGAGCTGAAA
- LCVR amino acid sequence of BC10 (CDRs 1-3 based
on Kabat system underlined)
SEQ ID NO: 154
ENVLTQSPAIMSASPGEKVTMTCSASSSISYMHWYQQKPGTSPKRWIYDTSKLAPGVPARFSGSGS
GTSYSLTISSVEAEDAATYYCQQYSGYPLTFGGGTKLELK
- LCDR1 nucleic acid sequence of BC10
SEQ ID NO: 155
AGTGCCAGCTCAAGTATAAGTTACATGCAC
- LCDR1 amino acid sequence of BC10 (as defined by
the Kabat system)
SEQ ID NO: 156
SASSSISYMH
- LCDR2 nucleic acid sequence of BC10
SEQ ID NO: 157
GACACATCCAAACTGGCTCCT
- LCDR2 amino acid sequence of BC10 (as defined by
the Kabat system)
SEQ ID NO: 158
DTSKLAP
- LCDR3 nucleic acid sequence of BC10
SEQ ID NO: 159
CAGCAGTACAGTGGTTACCCACTGACG
- LCDR3 amino acid sequence of BC10 (as defined by
the Kabat system)
SEQ ID NO: 160
QQYSGYPLT
- HCVR nucleic acid sequence of BD9
SEQ ID NO: 161
CAGGTGCAGCTGCAGCAGTCAGGGGCTGAGCTGGTAAAGCCTGGGGCTTCAGTGAAGTTGTCCTGC
AAGGCTTCTGGCTACACTTTTACCAGCTACTGGATACACTGGGTGAAGCAGAGGCCTGGACAAGGC
CTTGAGTGGATTGGAATGATTCATCCAAAGAGTGGAAGTATTAACTACAATGAGAAGTTCAGGAGC
AAGGCCACACTGACTGTAGACAAATCCTCCAGCACAGCCTACATGCAACTCAGCAGGCTGACATCT
GAGGACTCTGCGGTCTATTTCTGTGCAAGCGGGGGCTACGGTAGTAGCCTTGACTACTGGGGCCAA
GGGACCACGGTCACCGTCTCCTCA
- HCVR amino acid sequence of BD9 (CDRs 1-3 based
on the Kabat system underlined)
SEQ ID NO: 162
QVQLQQSGAELVKPGASVKLSCKASGYTFTSYWIHWVKQRPGQGLEWIGMIHPKSGSINYNEKFRS
KATLTVDKSSSTAYMQLSRLTSEDSAVYFCASGGYGSSLDYWGQGTTVTVSS
- HCDR1 nucleic acid sequence of BD9
SEQ ID NO: 163
GGCTACACTTTTACCAGCTAC
- HCDR1 amino acid sequence of BD9 (as defined by
the Kabat system)
SEQ ID NO: 164
GYTFTSY
- HCDR2 nucleic acid sequence of BD9
SEQ ID NO: 165
CATCCAAAGAGTGGAAGT
- HCDR2 amino acid sequence of BD9 (as defined by
the Kabat system)
SEQ ID NO: 166
HPKSGS
- HCDR3 nucleic acid sequence of BD9
SEQ ID NO: 167
GGGGGCTACGGTAGTAGCCTTGACTAC
- HCDR3 amino acid sequence of BD9 (as defined by
the Kabat system)
SEQ ID NO: 168
GGYGSSLDY
- LCVR nucleic acid sequence of BD9
SEQ ID NO: 169
GAAAATGTGCTCACCCAGTCTCCAGCAATCATGTCTGCATCTCCAGGGGAGAAGGTCACCATGACC
TGCAGTGCCAGCTCAAGTGTAAGTTACATGTACTGGTACCAGCAGAAGCCAGGATCCTCCCCCAGA
CTCCTGATTTATGACACATCCAACCTGGCTTCTGGAGTCCCTGCTCGCTTCAGTGGCAGTGGGTCT
GGGACCTCTTACTCTCTCACAATCAGCAGCATGGAGGCTGTAGATGCTGCCACTTATTACTGCCAG
CAGTGGAGTAGTGACCCGCTCACGTTCGGTGCTGGGACCAAGCTGGAGCTGAAA
- LCVR amino acid sequence of BD9 (CDRs 1-3 based
on Kabat system underlined)
SEQ ID NO: 170
ENVLTQSPAIMSASPGEKVTMTCSASSSVSYMYWYQQKPGSSPRLLIYDTSNLASGVPARFSGSGS
GTSYSLTISSMEAVDAATYYCQQWSSDPLTFGAGTKLELK
- LCDR1 nucleic acid sequence of BD9
SEQ ID NO: 171
AGTGCCAGCTCAAGTGTAAGTTACATGTAC
- LCDR1 amino acid sequence of BD9 (as defined by
the Kabat system)
SEQ ID NO: 172
SASSSVSYMY
- LCDR2 nucleic acid sequence of BD9
SEQ ID NO: 173
GACACATCCAACCTGGCTTCT
- LCDR2 amino acid sequence of BD9 (as defined by
the Kabat system)
SEQ ID NO: 174
DTSNLAS
- LCDR3 nucleic acid sequence of BD9
SEQ ID NO: 175
CAGCAGTGGAGTAGTGACCCGCTCACG
- LCDR3 amino acid sequence of BD9 (as defined by
the Kabat system)
SEQ ID NO: 176
QQWSSDPLT
- HCVR nucleic acid sequence of BF2
SEQ ID NO: 177
CAGGTCAAGCTGCAGCAGTCAGGGGCTGAGCTGGTAAAGCCTGGGGCTTCAGTGAAGTTGTCCTGC
AAGGCTTCTGGCTACACCTTCCCCAACTACTGGATAACCTGGGTGAAGCAGAGGCCTGGACAAGGC
CTTGAGTGGATTGGAATGATTCATCCAAAGAGTGGAAGTATTAACTACAATGAGAAGTTCAGGAGC
AAGGCCACACTGACTGTAGACAAATCTTCCAGCACAGCCTACATGCAACTCAGCAGGCTGACATCT
GAGGACTCTGCGGTCTATTACTGTGCAAGCGGGGGCTACGGTAGTAGCCTTGACTACTGGGGCCAA
GGGACCACGGTCACCGTCTCCTCA
- HCVR amino acid sequence of BF2 (CDRs 1-3 based
on the Kabat system underlined)
SEQ ID NO: 178
QVKLQQSGAELVKPGASVKLSCKASGYTFPNYWITWVKQRPGQGLEWIGMIHPKSGSINYNEKFRS
KATLTVDKSSSTAYMQLSRLTSEDSAVYYCASGGYGSSLDYWGQGTTVTVSS
- HCDR1 nucleic acid sequence of BF2
SEQ ID NO: 179
GGCTACACCTTCCCCAACTAC
- HCDR1 amino acid sequence of BF2 (as defined by
the Kabat system)
SEQ ID NO: 180
GYTFPNY
- HCDR2 nucleic acid sequence of BF2
SEQ ID NO: 181
CATCCAAAGAGTGGAAGT
- HCDR2 amino acid sequence of BF2 (as defined by
the Kabat system)
SEQ ID NO: 182
HPKSGS
- HCDR3 nucleic acid sequence of BF2
SEQ ID NO: 183
GGGGGCTACGGTAGTAGCCTTGACTAC
- HCDR3 amino acid sequence of BF2 (as defined by
the Kabat system)
SEQ ID NO: 184
GGYGSSLDY
- LCVR nucleic acid sequence of BF2
SEQ ID NO: 185
GAAAATGTGCTCACCCAGTCTCCAGCAATCATGTCTGCATCTCTAGGGGAGAAGGTCACCATGAGC
TGCAGGGCCAGCTCAAGTGTAAGTTACATGTACTGGTACCAGCAGAAGCCAAGATCCTCCCCCAAA
CCCTGGATTTATCTCACATCCAAACTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCATTCACGTTCGGCTCGGGGACCAAGCTGGAAATAAAA
- LCVR amino acid sequence of BF2 (CDRs 1-3 based
on Kabat system underlined)
SEQ ID NO: 186
ENVLTQSPAIMSASLGEKVTMSCRASSSVSYMYWYQQKPRSSPKPWIYLTSKLASGVPARFSGSGS
GTSYSLTISSMEAEDAATYYCQQWSSNPFTFGSGTKLEIK
- LCDR1 nucleic acid sequence of BF2
SEQ ID NO: 187
AGGGCCAGCTCAAGTGTAAGTTACATGTAC
- LCDR1 amino acid sequence of BF2 (as defined by
the Kabat system)
SEQ ID NO: 188
RASSSVSYMY
- LCDR2 nucleic acid sequence of BF2
SEQ ID NO: 189
CTCACATCCAAACTGGCTTCT
- LCDR2 amino acid sequence of BF2 (as defined by
the Kabat system)
SEQ ID NO: 190
LTSKLAS
- LCDR3 nucleic acid sequence of BF2
SEQ ID NO: 191
CAGCAGTGGAGTAGTAACCCATTCACG
- LCDR3 amino acid sequence of BF2 (as defined by
the Kabat system)
SEQ ID NO: 192
QQWSSNPFT
- HCVR nucleic acid sequence of BF9
SEQ ID NO: 193
CAGGTGAAGCTGCAGCAGTCTGGGGCTGAGCTGGTAAAGCCTGGGGCTTCAGTGAAGTTGTCCTGC
AAGGCTTCTGGCTACACTTTCACCAGCTACTGGGTGCACTGGGTGAAGCAGAGGCCTGGACAAGGC
CTTGAGTGGATTGGAATGATTCATCCTAAAAGTGGTAGTGCTGACTACAATGAGAAGTTCACGACC
AAGGCCACACTGACTGCAGACAAATCCTCCAGCACAGCCTACATGCAACTCAGCAGTCTGACATCT
GAGGACTCTGCGGTCTATTACTGTGCAAGAGGGGGCTACGGTAGCCCCTTTGACTACTGGGGCCAA
GGGACCACGGTCACCGTCTCCTCA
- HCVR amino acid sequence of BF9 (CDRs 1-3 based
on the Kabat system underlined)
SEQ ID NO: 194
QVKLQQSGAELVKPGASVKLSCKASGYTFTSYWVHWVKQRPGQGLEWIGMIHPKSGSADYNEKFTT
KATLTADKSSSTAYMQLSSLTSEDSAVYYCARGGYGSPFDYWGQGTTVTVSS
- HCDR1 nucleic acid sequence of BF9
SEQ ID NO: 195
GGCTACACTTTCACCAGCTAC
- HCDR1 amino acid sequence of BF9 (as defined by
the Kabat system)
SEQ ID NO: 196
GYTFTSY
- HCDR2 nucleic acid sequence of BF9
SEQ ID NO: 197
CATCCTAAAAGTGGTAGT
- HCDR2 amino acid sequence of BF9 (as defined by
the Kabat system)
SEQ ID NO: 198
HPKSGS
- HCDR3 nucleic acid sequence of BF9
SEQ ID NO: 199
GGGGGCTACGGTAGCCCCTTTGACTAC
- HCDR3 amino acid sequence of BF9 (as defined by
the Kabat system)
SEQ ID NO: 200
GGYGSPFDY
- LCVR nucleic acid sequence of BF9
SEQ ID NO: 201
GAAAATGTGCTCACCCAGTCTCCAGCAATCATGTCTGCATCTCCAGGGGAGAAGGTCACCATGACC
TGCAGTGCCAGCTCAAGTGTAAGTTACATGTACTGGTACCAGCAGAAGCCAGGATCCTCCCCCAGA
CTCCTGATTTATGACACATCCAACCTGGCTTCTGGAGCCCCTGTTCGCTTCAGTGGCCGTGGGTCT
GGGACCTCTTACTCTCTCACAATCAGCAGCATGGAGGCTGAAGATGCTGCCACTTATTACTGCCAG
CAGTGGAGTAATAACCCCCTCACGTTCGGTGCTGGGACCAAGCTGGAGCTGAAA
- LCVR amino acid sequence of BF9 (CDRs 1-3 based
on Kabat system underlined)
SEQ ID NO: 202
ENVLTQSPAIMSASPGEKVTMTCSASSSVSYMYWYQQKPGSSPRLLIYDTSNLASGAPVRFSGRGS
GTSYSLTISSMEAEDAATYYCQQWSNNPLTFGAGTKLELK
- LCDR1 nucleic acid sequence of BF9
SEQ ID NO: 203
AGTGCCAGCTCAAGTGTAAGTTACATGTAC
- LCDR1 amino acid sequence of BF9 (as defined by
the Kabat system)
SEQ ID NO: 204
SASSSVSYMY
- LCDR2 nucleic acid sequence of BF9
SEQ ID NO: 205
GACACATCCAACCTGGCTTCT
- LCDR2 amino acid sequence of BF9 (as defined by
the Kabat system)
SEQ ID NO: 206
DTSNLAS
- LCDR3 nucleic acid sequence of BF9
SEQ ID NO: 207
CAGCAGTGGAGTAATAACCCCCTCACG
- LCDR3 amino acid sequence of BF9 (as defined by
the Kabat system)
SEQ ID NO: 208
QQWSNNPLT
- HCVR nucleic acid sequence of BF12
SEQ ID NO: 209
CAGGTGAAACTGCAGCAGTCTGGGGCTGAGCTTGTGATGCCTGGGGCTTCAGTGACGCTGTCCTGC
AAGGCTTCTGGCTACACCTTCACCAGCTACTGGATGCACTGGGTGAAGCAGAGGCCTGGAAAAGGC
CTTGAGTGGATTGGAATGACTCATCCTAAAAGTAGTACTACTGACTACAATGAGAAGTTCAAGAGC
AAGGCCACACTGACTGCAGACAAATCCTCCAGCACGGCCTACATGCAGCTCAGCAGCCTGACTTCT
GAGGACTCTGCGGTCTATTACTGTGCAAGAGGGGGCTACGGTAGCCCCTTTGACTACTGGGGCCAA
GGGACCACGGTCACCGTCTCCTCA
- HCVR amino acid sequence of BF12 (CDRs 1-3 based
on the Kabat system underlined)
SEQ ID NO: 210
QVKLQQSGAELVMPGASVTLSCKASGYTFTSYWMHWVKQRPGKGLEWIGMTHPKSSTTDYNEKFKS
KATLTADKSSSTAYMQLSSLTSEDSAVYYCARGGYGSPFDYWGQGTTVTVSS
- HCDR1 nucleic acid sequence of BF12
SEQ ID NO: 211
GGCTACACCTTCACCAGCTAC
- HCDR1 amino acid sequence of BF12 (as defined by
the Kabat system)
SEQ ID NO: 212
GYTFTSY
- HCDR2 nucleic acid sequence of BF12
SEQ ID NO: 213
CATCCTAAAAGTAGTACT
- HCDR2 amino acid sequence of BF12 (as defined by
the Kabat system)
SEQ ID NO: 214
HPKSST
- HCDR3 nucleic acid sequence of BF12
SEQ ID NO: 215
GGGGGCTACGGTAGCCCCTTTGACTAC
- HCDR3 amino acid sequence of BF12 (as defined by
the Kabat system)
SEQ ID NO: 216
GGYGSPFDY
- LCVR nucleic acid sequence of BF12
SEQ ID NO: 217
CAAATTGTTCTCACCCAGTCTCCAGCAATCCTGTCTGCATCTCCAGGGGAGAAGGTCACCATGACC
TGCAGTGCCAGGTCAAGTGTAAGTTACATGTACTGGTACCAGCAGAAGCCAAGATCCTCCCCCAAA
CCCTGGATTTATCTCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCGCTCACGTTCGGTGCTGGGACCAAGCTGGAGCTGAAA
- LCVR amino acid sequence of BF12 (CDRs 1-3 based
on Kabat system underlined)
SEQ ID NO: 218
QIVLTQSPAILSASPGEKVTMTCSARSSVSYMYWYQQKPRSSPKPWIYLTSNLASGVPARFSGSGS
GTSYSLTISSMEAEDAATYYCQQWSSNPLTFGAGTKLELK
- LCDR1 nucleic acid sequence of BF12
SEQ ID NO: 219
AGTGCCAGGTCAAGTGTAAGTTACATGTAC
- LCDR1 amino acid sequence of BF12 (as defined by
the Kabat system)
SEQ ID NO: 220
SARSSVSYMY
- LCDR2 nucleic acid sequence of BF12
SEQ ID NO: 221
CTCACATCCAACCTGGCTTCT
- LCDR2 amino acid sequence of BF12 (as defined by
the Kabat system)
SEQ ID NO: 222
LTSNLAS
- LCDR3 nucleic acid sequence of BF12
SEQ ID NO: 223
AGCAGTGGAGTAGTAACCCGCTCACG
- LCDR3 amino acid sequence of BF12 (as defined by
the Kabat system)
SEQ ID NO: 224
QQWSSNPLT
- HCVR nucleic acid sequence of BG4
SEQ ID NO: 225
CAGGTCAAACTGCAGGAGTCAGGGGCTGAGCTTGTGAAGCCTGGGGCTTCAGTGAAGATGTCCTGC
AAGGCTTCTGGCTACACCTTCACCAACTACTGGATTCACTGGGTGAAGCAGAGGCCTGGACAAGGC
CTTGAGTGGATTGGAATGATTCATCCTAAGAGTGGTAGTATTACTTACAATGAGAAGTTCACGACC
AAGGCCACACTGACTGTCGACAAATCCTCCAGCACAGCCTACATGCAACTCAACAGCCTGACATCT
GAGGACTCTGCGGTCTATTACTGTGCAAGCGGGGGCTACGGTAGTAGCCTTGACTACTGGGGCCAA
GGGACCACGGTCACCGTCTCCTCA
- HCVR amino acid sequence of BG4 (CDRs 1-3 based
on the Kabat system underlined)
SEQ ID NO: 226
QVKLQESGAELVKPGASVKMSCKASGYTFTNYWIHWVKQRPGQGLEWIGMIHPKSGSITYNEKFTT
KATLTVDKSSSTAYMQLNSLTSEDSAVYYCASGGYGSSLDYWGQGTTVTVSS
- HCDR1 nucleic acid sequence of BG4
SEQ ID NO: 227
GGCTACACCTTCACCAACTAC
- HCDR1 amino acid sequence of BG4 (as defined by
the Kabat system)
SEQ ID NO: 228
GYTFTNY
- HCDR2 nucleic acid sequence of BG4
SEQ ID NO: 229
CATCCTAAGAGTGGTAGT
- HCDR2 amino acid sequence of BG4 (as defined by
the Kabat system)
SEQ ID NO: 230
HPKSGS
- HCDR3 nucleic acid sequence of BG4
SEQ ID NO: 231
GGGGGCTACGGTAGTAGCCTTGACTAC
- HCDR3 amino acid sequence of BG4 (as defined by
the Kabat system)
SEQ ID NO: 232
GGYGSSLDY
- LCVR nucleic acid sequence of BG4
SEQ ID NO: 233
GAAAATGTGCTCACCCAGTCTCCAGCAATCATGTCTGCATCTCCAGGGGAGAAGGTCACCATATCC
TGCAGTGCCAGCTCAAGTGTAAGTTACATGTACTGGTACCAGCAGAAGCCAGGATCCTCCCCCAAA
CCCTGGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTAACCCGCTCACGTTCGGTGCTGGGACCAAGTTGGAAATAAAA
- LCVR amino acid sequence of BG4 (CDRs 1-3 based
on Kabat system underlined)
SEQ ID NO: 234
ENVLTQSPAIMSASPGEKVTISCSASSSVSYMYWYQQKPGSSPKPWIYRTSNLASGVPARFSGSGS
GTSYSLTISSMEAEDAATYYCQQWSSNPLTFGAGTKLEIK
- LCDR1 nucleic acid sequence of BG4
SEQ ID NO: 235
AGTGCCAGCTCAAGTGTAAGTTACATGTAC
- LCDR1 amino acid sequence of BG4 (as defined by
the Kabat system)
SEQ ID NO: 236
SASSSVSYMY
- LCDR2 nucleic acid sequence of BG4
SEQ ID NO: 237
CGCACATCCAACCTGGCTTCT
- LCDR2 amino acid sequence of BG4 (as defined by
the Kabat system)
SEQ ID NO: 238
RTSNLAS
- LCDR3 nucleic acid sequence of BG4
SEQ ID NO: 239
CAGCAGTGGAGTAGTAACCCGCTCACG
- LCDR3 amino acid sequence of BG4 (as defined by
the Kabat system)
SEQ ID NO: 240
QQWSSNPLT
- HCVR nucleic acid sequence of BG5
SEQ ID NO: 241
CAGGTGAAGCTGCAGGAGTCTGGGGCTGAGCTTGTGATGCCTGGGGCTTCAGTGAAGCTGTCCTGC
AAAGCTTCTGGCTACACCTTCACCAGCTACTGGATGCACTGGGTAAAGCAGAGGCCTGGACAAGGC
CTTGAGTGGATCGGAATGATTGATCCAAAGAGTGGAAGTATTAACTACAATGAGAAGTTCAGGAGC
AAGGCCACACTGACTGTAGACAAGTCCTCCAGCACAGCCTACATGCAACTCAGCAGGCTGACATCT
GAGGACTCTGCGGTCTATTACTGTGCAAGCGGGGGCTACGGTAGTAGCCTTGACTACTGGGGCCAA
GGGACCACGGTCACCGTCTCCTCA
- HCVR amino acid sequence of BG5 (CDRs 1-3 based
on the Kabat system underlined)
SEQ ID NO: 242
QVKLQESGAELVMPGASVKLSCKASGYTFTSYWMHWVKQRPGQGLEWIGMIDPKSGSINYNEKFRS
KATLTVDKSSSTAYMQLSRLTSEDSAVYYCASGGYGSSLDYWGQGTTVTVSS
- HCDR1 nucleic acid sequence of BG5
SEQ ID NO: 243
GGCTACACCTTCACCAGCTAC
- HCDR1 amino acid sequence of BG5 (as defined by
the Kabat system)
SEQ ID NO: 244
GYTFTSY
- HCDR2 nucleic acid sequence of BG5
SEQ ID NO: 245
GATCCAAAGAGTGGAAGT
- HCDR2 amino acid sequence of BG5 (as defined by
the Kabat system)
SEQ ID NO: 246
DPKSGS
- HCDR3 nucleic acid sequence of BG5
SEQ ID NO: 247
GGGGGCTACGGTAGTAGCCTTGACTAC
- HCDR3 amino acid sequence of BG5 (as defined by
the Kabat system)
SEQ ID NO: 248
GGYGSSLDY
- LCVR nucleic acid sequence of BG5
SEQ ID NO: 249
GAAAATGTGCTCACCCAGTCCCCAGCAATCATGTCTGCATCTCCAGGGGAGAAGGTCACCATGACC
TGCAGTGCCAGCTCAAGTGTAAGTTACATGTACTGGTACCAGCAGAAGCCAGGATCCTCCCCCAAA
CCCTGGATTTATCGCACATCCAACCTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTAGTGACCCGCTCACGTTCGGTGCTGGCACCAAGCTGGAAATCAAA
- LCVR amino acid sequence of BG5 (CDRs 1-3 based
on Kabat system underlined)
SEQ ID NO: 250
ENVLTQSPAIMSASPGEKVTMTCSASSSVSYMYWYQQKPGSSPKPWIYRTSNLASGVPARFSGSGS
GTSYSLTISSMEAEDAATYYCQQWSSDPLTFGAGTKLEIK
- LCDR1 nucleic acid sequence of BG5
SEQ ID NO: 251
AGTGCCAGCTCAAGTGTAAGTTACATGTAC
- LCDR1 amino acid sequence of BG5 (as defined by
the Kabat system)
SEQ ID NO: 252
SASSSVSYMY
- LCDR2 nucleic acid sequence of BG5
SEQ ID NO: 253
CGCACATCCAACCTGGCTTCT
- LCDR2 amino acid sequence of BG5 (as defined by
the Kabat system)
SEQ ID NO: 254
RTSNLAS
- LCDR3 nucleic acid sequence of BG5
SEQ ID NO: 255
CAGCAGTGGAGTAGTGACCCGCTCACG
- LCDR3 amino acid sequence of BG5 (as defined by
the Kabat system)
SEQ ID NO: 256
QQWSSDPLT
- HCVR nucleic acid sequence of BH6
SEQ ID NO: 257
CAGGTCAAACTGCAGCAGTCAGGGGCTGACCTGGTAAAGCCTGGGGCTTCAGTGAAGTTGTCCTGC
AAGGCTTCTGGCTACACTTTCACCAGCTACTGGATGCACTGGGTGAAGCAGAGGCCTGGACAAGGC
CTTGAGTGGATTGGAATGATTCATCCTAATAGTGATGGTACTAACTACAATGAGAAGTTCAAGAAC
AGGGCCACACTGACTGTGGACAAATCCTCCAGCACGGCCTACATGCAACTCAACAGCCTGACATCT
GAAGACTCTGCGGTCTATTACTGTGCAAGATGGTCCTATGGTATGGACTCCTGGGGCCAAGGGACC
ACGGTCACCGTCTCCTCA
- HCVR amino acid sequence of BH6 (CDRs 1-3 based
on the Kabat system underlined)
SEQ ID NO: 258
QVKLQQSGADLVKPGASVKLSCKASGYTFTSYWMHWVKQRPGQGLEWIGMIHPNSDGTNYNEKFKN
RATLTVDKSSSTAYMQLNSLTSEDSAVYYCARWSYGMDSWGQGTTVTVSS
- HCDR1 nucleic acid sequence of BH6
SEQ ID NO: 259
GGCTACACTTTCACCAGCTAC
- HCDR1 amino acid sequence of BH6 (as defined by
the Kabat system)
SEQ ID NO: 260
GYTFTSY
- HCDR2 nucleic acid sequence of BH6
SEQ ID NO: 261
CATCCTAATAGTGATGGT
- HCDR2 amino acid sequence of BH6 (as defined by
the Kabat system)
SEQ ID NO: 262
HPNSDG
- HCDR3 nucleic acid sequence of BH6
SEQ ID NO: 263
TGGTCCTATGGTATGGACTCC
- HCDR3 amino acid sequence of BH6 (as defined by
the Kabat system)
SEQ ID NO: 264
WSYGMDS
- LCVR nucleic acid sequence of BH6
SEQ ID NO: 265
GACATTGTGCTAACCCAGTCTCCAGCTTCTTTGGCTGTGTCTCTAGGGCAGAGGGCCACCATCACC
TGCAGTGCCAGCTCAAGTGTAAGTTACATGCACTGGTACCAGCAGAAGTCAGGCACCTCCCCCAAA
AGATGGATTTATGACACATCCAAACTGGCTTCTGGAGTCCCTGCTCGCTTCAGTGGCAGTGGGTCT
GGGACCTCTTACTCTCTCACAATCAGCAGCATGGAGGCTGAAGATGCTGCCACTTATTACTGCCAG
CAGTGGAGTCGTAACCCACTCACGTTCGGTGCTGGGACCAAGTTGGAAATAAAA
- LCVR amino acid sequence of BH6 (CDRs 1-3 based
on Kabat system underlined)
SEQ ID NO: 266
DIVLTQSPASLAVSLGQRATITCSASSSVSYMHWYQQKSGTSPKRWIYDTSKLASGVPARFSGSGS
GTSYSLTISSMEAEDAATYYCQQWSRNPLTFGAGTKLEIK
- LCDR1 nucleic acid sequence of BH6
SEQ ID NO: 267
AGTGCCAGCTCAAGTGTAAGTTACATGCAC
- LCDR1 amino acid sequence of BH6 (as defined by
the Kabat system)
SEQ ID NO: 268
SASSSVSYMH
- LCDR2 nucleic acid sequence of BH6
SEQ ID NO: 269
GACACATCCAAACTGGCTTCT
- LCDR2 amino acid sequence of BH6 (as defined by
the Kabat system)
SEQ ID NO: 270
DTSKLAS
- LCDR3 nucleic acid sequence of BH6
SEQ ID NO: 271
CAGCAGTGGAGTCGTAACCCACTCACG
- LCDR3 amino acid sequence of BH6 (as defined by
the Kabat system)
SEQ ID NO: 272
QQWSRNPLT
- linker
SEQ ID NO: 273
GGGGSGGGGSGGGGS

### Aspects of the Invention

1. An antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, wherein the antigen binding molecule comprises the complementarity determining region (CDR) sequences of:
(a) the heavy chain variable domain sequence of SEQ ID NO: 2 and the light chain variable domain sequence of SEQ ID NO: 10; or
(b) the heavy chain variable domain sequence of SEQ ID NO: 18 and the light chain variable domain sequence of SEQ ID NO: 26; or
(c) the heavy chain variable domain sequence of SEQ ID NO: 34 and the light chain variable domain sequence of SEQ ID NO: 42; or
(d) the heavy chain variable domain sequence of SEQ ID NO: 50 and the light chain variable domain sequence of SEQ ID NO: 58; or
(e) the heavy chain variable domain sequence of SEQ ID NO: 66 and the light chain variable domain sequence of SEQ ID NO: 74; or
(f) the heavy chain variable domain sequence of SEQ ID NO: 82 and the light chain variable domain sequence of SEQ ID NO: 90; or
(g) the heavy chain variable domain sequence of SEQ ID NO: 98 and the light chain variable domain sequence of SEQ ID NO: 106; or
(h) the heavy chain variable domain sequence of SEQ ID NO: 114 and the light chain variable domain sequence of SEQ ID NO: 122; or
(i) the heavy chain variable domain sequence of SEQ ID NO: 130 and the light chain variable domain sequence of SEQ ID NO: 138; or
(j) the heavy chain variable domain sequence of SEQ ID NO: 146 and the light chain variable domain sequence of SEQ ID NO: 154; or
(k) the heavy chain variable domain sequence of SEQ ID NO: 162 and the light chain variable domain sequence of SEQ ID NO: 170; or
(l) the heavy chain variable domain sequence of SEQ ID NO: 178 and the light chain variable domain sequence of SEQ ID NO: 186; or
(m) the heavy chain variable domain sequence of SEQ ID NO: 194 and the light chain variable domain sequence of SEQ ID NO: 202; or
(n) the heavy chain variable domain sequence of SEQ ID NO: 210 and the light chain variable domain sequence of SEQ ID NO: 218; or
(o) the heavy chain variable domain sequence of SEQ ID NO: 226 and the light chain variable domain sequence of SEQ ID NO: 234; or
(p) the heavy chain variable domain sequence of SEQ ID NO: 242 and the light chain variable domain sequence of SEQ ID NO: 250; or
(q) the heavy chain variable domain sequence of SEQ ID NO: 258 and the light chain variable domain sequence of SEQ ID NO: 266.

2. An antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, and wherein:
(a) HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; or
(b) HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; or
(c) HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; or
(d) HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; or
(e) HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; or
(f) HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; or
(g) HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; or
(h) HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; or
(i) HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; or
(j) HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; or
(k) HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; or
(l) HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; or
(m) HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; or
(n) HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; or
(o) HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; or
(p) HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; or
(q) HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272.

3. An antigen binding molecule that comprises a binding domain that specifically binds to B7H3, wherein the binding domain comprises a heavy chain variable domain and/or a light chain variable domain:
(a) wherein the heavy chain variable domain comprises a sequence of SEQ ID NO: 2 or a sequence having at least 90% identity thereto and wherein the light chain variable domain comprises a sequence of SEQ ID NO: 10 or a sequence having at least
(b) wherein the heavy chain variable domain comprises a sequence of SEQ ID NO: 18 or a sequence having at least 90% identity thereto and wherein the light chain variable domain comprises a sequence of SEQ ID NO: 26 or a sequence having at least 90% identity thereto; or
(c) wherein the heavy chain variable domain comprises a sequence of SEQ ID NO: 34 or a sequence having at least 90% identity thereto and wherein the light chain variable domain comprises a sequence of SEQ ID NO: 42 or a sequence having at least 90% identity thereto; or
(d) wherein the heavy chain variable domain comprises a sequence of SEQ ID NO: 50 or a sequence having at least 90% identity thereto and wherein the light chain variable domain comprises a sequence of SEQ ID NO: 58 or a sequence having at least 90% identity thereto; or
(e) wherein the heavy chain variable domain comprises a sequence of SEQ ID NO: 66 or a sequence having at least 90% identity thereto and wherein the light chain variable domain comprises a sequence of SEQ ID NO: 74 or a sequence having at least 90% identity thereto; or
(f) wherein the heavy chain variable domain comprises a sequence of SEQ ID NO: 82 or a sequence having at least 90% identity thereto and wherein the light chain variable domain comprises a sequence of SEQ ID NO: 90 or a sequence having at least 90% identity thereto; or
(g) wherein the heavy chain variable domain comprises a sequence of SEQ ID NO: 98 or a sequence having at least 90% identity thereto and wherein the light chain variable domain comprises a sequence of SEQ ID NO: 106 or a sequence having at least 90% identity thereto; or
(h) wherein the heavy chain variable domain comprises a sequence of SEQ ID NO: 114 or a sequence having at least 90% identity thereto and wherein the light chain variable domain comprises a sequence of SEQ ID NO: 122 or a sequence having at least 90% identity thereto; or
(i) wherein the heavy chain variable domain comprises a sequence of SEQ ID NO: 130 or a sequence having at least 90% identity thereto and wherein the light chain variable domain comprises a sequence of SEQ ID NO: 138 or a sequence having at least
(j) wherein the heavy chain variable domain comprises a sequence of SEQ ID NO: 146 or a sequence having at least 90% identity thereto and wherein the light chain variable domain comprises a sequence of SEQ ID NO: 154 or a sequence having at least 90% identity thereto; or
(k) wherein the heavy chain variable domain comprises a sequence of SEQ ID NO: 162 or a sequence having at least 90% identity thereto and wherein the light chain variable domain comprises a sequence of SEQ ID NO: 170 or a sequence having at least 90% identity thereto; or
(l) wherein the heavy chain variable domain comprises a sequence of SEQ ID NO: 178 or a sequence having at least 90% identity thereto and wherein the light chain variable domain comprises a sequence of SEQ ID NO: 186 or a sequence having at least 90% identity thereto; or
(m) wherein the heavy chain variable domain comprises a sequence of SEQ ID NO: 194 or a sequence having at least 90% identity thereto and wherein the light chain variable domain comprises a sequence of SEQ ID NO: 202 or a sequence having at least 90% identity thereto; or
(n) wherein the heavy chain variable domain comprises a sequence of SEQ ID NO: 210 or a sequence having at least 90% identity thereto and wherein the light chain variable domain comprises a sequence of SEQ ID NO: 218 or a sequence having at least 90% identity thereto; or
(o) wherein the heavy chain variable domain comprises a sequence of SEQ ID NO: 226 or a sequence having at least 90% identity thereto and wherein the light chain variable domain comprises a sequence of SEQ ID NO: 234 or a sequence having at least 90% identity thereto; or
(p) wherein the heavy chain variable domain comprises a sequence of SEQ ID NO: 242 or a sequence having at least 90% identity thereto and wherein the light chain variable domain comprises a sequence of SEQ ID NO: 250 or a sequence having at least 90% identity thereto; or
(q) wherein the heavy chain variable domain comprises a sequence of SEQ ID NO: 258 or a sequence having at least 90% identity thereto and wherein the light chain variable domain comprises a sequence of SEQ ID NO: 266 or a sequence having at least 90% identity thereto.

4. The antigen binding molecule according to aspect 1 or 3, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, and wherein:
(a) HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; or
(b) HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; or
(c) HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; or
(d) HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; or
(e) HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; or
(f) HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; or
(g) HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; or
(h) HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; or
(i) HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; or
(j) HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; or
(k) HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; or
(l) HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; or
(m) HCDR1 comprises the sequence of SEQ ID NO: 196, HCDR2 comprises the sequence of SEQ ID NO: 198, HCDR3 comprises the sequence of SEQ ID NO: 200, LCDR 1 comprises the sequence of SEQ ID NO: 204, LCDR2 comprises the sequence of SEQ ID NO: 206, and LCDR3 comprises the sequence of SEQ ID NO: 208; or
(n) HCDR1 comprises the sequence of SEQ ID NO: 212, HCDR2 comprises the sequence of SEQ ID NO: 214, HCDR3 comprises the sequence of SEQ ID NO: 216, LCDR 1 comprises the sequence of SEQ ID NO: 220, LCDR2 comprises the sequence of SEQ ID NO: 222, and LCDR3 comprises the sequence of SEQ ID NO: 224; or
(o) HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; or
(p) HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; or
(q) HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272.

5. The antigen binding molecule according to any one of the preceding aspects, wherein the antigen binding molecule specifically binds to human B7H3.

6. The antigen binding molecule according to aspect 5, wherein the antigen binding molecule specifically binds to the human B7H3 isoform 4IgB7-H3 or 2IgB7-H3.

7. The antigen binding molecule according to any one of aspects 1 to 4, wherein the antigen binding molecule specifically binds to isoform T-B7-H3.

8. The antigen binding molecule according to any one of aspects 1 to 4, wherein the antigen binding molecule has specificity for isoforms T-B7-H3, 4IgB7-H3, and 2IgB7-H3.

9. The antigen binding molecule according to any one of the preceding aspects, wherein the binding domain is human or humanised.

10. The antigen binding molecule according to any one of the preceding aspects, wherein the heavy chain variable domain and/or the light chain variable domain are human or humanised.

11. The antigen binding molecule according to any one of the preceding aspects, wherein the antigen binding molecule is a single domain fragment, a Fab fragment, a Fab′ fragment, a F(ab)′2 fragment, a single chain Fab (scFab) fragment, a single chain Fv protein (scFv), a tandem scFv protein, a disulfide stabilized Fv protein (dsFv), or a scFv-Fc protein.

12. The antigen binding molecule according to aspect 11, wherein the antigen binding molecule is a scFv.

13. The antigen binding molecule according to aspect 12, wherein the scFv further comprises a linker having a sequence of SEQ ID NO: 273, wherein the heavy chain variable domain is connected to the light chain variable domain via the linker, and wherein:
(a) the heavy chain variable domain comprises a sequence of SEQ ID NO: 2 and the light chain variable domain comprises a sequence of SEQ ID NO: 10; or
(b) the heavy chain variable domain comprises a sequence of SEQ ID NO: 18 and the light chain variable domain comprises a sequence of SEQ ID NO: 26; or
(c) the heavy chain variable domain comprises a sequence of SEQ ID NO: 34 and the light chain variable domain comprises a sequence of SEQ ID NO: 42; or
(d) the heavy chain variable domain comprises a sequence of SEQ ID NO: 50 and the light chain variable domain comprises a sequence of SEQ ID NO: 58; or
(e) the heavy chain variable domain comprises a sequence of SEQ ID NO: 66 and the light chain variable domain comprises a sequence of SEQ ID NO: 74; or
(f) the heavy chain variable domain comprises a sequence of SEQ ID NO: 82 and the light chain variable domain comprises a sequence of SEQ ID NO: 90; or
(g) the heavy chain variable domain comprises a sequence of SEQ ID NO: 98 and the light chain variable domain comprises a sequence of SEQ ID NO: 106; or
(h) the heavy chain variable domain comprises a sequence of SEQ ID NO: 114 and the light chain variable domain comprises a sequence of SEQ ID NO: 122; or
(i) the heavy chain variable domain comprises a sequence of SEQ ID NO: 130 and the light chain variable domain comprises a sequence of SEQ ID NO: 138; or
(j) the heavy chain variable domain comprises a sequence of SEQ ID NO: 146 and the light chain variable domain comprises a sequence of SEQ ID NO: 154; or
(k) the heavy chain variable domain comprises a sequence of SEQ ID NO: 162 and the light chain variable domain comprises a sequence of SEQ ID NO: 170; or
(l) the heavy chain variable domain comprises a sequence of SEQ ID NO: 178 and the light chain variable domain comprises a sequence of SEQ ID NO: 186; or
(m) the heavy chain variable domain comprises a sequence of SEQ ID NO: 194 and the light chain variable domain comprises a sequence of SEQ ID NO: 202; or
(n) the heavy chain variable domain comprises a sequence of SEQ ID NO: 210 and the light chain variable domain comprises a sequence of SEQ ID NO: 218; or
(o) the heavy chain variable domain comprises a sequence of SEQ ID NO: 226 and the light chain variable domain comprises a sequence of SEQ ID NO: 234; or
(p) the heavy chain variable domain comprises a sequence of SEQ ID NO: 242 and the light chain variable domain comprises a sequence of SEQ ID NO: 250; or
(q) the heavy chain variable domain comprises a sequence of SEQ ID NO: 258 and the light chain variable domain comprises a sequence of SEQ ID NO: 266.

14. The antigen binding molecule according to any one of the preceding aspects, wherein the antigen binding molecule is a multispecific molecule, optionally wherein the antigen binding molecule is a bispecific or trispecific molecule.

15. The antigen binding molecule according to aspect 14, wherein the antigen binding molecule comprises a first binding domain which specifically binds to B7H3 and a further binding domain which specifically binds to a second antigen.

16. The antigen binding molecule according to aspect 15, wherein the further binding domain specifically binds to CD3 on the surface of a T cell.

17. The antigen binding molecule according to aspect 15 or 16, wherein the antigen binding molecule comprises two scFvs.

18. The antigen binding molecule according to aspect 16 or 17, wherein the antigen binding molecule is a bi-specific T-cell engager (BiTE).

19. A chimeric antigen receptor (CAR) which comprises an antigen binding molecule according to any one of the preceding aspects which specifically binds to B7H3.

20. The CAR according to aspect 19, wherein the CAR further comprises a hinge region, a transmembrane domain, and an intracellular signalling domain.

21. The CAR according to aspect 19 or 20, wherein the hinge region is derived from CD8.

22. The CAR according to any one of aspects 19 to 21, wherein the transmembrane domain is derived from CD8 or CD28.

23. The CAR according to any one of aspects 20 to 22, wherein the intracellular signalling domain comprises a co-stimulatory domain, optionally wherein the co-stimulatory domain is derived from CD28 or 4-1BB.

24. The CAR according to any one of aspects 20 to 23, wherein the intracellular signalling domain comprises a CD3-zeta.

25. A cell which comprises the CAR according to any one of aspects 19 to 24, wherein the cell is a T cell.

26. The cell according to aspect 25, wherein the T cell is an alpha-beta T cell or a gamma-delta T cell.

27. A nucleic acid molecule comprising a nucleotide sequence encoding the antigen binding molecule according to any one of aspects 1 to 18 or the CAR according to any one of aspects 19 to 24.

28. A nucleic acid molecule comprising a nucleotide sequence encoding the heavy chain variable domain or the light chain variable domain according to any one of aspects 1 to 18.

29. An expression vector comprising the nucleic acid molecule according to aspect 27 or 28.

30. A host cell comprising the nucleic acid molecule according to aspect 27 or 28 or the vector of aspect 29.

31. An antibody-drug conjugate (ADC) comprising the antigen binding molecule according to any one of aspects 1 to 18 linked to a drug.

32. The ADC according to aspect 31, wherein the drug is an anti-cancer agent, a cytotoxic agent, a cytostatic agent, optionally wherein the drug is selected from a pyrrolobenzodiazepine (PBD) and monomethyl auristatin E (MMAE).

33. A pharmaceutical composition comprising the antigen binding molecule according to any one of aspects 1 to 18, the CAR according to any one of aspects 19 to 24, the cell according to aspect 25 or 26, or the ADC according to aspect 31 or 32, and optionally a pharmaceutically acceptable carrier.

34. A method of treating cancer, wherein the method comprises administering the antigen binding molecule according to any one of aspects 1 to 18, the CAR according to any one of aspects 19 to 24, the cell according to aspect 25 or 26, the ADC according to aspect 31 or 32, or the pharmaceutical composition according to aspect 33, to a subject in need thereof.

35. The antigen binding molecule according to any one of aspects 1 to 18, the CAR according to any one of aspects 19 to 24, the cell according to aspect 25 or 26, or the ADC according to aspect 31 or 32, or the pharmaceutical composition according to aspect 33 for use in a method of treating cancer.

36 The method according to aspect 34 or the use according to aspect 35, wherein the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC).

37. A method of detecting cancer in a subject, comprising:
contacting a biological sample from the subject with the antigen binding molecule of any one of aspects 1 to 18 and detecting antigen binding molecule bound to the sample, wherein binding of the antigen binding molecule to the sample indicates that the subject has cancer, optionally wherein the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC).

38. The method of aspect 37, wherein the antigen binding molecule specifically binds to human B7H3, and wherein the binding of the antigen binding molecule indicates that the subject has cancer.

1. An antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, wherein the antigen binding molecule comprises the complementarity determining region (CDR) sequences of:
(a) the heavy chain variable domain sequence of SEQ ID NO: 66 and the light chain variable domain sequence of SEQ ID NO: 74; or
(b) the heavy chain variable domain sequence of SEQ ID NO: 2 and the light chain variable domain sequence of SEQ ID NO: 10; or
(c) the heavy chain variable domain sequence of SEQ ID NO: 18 and the light chain variable domain sequence of SEQ ID NO: 26; or
(d) the heavy chain variable domain sequence of SEQ ID NO: 34 and the light chain variable domain sequence of SEQ ID NO: 42; or
(e) the heavy chain variable domain sequence of SEQ ID NO: 50 and the light chain variable domain sequence of SEQ ID NO: 58; or
(f) the heavy chain variable domain sequence of SEQ ID NO: 82 and the light chain variable domain sequence of SEQ ID NO: 90; or
(g) the heavy chain variable domain sequence of SEQ ID NO: 98 and the light chain variable domain sequence of SEQ ID NO: 106; or
(h) the heavy chain variable domain sequence of SEQ ID NO: 114 and the light chain variable domain sequence of SEQ ID NO: 122; or
(i) the heavy chain variable domain sequence of SEQ ID NO: 130 and the light chain variable domain sequence of SEQ ID NO: 138; or
(j) the heavy chain variable domain sequence of SEQ ID NO: 146 and the light chain variable domain sequence of SEQ ID NO: 154; or
(k) the heavy chain variable domain sequence of SEQ ID NO: 162 and the light chain variable domain sequence of SEQ ID NO: 170; or
(l) the heavy chain variable domain sequence of SEQ ID NO: 178 and the light chain variable domain sequence of SEQ ID NO: 186; or
(m) the heavy chain variable domain sequence of SEQ ID NO: 194 and the light chain variable domain sequence of SEQ ID NO: 202; or
(n) the heavy chain variable domain sequence of SEQ ID NO: 210 and the light chain variable domain sequence of SEQ ID NO: 218; or
(o) the heavy chain variable domain sequence of SEQ ID NO: 226 and the light chain variable domain sequence of SEQ ID NO: 234; or
(p) the heavy chain variable domain sequence of SEQ ID NO: 242 and the light chain variable domain sequence of SEQ ID NO: 250; or
(q) the heavy chain variable domain sequence of SEQ ID NO: 258 and the light chain variable domain sequence of SEQ ID NO: 266.

2. An antigen binding molecule that comprises a binding domain that specifically binds to B7H3, wherein the binding domain comprises a heavy chain variable domain and/or a light chain variable domain, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, and wherein:
(a) HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; or
(b) HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; or
(c) HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; or
(d) HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; or
(e) HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; or
(f) HCDR1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; or
(g) HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; or
(h) HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; or
(i) HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; or
(j) HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; or
(k) HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; or
(l) HCDR1 comprises the sequence of SEQ ID NO: 180, HCDR2 comprises the sequence of SEQ ID NO: 182, HCDR3 comprises the sequence of SEQ ID NO: 184, LCDR 1 comprises the sequence of SEQ ID NO: 188, LCDR2 comprises the sequence of SEQ ID NO: 190, and LCDR3 comprises the sequence of SEQ ID NO: 192; or
(m) HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; or
(n) HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; or
(o) HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; or
(p) HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; or
(q) HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272.

3. An antigen binding molecule that comprises a binding domain that specifically binds to B7H3, wherein the binding domain comprises a heavy chain variable domain and/or a light chain variable domain:
(a) wherein the heavy chain variable domain comprises a sequence of SEQ ID NO: 66 or a sequence having at least 90% identity thereto and wherein the light chain variable domain comprises a sequence of SEQ ID NO: 74 or a sequence having at least 90% identity thereto; or
(b) wherein the heavy chain variable domain comprises a sequence of SEQ ID NO: 2 or a sequence having at least 90% identity thereto and wherein the light chain variable domain comprises a sequence of SEQ ID NO: 10 or a sequence having at least 90% identity thereto; or
(c) wherein the heavy chain variable domain comprises a sequence of SEQ ID NO: 18 or a sequence having at least 90% identity thereto and wherein the light chain variable domain comprises a sequence of SEQ ID NO: 26 or a sequence having at least 90% identity thereto; or
(d) wherein the heavy chain variable domain comprises a sequence of SEQ ID NO: 34 or a sequence having at least 90% identity thereto and wherein the light chain variable domain comprises a sequence of SEQ ID NO: 42 or a sequence having at least 90% identity thereto; or
(e) wherein the heavy chain variable domain comprises a sequence of SEQ ID NO: 50 or a sequence having at least 90% identity thereto and wherein the light chain variable domain comprises a sequence of SEQ ID NO: 58 or a sequence having at least 90% identity thereto; or
(f) wherein the heavy chain variable domain comprises a sequence of SEQ ID NO: 82 or a sequence having at least 90% identity thereto and wherein the light chain variable domain comprises a sequence of SEQ ID NO: 90 or a sequence having at least 90% identity thereto; or
(g) wherein the heavy chain variable domain comprises a sequence of SEQ ID NO: 98 or a sequence having at least 90% identity thereto and wherein the light chain variable domain comprises a sequence of SEQ ID NO: 106 or a sequence having at least 90% identity thereto; or
(h) wherein the heavy chain variable domain comprises a sequence of SEQ ID NO: 114 or a sequence having at least 90% identity thereto and wherein the light chain variable domain comprises a sequence of SEQ ID NO: 122 or a sequence having at least 90% identity thereto; or
(i) wherein the heavy chain variable domain comprises a sequence of SEQ ID NO: 130 or a sequence having at least 90% identity thereto and wherein the light chain variable domain comprises a sequence of SEQ ID NO: 138 or a sequence having at least 90% identity thereto; or
(j) wherein the heavy chain variable domain comprises a sequence of SEQ ID NO: 146 or a sequence having at least 90% identity thereto and wherein the light chain variable domain comprises a sequence of SEQ ID NO: 154 or a sequence having at least 90% identity thereto; or
(k) wherein the heavy chain variable domain comprises a sequence of SEQ ID NO: 162 or a sequence having at least 90% identity thereto and wherein the light chain variable domain comprises a sequence of SEQ ID NO: 170 or a sequence having at least 90% identity thereto; or
(l) wherein the heavy chain variable domain comprises a sequence of SEQ ID NO: 178 or a sequence having at least 90% identity thereto and wherein the light chain variable domain comprises a sequence of SEQ ID NO: 186 or a sequence having at least 90% identity thereto; or
(m) wherein the heavy chain variable domain comprises a sequence of SEQ ID NO: 194 or a sequence having at least 90% identity thereto and wherein the light chain variable domain comprises a sequence of SEQ ID NO: 202 or a sequence having at least 90% identity thereto; or
(n) wherein the heavy chain variable domain comprises a sequence of SEQ ID NO: 210 or a sequence having at least 90% identity thereto and wherein the light chain variable domain comprises a sequence of SEQ ID NO: 218 or a sequence having at least 90% identity thereto; or
(o) wherein the heavy chain variable domain comprises a sequence of SEQ ID NO: 226 or a sequence having at least 90% identity thereto and wherein the light chain variable domain comprises a sequence of SEQ ID NO: 234 or a sequence having at least 90% identity thereto; or
(p) wherein the heavy chain variable domain comprises a sequence of SEQ ID NO: 242 or a sequence having at least 90% identity thereto and wherein the light chain variable domain comprises a sequence of SEQ ID NO: 250 or a sequence having at least 90% identity thereto; or
(q) wherein the heavy chain variable domain comprises a sequence of SEQ ID NO: 258 or a sequence having at least 90% identity thereto and wherein the light chain variable domain comprises a sequence of SEQ ID NO: 266 or a sequence having at least 90% identity thereto.

4. The antigen binding molecule according to claim 1 or 3, wherein the heavy chain variable domain comprises a heavy chain complementarity determining region (HCDR) 1, a HCDR2, and a HCDR3, and the light chain variable domain comprises a light chain complementarity determining region (LCDR) 1, a LCDR2, and a LCDR3, and wherein:
(a) HCDR1 comprises the sequence of SEQ ID NO: 68, HCDR2 comprises the sequence of SEQ ID NO: 70, HCDR3 comprises the sequence of SEQ ID NO: 72, LCDR1 comprises the sequence of SEQ ID NO: 76, LCDR2 comprises the sequence of SEQ ID NO: 78, and LCDR3 comprises the sequence of SEQ ID NO: 80; or
(b) HCDR1 comprises the sequence of SEQ ID NO: 4, HCDR2 comprises the sequence of SEQ ID NO: 6, HCDR3 comprises the sequence of SEQ ID NO: 8, LCDR1 comprises the sequence of SEQ ID NO: 12, LCDR2 comprises the sequence of SEQ ID NO: 14, and LCDR3 comprises the sequence of SEQ ID NO: 16; or
(c) HCDR1 comprises the sequence of SEQ ID NO: 20, HCDR2 comprises the sequence of SEQ ID NO: 22, HCDR3 comprises the sequence of SEQ ID NO: 24, LCDR1 comprises the sequence of SEQ ID NO: 28, LCDR2 comprises the sequence of SEQ ID NO: 30, and LCDR3 comprises the sequence of SEQ ID NO: 32; or
(d) HCDR1 comprises the sequence of SEQ ID NO: 36, HCDR2 comprises the sequence of SEQ ID NO: 38, HCDR3 comprises the sequence of SEQ ID NO: 40, LCDR1 comprises the sequence of SEQ ID NO: 44, LCDR2 comprises the sequence of SEQ ID NO: 46, and LCDR3 comprises the sequence of SEQ ID NO: 48; or
(e) HCDR1 comprises the sequence of SEQ ID NO: 52, HCDR2 comprises the sequence of SEQ ID NO: 54, HCDR3 comprises the sequence of SEQ ID NO: 56, LCDR1 comprises the sequence of SEQ ID NO: 60, LCDR2 comprises the sequence of SEQ ID NO: 62, and LCDR3 comprises the sequence of SEQ ID NO: 64; or
(f) HCDR 1 comprises the sequence of SEQ ID NO: 84, HCDR2 comprises the sequence of SEQ ID NO: 86, HCDR3 comprises the sequence of SEQ ID NO: 88, LCDR1 comprises the sequence of SEQ ID NO: 92, LCDR2 comprises the sequence of SEQ ID NO: 94, and LCDR3 comprises the sequence of SEQ ID NO: 96; or
(g) HCDR1 comprises the sequence of SEQ ID NO: 100, HCDR2 comprises the sequence of SEQ ID NO: 102, HCDR3 comprises the sequence of SEQ ID NO: 104, LCDR1 comprises the sequence of SEQ ID NO: 108, LCDR2 comprises the sequence of SEQ ID NO: 110, and LCDR3 comprises the sequence of SEQ ID NO: 112; or
(h) HCDR1 comprises the sequence of SEQ ID NO: 116, HCDR2 comprises the sequence of SEQ ID NO: 118, HCDR3 comprises the sequence of SEQ ID NO: 120, LCDR1 comprises the sequence of SEQ ID NO: 124, LCDR2 comprises the sequence of SEQ ID NO: 126, and LCDR3 comprises the sequence of SEQ ID NO: 128; or
(i) HCDR1 comprises the sequence of SEQ ID NO: 132, HCDR2 comprises the sequence of SEQ ID NO: 134, HCDR3 comprises the sequence of SEQ ID NO: 136, LCDR1 comprises the sequence of SEQ ID NO: 140, LCDR2 comprises the sequence of SEQ ID NO: 142, and LCDR3 comprises the sequence of SEQ ID NO: 144; or
(j) HCDR1 comprises the sequence of SEQ ID NO: 148, HCDR2 comprises the sequence of SEQ ID NO: 150, HCDR3 comprises the sequence of SEQ ID NO: 152, LCDR1 comprises the sequence of SEQ ID NO: 156, LCDR2 comprises the sequence of SEQ ID NO: 158, and LCDR3 comprises the sequence of SEQ ID NO: 160; or
(k) HCDR1 comprises the sequence of SEQ ID NO: 164, HCDR2 comprises the sequence of SEQ ID NO: 166, HCDR3 comprises the sequence of SEQ ID NO: 168, LCDR1 comprises the sequence of SEQ ID NO: 172, LCDR2 comprises the sequence of SEQ ID NO: 174, and LCDR3 comprises the sequence of SEQ ID NO: 176; or
(l) HCDR1 comprises the sequence of SEQ ID NO: 180, HCDR2 comprises the sequence of SEQ ID NO: 182, HCDR3 comprises the sequence of SEQ ID NO: 184, LCDR1 comprises the sequence of SEQ ID NO: 188, LCDR2 comprises the sequence of SEQ ID NO: 190, and LCDR3 comprises the sequence of SEQ ID NO: 192; or
(m) HCDR1 comprises the sequence of SEQ ID NO: 196, HCDR2 comprises the sequence of SEQ ID NO: 198, HCDR3 comprises the sequence of SEQ ID NO: 200, LCDR1 comprises the sequence of SEQ ID NO: 204, LCDR2 comprises the sequence of SEQ ID NO: 206, and LCDR3 comprises the sequence of SEQ ID NO: 208; or
(n) HCDR1 comprises the sequence of SEQ ID NO: 212, HCDR2 comprises the sequence of SEQ ID NO: 214, HCDR3 comprises the sequence of SEQ ID NO: 216, LCDR1 comprises the sequence of SEQ ID NO: 220, LCDR2 comprises the sequence of SEQ ID NO: 222, and LCDR3 comprises the sequence of SEQ ID NO: 224; or
(o) HCDR1 comprises the sequence of SEQ ID NO: 228, HCDR2 comprises the sequence of SEQ ID NO: 230, HCDR3 comprises the sequence of SEQ ID NO: 232, LCDR1 comprises the sequence of SEQ ID NO: 236, LCDR2 comprises the sequence of SEQ ID NO: 238, and LCDR3 comprises the sequence of SEQ ID NO: 240; or
(p) HCDR1 comprises the sequence of SEQ ID NO: 244, HCDR2 comprises the sequence of SEQ ID NO: 246, HCDR3 comprises the sequence of SEQ ID NO: 248, LCDR1 comprises the sequence of SEQ ID NO: 252, LCDR2 comprises the sequence of SEQ ID NO: 254, and LCDR3 comprises the sequence of SEQ ID NO: 256; or
(q) HCDR1 comprises the sequence of SEQ ID NO: 260, HCDR2 comprises the sequence of SEQ ID NO: 262, HCDR3 comprises the sequence of SEQ ID NO: 264, LCDR1 comprises the sequence of SEQ ID NO: 268, LCDR2 comprises the sequence of SEQ ID NO: 270, and LCDR3 comprises the sequence of SEQ ID NO: 272.

5. The antigen binding molecule according to any one of the preceding claims, wherein the antigen binding molecule specifically binds to human B7H3.

6. The antigen binding molecule according to claim 5, wherein the antigen binding molecule specifically binds to the human B7H3 isoform 4IgB7-H3 or 2IgB7-H3.

7. The antigen binding molecule according to any one of claims 1 to 4, wherein (i) the antigen binding molecule specifically binds to isoform T-B7-H3; or (ii) the antigen binding molecule has specificity for isoforms T-B7-H3, 4IgB7-H3, and 2IgB7-H3.

8. The antigen binding molecule according to any one of the preceding claims, wherein:
(i) the binding domain is human or humanised; and/or
(ii) the heavy chain variable domain and/or the light chain variable domain are human or humanised; and/or
(iii) the antigen binding molecule is a single domain fragment, a Fab fragment, a Fab′ fragment, a F(ab)′2 fragment, a single chain Fab (scFab) fragment, a single chain Fv protein (scFv), a tandem scFv protein, a disulfide stabilized Fv protein (dsFv), or a scFv-Fc protein.

9. The antigen binding molecule according to claim 8 (iii), wherein the antigen binding molecule is a scFv, optionally wherein the scFv further comprises a linker having a sequence of SEQ ID NO: 273, wherein the heavy chain variable domain is connected to the light chain variable domain via the linker, and wherein:
(a) the heavy chain variable domain comprises a sequence of SEQ ID NO: 66 and the light chain variable domain comprises a sequence of SEQ ID NO: 74; or
(b) the heavy chain variable domain comprises a sequence of SEQ ID NO: 2 and the light chain variable domain comprises a sequence of SEQ ID NO: 10; or
(c) the heavy chain variable domain comprises a sequence of SEQ ID NO: 18 and the light chain variable domain comprises a sequence of SEQ ID NO: 26; or
(d) the heavy chain variable domain comprises a sequence of SEQ ID NO: 34 and the light chain variable domain comprises a sequence of SEQ ID NO: 42; or
(e) the heavy chain variable domain comprises a sequence of SEQ ID NO: 50 and the light chain variable domain comprises a sequence of SEQ ID NO: 58; or
(f) the heavy chain variable domain comprises a sequence of SEQ ID NO: 82 and the light chain variable domain comprises a sequence of SEQ ID NO: 90; or
(g) the heavy chain variable domain comprises a sequence of SEQ ID NO: 98 and the light chain variable domain comprises a sequence of SEQ ID NO: 106; or
(h) the heavy chain variable domain comprises a sequence of SEQ ID NO: 114 and the light chain variable domain comprises a sequence of SEQ ID NO: 122; or
(i) the heavy chain variable domain comprises a sequence of SEQ ID NO: 130 and the light chain variable domain comprises a sequence of SEQ ID NO: 138; or
(j) the heavy chain variable domain comprises a sequence of SEQ ID NO: 146 and the light chain variable domain comprises a sequence of SEQ ID NO: 154; or
(k) the heavy chain variable domain comprises a sequence of SEQ ID NO: 162 and the light chain variable domain comprises a sequence of SEQ ID NO: 170; or
(l) the heavy chain variable domain comprises a sequence of SEQ ID NO: 178 and the light chain variable domain comprises a sequence of SEQ ID NO: 186; or
(m) the heavy chain variable domain comprises a sequence of SEQ ID NO: 194 and the light chain variable domain comprises a sequence of SEQ ID NO: 202; or
(n) the heavy chain variable domain comprises a sequence of SEQ ID NO: 210 and the light chain variable domain comprises a sequence of SEQ ID NO: 218; or
(o) the heavy chain variable domain comprises a sequence of SEQ ID NO: 226 and the light chain variable domain comprises a sequence of SEQ ID NO: 234; or
(p) the heavy chain variable domain comprises a sequence of SEQ ID NO: 242 and the light chain variable domain comprises a sequence of SEQ ID NO: 250; or
(q) the heavy chain variable domain comprises a sequence of SEQ ID NO: 258 and the light chain variable domain comprises a sequence of SEQ ID NO: 266.

10. The antigen binding molecule according to any one of the preceding claims, wherein the antigen binding molecule is a multispecific molecule, optionally wherein the antigen binding molecule is a bispecific or trispecific molecule.

11. The antigen binding molecule according to claim 10, wherein the antigen binding molecule comprises a first binding domain which specifically binds to B7H3 and a further binding domain which specifically binds to a second antigen, optionally wherein the further binding domain specifically binds to CD3 on the surface of a T cell.

12. The antigen binding molecule according to claim 11, wherein the antigen binding molecule comprises two scFvs.

13. The antigen binding molecule according to claim 11 or 12, wherein the antigen binding molecule is a bi-specific T-cell engager (BiTE).

14. A chimeric antigen receptor (CAR) or chimeric co-stimulatory receptor (CCR), which comprises an antigen binding molecule according to any one of the preceding claims which specifically binds to B7H3.

15. The CAR or CCR according to claim 14, wherein:
(i) the CAR or CCR further comprises a hinge region, a transmembrane domain, and an intracellular signalling domain; and/or
(ii) the hinge region is derived from CD8; and/or
(iii) the transmembrane domain is derived from CD8 or CD28.

16. The CAR or CCR according to claim 15, wherein:
(a) the intracellular signalling domain of the CAR comprises a co-stimulatory domain, optionally wherein the co-stimulatory domain is derived from CD28 or 4-1BB, further optionally wherein the intracellular signalling domain comprises a CD3-zeta; or
(b) the intracellular signalling domain of the CCR comprises a co-stimulatory domain, optionally wherein the co-stimulatory domain is derived from CD28 or 4-1BB.

17. A cell which comprises the CAR or CCR according to claim 15 or 16, wherein the cell is a T cell, optionally wherein the T cell is an alpha-beta T cell or a gamma-delta T cell.

18. A nucleic acid molecule comprising a nucleotide sequence encoding (i) the antigen binding molecule according to any one of claims 1 to 13 or the CAR or CCR according to any one of claims 14 to 16, or (ii) the heavy chain variable domain or the light chain variable domain according to any one of claims 1 to 13.

19. An expression vector comprising the nucleic acid molecule according to claim 18.

20. A host cell comprising the nucleic acid molecule according to claim 18 or the vector of claim 19.

21. An antibody-drug conjugate (ADC) comprising the antigen binding molecule according to any one of claims 1 to 13 linked to a drug, optionally wherein the drug is an anti-cancer agent, a cytotoxic agent, a cytostatic agent, optionally wherein the drug is selected from a pyrrolobenzodiazepine (PBD) and monomethyl auristatin E (MMAE).

22. A pharmaceutical composition comprising the antigen binding molecule according to any one of claims 1 to 13, the CAR or CCR according to any one of claims 14 to 16, the cell according to claim 17, or the ADC according to claim 20 or 21, and optionally a pharmaceutically acceptable carrier.

23. The antigen binding molecule according to any one of claims 1 to 13, the CAR or CCR according to any one of claims 14 to 16, the cell according to claim 17, or the ADC according to claim 20 or 21, or the pharmaceutical composition according to claim 22 for use in a method of treating cancer.

24. The antigen binding molecule for use according to claim 23, wherein the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC).

25. A method of detecting cancer in a subject, comprising:
contacting a biological sample from the subject with the antigen binding molecule of any one of claims 1 to 13 and detecting antigen binding molecule bound to the sample, wherein binding of the antigen binding molecule to the sample indicates that the subject has cancer, optionally wherein the cancer is selected from the group consisting of solid tumours, neuroblastoma, medulloblastoma, glioblastoma, DIPG, osteosarcoma, rhabdomyomyosarcoma, haematological malignancies, acute myeloid leukaemia, Desmoplastic Small Round Cell Tumour (DSRCT), melanoma, carcinomas of the breast, prostate, colon, lung, renal or pancreas, or oral Squamous Cell Carcinoma (SCC), further optionally wherein the antigen binding molecule specifically binds to human B7H3, and wherein the binding of the antigen binding molecule indicates that the subject has cancer.
